(19) United States
(12) Patent Application Publication
(10) Pub. No.: US 2024/0174735 A1
(43) Pub. Date: May 30, 2024

(54) ANTIBODIES AND METHODS FOR THE DIAGNOSIS AND TREATMENT OF EPSTEIN BARR VIRUS INFECTION

(71) Applicants: The U.S.A., as represented by the Secretary, Department of Health and Human Services, Bethesda, MD (US); The Henry M. Jackson Foundation for the Advancement of Military Medicine, Inc., Bethesda, MD (US)

(72) Inventors: Wei Bu, Potomac, MD (US); Masaru Kanekiyo, North Bethesda, MD (US); Michael Gordon Joyce, Washington, DC (US); Jeffrey I. Cohen, Silver Spring, MD (US)

(73) Assignees: The U.S.A., as represented by the Secretary, Department of Health and Human Services, Bethesda, MD (US); The Henry M. Jackson Foundation for the Advancement of Military Medicine, Inc., Bethesda, MD (US)

(21) Appl. No.: 18/440,344

(22) Filed: Feb. 13, 2024

Related U.S. Application Data
(63) Continuation of application No. 17/553,139, filed on Dec. 16, 2021, now Pat. No. 11,926,656.
Application No. 17/553,139 is a division of application No. 16/608,386, filed on Oct. 25, 2019, now Pat. No. 11,236,151.
(60) Provisional application No. 62/490,023, filed on Apr. 25, 2017.

(51) Int. Cl. C07K 16/08; C07K 14/05; A61K 38/00

(52) U.S. Cl. CPC C07K 16/085; C07K 14/05; A61K 38/00; C07K 2317/10; C07K 2317/21; C07K 2317/565

(57) ABSTRACT

Antibodies and compositions of matter useful for the detection, diagnosis and treatment of Epstein Barr Virus (EBV) infection in mammals, and methods of using those compositions of matter for the detection, diagnosis and treatment of EBV infection are described. Also described are proteins, referred to as anti-gp350 antibody probes, and anti-gp350 B-cell probes, that maintain the epitope structure of the CR2-binding region of gp350, but do not bind CR2.

### CROSS-REFERENCE TO RELATED APPLICATIONS

This is a continuation application of U.S. application Ser. No. 17/553,139, filed Dec. 16, 2021, which is a divisional application of U.S. application Ser. No. 16/608,386, filed Oct. 25, 2019, issued as U.S. Pat. No. 11,236,151 on Feb. 1, 2022, which is a national stage application under 35 U.S.C. § 371 and claims the benefit of PCT Application No. PCT/US2018/029463 having an international filing date of Apr. 25, 2018, which designated the United States, which PCT application claims the benefit of priority under 35 U.S.C. § 119(e) to U.S. Provisional Patent Application No. 62/490,023, filed Apr. 25, 2017. The entire disclosures of the above-listed applications are incorporated herein by reference.

### FIELD OF THE INVENTION

The present invention is directed to compositions of matter useful for the diagnosis and treatment of Epstein Barr Virus infections in mammals and to methods of using those compositions of matter for the same.

### INCORPORATION OF ELECTRONIC SEQUENCE LISTING

The electronic sequence listing, submitted herewith as an XML file named Sequence.xml (342,344 bytes), created on Jan. 30, 2024, is herein incorporated by reference in its entirety.

### BACKGROUND

Epstein-Barr-Virus (EBV) is a human herpes virus that infects over 90% of the population world-wide with a life-long persistence in its host. In most cases, primary infection occurs during early childhood and is usually asymptomatic. In contrast, if infection is retarded and happens during adolescence or adulthood, it is regularly symptomatic, causing a benign, normally self-limiting, lymphoproliferative syndrome termed infectious mononucleosis (IM) in up to 50% of cases. Although the disease is normally self-limiting, prolonged forms of chronic active EBV infection (CAEBV) with fatal outcome have been reported. EBV infection also significantly increases the risk of developing Hodgkin disease and other types of lymphoma later in life. EBV infection is also an independent risk factor for multiple sclerosis later in life. In addition, EBV is causally associated with a heterogeneous group of malignant diseases like nasopharyngeal carcinoma, gastric carcinoma, and various types of lymphoma, and the WHO classifies EBV as a class I carcinogen.

Besides the above described medical conditions caused by EBV, patients with primary or secondary immune defects, like transplant recipients, are at elevated risk for EBV-associated diseases because of the detrimental effect of immunosuppressive agents on the immune-control of EBV-infected B-cells. EBV-associated post-transplant lymphoproliferative disorder (PTLD) is an important form of posttransplant complications, occurring in up to 20% of organ recipients. Importantly, immunocompromised transplant recipients who are immunologically naive for EBV at the onset of immunosuppression are at a particular high risk of developing life-threatening EBV positive PTLD due to a primary EBV infection, e.g. often caused after transplantation via transmission of the virus through a donor organ due to the high prevalence of EBV. Due to impaired T-cell immunity that results from exposure to immunosuppressive drugs, these patients are unable to effectively prime EBV-specific T-cells that play a critical role in controlling proliferation of EBV-infected B-cells. In contrast, patients who are EBV-seropositive at transplant have a much lower risk for developing PTLD, demonstrating the essential role of EBV-specific T-cells in eliminating virally infected cells. In general, patients who are EBV-seronegative before transplantation are at a much higher risk to develop EBV-associated diseases, since transmission of donor EBV in transplanted organs or natural infection with the virus causes lymphoproliferative disease in EBV-seronegative recipients after transplantation. As with many virus-associated diseases a promising approach for diagnosing and/or treating virus infection and its consequences in the host is the use of antibodies that specifically recognize the virus. This is also true in the case of reducing the high risk of PTLD in seronegative patients by identifying them and treating them prior to the transplantation.

These EBV-associated diseases highlight the need for a better understanding of herpes viruses and their role in mammalian diseases. As part of this understanding, there is a great need for additional diagnostic and therapeutic agents capable of detecting the presence of EBV in a mammal and effectively inhibiting EBV infection and replication. Accordingly, it is an objective of the present invention to specifically identify EBV-associated polypeptides and to use that identification specificity to produce compositions of matter useful in the therapeutic treatment and diagnostic detection of EBV in mammals.

### SUMMARY

The invention is in part based on a variety of antibodies to Epstein Barr virus (EBV) glycoprotein 350 (gp350 protein; viral envelope glycoprotein that initiates EBV infection by binding to the B cell surface receptor, CR2) and their use in the detection and diagnosis during active EBV infection. The inventors isolated monoclonal antibodies (mAbs) from macaque monkeys immunized with a gp350 nanoparticle vaccine. Characterization using multiple methods with active virus and gp350 revealed the mAbs of this disclosure (including those monoclonal antibodies produced by the B03, E04, D09, C02, H02, B04, B05, D06, D07, A03, and G12 clones) were EBV-specific, and have as much as 100-fold greater EBV neutralizing activity than 72A1, a murine antibody that targets the putative CR2-binding site on gp350 and is the most potent EBV-neutralizing antibody reported to date. Thus, the mAbs of this disclosure can recognize both cell-associated and secreted forms of native gp350 from infected cell culture and are therefore high value mAbs with potential uses in immunoassay development and as immunodiagnostic reagents for clinical sample and tissue confirmation of EBV, and treatment or prevention of EBV-associated diseases and disorders.

This disclosure provides an antibody which binds, preferably specifically, to an EBV gp350 protein. Optionally, the antibody is a monoclonal antibody, antibody fragment, chimeric antibody, humanized antibody, single-chain antibody or antibody that competitively inhibits the binding of an anti-EBV gp350 protein antibody to its respective antigenic epitope. The antibodies of this disclosure may optionally be produced in CHO cells or bacterial cells and preferably inhibit the growth or proliferation of or induce the death of a cell to which they bind. For diagnostic purposes, the antibodies of this disclosure may be detectably labeled, attached to a solid support, or the like, such as a lateral flow assay device which provides for point-of-care detection and/or diagnosis of EBV infection.

This disclosure also provides vectors comprising DNA encoding any of the herein described antibodies. Host cells comprising any such vector are also provided. By way of example, the host cells may be CHO cells, E. coli cells, or yeast cells. A process for producing any of the herein described antibodies is further provided and comprises culturing host cells under conditions suitable for expression of the desired antibody and recovering the desired antibody from the cell culture.

The disclosure also provides a composition of matter comprising an anti-EBV gp350 antibody as described herein, in combination with a carrier. Optionally, the carrier is a pharmaceutically acceptable carrier.

This disclosure also provides an article of manufacture comprising a container and a composition of matter contained within the container, wherein the composition of matter may comprise an anti-EBV gp350 antibody as described herein. The article may optionally comprise a label affixed to the container, or a package insert included with the container, that refers to the use of the composition of matter for the therapeutic treatment or diagnostic detection of an EBV infection.

This disclosure also provides the use of an anti-EBV gp350 polypeptide antibody as described herein, for the preparation of a medicament useful in the treatment of a condition which is responsive to the anti-EBV gp350 protein antibody.

This disclosure also provides any isolated antibody comprising one or more of the complementary determining regions (CDRs), including a CDR-L1, CDR-L2, CDR-L3, CDR-H1, CDR-H2, or CDR-H3 sequence disclosed herein, or any antibody that binds to the same epitope as such antibody.

This disclosure also provides a method for inhibiting the growth of a cell that expresses an EBV gp350 protein, including contacting the cell with an antibody that binds to the EBV gp350 protein, wherein the binding of the antibody to the EBV gp350 protein causes inhibition of the growth of the cell expressing the EBV gp350 protein. In these methods, the cell may be one or more of a B-lymphocyte and an epithelial cell. Binding of the antibody to the EBV gp350 protein causes death of the cell expressing the EBV gp350 protein. Optionally, the antibody is a monoclonal antibody, antibody fragment, chimeric antibody, humanized antibody, or single-chain antibody. Antibodies employed in the methods of this disclosure may optionally be conjugated to a growth inhibitory agent or cytotoxic agent such as a toxin. The antibodies employed in the methods of this disclosure may optionally be produced in CHO cells or bacterial cells.

This disclosure also provides a method of therapeutically treating a mammal having an EBV infection by administering to the mammal a therapeutically effective amount of an antibody that binds to the EBV gp350 protein, thereby resulting in the effective therapeutic treatment of the infection in the mammal. In these therapeutic methods, the antibody may be a monoclonal antibody, antibody fragment, chimeric antibody, humanized antibody, or single-chain antibody. Antibodies employed in these methods may optionally be conjugated to a growth inhibitory agent or cytotoxic agent such as a toxin. The antibodies employed in these methods of this disclosure may optionally be produced in CHO cells or bacterial cells.

This disclosure also provides is a method of determining the presence of an EBV gp350 protein in a sample suspected of containing the EBV gp350 protein, by exposing the sample to an antibody that binds to the EBV gp350 protein and determining binding of the antibody to the EBV gp350 protein in the sample, wherein the presence of such binding is indicative of the presence of the EBV gp350 protein in the sample. Optionally, the sample may contain cells (which may be fibroblasts, keratinocytes, or dendritic cells) suspected of expressing the EBV gp350 protein. The antibody employed in these methods may optionally be detectably labeled, attached to a solid support, or the like.

This disclosure also provides methods of diagnosing the presence of an EBV infection in a mammal, by detecting the level of an EBV gp350 protein in a test sample of tissue cells obtained from the mammal, wherein detection of the EBV gp350 protein in the test sample is indicative of the presence of EBV infection in the mammal from which the test sample was obtained.

This disclosure also provides methods of diagnosing the presence of an EBV infection in a mammal, by contacting a test sample comprising tissue cells obtained from the mammal with an antibody that binds to an EBV gp350 protein and detecting the formation of a complex between the antibody and the EBV gp350 protein in the test sample, wherein the formation of a complex is indicative of the presence of an EBV infection in the mammal. Optionally, the antibody employed is detectably labeled, attached to a solid support, or the like. In these methods, the test sample of tissue cells may be obtained from an individual suspected of having a viral infection.

This disclosure also provides a method of treating or preventing an EBV infection-related disorder by administering to a subject in need of such treatment an effective amount of an antagonist of an EBV gp350 protein. The EBV infection-related disorder may be infectious mononucleosis (glandular fever), particular forms of cancer, such as Hodgkin's lymphoma, Burkitt's lymphoma, gastric cancer, nasopharyngeal carcinoma, and conditions associated with human immunodeficiency virus (HIV), such as hairy leukoplakia and central nervous system lymphomas, autoimmune diseases, including dermatomyositis, systemic lupus erythematosus, rheumatoid arthritis, Sjögren's syndrome, and multiple sclerosis, and post-transplant lymphoproliferative disorder (PTLD). In these methods, the antagonist of the EBV gp350 protein is an anti-EBV gp350 protein antibody of this disclosure. Effective treatment or prevention of the disorder may be a result of direct killing or growth inhibition of cells that express an EBV gp350 protein or by antagonizing the production of EBV gp350 protein.

This disclosure also provides methods of binding an antibody to a cell that expresses an EBV gp350 protein, by contacting a cell that expresses an EBV gp350 protein with the antibody of this disclosure under conditions which are suitable for binding of the antibody to the EBV gp350 protein and allowing binding therebetween. The antibody may be labeled with a molecule or compound that is useful for qualitatively and/or quantitatively determining the location and/or amount of binding of the antibody to the cell.

This disclosure also provides for the use of an EBV gp350 protein, a nucleic acid encoding an EBV gp350 protein, or a vector or host cell comprising that nucleic acid, or an anti-EBV gp350 protein antibody in the preparation of a medicament useful for (i) the therapeutic treatment or diagnostic detection of an EBV infection, or (ii) the therapeutic treatment or prevention of an EBV infection-related disorder.

This disclosure also provides a method for inhibiting the production of additional viral particles in an EBV-infected mammal or cell, wherein the growth of the EBV infected cell is at least in part dependent upon the expression of an EBV gp350 protein (wherein the EBV gp350 protein may be expressed either within the infected cell itself or a cell that produces polypeptide(s) that have a growth potentiating effect on the infected cells), by contacting the EBV gp350 protein with an antibody that binds to the EBV gp350 protein, thereby antagonizing the growth-potentiating activity of the EBV gp350 protein and, in turn, inhibiting the growth of the infected cell. Preferably the growth of the infected cell is completely inhibited. More preferably, binding of the antibody to the EBV gp350 protein induces the death of the infected cell. Optionally, the antibody is a monoclonal antibody, antibody fragment, chimeric antibody, humanized antibody, or single-chain antibody. Antibodies employed in these methods may optionally be conjugated to a growth inhibitory agent or cytotoxic agent such as a toxin, or the like. The antibodies employed in the methods of this disclosure may optionally be produced in CHO cells or bacterial cells.

This disclosure also provides methods of treating a viral infection in a mammal, wherein the infection is at least in part dependent upon the expression of an EBV gp350 protein, by administering to the mammal a therapeutically effective amount of an antibody that binds to the EBV gp350 protein, thereby antagonizing the activity of the EBV gp350 protein and resulting in the effective therapeutic treatment of the infection in the mammal. Optionally, the antibody is a monoclonal antibody, antibody fragment, chimeric antibody, humanized antibody, or single-chain antibody. Antibodies employed in these methods may optionally be conjugated to a growth inhibitory agent or cytotoxic agent such as a toxin, or the like. The antibodies employed in the methods of this disclosure may optionally be produced in CHO cells or bacterial cells.

This disclosure also provides a protein useful for identifying an anti-gp350 antibody, or an anti-gp350 B-cell. Such a protein comprises: a) a polypeptide sequence at least 80%, at least 85%, at least 90%, at least 95%, at least 97% or at least 99% identical to the amino acid sequence of the CR2-binding region of EBV gp350; or b) a polypeptide sequence at comprising at least 100, at least 150, at least 200, at least 250, at last 300, at least 350, at least 400, or at least 425 contiguous amino acids from the amino acid sequence of the CR2-binding region of EBV gp350; wherein the protein can bind an anti-gp350 antibody, or a B-cell expressing an anti- the gp350 B-cell receptor (BCR), wherein binding of the B-cell is through the BCR, and wherein the protein is unable to bind CR2. In certain aspects, at least one amino acid residue corresponding to amino acid Q122, P158, I160, K161, W162, D163, N164, or D296, of SEQ ID NO:124 has been deleted, or has been substituted with one or more amino acid residues. In certain aspects, the amino acid sequence of the CR2-binding region comprises SEQ ID NO:125.

This disclosure also provides a comprising an amino acid sequence at least 80% identical, at least 85% identical, at least 90% identical, at least 95% identical, at least 97% identical, or at least 99% identical to the amino acid sequence of an EBV gp350 CR2-binding region, wherein the amino acid corresponding to amino acid Q122 of SEQ ID NO:124 has been deleted, or has been substituted with one or more amino acid residues. In certain aspects, the one or more amino acid residue is selected from the group consisting of methionine, leucine, isoleucine, glycine, alanine, valine, phenylalanine, tyrosine, tryptophan, lysine, arginine, histidine, glutamate, and aspartate. In certain aspects, the amino acid corresponding to amino acid Q122 of SEQ ID NO:124 has been substituted with asparagine.

This disclosure also provides a comprising an amino acid sequence at least 80% identical, at least 85% identical, at least 90% identical, at least 95% identical, at least 97% identical, or at least 99% identical to the amino acid sequence of an EBV gp350 CR2-binding region, wherein the amino acid corresponding to amino acid P158 of SEQ ID NO:124 has been deleted, or has been substituted with one or more amino acid residues. In certain aspects, the one or more amino acid residues is selected from the group consisting of methionine, leucine, isoleucine, glycine, alanine, valine, phenylalanine, tyrosine, tryptophan, lysine, arginine, histidine, glutamate, and aspartate. In certain aspects, the amino acid corresponding to amino acid P158 of SEQ ID NO:124 has been substituted with arginine.

This disclosure also provides a comprising an amino acid sequence at least 80% identical, at least 85% identical, at least 90% identical, at least 95% identical, at least 97% identical, or at least 99% identical to the amino acid sequence of an EBV gp350 CR2-binding region, wherein the amino acid corresponding to amino acid I160 of SEQ ID NO:124 has been deleted, or has been substituted with one or more amino acid residues. In certain aspects, the one or more amino acid residue is selected from the group consisting of serine, threonine, cysteine, proline, asparagine, glutamine, tyrosine, phenylalanine, tryptophan, glutamate, aspartate, arginine, glutamine, histidine and lysine. In certain aspects, the amino acid corresponding to amino acid I160 of SEQ ID NO:124 has been substituted with arginine.

This disclosure also provides a comprising an amino acid sequence at least 80% identical, at least 85% identical, at least 90% identical, at least 95% identical, at least 97% identical, or at least 99% identical to the amino acid sequence of an EBV gp350 CR2-binding region, wherein the amino acid corresponding to amino acid W162 of SEQ ID NO:124 has been deleted, or has been substituted with one or more amino acid residues. In certain aspects, the one or more amino acid residue is selected from the group consisting of serine, threonine, cysteine, proline, asparagine, glutamine, aspartate, glutamate, asparagine, arginine, glutamate, histidine, glycine, alanine, valine, leucine, methionine, isoleucine, and lysine. In certain aspects, the amino acid corresponding to amino acid W162 of SEQ ID NO:124 has been substituted with alanine.

This disclosure also provides a comprising an amino acid sequence at least 80% identical, at least 85% identical, at least 90% identical, at least 95% identical, at least 97% identical, or at least 99% identical to the amino acid sequence of an EBV gp350 CR2-binding region, wherein the amino acid corresponding to amino acid D163 of SEQ ID NO:124 has been deleted, or has been substituted with one or more amino acid residues. In certain aspects, the one or more amino acid residue is selected from the group consisting of tyrosine, phenylalanine, tryptophan, serine, threonine, cysteine, proline, asparagine, glutamine, lysine, arginine, histidine, glycine, alanine, valine, leucine, methionine, and isoleucine. In certain aspects, the amino acid corresponding to amino acid D163 of SEQ ID NO:124 has been substituted with asparagine. In certain aspects, at least one amino acid has been inserted between the amino acid corresponding to amino acid N164 of SEQ ID NO:124 and the amino acid corresponding to C165 of SEQ ID NO:124. In certain aspects, the at least one amino acid is selected from the group consisting of serine, threonine, cysteine, proline, asparagine, and glutamine. In certain aspects, a serine residue has been inserted between the amino acid corresponding to amino acid N164 of SEQ ID NO:124 and the amino acid corresponding to C165 of SEQ ID NO:124.

This disclosure also provides a comprising an amino acid sequence at least 80% identical, at least 85% identical, at least 90% identical, at least 95% identical, at least 97% identical, or at least 99% identical to the amino acid sequence of an EBV gp350 CR2-binding region, wherein the amino acid corresponding to amino acid N164 of SEQ ID NO:124 has been deleted, or has been substituted with one or more amino acid residues. In certain aspects, the one or more amino acid residue is selected from the group consisting of methionine, leucine, isoleucine, glycine, alanine, valine, phenylalanine, tyrosine, tryptophan, lysine, arginine, histidine, glutamate, serine, and aspartate. In certain aspects, the amino acid corresponding to amino acid N164 of SEQ ID NO:124 has been substituted with serine.

This disclosure also provides a comprising an amino acid sequence at least 80% identical, at least 85% identical, at least 90% identical, at least 95% identical, at least 97% identical, or at least 99% identical to the amino acid sequence of an EBV gp350 CR2-binding region, wherein the amino acid corresponding to amino acid D296 of SEQ ID NO:124 has been deleted, or has been substituted with one or more amino acid residues. In certain aspects, the one or more amino acid residue is selected from the group consisting of tyrosine, phenylalanine, tryptophan, serine, threonine, cysteine, proline, asparagine, glutamine, lysine, arginine, histidine, glycine, alanine, valine, leucine, methionine, and isoleucine. In certain aspects, the amino acid corresponding to amino acid D296 of SEQ ID NO:124 has been substituted with arginine. In certain aspects, the amino acid corresponding to amino acid I160 of SEQ ID NO:124 has been deleted, or has been substituted with at least one amino acid residue. In certain aspects, the at least one amino acid residue is selected from the group consisting of serine, threonine, cysteine, proline, asparagine, glutamine, tyrosine, phenylalanine, tryptophan, glutamate, aspartate, arginine, glutamine, histidine and lysine. In certain aspects, the amino acid corresponding to amino acid I160 of SEQ ID NO:124 has been substituted with arginine. In further aspects, the amino acid corresponding to amino acid P158 of SEQ ID NO:124 has been deleted, or has been substituted with at least one amino acid residue. In certain aspects, the at least one amino acid residue is selected from the group consisting of methionine, leucine, isoleucine, glycine, alanine, valine, phenylalanine, tyrosine, tryptophan, lysine, arginine, histidine, glutamate, and aspartate. In certain aspects, the amino acid corresponding to amino acid P158 of SEQ ID NO:124 has been substituted with arginine or asparagine. In certain aspects, the amino acid corresponding to amino acid I160 of SEQ ID NO:124, has been substituted with a threonine or an arginine.

This disclosure also provides a comprising an amino acid sequence at least 80% identical, at least 85% identical, at least 90% identical, at least 95% identical, at least 97% identical, or at least 99% identical to the amino acid sequence of an EBV gp350 CR2-binding region, wherein one or more amino acids has been inserted between the amino acid corresponding to amino acid N164 of SEQ ID NO:124 and the amino acid corresponding to C165 of SEQ ID NO:124. Inc certain aspects, the one or more amino acids are selected from the group consisting of serine, threonine, cysteine, proline, asparagine, and glutamine. In certain aspects, a serine residue has been inserted between the amino acid corresponding to amino acid N164 of SEQ ID NO:124 and the amino acid corresponding to C165 of SEQ ID NO:124.

This disclosure also provides a comprising an amino acid sequence at least 80% identical, at least 85% identical, at least 90% identical, at least 95% identical, at least 97% identical, or at least 99% identical to the amino acid sequence of an EBV gp350 CR2-binding region, wherein the amino acids corresponding to amino acids W162-N164 of SEQ ID NO:124 have been deleted, or have been substituted with one or more amino acid residues. In certain aspects, the one or more amino acid residue, is selected from the group consisting of serine, threonine, cysteine, proline, asparagine, glutamine, aspartate, glutamate, asparagine, arginine, glutamate, histidine, glycine, alanine, valine, leucine, methionine, isoleucine, and lysine. In certain aspects, the amino acids corresponding to amino acids W162-N164 of SEQ ID NO:124 have each been substituted with an alanine residue.

This disclosure also provides a protein having an amino acid sequence at least 85%, at least 90%, at least 95%, at least 97% or at least 999% identical to an amino acid sequence selected from the group consisting of SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:145, SEQ ID NO:147, SEQ ID NO:149, SEQ ID NO:151, SEQ ID NO:153, SEQ ID NO:155, SEQ ID NO:157, and SEQ ID NO:159. In certain aspects, the protein comprises an amino acid sequence selected from the group consisting of SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:145, SEQ ID NO:147, SEQ ID NO:149, SEQ ID NO:151, SEQ ID NO:153, SEQ ID NO:155, SEQ ID NO:157, and SEQ ID NO:159.

This disclosure also provides a nucleic acid molecule encoding a protein having an amino acid sequence at least 85%, at least 90%, at least 95%, at least 97% or at least 999% identical to an amino acid sequence selected from the group consisting of SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:145, SEQ ID NO:147, SEQ ID NO:149, SEQ ID NO:151, SEQ ID NO:153, SEQ ID NO:155, SEQ ID NO:157, and SEQ ID NO:159. In certain aspects, the nucleic acid molecule encodes a protein comprises an amino acid sequence selected from the group consisting of SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:145, SEQ ID NO:147, SEQ ID NO:149, SEQ ID NO:151, SEQ ID NO:153, SEQ ID NO:155, SEQ ID NO: 157, and SEQ ID NO: 159. In certain aspects, the nucleic acid molecule comprises a sequence selected from the group consisting of SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 146, SEQ ID NO: 148, SEQ ID NO: 150, SEQ ID NO: 152, SEQ ID NO: 154, SEQ ID NO: 156, SEQ ID NO:158, and SEQ ID NO:160.

This disclosure also provides a method of identifying an anti-gp350 antibody, comprising contacting a protein of the invention with a solution comprising antibodies, and isolating antibodies that specifically bind to the protein. In certain aspects, the protein comprises a His-tag, an epitope tag, or a detectable label.

This disclosure also provides a method of identifying an ant-gp350 B-cell, comprising contacting a protein of the invention with a solution comprising B-cells, and isolating B-cells that specifically bind to the protein. In certain aspects, the protein comprises a His-tag, an epitope tag, or a detectable label.

Further embodiments will be evident to the skilled artisan upon a reading of the present specification.

This disclosure contains the following Sequences:

| SEQ | | |
|---|---|---|
| ID NO | Description | Sequence |
| 1 | B03 clone Heavy Chain aa | QVQLQESGPGLVKPAETLSLTCTVSGGSFSSYWWG |
| | sequence | WIRQSPGKGLEWIGHISSGGNNYLNPSLKSRVTLSLD |
| | | TSKNQFSLKLNSVTAADSAVYYCARAPRIVVRGRYF |
| | | DQWGQGVLVTVSS |
| 2 | B03 clone Heavy Chain | caggtgcagctgcaggagtcgggcccaggactggtgaagcctgcggagaccct |
| | nucleotide sequence | gtccctcacctgcactgtctctggtggctctttcagcagttactggtggggctggatc |
| | | cgtcagtccccagggaagggactggagtggattgggcatatcagtagtggtggaa |
| | | acaactaccttaatccgtccctcaagagtcgagtcaccctgtcactagacacgtcca |
| | | agaaccagttctccctgaagctgaactctgtgaccgccgcggactcggccgtgtat |
| | | tactgtgccagagccccccgtattgttgttagaggccgatactttgaccaatggggc |
| | | cagggagtcctggtcaccgtctcctca |
| 3 | B03 clone Heavy Chain CDR1 | GGSFSSYW |
| 4 | B03 clone Heavy Chain CDR2 | ISSGGNN |
| 5 | B03 clone Heavy Chain CDR3 | APAPRIVVRGRYFDQW |
| 6 | B03 clone Kappa Chain aa | DIQMTQSPSSLSASVGDTVTITCRASQGINIYLNWFQ |
| | sequence | QRPGKAPKLLIYAATTLQSGVPSRFSGSGSGTDFTLTI |
| | | SSLQPEDFATYYCLQCESYPLTFGGGTKVEIK |
| 7 | B03 clone Kappa Chain | gacatccagatgacccagtctccatcctccctgtctgcatctgtaggagacactgtc |
| | nucleotide sequence | accatcacttgccgggcaagtcagggtatcaatatctacctgaattggtttcagcag |
| | | agaccagggaaagcccctaaactcctgatctatgctgcgaccactttacaaagtgg |
| | | ggtcccatcaagattcagcggcagtggatctgggacagatttcactctcaccatcag |
| | | cagcctgcagcctgaagatttcgcaacttattactgtctacagtgtgaaagttatccg |
| | | ctcactttcggcggagggaccaaggtggagatcaaa |
| 8 | B03 clone Kappa Chain CDR1 | QGINIY |
| 9 | B03 clone Kappa Chain CDR2 | AAT |
| 10 | B03 clone Kappa Chain CDR3 | LQCESYPLT |
| 11 | E04 clone Heavy Chain aa | QVQLQESGPGLVKPSETLSLTCTVSGGSISGAYYYW |
| | sequence | SWIRQPPGKGLDWIGYIYGSFGSAYYNPSLKSRATIS |
| | | KDTPKNQFSLKLSSVTAADTAVYYCARGRRLGYSN |
| | | WFDVWGPGVLVTVSS |
| 12 | E04 clone Heavy Chain | caggtgcaactgcaggagtcgggcccaggactggtgaagccttcggagaccctg |
| | nucleotide sequence | tccctcacctgcactgtctctggtggctccatcagcggtGCTTACtactactgg |
| | | agctggattcgacagcccccggggaagggactggactggattggatatatctatg |
| | | gaagttttgggagtgcctactacaacccctccctcaagagtcgagccaccatttcaa |
| | | aagacacgcccaagaaccagttctccctgaaactgagctctgtgaccgccgcgga |
| | | cacggccgtgtattactgtgcgagaggaaggcgactaggctattcgaactggttcg |
| | | atgtctggggcccgggagtcctggtcaccgtctcctca |
| 13 | E04 clone Heavy Chain CDR1 | GGSISGAYYY |
| 14 | E04 clone Heavy Chain CDR2 | IYGSFGSA |
| 15 | E04 clone Heavy Chain CDR3 | ARGRRLGYSNWFDVW |
| 16 | E04 clone Kappa Chain aa | DIQMTQSPSSLSASVGDKVTITCRTSQDVSSYLAWY |
| | sequence | QQKPGKAPQLLIYAASSLQSGVPSRFTGSGSGAEFTL |
| | | TISSLQPEDFASYYCQQYKNLPLTFGGGTKVEIK |
| 17 | E04 clone Kappa Chain | gacatccagatgacccagtctccatcttccctgtctgcatctgtaggagacaaagtc |
| | nucleotide sequence | accatcacttgtcggacaagtcaggacgttagcagttatttagcctggtatcagcag |
| | | aaaccagggaaagcccctcagctcctgatctatgctgcatccagtttgcaaagtgg |
| | | ggtcccatcaaggttcaccggcagtggatctggggcagaattcactctcaccatca |
| | | gcagccttcagcctgaagattttgcatcatattactgtcaacagtataaaaatctcccg |
| | | ctcactttcggcggagggaccaaagtggagatcaaa |
| 18 | E04 clone Kappa Chain CDR1 | QDVSSY |
| 19 | E04 clone Kappa Chain CDR2 | AAS |
| 20 | E04 clone Kappa Chain CDR3 | QQYKNLPLT |
| 21 | D09 clone Heavy Chain aa | QVQLQESGPGLVKPSETLSLTCDVSGGSFSGDFYWS |
| | sequence | WIRQPPGKGLDWIGNIHGSSAGTKYKPSLKSRVTISK |
| | | DTSKNQFSLKLSSVTAADTAVYYCTRGPLSRIVAGF |
| | | GRGINWFDVWGPGVLVTVSS |
| 22 | D09 clone Heavy Chain | caggtgcagctgcaggagtcgggcccaggactggtgaagccttcggagaccctg |
| | nucleotide sequence | tccctcacctgcgatgtctctggtggctccttcagcggtgatTTCtactggagctg |
| | | gatccgccagcccccagggaagggactggactggattgggaatatccatggcag |
| | | cagtgcgggcaccaaatacaagccctccctcaagagtcgagtcaccatttcaaaa |
| | | gacacgtccaagaaccagttctccctgaaactgagctctgtgaccgccgcggaca |
| | | cggccgtctattactgtacgagaggcccccttagtaggatagtagctggttttggga |
| | | gggggattaactggttcgatgtctggggcccgggagtcctggtcaccgtctcctca |
| 23 | D09 clone Heavy Chain CDR1 | GGSFSGDFY |
| 24 | D09 clone Heavy Chain CDR2 | IHGSSAGT |
| 25 | D09 clone Heavy Chain CDR3 | TRGPLSRIVAGFGRGINWFDVW |
| 26 | D09 clone Lambda Chain aa | QPVLTQPTSLSASPGASVRLSCTLSSGINVGSYSIFWY |
| | sequence | QQKPGSPPRYLLFYFSDSSKHQGSGVPSRFSGSKDTS |
| | | ANAGLLLISGLQSEDEADYYCAIWHSSASVLFGGGT |
| | | RLTVL |
| 27 | D09 clone Lambda Chain | cagcctgtgctgacccagccaacctccctctcagcatctccgggagcatcagtcag |
| | nucleotide sequence | actcagctgcaccttgagcagtggcatcaatgttggtagttacagcatattctggtac |
| | | cagcagaagccagggagtcctccccggtaccttctgttctatttctcagactcaagta |
| | | agcaccagggctctggagtccccagccgtttctctggatccaaggatacttcagcc |
| | | aatgcagggcttttactgatctctgggctccagtctgaagatgaggctgactattact |
| | | gtgccatatggcacagcagcgcttctgtgttattcggaggagggacccggctgaca |
| | | gtacta |
| 28 | D09 clone Lambda Chain | TLSSGINVGSYSIF |
| | CDR1 | |
| 29 | D09 clone Lambda Chain | YFSDSSK |
| | CDR2 | |
| 30 | D09 clone Lambda Chain | AIWHSSASVL |
| | CDR3 | |
| 31 | C02 clone Heavy Chain aa | QLQLQESGPGLVKPSETLSLTCAVSGGSISGYYWSWI |
| | sequence | RQPPGKGPEWIGFIDGNTVGTNYNPSLKSRVTLSKDT |
| | | SKNQFSLKVSSVTAADTAVYYCARKPLRRYFWFDV |
| | | WGPGVLVTVSS |
| 32 | C02 clone Heavy Chain | cagctgcagctgcaggagtcgggcccaggactggtgaagccttcggagaccctg |
| | nucleotide sequence | tccctcacctgcgctgtctctggtggctccatcagcggttactactggagttggattc |
| | | gccagcccccagggaagggaccggagtggattgggtttattgatggtaatactgtg |
| | | ggcaccaactacaacccctccctcaagagtcgagtcaccattcaaaagacacgtc |
| | | caagaatcagttctccctgaaggtgagttctgtgaccgccgcggacacggccgtgt |
| | | attactgtgcgaggaagccgctacgccgttatttctggttcgatgtctggggcccgg |
| | | gagtcctggtcaccgtctcctca |
| 33 | C02 clone Heavy Chain CDR1 | GGSISGYY |
| 34 | C02 clone Heavy Chain CDR2 | IDGNTVGT |
| 35 | C02 clone Heavy Chain CDR3 | ARKPLRRYFWFDVW |
| 36 | C02 clone Lambda Chain aa | QSVLTQPPSVSGDPGQRVTISCTGSSSNIGAGYYVYW |
| | sequence | YQQFPGTAPKLLIYQDNKRPSGVSDRFSGSKSGTSAS |
| | | LTITGLQPGDEADYYCSAWDSSLSAVMFGRGTRLTV |
| | | L |
| 37 | C02 clone Lambda Chain | cagtctgtgctgacgcagccgccctcagtgtctggggaccccgggcagagggtc |
| | nucleotide sequence | accatctcgtgcactgggagcagctccaacatcgggGCGggttattatgtatact |
| | | ggtaccagcagttcccaggaacggcccccaaactcctcatctatcaagataataag |
| | | cgaccctcaggggtttctgaccgattctctggctccaagtctggtacctcagcctcc |
| | | ctgaccatcactgggctccagcctggggatgaggctgattattactgctcagcatgg |
| | | gatagcagcctgagtgctgttatgttcggaagaggcaccaggctgacagtacta |
| 38 | C02 clone Lambda Chain | SSNIGAGYY |
| | CDR1 | |
| 39 | C02 clone Lambda Chain | QDN |
| | CDR2 | |
| 40 | C02 clone Lambda Chain | SAWDSSLSAVM |
| | CDR3 | |
| 41 | H02 clone Heavy Chain aa | QLQLQESGPGVVKPSETLSLTCTISGGSFSTYYWTWI |
| | sequence | RQPPGKGLEWVGYIGNGGRSLNYNPSLKSRITLSVD |
| | | ASKNQFSLKVTSVTAADTAVYYCGRARGLRGNWFD |
| | | VWGPGVLVTVSS |
| 42 | H02 clone Heavy Chain | caactgcagttgcaggagtcgggcccaggagtggtgaagccttcggagaccctgt |
| | nucleotide sequence | ccctcacctgcactatctctggtggctccttcagtacttactactggacctggattcgc |
| | | cagcccccagggaagggactggagtgggttgggtatatcggtaatggtggtcgta |
| | | gcctcaactacaacccctccctcaagagtcgcatcaccctgtcagtagacgcgtcc |
| | | aagaaccagttctccctgaaggtgacctctgtgaccgccgcggacacggccgtct |
| | | attactgtgggagagccaggggactccgcggaaactggttcgatgtctggggccc |
| | | gggagtcctggtcaccgtctcctca |
| 43 | H02 clone Heavy Chain CDR1 | GGSFSTYY |
| 44 | H02 clone Heavy Chain CDR2 | IGNGGRSL |
| 45 | H02 clone Heavy Chain CDR3 | GRARGLRGNWFDVW |
| 46 | H02 clone Lambda Chain aa | QAALTQPPSVSGSPGQSVTISCTGTSSDIGGYNYVSW |
| | sequence | YQQHPGKAPKVMIYEVSKRPSGVSDRFSGSKSGNIA |
| | | SLTISGLQAEDEADYYCSSYAGSNTFLFGGGTRLTVL |
| 47 | H02 clone Lambda Chain | caggctgccctgactcagcctccctctgtgtctgggtctcctggacagtcggtcacc |
| | nucleotide sequence | atctcctgcactggaaccagcagtgacatcggtggttataactatgtctcctggtacc |
| | | aacaacacccaggcaaagcccccaaagtcatgatttatgaggtcagtaagcggcc |
| | | ctcaggggtctctgatcgcttctctggttccaaatctggcaacatagcctccctgacc |
| | | atctctgggctccaggctgaggacgaggctgattattactgcagctcatatgcaggc |
| | | agcaacactttcttattcggaggagggacccggctgacagtacta |
| 48 | H02 clone Lambda Chain | SSDIGGYNY |
| | CDR1 | |
| 49 | H02 clone Lambda Chain | EVS |
| | CDR2 | |
| 50 | H02 clone Lambda Chain | SSYAGSNTFL |
| | CDR3 | |
| 51 | A03 clone Heavy Chain aa | QVQLQESGPGLVKPSETLSLTCAVSGGSISSNYWSWI |
| | sequence | RQPPGKGLEWIGRIYGSGGSTDYNPSLKSRVTISTDT |
| | | SKNQFSLKVSSVTAADTAVYYCARVRIQWVQLRGW |
| | | FDVWGPGVLVTVSS |
| 52 | A03 clone Heavy Chain | caggtgcagctgcaggagtcgggcccaggactggtgaagccttcggagaccctg |
| | nucleotide sequence | tccctcacctgcgctgtctctggtggctccatcagcagtaactactggagctggatc |
| | | cgccagcccccagggaagggactggagtggattggacgtatctatggtagtggtg |
| | | ggagcaccgactacaacccctccctcaagagtcgagtcaccatttcaacagacac |
| | | gtccaagaaccagttctccctgaaggtgagctctgtgaccgccgcggacacggcc |
| | | gtgtattactgtgcgagagtgcggatacagtgggtacagttgcgaggctggttcgat |
| | | gtctggggcccgggagtcctggtcaccgtctcctca |
| 53 | A03 clone Heavy Chain CDR1 | GGSISSNYWS |
| 54 | A03 clone Heavy Chain CDR2 | IYGSGGST |
| 55 | A03 clone Heavy Chain CDR3 | ARVRIQWVQLRGWFDVW |
| 56 | A03 clone Kappa Chain aa | YIQMTQSPSSLSASVGDTVTFTGPASQSFSSSLAWYQ |
| | sequence | QKPGKAPNLLIYSASSLQCGVRSRFSGSKSGTDFTLTI |
| | | SSLQPEDIASYYCQQYYSYPFTFGPGTKLDIK |
| 57 | A03 clone Kappa Chain | Tacatacagatgacgcagtctccatcctccctgtctgcatctgtaggagacacagtc |
| | nucleotide sequence | accttcactggccccgcaagtcagagctttagtagtagtttagcctggtatcagcag |
| | | aaaccagggaaagcccctaacctcctgatctatagtgcatccagtttgcaatgtggg |
| | | gttcgttcgaggttcagtggcagtaagtctgggacagatttcactctcaccatcagca |
| | | gcctgcagcctgaagatattgctagttattactgtcaacagtattacagttatccattca |
| | | ctttcggccccgggaccaaactggatatcaaa |
| 58 | A03 clone Kappa Chain CDR1 | QSFSSS |
| 59 | A03 clone Kappa Chain CDR2 | SAS |
| 60 | A03 clone Kappa Chain CDR3 | QQYYSYPFT |
| 61 | B04 clone Heavy Chain aa | QVQLQESGPGLVRPSETLSLTCAVSGGSISSNYWSWI |
| | sequence | RQPPGKGLEWIGYISGSTGSTYQNPSLKSRVTVSKDT |
| | | SKNQFSLKLNSVTAADTAVYYCARSGRRGSSLDLW |
| | | GRGVLVTVSS |
| 62 | B04 clone Heavy Chain | Caggtgcagctgcaggagtcgggcccaggactggtgaggccttcggagaccct |
| | nucleotide sequence | atccctcacctgcgctgtctctggtggctccatcagcagtaactactggagctggatt |
| | | cgccagcccccagggaaggggctggagtggattgggtatatctctggtagtactg |
| | | ggagcacctaccagaacccctccctcaagagtcgagtcaccgtttcaaaagacac |
| | | gtctaagaaccagttctccctgaagctgaattctgtgaccgccgcggacacggccg |
| | | tgtattactgtgcgagaagtgggagaagaggcagctcattggatttgtggggccgg |
| | | ggagttctggtcaccgtctcctca |
| 63 | B04 clone Heavy Chain CDR1 | GGSISSNY |
| 64 | B04 clone Heavy Chain CDR2 | ISGSTGST |
| 65 | B04 clone Heavy Chain CDR3 | ARSGRRGSSLDLW |
| 66 | B04 clone Lambda Chain aa | QSVLTQPPSVSGDPGQRVTISCTGSSSNIGAGYYVYW |
| | sequence | YQQFPGTAPKLLIYQDNKRPSGVSDRFSGSKSGTSAS |
| | | LTITGLQPGDEADYYCSAWDSSLSAVFFGGGTRLTV |
| | | L |
| 67 | B04 clone Lambda Chain | cagtctgtgctgacgcagccgccctcagtgtctggggaccccgggcagagggtc |
| | nucleotide sequence | accatctcgtgcactgggagcagctccaacatcgggGCGggttattatgtatact |
| | | ggtaccagcagttcccaggaacggcccccaaactcctcatctatcaagataataag |
| | | cgaccctcaggggtttctgaccgattctctggctccaagtctggtacctcagcctcc |
| | | ctgaccatcactgggctccagcctggggatgaggctgattattactgctcagcatgg |
| | | gatagcagcctgagtgctgtgttcttcggaggagggacccggctgacagtacta |
| 68 | B04 clone Lambda Chain | SSNIGAGYY |
| | CDR1 | |
| 69 | B04 clone Lambda Chain | QDN |
| | CDR2 | |
| 70 | B04 clone Lambda Chain | SAWDSSLSAVF |
| | CDR3 | |
| 71 | B05 clone Heavy Chain aa | QLQESGPGLVKPSETLSLTCTVSGGSISDTYRWSWIR |
| | sequence | QSPGKGLEWIAYIYGTTTSTNYNPSLKSRLTISKDTS |
| | | KNQFSLNLRSVTAADTAVYYCARGDSGGRSAHVFH |
| | | FWGQGLRVTVSS |
| 72 | B05 clone Heavy Chain | cagctgcaggagtcgggcccaggactggtgaagccttcggagaccctgtccctca |
| | nucleotide sequence | cctgcactgtctctggtggctccatcagcgatacttacCGGtggagctggattcgc |
| | | cagtccccagggaagggactggagtggattgcctacatctatggtactactacgag |
| | | caccaactacaacccctccctcaagagtcgactcaccatttcaaaagacacgtcca |
| | | agaaccagttctccttgaacctgaggtctgtgaccgccgcggacacggccgtgtat |
| | | tactgtgcgagaggggatagcggtggccggtcagcgcatgtttttcatttctggggc |
| | | caagggctcagggtcaccgtctcttca |
| 73 | B05 clone Heavy Chain CDR1 | GGSISDTYR |
| 74 | B05 clone Heavy Chain CDR2 | IYGTTTST |
| 75 | B05 clone Heavy Chain CDR3 | ARGDSGGRSAHVFHFW |
| 76 | B05 clone Lambda Chain aa | QSVLTQPPSVFGDPGQRITISCTGSSSNIGAGYYVYW |
| | sequence | YQQFPGTAPKLLIYQDNKRPSGVSDRFSGSKSGSSAS |
| | | LTITGLQPGDEADYYCSAWDSSLSVRVFGGGTRLTV |
| | | L |
| 77 | B05 clone Lambda Chain | cagagtgttctgacgcagccgccctcagtgtttggggaccccgggcagaggatca |
| | nucleotide sequence | ccatctcgtgcactgggagcagctccaacatcgggGCGggttattatgtatactg |
| | | gtaccagcagttcccaggaacggcccccaaactcctcatctatcaagataataagc |
| | | gaccctcaggggtttctgaccgattttctggctccaagtctggttcctcagcctccct |
| | | gaccatcactgggctccagcctggggatgaggctgattattactgctcagcatggg |
| | | atagcagcctgagtgtacgggttttcggaggagggacccggctgacagtacta |
| 78 | B05 clone Lambda Chain | SSNIGAGYY |
| | CDR1 | |
| 79 | B05 clone Lambda Chain | QDN |
| | CDR2 | |
| 80 | B05 clone Lambda Chain | SAWDSSLSVRV |
| | CDR3 | |
| 81 | D06 clone Heavy Chain aa | EVQLVESGGGLVQPGGSLRLSCAASGFTFSSYGMN |
| | sequence | WVRQAPGKGLEWVSFITNTGKTTYYADSVRGRFTIS |
| | | RDNAKKSVSLQMSSLRAEDTAVYYCTRGRGRHGWS |
| | | SGVFDFWGQGLRVTVS |
| 82 | D06 clone Heavy Chain | Gaggtgcaactggtggagtctggaggaggcttggtccagcctggagggtccctg |
| | nucleotide sequence | agactctcctgtgcagcctctggattcaccttcagtagttacggcatgaactgggtcc |
| | | gccaggctccgggaaaggggctggagtgggtctcattcattactaacactggtaaa |
| | | accacatactacgctgactctgtgaggggccgattcaccatctccagagacaacgc |
| | | caagaagtcggtgtctctacaaatgagtagcctgagagccgaggacacggccgtc |
| | | tattactgtactaggggaagaggtagacacggctggtccagtggtgtttttgatttctg |
| | | gggccaaggtctcagggtcaccgtctcttc |
| 83 | D06 clone Heavy Chain CDR1 | GFTFSSYG |
| 84 | D06 clone Heavy Chain CDR2 | ITNTGKTT |
| 85 | D06 clone Heavy Chain CDR3 | TGRGRHGWSSGVFDFW |
| 86 | D06 clone Lambda Chain aa | QSVLTQPPSVFGDPGQRITISCTGSSSNIGAGYYVYW |
| | sequence | YQQFPGTAPKLLIYQDNKRPSGVSDRFSGSKFGSSAS |
| | | LTITGVQRGDEGDYYCSAWDSSLSVRVLGGGTRLTV |
| | | L |
| 87 | D06 clone Lambda Chain | cagtctgttctgacgcagccgccctcagtgttcggggaccccgggcagaggatca |
| | nucleotide sequence | ccatttcgtgcactgggagcagctccaacatcgggGCGggttattatgtatactg |
| | | gtaccagcagttcccaggaacggcccccaaactcctcatctatcaagataataagc |
| | | gaccctcaggggtttctgaccgattctctggctccaagtttggttcctcagcctccct |
| | | gaccatcactggggtccagcgtggggatgagggtgattattactgctcagcatggg |
| | | atagcagcctgagtgtacgggttttgggaggagggacccggctgacagtacta |
| 88 | D06 clone Lambda Chain | SSNIGAGYY |
| | CDR1 | |
| 89 | D06 clone Lambda Chain | QDN |
| | CDR2 | |
| 90 | D06 clone Lambda Chain | SAWDSSLSVRV |
| | CDR3 | |
| 91 | D07 clone Heavy Chain aa | EVQLVESGGGLVQPGGSLRLSCVASGFTFSDRYIDW |
| | sequence | VRQAPGKGLEWVSTISTGSGDTALYSDSVKGRFTISR |
| | | DNAKNTLYLQMNSLRAEDTAVYYCARHSGTFYTHF |
| | | DYWGQGVLVTVSS |
| 92 | D07 clone Heavy Chain | gaagtgcagttggtggagtctgggggaggcttggtacagccgggggggtccctg |
| | nucleotide sequence | agactctcctgtgtagcctctggattcaccttcagtgaccgctacatagactgggtcc |
| | | gccaggctccagggaagggcctggagtgggtctcaactattagcactggtAGT |
| | | ggtgataccgcattgtactcagactctgtcaagggccgattcaccatctccagagac |
| | | aacgccaagaacacactgtatctgcaaatgaacagcctgagagccgaagacacg |
| | | gctgtctattactgtgcgagacatagtggtactttttacacccactttgactactgggg |
| | | ccagggagtcctggtcaccgtctcctca |
| 93 | D07 clone Heavy Chain CDR1 | GFTFSDRY |
| 94 | D07 clone Heavy Chain CDR2 | ISTGSGDTA |
| 95 | D07 clone Heavy Chain CDR3 | ARHSGTFYTHFDYW |
| 96 | D07 clone Kappa Chain aa | DIQMTQSPSSLSASVGDTVTFTCRASRSISSWLAWYQ |
| | sequence | QKPGRAPKVLIYKASSLQSGVPSRFSGSGSGTDFTLTI |
| | | SSLQSEDFATYYCQQYSSRPPTFGQGTKVEIR |
| 97 | D07 clone Kappa Chain | Gacatccagatgacccagtctccatcctccctgtctgcatctgtaggagacacagt |
| | nucleotide sequence | caccttcacctgccgggcgagtcggagtattagcagctggttagcctggtatcagc |
| | | agaaaccagggagagcccctaaagtcctgatctataaggcgtccagtttgcaaagt |
| | | ggggttccttcaaggttcagcggcagtggatctgggacagacttcactctcaccatc |
| | | agcagcctacagtctgaagattttgcaacatattattgtcaacagtatagtagtcgcc |
| | | ctccgacgttcggccaagggaccaaggtggaaatcaga |
| 98 | D07 clone Kappa Chain CDR1 | RSISSW |
| 99 | D07 clone Kappa Chain CDR2 | KAS |
| 100 | D07 clone Kappa Chain CDR3 | QQYSSRPPT |
| 101 | G12 clone Heavy Chain aa | QVQLQESGPGLLKPSETLSLTCAVSGGSFSSFWWSW |
| | sequence | LRQPPEKGLEWIGEINGDSGSTNYNPSLKSRVTISKD |
| | | ASKNQFSLKLTSVTAADTAVFYCARVRRILRSLDVW |
| | | GRGVLVTVSS |
| 102 | G12 clone Heavy Chain | Caggtgcagctgcaggagtcgggcccaggactgctgaagccttcggagaccct |
| | nucleotide sequence | gtccctcacctgcgctgtctctggtggctccttcagtagtttctggtggagctggctc |
| | | cgccagcccccagaaaagggactggagtggattggggagatcaatggtgatagt |
| | | gggagcaccaactacaacccctccctcaagagtcgagtcaccatttcaaaagacg |
| | | cgtccaagaaccagttctccctgaaactgacctctgtgaccgccgcggacacggc |
| | | cgttttttactgtgcgagagttcggcgaattctgaggtcattggatgtctggggccgg |
| | | ggagttctggtcaccgtctcctca |
| 103 | G12 clone Heavy Chain CDR1 | GGSFSSFW |
| 104 | G12 clone Heavy Chain CDR2 | INGDSGST |
| 105 | G12 clone Heavy Chain CDR3 | ARVRRILRSLDVW |
| 106 | G12 clone Kappa Chain aa | DIQMTQSPFSLFAFVGDRVTITCQASQGISHLLAWYQ |
| | sequence | QKPGKAPKLLIYSASTLQSGVPSRFSGSGFGTEFTLTI |
| | | SSLQPEDFATYYCQQHNSYPRTFGQGTKVEIK |
| 107 | G12 clone Kappa Chain | Gacatccagatgacccagtctcctttttccttgtttgcatttgtaggagacagagtcac |
| | nucleotide sequence | catcacttgccaagccagtcagggtattagccacttgttagcttggtatcagcagaa |
| | | accagggaaagcccctaagctccttatttattctgcatccactttgcaaagtggggtc |
| | | ccatcaaggttcagcggcagtggatttgggacggaattcactctcaccatcagcag |
| | | cctgcagcctgaagattttgcaacttattactgtcaacagcataatagttaccctcgga |
| | | cgttcggccaagggaccaaggtggaaatcaaa |
| 108 | G12 clone Kappa Chain CDR1 | QGISHL |
| 109 | G12 clone Kappa Chain CDR2 | SAS |
| 110 | G12 clone Kappa Chain CDR3 | QQHNSYPRT |
| 111 | Macaque HC backbone | tcgcgcgttt cggtgatgac ggtgaaaacc tctgacacat gcagctcccg |
| | Nucleotide sequence | gagacggtca cagcttgtct gtaagcggat gccgggagca gacaagcccg |
| | | tcagggcgcg tcagcgggtg ttggcgggtg tcggggctgg cttaactatg |
| | | cggcatcaga gcagattgta ctgagagtgc accatatgcg gtgtgaaata |
| | | ccgcacagat gcgtaaggag aaaataccgc atcagattgg |
| | | ctattggcca ttgcatacgt tgtatccata tcataatatg tacatttata |
| | | ttggctcatg tccaacatta ccgccatgtt gacattgatt attgactagt |
| | | tattaatagt aatcaattac ggggtcatta gttcatagcc catatatgga |
| | | gttccgcgtt acataactta cggtaaatgg cccgcctggc tgaccgccca |
| | | acgacccccg cccattgacg tcaataatga cgtatgttcc catagtaacg |
| | | ccaataggga ctttccattg acgtcaatgg gtggagtatt tacggtaaac |
| | | tgcccacttg gcagtacatc aagtgtatca tatgccaagt acgcccccta |
| | | ttgacgtcaa tgacggtaaa tggcccgcct ggcattatgc ccagtacatg |
| | | accttatggg actttcctac ttggcagtac atctacgtat tagtcatcgc |
| | | tattaccatg gtgatgcggt tttggcagta catcaatggg cgtggatagc |
| | | ggtttgactc acggggattt ccaagtctcc accccattga cgtcaatggg |
| | | agtttgtttt ggcaccaaaa tcaacgggac tttccaaaat gtcgtaacaa |
| | | ctccgcccca ttgacgcaaa tgggcggtag gcgtgtacgg tgggaggtct |
| | | atataagcag agctcgttta gtgaaccgtc agatcgcctg gagacgccat |
| | | ccacgctgtt ttgacctcca tagaagacac cgggaccgat ccagcctcca |
| | | tcggctcgca tctctccttc acgcgcccgc cgccctacct gaggccgcca |
| | | tccacgccgg ttgagtcgcg ttctgccgcc tcccgcctgt ggtgcctcct |
| | | gaactgcgtc cgccgtctag gtaagtttaa agctcaggtc gagaccgggc |
| | | ctttgtccgg cgctcccttg gagcctacct agactcagcc ggctctccac |
| | | gctttgcctg accctgcttg ctcaactcta gttaacggtg gagggcagtg |
| | | tagtctgagc agtactcgtt gctgccgcgc gcgccaccag acataatagc |
| | | tgacagacta acagactgtt cctttccatg ggtcttttct gcagtcaccg |
| | | tcgtcgacac gtgtgatcag atatcgcggc cgctctagac caccatggga |
| | | tggtcatgta tcatcctttt tctagtagca actgcaaccg gtgtacattc |
| | | ccaggtgcag ctggtgcagc tgcacgagtc gggcccagga ctggtgaagc |
| | | cttcggagac cctgtccctc acctgcgctg tctctggtgg ctctatcagc |
| | | agtagctact ggagctggat ccgccaggcc ccagggaagg gactggagtg |
| | | gattgggtat gtctatggta gtggtcgtga caccaacgac aacccctccc |
| | | tcaagagtcg agtcaccctg tcagtagaca cgtccaagaa ccagctctcc |
| | | ctgaagctga gatctgtgac cgccgcggac acggccgtgt attactgtgc |
| | | gagcagcggc tggcctcctg ggttggacta ctggggccag ggagtcacgg |
| | | tcaccgtctc ctcagctagc accaagggcc ctagtgtgtt tcctctggcc |
| | | cctagcagca gaagcacatc tgaatctaca gccgccctgg gctgcctggt |
| | | gaaagattac ttccccgagc ccgtgaccgt gtcttggaat agcggctctc |
| | | tgaccagcgg cgtgcacaca tttccagctg tgctgcagag |
| | | cagcggcctg tattctctga gcagcgtggt gacagtgcca agcagctctc |
| | | tgggcaccca gacctacgtg tgcaacgtga accacaagcc cagcaacacc |
| | | aaggtggaca agcgggtgga aatcaagacc tgtggcggcg gaagcaagcc |
| | | tcctacctgt cctccttgta ccagccctga actgctgggc ggacctagcg |
| | | tgttcctgtt ccccccaaag cccaaggaca ccctgatgat cagcagaacc |
| | | cccgaagtga cctgcgtggt ggtggatgtg tcccaggaag atcccgacgt |
| | | gaagttcaat tggtacgtga acggcgccga ggtgcaccat gcccagacaa |
| | | agcccagaga gacacagtac aacagcacct accgggtggt gtctgtgctg |
| | | accgtgacac accaggactg gctgaacggc aaagagtaca catgcaaggt |
| | | gtccaacaag gccctgcctg cccccatcca gaaaaccatc agcaaggaca |
| | | agggccagcc cagagaacct caggtgtaca cactgccccc |
| | | cagcagagag gaactgacca agaatcaggt gtccctgacc tgtctggtga |
| | | aaggcttcta ccccagcgac atcgtggtgg aatgggagtc tagcggacag |
| | | cccgagaaca cctacaagac cacccctcca gtgctggata gcgacggcag |
| | | ctacttcctg tacagcaagc tgaccgtgga caagagcaga tggcagcagg |
| | | gcaacgtgtt cagctgctct gtgatgcacg aggccctgca caaccactac |
| | | acccagaagt ctctgagcct gagccccgga aagtgatgat gaacacgtgg |
| | | gatccagatc tgctgtgcct tctagttgcc agccatctgt tgtttgcccc |
| | | tcccccgtgc cttccttgac cctggaaggt gccactccca ctgtcctttc |
| | | ctaataaaat gaggaaattg catcgcattg tctgagtagg tgtcattcta |
| | | ttctgggggg tggggtgggg caggacagca agggggagga ttgggaagac |
| | | aatagcaggc atgctgggga tgcggtgggc tctatgggta cccaggtgct |
| | | gaagaattga cccggttcct cctgggccag aaagaagcag gcacatcccc |
| | | ttctctgtga cacaccctgt ccacgcccct ggttcttagt tccagcccca |
| | | ctcataggac actcatagct caggagggct ccgccttcaa tcccacccgc |
| | | taaagtactt ggagcggtct ctccctccct catcagccca ccaaaccaaa |
| | | cctagcctcc aagagtggga agaaattaaa gcaagatagg ctattaagtg |
| | | cagagggaga gaaaatgcct ccaacatgtg aggaagtaat |
| | | gagagaaatc atagaatttt aaggccatga tttaaggcca tcatggcctt |
| | | aatcttccgc ttcctcgctc actgactcgc tgcgctcggt cgttcggctg |
| | | cggcgagcgg tatcagctca ctcaaaggcg gtaatacggt tatccacaga |
| | | atcaggggat aacgcaggaa agaacatgtg agcaaaaggc cagcaaaagg |
| | | ccaggaaccg taaaaaggcc gcgttgctgg cgtttttcca taggctccgc |
| | | ccccctgacg agcatcacaa aaatcgacgc tcaagtcaga ggtggcgaaa |
| | | cccgacagga ctataaagat accaggcgtt tccccctgga agctccctcg |
| | | tgcgctctcc tgttccgacc ctgccgctta ccggatacct gtccgccttt |
| | | ctcccttcgg gaagcgtggc gctttctcat agctcacgct gtaggtatct |
| | | cagttcggtg taggtcgttc gctccaagct gggctgtgtg cacgaacccc |
| | | ccgttcagcc cgaccgctgc gccttatccg gtaactatcg tcttgagtcc |
| | | aacccggtaa gacacgactt atcgccactg gcagcagcca ctggtaacag |
| | | gattagcaga gcgaggtatg taggcggtgc tacagagttc ttgaagtggt |
| | | ggcctaacta cggctacact agaagaacag tatttggtat ctgcgctctg |
| | | ctgaagccag ttaccttcgg aaaaagagtt ggtagctctt gatccggcaa |
| | | acaaaccacc gctggtagcg gtggtttttt tgtttgcaag cagcagatta |
| | | cgcgcagaaa aaaaggatct caagaagatc ctttgatctt ttctacgggg |
| | | tctgacgctc agtggaacga aaactcacgt taagggattt tggtcatgag |
| | | attatcaaaa aggatcttca cctagatcct tttaaattaa aaatgaagtt |
| | | ttaaatcaat ctaaagtata tatgagtaaa cttggtctga cagttaccaa |
| | | tgcttaatca gtgaggcacc tatctcagcg atctgtctat ttcgttcatc |
| | | catagttgcc tgactcgggg ggggggggcg ctgaggtctg cctcgtgaag |
| | | aaggtgttgc tgactcatac caggcctgaa tcgccccatc atccagccag |
| | | aaagtgaggg agccacggtt gatgagagct ttgttgtagg tggaccagtt |
| | | ggtgattttg aacttttgct ttgccacgga acggtctgcg ttgtcgggaa |
| | | gatgcgtgat ctgatccttc aactcagcaa aagttcgatt tattcaacaa |
| | | agccgccgtc ccgtcaagtc agcgtaatgc tctgccagtg ttacaaccaa |
| | | ttaaccaatt ctgattagaa aaactcatcg agcatcaaat gaaactgcaa |
| | | tttattcata tcaggattat caataccata tttttgaaaa agccgtttct |
| | | gtaatgaagg agaaaactca ccgaggcagt tccataggat ggcaagatcc |
| | | tggtatcggt ctgcgattcc gactcgtcca acatcaatac aacctattaa |
| | | tttcccctcg tcaaaaataa ggttatcaag tgagaaatca ccatgagtga |
| | | cgactgaatc cggtgagaat ggcaaaagct tatgcatttc tttccagact |
| | | tgttcaacag gccagccatt acgctcgtca tcaaaatcac tcgcatcaac |
| | | caaaccgtta ttcattcgtg attgcgcctg agcgagacga aatacgcgat |
| | | cgctgttaaa aggacaatta caaacaggaa tcgaatgcaa ccggcgcagg |
| | | aacactgcca gcgcatcaac aatattttca cctgaatcag gatattcttc |
| | | taatacctgg aatgctgttt tcccggggat cgcagtggtg agtaaccatg |
| | | catcatcagg agtacggata aaatgcttga tggtcggaag aggcataaat |
| | | tccgtcagcc agtttagtct gaccatctca tctgtaacat cattggcaac |
| | | gctacctttg ccatgtttca gaaacaactc tggcgcatcg ggcttcccat |
| | | acaatcgata gattgtcgca cctgattgcc cgacattatc gcgagcccat |
| | | ttatacccat ataaatcagc atccatgttg gaatttaatc gcggcctcga |
| | | gcaagacgtt tcccgttgaa tatggctcat aacacccctt gtattactgt |
| | | ttatgtaagc agacagtttt attgttcatg atgatatatt tttatcttgt |
| | | gcaatgtaac atcagagatt ttgagacaca acgtggcttt cccccccccc |
| | | ccattattga agcatttatc agggttattg tctcatgagc ggatacatat |
| | | ttgaatgtat ttagaaaaat aaacaaatag gggttccgcg cacatttccc |
| | | cgaaaagtgc cacctgacgt ctaagaaacc attattatca tgacattaac |
| | | ctataaaaat aggcgtatca cgaggccctt tcgtc |
| 112 | Macaque heavy chain | MGWSCIILFLVATATGVHSQVQLVQLHESGPGLVKP |
| | backbone aa sequence | SETLSLTCAVSGGSISSSYWSWIRQAPGKGLEWIGYV |
| | | YGSGRDTNDNPSLKSRVTLSVDTSKNQLSLKLRSVT |
| | | AADTAVYYCASSGWPPGLDYWGQGVTVTVSSASTK |
| | | GPSVFPLAPSSRSTSESTAALGCLVKDYFPEPVTVSW |
| | | NSGSLTSGVHTFPAVLQSSGLYSLSSVVTVPSSSLGT |
| | | QTYVCNVNHKPSNTKVDKRVEIKTCGGGSKPPTCPP |
| | | CTSPELLGGPSVFLFPPKPKDTLMISRTPEVTCVVVD |
| | | VSQEDPDVKFNWYVNGAEVHHAQTKPRETQYNSTY |
| | | RVVSVLTVTHQDWLNGKEYTCKVSNKALPAPIQKTI |
| | | SKDKGQPREPQVYTLPPSREELTKNQVSLTCLVKGF |
| | | YPSDIVVEWESSGQPENTYKTTPPVLDSDGSYFLYSK |
| | | LTVDKSRWQQGNVFSCSVMHEALHNHYTQKSLSLS |
| | | PGK |
| 113 | Macaque Kappa chain | tcgcgcgttt cggtgatgac ggtgaaaacc tctgacacat gcagctcccg |
| | backbone nucleotide | gagacggtca cagcttgtct gtaagcggat gccgggagca gacaagcccg |
| | sequence | tcagggcgcg tcagcgggtg ttggcgggtg tcggggctgg cttaactatg |
| | | cggcatcaga gcagattgta ctgagagtgc accatatgcg gtgtgaaata |
| | | ccgcacagat gcgtaaggag aaaataccgc atcagattgg ctattggcca |
| | | ttgcatacgt tgtatccata tcataatatg tacatttata ttggctcatg |
| | | tccaacatta ccgccatgtt gacattgatt attgactagt tattaatagt |
| | | aatcaattac ggggtcatta gttcatagcc catatatgga gttccgcgtt |
| | | acataactta cggtaaatgg cccgcctggc tgaccgccca acgacccccg |
| | | cccattgacg tcaataatga cgtatgttcc catagtaacg ccaataggga |
| | | ctttccattg acgtcaatgg gtggagtatt tacggtaaac tgcccacttg |
| | | gcagtacatc aagtgtatca tatgccaagt acgcccccta ttgacgtcaa |
| | | tgacggtaaa tggcccgcct ggcattatgc ccagtacatg accttatggg |
| | | actttcctac ttggcagtac atctacgtat tagtcatcgc tattaccatg |
| | | gtgatgcggt tttggcagta catcaatggg cgtggatagc ggtttgactc |
| | | acggggattt ccaagtctcc accccattga cgtcaatggg agtttgtttt |
| | | ggcaccaaaa tcaacgggac tttccaaaat gtcgtaacaa ctccgcccca |
| | | ttgacgcaaa tgggcggtag gcgtgtacgg tgggaggtct atataagcag |
| | | agctcgttta gtgaaccgtc agatcgcctg gagacgccat ccacgctgtt |
| | | ttgacctcca tagaagacac cgggaccgat ccagcctcca tcggctcgca |
| | | tctctccttc acgcgcccgc cgccctacct gaggccgcca tccacgccgg |
| | | ttgagtcgcg ttctgccgcc tcccgcctgt ggtgcctcct gaactgcgtc |
| | | cgccgtctag gtaagtttaa agctcaggtc gagaccgggc ctttgtccgg |
| | | cgctcccttg gagcctacct agactcagcc ggctctccac gctttgcctg |
| | | accctgcttg ctcaactcta gttaacggtg gagggcagtg tagtctgagc |
| | | agtacacgtt gctgccgcgc gcgccaccag acataatagc tgacagacta |
| | | acagactgtt cctttccatg ggtcttttct gcagtcaccg tcgtcgacac |
| | | gtgtgatcag atatcgcggc cgctctagac caccatggga tggtcatgta |
| | | tcatcctttt tctagtagca actgcaaccg gtgtacattc agaaattgtg |
| | | ttgacacagt ctccaggcac cctgtctttg tctccagggg aaacagccat |
| | | catctcttgt cggaccagtc agtatggttc cttagcctgg tatcaacaga |
| | | ggcccggcca ggcccccagg ctcgtcatct attcgggctc tactcgggcc |
| | | gctggcatcc cagacaggtt cagcggcagt cggtgggggc cagactacaa |
| | | tctcaccatc agcaacctgg agtcgggaga ttttggtgtt tattattgcc |
| | | agcagtatga attttttggc caggggacca aggtccaggt |
| | | cgacattaaa cgtacggtgg ctgcaccatc tgtcttcatc ttcccgccat |
| | | ctgaggatca ggtgaaatct ggaactgtct ctgttgtgtg cctgctgaat |
| | | aacttctatc ccagagaggc cagcgtaaag tggaaggtgg atggtgccct |
| | | caaaacgggt aactcccagg agagtgtcac agagcaggac agcaaggaca |
| | | acacctacag cctgagcagc accctgacgc tgagcagcac agagtaccag |
| | | agtcacaaag tctatgcctg cgaagtcacc catcagggcc tgagctcgcc |
| | | cgtcaccaag agcttcaaca gaggagagtg ttagggatcc agatctgctg |
| | | tgccttctag ttgccagcca tctgttgttt gcccctcccc cgtgccttcc |
| | | ttgaccctgg aaggtgccac tcccactgtc ctttcctaat aaaatgagga |
| | | aattgcatcg cattgtctga gtaggtgtca ttctattctg gggggtgggg |
| | | tggggcagga cagcaagggg gaggattggg aagacaatag |
| | | caggcatgct ggggatgcgg tgggctctat gggtacccag gtgctgaaga |
| | | attgacccgg ttcctcctgg gccagaaaga agcaggcaca tccccttctc |
| | | tgtgacacac cctgtccacg cccctggttc ttagttccag ccccactcat |
| | | aggacactca tagctcagga gggctccgcc ttcaatccca cccgctaaag |
| | | tacatggagc ggtctctccc tccctcatca gcccaccaaa ccaaacctag |
| | | cctccaagag tgggaagaaa ttaaagcaag ataggctatt aagtgcagag |
| | | ggagagaaaa tgcctccaac atgtgaggaa gtaatgagag aaatcataga |
| | | attttaaggc catgatttaa ggccatcatg gccttaatct tccgcttcct |
| | | cgctcactga ctcgctgcgc tcggtcgttc ggctgcggcg agcggtatca |
| | | gctcactcaa aggcggtaat acggttatcc acagaatcag gggataacgc |
| | | aggaaagaac atgtgagcaa aaggccagca aaaggccagg |
| | | aaccgtaaaa aggccgcgtt gctggcgttt ttccataggc tccgcccccc |
| | | tgacgagcat cacaaaaatc gacgctcaag tcagaggtgg cgaaacccga |
| | | caggactata aagataccag gcgtttcccc ctggaagctc cctcgtgcgc |
| | | tctcctgttc cgaccctgcc gcttaccgga tacctgtccg cctttctccc |
| | | ttcgggaagc gtggcgcttt ctcatagctc acgctgtagg |
| | | tatctcagtt cggtgtaggt cgttcgctcc aagctgggct gtgtgcacga |
| | | accccccgtt cagcccgacc gctgcgcctt atccggtaac tatcgtcttg |
| | | agtccaaccc ggtaagacac gacttatcgc cactggcagc agccactggt |
| | | aacaggatta gcagagcgag gtatgtaggc ggtgctacag agttcttgaa |
| | | gtggtggcct aactacggct acactagaag aacagtattt |
| | | ggtatctgcg ctctgctgaa gccagttacc ttcggaaaaa gagttggtag |
| | | ctcttgatcc ggcaaacaaa ccaccgctgg tagcggtggt ttttttgttt |
| | | gcaagcagca gattacgcgc agaaaaaaag gatctcaaga agatcctttg |
| | | atcttttcta cggggtctga cgctcagtgg aacgaaaact cacgttaagg |
| | | gattttggtc atgagattat caaaaaggat cttcacctag atccttttaa |
| | | attaaaaatg aagttttaaa tcaatctaaa gtatatatga gtaaacttgg |
| | | tctgacagtt accaatgctt aatcagtgag gcacctatct cagcgatctg |
| | | tctatttcgt tcatccatag ttgcctgact cggggggggg gggcgctgag |
| | | gtctgcctcg tgaagaaggt gttgctgact cataccaggc ctgaatcgcc |
| | | ccatcatcca gccagaaagt gagggagcca cggttgatga gagctttgtt |
| | | gtaggtggac cagttggtga ttttgaactt ttgctttgcc acggaacggt |
| | | ctgcgttgtc gggaagatgc gtgatctgat ccttcaactc agcaaaagtt |
| | | cgatttattc aacaaagccg ccgtcccgtc aagtcagcgt aatgctctgc |
| | | cagtgttaca accaattaac caattctgat tagaaaaact catcgagcat |
| | | caaatgaaac tgcaatttat tcatatcagg attatcaata ccatattttt |
| | | gaaaaagccg tttctgtaat gaaggagaaa actcaccgag gcagttccat |
| | | aggatggcaa gatcctggta tcggtctgcg attccgactc |
| | | gtccaacatc aatacaacct attaatttcc cctcgtcaaa aataaggtta |
| | | tcaagtgaga aatcaccatg agtgacgact gaatccggtg agaatggcaa |
| | | aagcttatgc atttctttcc agacttgttc aacaggccag ccattacgct |
| | | cgtcatcaaa atcactcgca tcaaccaaac cgttattcat tcgtgattgc |
| | | gcctgagcga gacgaaatac gcgatcgctg ttaaaaggac |
| | | aattacaaac aggaatcgaa tgcaaccggc gcaggaacac tgccagcgca |
| | | tcaacaatat tttcacctga atcaggatat tcttctaata cctggaatgc |
| | | tgttttcccg gggatcgcag tggtgagtaa ccatgcatca tcaggagtac |
| | | ggataaaatg cttgatggtc ggaagaggca taaattccgt cagccagttt |
| | | agtctgacca tctcatctgt aacatcattg gcaacgctac ctttgccatg |
| | | tttcagaaac aactctggcg catcgggctt cccatacaat cgatagattg |
| | | tcgcacctga ttgcccgaca ttatcgcgag cccatttata cccatataaa |
| | | tcagcatcca tgttggaatt taatcgcggc ctcgagcaag acgtttcccg |
| | | ttgaatatgg ctcataacac cccttgtatt actgtttatg taagcagaca |
| | | gttttattgt tcatgatgat atatttttat cttgtgcaat gtaacatcag agattttgag |
| | | acacaacgtg gctttccccc cccccccatt attgaagcat ttatcagggt |
| | | tattgtctca tgagcggata catatttgaa tgtatttaga aaaataaaca |
| | | aataggggtt ccgcgcacat ttccccgaaa agtgccacct gacgtctaag |
| | | aaaccattat tatcatgaca ttaacctata aaaataggcg tatcacgagg |
| | | ccctttcgtc |
| 114 | Macaque Kappa chain | MGWSCIILFLVATATGVHSEIVLTQSPGTLSLSPGETA |
| | backbone aa sequence | IISCRTSQYGSLAWYQQRPGQAPRLVIYSGSTRAAGI |
| | | PDRFSGSRWGPDYNLTISNLESGDFGVYYCQQYEFF |
| | | GQGTKVQVDIKRTVAAPSVFIFPPSEDQVKSGTVSVV |
| | | CLLNNFYPREASVKWKVDGALKTGNSQESVTEQDS |
| | | KDNTYSLSSTLTLSSTEYQSHKVYACEVTHQGLSSPV |
| | | TKSFNRGEC |
| 115 | Macaque Light chain | MGWSCIILFLVATATGVHSQSALTQPPSVSGSPGQSV |
| | backbone aa sequence | TISCTGTSSDVDGYNYVSWYQQHPGKAPKLMIYGVS |
| | | NRPSGVSDRFSGSKSGNTASLTISGLQAEDEADYYCC |
| | | SSTTSYTYIFGTGTKVTVLGQPKAAPSVTLFPPSSEEL |
| | | QANKATLVCLISDFYPGAVEVAWKADGSAVNAGVE |
| | | TTKPSKQSNNKYAASSYLSLTSDQWKSHKSYSCQVT |
| | | HEGSTVEKTVAPAECS |
| 116 | Macaque Heavy chain | tcgcgcgttt cggtgatgac ggtgaaaacc tctgacacat gcagctcccg |
| | backbone nucleotide | gagacggtca cagcttgtct gtaagcggat gccgggagca gacaagcccg |
| | sequence-H02 | tcagggcgcg tcagcgggtg ttggcgggtg tcggggctgg cttaactatg |
| | | cggcatcaga gcagattgta ctgagagtgc accatatgcg gtgtgaaata |
| | | ccgcacagat gcgtaaggag aaaataccgc atcagattgg |
| | | ctattggcca ttgcatacgt tgtatccata tcataatatg tacatttata |
| | | ttggctcatg tccaacatta ccgccatgtt gacattgatt attgactagt |
| | | tattaatagt aatcaattac ggggtcatta gttcatagcc catatatgga |
| | | gttccgcgtt acataactta cggtaaatgg cccgcctggc tgaccgccca |
| | | acgacccccg cccattgacg tcaataatga cgtatgttcc |
| | | catagtaacg ccaataggga ctttccattg acgtcaatgg gtggagtatt |
| | | tacggtaaac tgcccacttg gcagtacatc aagtgtatca tatgccaagt |
| | | acgcccccta ttgacgtcaa tgacggtaaa tggcccgcct ggcattatgc |
| | | ccagtacatg accttatggg actttcctac ttggcagtac atctacgtat |
| | | tagtcatcgc tattaccatg gtgatgcggt tttggcagta catcaatggg |
| | | cgtggatagc ggtttgactc acggggattt ccaagtctcc accccattga |
| | | cgtcaatggg agtttgtttt ggcaccaaaa tcaacgggac tttccaaaat |
| | | gtcgtaacaa ctccgcccca ttgacgcaaa tgggcggtag gcgtgtacgg |
| | | tgggaggtct atataagcag agctcgttta gtgaaccgtc agatcgcctg |
| | | gagacgccat ccacgctgtt ttgacctcca tagaagacac cgggaccgat |
| | | ccagcctcca tcggctcgca tctctccttc acgcgcccgc |
| | | cgccctacct gaggccgcca tccacgccgg ttgagtcgcg ttctgccgcc |
| | | tcccgcctgt ggtgcctcct gaactgcgtc cgccgtctag gtaagtttaa |
| | | agctcaggtc gagaccgggc ctttgtccgg cgctcccttg gagcctacct |
| | | agactcagcc ggctctccac gctttgcctg accctgcttg ctcaactcta |
| | | gttaacggtg gagggcagtg tagtctgagc agtactcgtt gctgccgcgc |
| | | gcgccaccag acataatagc tgacagacta acagactgtt cctttccatg |
| | | ggtcttttct gcagtcaccg tcgtcgacac gtgtgatcag atatcgcggc |
| | | cgctctagac caccatggga tggtcatgta tcatcctttt tctagtagca |
| | | actgcaaccg gtgtacattc ccaactgcag ttgcaggagt cgggcccagg |
| | | agtggtgaag ccttcggaga ccctgtccct cacctgcact atctctggtg |
| | | gctccttcag tacttactac tggacctgga ttcgccagcc cccagggaag |
| | | ggactggagt gggttgggta tatcggtaat ggtggtcgta gcctcaacta |
| | | caacccctcc ctcaagagtc gcatcaccct gtcagtagac gcgtccaaga |
| | | accagttctc cctgaaggtg acctctgtga ccgccgcgga cacggccgtc |
| | | tattactgtg ggagagccag gggactccgc ggaaactggt tcgatgtctg |
| | | gggcccggga gtcctggtca ccgtctcctc agctagcacc aagggcccta |
| | | gtgtgtttcc tctggcccct agcagcagaa gcacatctga atctacagcc |
| | | gccctgggct gcctggtgaa agattacttc cccgagcccg tgaccgtgtc |
| | | ttggaatagc ggctctctga ccagcggcgt gcacacattt ccagctgtgc |
| | | tgcagagcag cggcctgtat tctctgagca gcgtggtgac agtgccaagc |
| | | agctctctgg gcacccagac ctacgtgtgc aacgtgaacc acaagcccag |
| | | caacaccaag gtggacaagc gggtggaaat caagacctgt ggcggcggaa |
| | | gcaagcctcc tacctgtcct ccttgtacca gccctgaact gctgggcgga |
| | | cctagcgtgt tcctgttccc cccaaagccc aaggacaccc tgatgatcag |
| | | cagaaccccc gaagtgacct gcgtggtggt ggatgtgtcc caggaagatc |
| | | ccgacgtgaa gttcaattgg tacgtgaacg gcgccgaggt gcaccatgcc |
| | | cagacaaagc ccagagagac acagtacaac agcacctacc gggtggtgtc |
| | | tgtgctgacc gtgacacacc aggactggct gaacggcaaa gagtacacat |
| | | gcaaggtgtc caacaaggcc ctgcctgccc ccatccagaa aaccatcagc |
| | | aaggacaagg gccagcccag agaacctcag gtgtacacac tgccccccag |
| | | cagagaggaa ctgaccaaga atcaggtgtc cctgacctgt ctggtgaaag |
| | | gcttctaccc cagcgacatc gtggtggaat gggagtctag cggacagccc |
| | | gagaacacct acaagaccac ccctccagtg ctggatagcg acggcagcta |
| | | cttcctgtac agcaagctga ccgtggacaa gagcagatgg cagcagggca |
| | | acgtgttcag ctgctctgtg atgcacgagg ccctgcacaa ccactacacc |
| | | cagaagtctc tgagcctgag ccccggaaag tgatgatgaa cacgtgggat |
| | | ccagatctgc tgtgccttct agttgccagc catctgttgt ttgcccctcc |
| | | cccgtgcctt ccttgaccct ggaaggtgcc actcccactg tcctttccta |
| | | ataaaatgag gaaattgcat cgcattgtct gagtaggtgt cattctattc |
| | | tggggggtgg ggtggggcag gacagcaagg gggaggattg ggaagacaat |
| | | agcaggcatg ctggggatgc ggtgggctct atgggtaccc aggtgctgaa |
| | | gaattgaccc ggttcctcct gggccagaaa gaagcaggca catccccttc |
| | | tctgtgacac accctgtcca cgcccctggt tcttagttcc agccccactc |
| | | ataggacact catagctcag gagggctccg ccttcaatcc cacccgctaa |
| | | agtacttgga gcggtctctc cctccctcat cagcccacca aaccaaacct |
| | | agcctccaag agtgggaaga aattaaagca agataggcta ttaagtgcag |
| | | agggagagaa aatgcctcca acatgtgagg aagtaatgag agaaatcata |
| | | gaattttaag gccatgattt aaggccatca tggccttaat cttccgcttc |
| | | ctcgctcact gactcgctgc gctcggtcgt tcggctgcgg cgagcggtat |
| | | cagctcactc aaaggcggta atacggttat ccacagaatc aggggataac |
| | | gcaggaaaga acatgtgagc aaaaggccag caaaaggcca ggaaccgtaa |
| | | aaaggccgcg ttgctggcgt ttttccatag gctccgcccc cctgacgagc |
| | | atcacaaaaa tcgacgctca agtcagaggt ggcgaaaccc gacaggacta |
| | | taaagatacc aggcgtttcc ccctggaagc tccctcgtgc gctctcctgt |
| | | tccgaccctg ccgcttaccg gatacctgtc cgcctttctc ccttcgggaa |
| | | gcgtggcgct ttctcatagc tcacgctgta ggtatctcag ttcggtgtag |
| | | gtcgttcgct ccaagctggg ctgtgtgcac gaaccccccg ttcagcccga |
| | | ccgctgcgcc ttatccggta actatcgtct tgagtccaac ccggtaagac |
| | | acgacttatc gccactggca gcagccactg gtaacaggat tagcagagcg |
| | | aggtatgtag gcggtgctac agagttcttg aagtggtggc ctaactacgg |
| | | ctacactaga agaacagtat ttggtatctg cgctctgctg aagccagtta |
| | | ccttcggaaa aagagttggt agctcttgat ccggcaaaca aaccaccgct |
| | | ggtagcggtg gtttttttgt ttgcaagcag cagattacgc gcagaaaaaa |
| | | aggatctcaa gaagatcctt tgatcttttc tacggggtct gacgctcagt |
| | | ggaacgaaaa ctcacgttaa gggattttgg tcatgagatt atcaaaaagg |
| | | atcttcacct agatcctttt aaattaaaaa tgaagtttta aatcaatcta |
| | | aagtatatat gagtaaactt ggtctgacag ttaccaatgc ttaatcagtg |
| | | aggcacctat ctcagcgatc tgtctatttc gttcatccat agttgcctga |
| | | ctcggggggg gggggcgctg aggtctgcct cgtgaagaag gtgttgctga |
| | | ctcataccag gcctgaatcg ccccatcatc cagccagaaa gtgagggagc |
| | | cacggttgat gagagctttg ttgtaggtgg accagttggt gattttgaac |
| | | ttttgctttg ccacggaacg gtctgcgttg tcgggaagat gcgtgatctg |
| | | atccttcaac tcagcaaaag ttcgatttat tcaacaaagc cgccgtcccg |
| | | tcaagtcagc gtaatgctct gccagtgtta caaccaatta accaattctg |
| | | attagaaaaa ctcatcgagc atcaaatgaa actgcaattt attcatatca |
| | | ggattatcaa taccatattt ttgaaaaagc cgtttctgta atgaaggaga |
| | | aaactcaccg aggcagttcc ataggatggc aagatcctgg |
| | | tatcggtctg cgattccgac tcgtccaaca tcaatacaac ctattaattt |
| | | cccctcgtca aaaataaggt tatcaagtga gaaatcacca tgagtgacga |
| | | ctgaatccgg tgagaatggc aaaagcttat gcatttcttt ccagacttgt |
| | | tcaacaggcc agccattacg ctcgtcatca aaatcactcg catcaaccaa |
| | | accgttattc attcgtgatt gcgcctgagc gagacgaaat acgcgatcgc |
| | | tgttaaaagg acaattacaa acaggaatcg aatgcaaccg gcgcaggaac |
| | | actgccagcg catcaacaat attttcacct gaatcaggat attcttctaa |
| | | tacctggaat gctgttttcc cggggatcgc agtggtgagt aaccatgcat |
| | | catcaggagt acggataaaa tgcttgatgg tcggaagagg cataaattcc |
| | | gtcagccagt ttagtctgac catctcatct gtaacatcat tggcaacgct |
| | | acctttgcca tgtttcagaa acaactctgg cgcatcgggc |
| | | ttcccataca atcgatagat tgtcgcacct gattgcccga cattatcgcg |
| | | agcccattta tacccatata aatcagcatc catgttggaa tttaatcgcg |
| | | gcctcgagca agacgtttcc cgttgaatat ggctcataac accccttgta |
| | | ttactgttta tgtaagcaga cagttttatt gttcatgatg atatattttt atcttgtgca |
| | | atgtaacatc agagattttg agacacaacg tggctttccc ccccccccca |
| | | ttattgaagc atttatcagg gttattgtct catgagcgga tacatatttg |
| | | aatgtattta gaaaaataaa caaatagggg ttccgcgcac atttccccga |
| | | aaagtgccac ctgacgtcta agaaaccatt attatcatga cattaaccta |
| | | taaaaatagg cgtatcacga ggccctttcg tc |
| 117 | Macaque Heavy chain | MGWSCIILFLVATATGVHSQLQLQESGPGVVKPSET |
| | backbone aa sequence-H02 | LSLTCTISGGSFSTYYWTWIRQPPGKGLEWVGYIGN |
| | | GGRSLNYNPSLKSRITLSVDASKNQFSLKVTSVTAAD |
| | | TAVYYCGRARGLRGNWFDVWGPGVLVTVSSASTK |
| | | GPSVFPLAPSSRSTSESTAALGCLVKDYFPEPVTVSW |
| | | NSGSLTSGVHTFPAVLQSSGLYSLSSVVTVPSSSLGT |
| | | QTYVCNVNHKPSNTKVDKRVEIKTCGGGSKPPTCPP |
| | | CTSPELLGGPSVFLFPPKPKDTLMISRTPEVTCVVVD |
| | | VSQEDPDVKFNWYVNGAEVHHAQTKPRETQYNSTY |
| | | RVVSVLTVTHQDWLNGKEYTCKVSNKALPAPIQKTI |
| | | SKDKGQPREPQVYTLPPSREELTKNQVSLTCLVKGF |
| | | YPSDIVVEWESSGQPENTYKTTPPVLDSDGSYFLYSK |
| | | LTVDKSRWQQGNVFSCSVMHEALHNHYTQKSLSLS |
| | | PGK |
| 118 | Macaque Light chain | tcgcgcgttt cggtgatgac ggtgaaaacc tctgacacat gcagctcccg |
| | backbone nucleotide | gagacggtca cagcttgtct gtaagcggat gccgggagca gacaagcccg |
| | sequence-H02 | tcagggcgcg tcagcgggtg ttggcgggtg tcggggctgg cttaactatg |
| | | cggcatcaga gcagattgta ctgagagtgc accatatgcg gtgtgaaata |
| | | ccgcacagat gcgtaaggag aaaataccgc atcagattgg |
| | | ctattggcca ttgcatacgt tgtatccata tcataatatg tacatttata |
| | | ttggctcatg tccaacatta ccgccatgtt gacattgatt attgactagt |
| | | tattaatagt aatcaattac ggggtcatta gttcatagcc catatatgga |
| | | gttccgcgtt acataactta cggtaaatgg cccgcctggc tgaccgccca |
| | | acgacccccg cccattgacg tcaataatga cgtatgttcc catagtaacg |
| | | ccaataggga ctttccattg acgtcaatgg gtggagtatt tacggtaaac |
| | | tgcccacttg gcagtacatc aagtgtatca tatgccaagt acgcccccta |
| | | ttgacgtcaa tgacggtaaa tggcccgcct ggcattatgc ccagtacatg |
| | | accttatggg actttcctac ttggcagtac atctacgtat tagtcatcgc |
| | | tattaccatg gtgatgcggt tttggcagta catcaatggg cgtggatagc |
| | | ggtttgactc acggggattt ccaagtctcc accccattga cgtcaatggg |
| | | agtttgtttt ggcaccaaaa tcaacgggac tttccaaaat gtcgtaacaa |
| | | ctccgcccca ttgacgcaaa tgggcggtag gcgtgtacgg tgggaggtct |
| | | atataagcag agctcgttta gtgaaccgtc agatcgcctg gagacgccat |
| | | ccacgctgtt ttgacctcca tagaagacac cgggaccgat ccagcctcca |
| | | tcggctcgca tctctccttc acgcgcccgc cgccctacct gaggccgcca |
| | | tccacgccgg ttgagtcgcg ttctgccgcc tcccgcctgt ggtgcctcct |
| | | gaactgcgtc cgccgtctag gtaagtttaa agctcaggtc gagaccgggc |
| | | ctttgtccgg cgctcccttg gagcctacct agactcagcc ggctctccac |
| | | gctttgcctg accctgcttg ctcaactcta gttaacggtg gagggcagtg |
| | | tagtctgagc agtacacgtt gctgccgcgc gcgccaccag acataatagc |
| | | tgacagacta acagactgtt cctttccatg ggtcttttct gcagtcaccg |
| | | tcgtcgacac gtgtgatcag atatcgcggc cgctctagac caccatggga |
| | | tggtcatgta tcatcctttt tctagtagca actgcaaccg gtgtacattc |
| | | ccaggctgcc ctgactcagc ctccctctgt gtctgggtct cctggacagt |
| | | cggtcaccat ctcctgcact ggaaccagca gtgacatcgg tggttataac |
| | | tatgtctcct ggtaccaaca acacccaggc aaagccccca aagtcatgat |
| | | ttatgaggtc agtaagcggc cctcaggggt ctctgatcgc ttctctggtt |
| | | ccaaatctgg caacatagcc tccctgacca tctctgggct ccaggctgag |
| | | gacgaggctg attattactg cagctcatat gcaggcagca acactttctt |
| | | attcggagga gggacccggc tgacagtact aggtcagccc aaggctgccc |
| | | cctcggtcac tctcttcccg ccctcctctg aggagcttca agccaacaag |
| | | gccacactag tgtgtctgat cagtgacttc tacccgggag ccgtggaagt |
| | | ggcctggaag gcagatggca gcgctgtcaa cgcgggagtg gagaccacca |
| | | aaccctccaa acagagcaac aacaagtacg cggccagcag ctacctgagc |
| | | ctgacgtccg accagtggaa gtcccacaag agctacagct gccaggtcac |
| | | gcacgaaggg agcaccgtgg agaagacagt ggcccctgca gaatgttcat |
| | | agggatccag atctgctgtg ccttctagtt gccagccatc tgttgtttgc |
| | | ccctcccccg tgccttcctt gaccctggaa ggtgccactc ccactgtcct |
| | | ttcctaataa aatgaggaaa ttgcatcgca ttgtctgagt aggtgtcatt |
| | | ctattctggg gggtggggtg gggcaggaca gcaaggggga |
| | | ggattgggaa gacaatagca ggcatgctgg ggatgcggtg ggctctatgg |
| | | gtacccaggt gctgaagaat tgacccggtt cctcctgggc cagaaagaag |
| | | caggcacatc cccttctctg tgacacaccc tgtccacgcc cctggttctt |
| | | agttccagcc ccactcatag gacactcata gctcaggagg gctccgcctt |
| | | caatcccacc cgctaaagta catggagcgg tctctccctc cctcatcagc |
| | | ccaccaaacc aaacctagcc tccaagagtg ggaagaaatt aaagcaagat |
| | | aggctattaa gtgcagaggg agagaaaatg cctccaacat gtgaggaagt |
| | | aatgagagaa atcatagaat tttaaggcca tgatttaagg ccatcatggc |
| | | cttaatcttc cgcttcctcg ctcactgact cgctgcgctc ggtcgttcgg |
| | | ctgcggcgag cggtatcagc tcactcaaag gcggtaatac ggttatccac |
| | | agaatcaggg gataacgcag gaaagaacat gtgagcaaaa ggccagcaaa |
| | | aggccaggaa ccgtaaaaag gccgcgttgc tggcgttttt ccataggctc |
| | | cgcccccctg acgagcatca caaaaatcga cgctcaagtc agaggtggcg |
| | | aaacccgaca ggactataaa gataccaggc gtttccccct ggaagctccc |
| | | tcgtgcgctc tcctgttccg accctgccgc ttaccggata cctgtccgcc |
| | | tttctccctt cgggaagcgt ggcgctttct catagctcac gctgtaggta |
| | | tctcagttcg gtgtaggtcg ttcgctccaa gctgggctgt gtgcacgaac |
| | | cccccgttca gcccgaccgc tgcgccttat ccggtaacta tcgtcttgag |
| | | tccaacccgg taagacacga cttatcgcca ctggcagcag ccactggtaa |
| | | caggattagc agagcgaggt atgtaggcgg tgctacagag ttcttgaagt |
| | | ggtggcctaa ctacggctac actagaagaa cagtatttgg tatctgcgct |
| | | ctgctgaagc cagttacctt cggaaaaaga gttggtagct cttgatccgg |
| | | caaacaaacc accgctggta gcggtggttt ttttgtttgc aagcagcaga |
| | | ttacgcgcag aaaaaaagga tctcaagaag atcctttgat cttttctacg |
| | | gggtctgacg ctcagtggaa cgaaaactca cgttaaggga ttttggtcat |
| | | gagattatca aaaaggatct tcacctagat ccttttaaat taaaaatgaa |
| | | gttttaaatc aatctaaagt atatatgagt aaacttggtc tgacagttac |
| | | caatgcttaa tcagtgaggc acctatctca gcgatctgtc tatttcgttc |
| | | atccatagtt gcctgactcg gggggggggg gcgctgaggt |
| | | ctgcctcgtg aagaaggtgt tgctgactca taccaggcct gaatcgcccc |
| | | atcatccagc cagaaagtga gggagccacg gttgatgaga gctttgttgt |
| | | aggtggacca gttggtgatt ttgaactttt gctttgccac ggaacggtct |
| | | gcgttgtcgg gaagatgcgt gatctgatcc ttcaactcag caaaagttcg |
| | | atttattcaa caaagccgcc gtcccgtcaa gtcagcgtaa tgctctgcca |
| | | gtgttacaac caattaacca attctgatta gaaaaactca tcgagcatca |
| | | aatgaaactg caatttattc atatcaggat tatcaatacc atatttttga |
| | | aaaagccgtt tctgtaatga aggagaaaac tcaccgaggc agttccatag |
| | | gatggcaaga tcctggtatc ggtctgcgat tccgactcgt ccaacatcaa |
| | | tacaacctat taatttcccc tcgtcaaaaa taaggttatc aagtgagaaa |
| | | tcaccatgag tgacgactga atccggtgag aatggcaaaa |
| | | gcttatgcat ttctttccag acttgttcaa caggccagcc attacgctcg |
| | | tcatcaaaat cactcgcatc aaccaaaccg ttattcattc gtgattgcgc |
| | | ctgagcgaga cgaaatacgc gatcgctgtt aaaaggacaa ttacaaacag |
| | | gaatcgaatg caaccggcgc aggaacactg ccagcgcatc aacaatattt |
| | | tcacctgaat caggatattc ttctaatacc tggaatgctg ttttcccggg |
| | | gatcgcagtg gtgagtaacc atgcatcatc aggagtacgg ataaaatgct |
| | | tgatggtcgg aagaggcata aattccgtca gccagtttag tctgaccatc |
| | | tcatctgtaa catcattggc aacgctacct ttgccatgtt tcagaaacaa |
| | | ctctggcgca tcgggcttcc catacaatcg atagattgtc gcacctgatt |
| | | gcccgacatt atcgcgagcc catttatacc catataaatc agcatccatg |
| | | ttggaattta atcgcggcct cgagcaagac gtttcccgtt gaatatggct |
| | | cataacaccc cttgtattac tgtttatgta agcagacagt tttattgttc |
| | | atgatgatat atttttatct tgtgcaatgt aacatcagag attttgagac |
| | | acaacgtggc tttccccccc cccccattat tgaagcattt atcagggtta |
| | | ttgtctcatg agcggataca tatttgaatg tatttagaaa aataaacaaa |
| | | taggggttcc gcgcacattt ccccgaaaag tgccacctga cgtctaagaa |
| | | accattatta tcatgacatt aacctataaa aataggcgta tcacgaggcc |
| | | ctttcgtc |
| 119 | Macaque Light chain | MGWSCIILFLVATATGVHSQAALTQPPSVSGSPGQSV |
| | backbone aa sequence-H02 | TISCTGTSSDIGGYNYVSWYQQHPGKAPKVMIYEVS |
| | | KRPSGVSDRFSGSKSGNIASLTISGLQAEDEADYYCS |
| | | SYAGSNTFLFGGGTRLTVLGQPKAAPSVTLFPPSSEE |
| | | LQANKATLVCLISDFYPGAVEVAWKADGSAVNAGV |
| | | ETTKPSKQSNNKYAASSYLSLTSDQWKSHKSYSCQV |
| | | THEGSTVEKTVAPAECS |
| 120 | Humanized H02 heavy chain | tcgcgcgttt cggtgatgac ggtgaaaacc tctgacacat gcagctcccg |
| | backbone nucleotide | gagacggtca cagcttgtct gtaagcggat gccgggagca gacaagcccg |
| | sequence | tcagggcgcg tcagcgggtg ttggcgggtg tcggggctgg cttaactatg |
| | | cggcatcaga gcagattgta ctgagagtgc accatatgcg gtgtgaaata |
| | | ccgcacagat gcgtaaggag aaaataccgc atcagattgg ctattggcca |
| | | ttgcatacgt tgtatccata tcataatatg tacatttata ttggctcatg |
| | | tccaacatta ccgccatgtt gacattgatt attgactagt tattaatagt |
| | | aatcaattac ggggtcatta gttcatagcc catatatgga gttccgcgtt |
| | | acataactta cggtaaatgg cccgcctggc tgaccgccca acgacccccg |
| | | cccattgacg tcaataatga cgtatgttcc catagtaacg ccaataggga |
| | | ctttccattg acgtcaatgg gtggagtatt tacggtaaac tgcccacttg |
| | | gcagtacatc aagtgtatca tatgccaagt acgcccccta ttgacgtcaa |
| | | tgacggtaaa tggcccgcct ggcattatgc ccagtacatg accttatggg |
| | | actttcctac ttggcagtac atctacgtat tagtcatcgc tattaccatg |
| | | gtgatgcggt tttggcagta catcaatggg cgtggatagc ggtttgactc |
| | | acggggattt ccaagtctcc accccattga cgtcaatggg agtttgtttt |
| | | ggcaccaaaa tcaacgggac tttccaaaat gtcgtaacaa ctccgcccca |
| | | ttgacgcaaa tgggcggtag gcgtgtacgg tgggaggtct atataagcag |
| | | agctcgttta gtgaaccgtc agatcgcctg gagacgccat ccacgctgtt |
| | | ttgacctcca tagaagacac cgggaccgat ccagcctcca tcggctcgca |
| | | tctctccttc acgcgcccgc cgccctacct gaggccgcca tccacgccgg |
| | | ttgagtcgcg ttctgccgcc tcccgcctgt ggtgcctcct gaactgcgtc |
| | | cgccgtctag gtaagtttaa agctcaggtc gagaccgggc ctttgtccgg |
| | | cgctcccttg gagcctacct agactcagcc ggctctccac gctttgcctg |
| | | accctgcttg ctcaactcta gttaacggtg gagggcagtg tagtctgagc |
| | | agtactcgtt gctgccgcgc gcgccaccag acataatagc tgacagacta |
| | | acagactgtt cctttccatg ggtcttttct gcagtcaccg tcgtcgacac |
| | | gtgtgatcag atatcgcggc cgctctagac caccatggga tggtcatgta |
| | | tcatcctttt tctagtagca actgcaaccg gtgtacattc ccaactgcag |
| | | ttgcaggagt cgggcccagg agtggtgaag ccttcggaga ccctgtccct |
| | | cacctgcact atctctggtg gctccttcag tacttactac tggacctgga |
| | | ttcgccagcc cccagggaag ggactggagt gggttgggta tatcggtaat |
| | | ggtggtcgta gcctcaacta caacccctcc ctcaagagtc gcatcaccct |
| | | gtcagtagac gcgtccaaga accagttctc cctgaaggtg acctctgtga |
| | | ccgccgcgga cacggccgtc tattactgtg ggagagccag gggactccgc |
| | | ggaaactggt tcgatgtctg gggcccggga gtcctggtca ccgtctcctc |
| | | agctagcacc aagggcccat cggtcttccc cctggcaccc tcctccaaga |
| | | gcacctctgg gggcacagcg gccctgggct gcctggtcaa ggactacttc |
| | | cccgaaccgg tgacggtgtc gtggaactca ggcgccctga ccagcggcgt |
| | | gcacaccttc ccggctgtcc tacagtcctc aggactctac tccctcagca |
| | | gcgtggtgac cgtgccctcc agcagcttgg gcacccagac ctacatctgc |
| | | aacgtgaatc acaagcccag caacaccaag gtggacaaga aagttgagcc |
| | | caaatcttgt gacaaaactc acacatgccc accgtgccca gcacctgaac |
| | | tcctgggggg accgtcagtc ttcctcttcc ccccaaaacc caaggacacc |
| | | ctcatgatct cccggacccc tgaggtcaca tgcgtggtgg tggacgtgag |
| | | ccacgaagac cctgaggtca agttcaactg gtacgtggac ggcgtggagg |
| | | tgcataatgc caagacaaag ccgcgggagg agcagtacaa cagcacgtac |
| | | cgtgtggtca gcgtcctcac cgtcctgcac caggactggc tgaatggcaa |
| | | ggagtacaag tgcaaggtct ccaacaaagc cctcccagcc cccatcgaga |
| | | aaaccatctc caaagccaaa gggcagcccc gagaaccaca ggtgtacacc |
| | | ctgcccccat cccgggatga gctgaccaag aaccaggtca gcctgacctg |
| | | cctggtcaaa ggcttctatc ccagcgacat cgccgtggag tgggagagca |
| | | atgggcagcc ggagaacaac tacaagacca cgcctcccgt |
| | | gctggactcc gacggctcct tcttcctcta cagcaagctc accgtggaca |
| | | agagcaggtg gcagcagggg aacgtcttct catgctccgt gatgcatgag |
| | | gctctgcaca accactacac gcagaagagc ctctccctgt ctccgggtaa |
| | | atgatgagga tccagatctg ctgtgccttc tagttgccag ccatctgttg |
| | | tttgcccctc ccccgtgcct tccttgaccc tggaaggtgc cactcccact |
| | | gtcctttcct aataaaatga ggaaattgca tcgcattgtc tgagtaggtg |
| | | tcattctatt ctggggggtg gggtggggca ggacagcaag ggggaggatt |
| | | gggaagacaa tagcaggcat gctggggatg cggtgggctc tatgggtacc |
| | | caggtgctga agaattgacc cggttcctcc tgggccagaa agaagcaggc |
| | | acatcccctt ctctgtgaca caccctgtcc acgcccctgg ttcttagttc |
| | | cagccccact cataggacac tcatagctca ggagggctcc gccttcaatc |
| | | ccacccgcta aagtacttgg agcggtctct ccctccctca tcagcccacc |
| | | aaaccaaacc tagcctccaa gagtgggaag aaattaaagc aagataggct |
| | | attaagtgca gagggagaga aaatgcctcc aacatgtgag gaagtaatga |
| | | gagaaatcat agaattttaa ggccatgatt taaggccatc atggccttaa |
| | | tcttccgctt cctcgctcac tgactcgctg cgctcggtcg ttcggctgcg |
| | | gcgagcggta tcagctcact caaaggcggt aatacggtta tccacagaat |
| | | caggggataa cgcaggaaag aacatgtgag caaaaggcca gcaaaaggcc |
| | | aggaaccgta aaaaggccgc gttgctggcg tttttccata ggctccgccc |
| | | ccctgacgag catcacaaaa atcgacgctc aagtcagagg tggcgaaacc |
| | | cgacaggact ataaagatac caggcgtttc cccctggaag ctccctcgtg |
| | | cgctctcctg ttccgaccct gccgcttacc ggatacctgt ccgcctttct |
| | | cccttcggga agcgtggcgc tttctcatag ctcacgctgt aggtatctca |
| | | gttcggtgta ggtcgttcgc tccaagctgg gctgtgtgca cgaacccccc |
| | | gttcagcccg accgctgcgc cttatccggt aactatcgtc ttgagtccaa |
| | | cccggtaaga cacgacttat cgccactggc agcagccact ggtaacagga |
| | | ttagcagagc gaggtatgta ggcggtgcta cagagttctt gaagtggtgg |
| | | cctaactacg gctacactag aagaacagta tttggtatct gcgctctgct |
| | | gaagccagtt accttcggaa aaagagttgg tagctcttga tccggcaaac |
| | | aaaccaccgc tggtagcggt ggtttttttg tttgcaagca gcagattacg |
| | | cgcagaaaaa aaggatctca agaagatcct ttgatctttt ctacggggtc |
| | | tgacgctcag tggaacgaaa actcacgtta agggattttg gtcatgagat |
| | | tatcaaaaag gatcttcacc tagatccttt taaattaaaa atgaagtttt |
| | | aaatcaatct aaagtatata tgagtaaact tggtctgaca gttaccaatg |
| | | cttaatcagt gaggcaccta tctcagcgat ctgtctattt cgttcatcca |
| | | tagttgcctg actcgggggg ggggggcgct gaggtctgcc tcgtgaagaa |
| | | ggtgttgctg actcatacca ggcctgaatc gccccatcat ccagccagaa |
| | | agtgagggag ccacggttga tgagagcttt gttgtaggtg gaccagttgg |
| | | tgattttgaa cttttgcttt gccacggaac ggtctgcgtt gtcgggaaga |
| | | tgcgtgatct gatccttcaa ctcagcaaaa gttcgattta ttcaacaaag |
| | | ccgccgtccc gtcaagtcag cgtaatgctc tgccagtgtt acaaccaatt |
| | | aaccaattct gattagaaaa actcatcgag catcaaatga aactgcaatt |
| | | tattcatatc aggattatca ataccatatt tttgaaaaag ccgtttctgt |
| | | aatgaaggag aaaactcacc gaggcagttc cataggatgg caagatcctg |
| | | gtatcggtct gcgattccga ctcgtccaac atcaatacaa cctattaatt |
| | | tcccctcgtc aaaaataagg ttatcaagtg agaaatcacc atgagtgacg |
| | | actgaatccg gtgagaatgg caaaagctta tgcatttctt tccagacttg |
| | | ttcaacaggc cagccattac gctcgtcatc aaaatcactc gcatcaacca |
| | | aaccgttatt cattcgtgat tgcgcctgag cgagacgaaa tacgcgatcg |
| | | ctgttaaaag gacaattaca aacaggaatc gaatgcaacc ggcgcaggaa |
| | | cactgccagc gcatcaacaa tattttcacc tgaatcagga tattcttcta |
| | | atacctggaa tgctgttttc ccggggatcg cagtggtgag taaccatgca |
| | | tcatcaggag tacggataaa atgcttgatg gtcggaagag gcataaattc |
| | | cgtcagccag tttagtctga ccatctcatc tgtaacatca ttggcaacgc |
| | | tacctttgcc atgtttcaga aacaactctg gcgcatcggg cttcccatac |
| | | aatcgataga ttgtcgcacc tgattgcccg acattatcgc gagcccattt |
| | | atacccatat aaatcagcat ccatgttgga atttaatcgc ggcctcgagc |
| | | aagacgtttc ccgttgaata tggctcataa caccccttgt attactgttt |
| | | atgtaagcag acagttttat tgttcatgat gatatatttt tatcttgtgc |
| | | aatgtaacat cagagatttt gagacacaac gtggctttcc cccccccccc |
| | | attattgaag catttatcag ggttattgtc tcatgagcgg atacatattt |
| | | gaatgtattt agaaaaataa acaaataggg gttccgcgca catttccccg |
| | | aaaagtgcca cctgacgtct aagaaaccat tattatcatg acattaacct |
| | | ataaaaatag gcgtatcacg aggccctttc gtc |
| 121 | Humanized H02 heavy chain | MGWSCIILFLVATATGVHSQLQLQESGPGVVKPSET |
| | backbone aa sequence | LSLTCTISGGSFSTYYWTWIRQPPGKGLEWVGYIGN |
| | | GGRSLNYNPSLKSRITLSVDASKNQFSLKVTSVTAAD |
| | | TAVYYCGRARGLRGNWFDVWGPGVLVTVSSASTK |
| | | GPSVFPLAPSSKSTSGGTAALGCLVKDYFPEPVTVSW |
| | | NSGALTSGVHTFPAVLQSSGLYSLSSVVTVPSSSLGT |
| | | QTYICNVNHKPSNTKVDKKVEPKSCDKTHTCPPCPA |
| | | PELLGGPSVFLFPPKPKDTLMISRTPEVTCVVVDVSH |
| | | EDPEVKFNWYVDGVEVHNAKTKPREEQYNSTYRVV |
| | | SVLTVLHQDWLNGKEYKCKVSNKALPAPIEKTISKA |
| | | KGQPREPQVYTLPPSRDELTKNQVSLTCLVKGFYPS |
| | | DIAVEWESNGQPENNYKTTPPVLDSDGSFFLYSKLT |
| | | VDKSRWQQGNVFSCSVMHEALHNHYTQKSLSLSPG |
| | | K |
| 122 | Humanized H02 light chain | tcgcgcgttt cggtgatgac ggtgaaaacc tctgacacat gcagctcccg |
| | backbone aa sequence | gagacggtca cagcttgtct gtaagcggat gccgggagca gacaagcccg |
| | | tcagggcgcg tcagcgggtg ttggcgggtg tcggggctgg cttaactatg |
| | | cggcatcaga gcagattgta ctgagagtgc accatatgcg gtgtgaaata |
| | | ccgcacagat gcgtaaggag aaaataccgc atcagattgg ctattggcca |
| | | ttgcatacgt tgtatccata tcataatatg tacatttata ttggctcatg |
| | | tccaacatta ccgccatgtt gacattgatt attgactagt tattaatagt |
| | | aatcaattac ggggtcatta gttcatagcc catatatgga gttccgcgtt |
| | | acataactta cggtaaatgg cccgcctggc tgaccgccca acgacccccg |
| | | cccattgacg tcaataatga cgtatgttcc catagtaacg ccaataggga |
| | | ctttccattg acgtcaatgg gtggagtatt tacggtaaac tgcccacttg |
| | | gcagtacatc aagtgtatca tatgccaagt acgcccccta ttgacgtcaa |
| | | tgacggtaaa tggcccgcct ggcattatgc ccagtacatg accttatggg |
| | | actttcctac ttggcagtac atctacgtat tagtcatcgc tattaccatg |
| | | gtgatgcggt tttggcagta catcaatggg cgtggatagc ggtttgactc |
| | | acggggattt ccaagtctcc accccattga cgtcaatggg agtttgtttt |
| | | ggcaccaaaa tcaacgggac tttccaaaat gtcgtaacaa ctccgcccca |
| | | ttgacgcaaa tgggcggtag gcgtgtacgg tgggaggtct atataagcag |
| | | agctcgttta gtgaaccgtc agatcgcctg gagacgccat ccacgctgtt |
| | | ttgacctcca tagaagacac cgggaccgat ccagcctcca tcggctcgca |
| | | tctctccttc acgcgcccgc cgccctacct gaggccgcca tccacgccgg |
| | | ttgagtcgcg ttctgccgcc tcccgcctgt ggtgcctcct gaactgcgtc |
| | | cgccgtctag gtaagtttaa agctcaggtc gagaccgggc ctttgtccgg |
| | | cgctcccttg gagcctacct agactcagcc ggctctccac gctttgcctg |
| | | accctgcttg ctcaactcta gttaacggtg gagggcagtg tagtctgagc |
| | | agtactcgtt gctgccgcgc gcgccaccag acataatagc tgacagacta |
| | | acagactgtt cctttccatg ggtcttttct gcagtcaccg tcgtcgacac |
| | | gtgtgatcag atatcgcggc cgctctagac caccatggga tggtcatgta |
| | | tcatcctttt tctagtagca actgcaaccg gtgtacattc ccaggctgcc |
| | | ctgactcagc ctccctctgt gtctgggtct cctggacagt cggtcaccat |
| | | ctcctgcact ggaaccagca gtgacatcgg tggttataac tatgtctcct |
| | | ggtaccaaca acacccaggc aaagccccca aagtcatgat ttatgaggtc |
| | | agtaagcggc cctcaggggt ctctgatcgc ttctctggtt ccaaatctgg |
| | | caacatagcc tccctgacca tctctgggct ccaggctgag gacgaggctg |
| | | attattactg cagctcatat gcaggcagca acactttctt attcggagga |
| | | gggacccggc tgacagtCct aggtcagccc aaggctgccc cctcggtcac |
| | | tctgttcccg ccctcctctg aggagcttca agccaacaag gccacactgg |
| | | tgtgtctcat aagtgacttc tacccgggag ccgtgacagt ggcctggaag |
| | | gcagatagca gccccgtcaa ggcgggagtg gagaccacca caccctccaa |
| | | acaaagcaac aacaagtacg cggccagcag ctatctgagc ctgacgcctg |
| | | agcagtggaa gtcccacaga agctacagct gccaggtcac gcatgaaggg |
| | | agcaccgtgg agaagacagt ggcccctaca gaatgttcat gaggatccag |
| | | atctgctgtg ccttctagtt gccagccatc tgttgtttgc ccctcccccg |
| | | tgccttcctt gaccctggaa ggtgccactc ccactgtcct ttcctaataa |
| | | aatgaggaaa ttgcatcgca ttgtctgagt aggtgtcatt ctattctggg |
| | | gggtggggtg gggcaggaca gcaaggggga ggattgggaa gacaatagca |
| | | ggcatgctgg ggatgcggtg ggctctatgg gtacccaggt gctgaagaat |
| | | tgacccggtt cctcctgggc cagaaagaag caggcacatc cccttctctg |
| | | tgacacaccc tgtccacgcc cctggttctt agttccagcc ccactcatag |
| | | gacactcata gctcaggagg gctccgcctt caatcccacc cgctaaagta |
| | | cttggagcgg tctctccctc cctcatcagc ccaccaaacc aaacctagcc |
| | | tccaagagtg ggaagaaatt aaagcaagat aggctattaa gtgcagaggg |
| | | agagaaaatg cctccaacat gtgaggaagt aatgagagaa atcatagaat |
| | | tttaaggcca tgatttaagg ccatcatggc cttaatcttc cgcttcctcg |
| | | ctcactgact cgctgcgctc ggtcgttcgg ctgcggcgag cggtatcagc |
| | | tcactcaaag gcggtaatac ggttatccac agaatcaggg gataacgcag |
| | | gaaagaacat gtgagcaaaa ggccagcaaa aggccaggaa ccgtaaaaag |
| | | gccgcgttgc tggcgttttt ccataggctc cgcccccctg acgagcatca |
| | | caaaaatcga cgctcaagtc agaggtggcg aaacccgaca ggactataaa |
| | | gataccaggc gtttccccct ggaagctccc tcgtgcgctc tcctgttccg |
| | | accctgccgc ttaccggata cctgtccgcc tttctccctt cgggaagcgt |
| | | ggcgctttct catagctcac gctgtaggta tctcagttcg gtgtaggtcg |
| | | ttcgctccaa gctgggctgt gtgcacgaac cccccgttca gcccgaccgc |
| | | tgcgccttat ccggtaacta tcgtcttgag tccaacccgg taagacacga |
| | | cttatcgcca ctggcagcag ccactggtaa caggattagc agagcgaggt |
| | | atgtaggcgg tgctacagag ttcttgaagt ggtggcctaa ctacggctac |
| | | actagaagaa cagtatttgg tatctgcgct ctgctgaagc cagttacctt |
| | | cggaaaaaga gttggtagct cttgatccgg caaacaaacc accgctggta |
| | | gcggtggttt ttttgtttgc aagcagcaga ttacgcgcag aaaaaaagga |
| | | tctcaagaag atcctttgat cttttctacg gggtctgacg ctcagtggaa |
| | | cgaaaactca cgttaaggga ttttggtcat gagattatca aaaaggatct |
| | | tcacctagat ccttttaaat taaaaatgaa gttttaaatc aatctaaagt |
| | | atatatgagt aaacttggtc tgacagttac caatgcttaa tcagtgaggc |
| | | acctatctca gcgatctgtc tatttcgttc atccatagtt gcctgactcg |
| | | gggggggggg gcgctgaggt ctgcctcgtg aagaaggtgt tgctgactca |
| | | taccaggcct gaatcgcccc atcatccagc cagaaagtga gggagccacg |
| | | gttgatgaga gctttgttgt aggtggacca gttggtgatt ttgaactttt |
| | | gctttgccac ggaacggtct gcgttgtcgg gaagatgcgt gatctgatcc |
| | | ttcaactcag caaaagttcg atttattcaa caaagccgcc gtcccgtcaa |
| | | gtcagcgtaa tgctctgcca gtgttacaac caattaacca attctgatta |
| | | gaaaaactca tcgagcatca aatgaaactg caatttattc atatcaggat |
| | | tatcaatacc atatttttga aaaagccgtt tctgtaatga aggagaaaac |
| | | tcaccgaggc agttccatag gatggcaaga tcctggtatc ggtctgcgat |
| | | tccgactcgt ccaacatcaa tacaacctat taatttcccc tcgtcaaaaa |
| | | taaggttatc aagtgagaaa tcaccatgag tgacgactga atccggtgag |
| | | aatggcaaaa gcttatgcat ttctttccag acttgttcaa caggccagcc |
| | | attacgctcg tcatcaaaat cactcgcatc aaccaaaccg ttattcattc |
| | | gtgattgcgc ctgagcgaga cgaaatacgc gatcgctgtt aaaaggacaa |
| | | ttacaaacag gaatcgaatg caaccggcgc aggaacactg ccagcgcatc |
| | | aacaatattt tcacctgaat caggatattc ttctaatacc tggaatgctg |
| | | ttttcccggg gatcgcagtg gtgagtaacc atgcatcatc aggagtacgg |
| | | ataaaatgct tgatggtcgg aagaggcata aattccgtca gccagtttag |
| | | tctgaccatc tcatctgtaa catcattggc aacgctacct ttgccatgtt |
| | | tcagaaacaa ctctggcgca tcgggcttcc catacaatcg atagattgtc |
| | | gcacctgatt gcccgacatt atcgcgagcc catttatacc catataaatc |
| | | agcatccatg ttggaattta atcgcggcct cgagcaagac gtttcccgtt |
| | | gaatatggct cataacaccc cttgtattac tgtttatgta agcagacagt |
| | | tttattgttc atgatgatat atttttatct tgtgcaatgt aacatcagag |
| | | attttgagac acaacgtggc tttccccccc cccccattat tgaagcattt |
| | | atcagggtta ttgtctcatg agcggataca tatttgaatg tatttagaaa |
| | | aataaacaaa taggggttcc gcgcacattt ccccgaaaag tgccacctga |
| | | cgtctaagaa accattatta tcatgacatt aacctataaa aataggcgta |
| | | tcacgaggcc ctttcgtc |
| 123 | Humanized H02 light chain | MGWSCIILFLVATATGVHSQAALTQPPSVSGSPGQSV |
| | backbone aa sequence | TISCTGTSSDIGGYNYVSWYQQHPGKAPKVMIYEVS |
| | | KRPSGVSDRFSGSKSGNIASLTISGLQAEDEADYYCS |
| | | SYAGSNTFLFGGGTRLTVLGQPKAAPSVTLFPPSSEE |
| | | LQANKATLVCLISDFYPGAVTVAWKADSSPVKAGV |
| | | ETTTPSKQSNNKYAASSYLSLTPEQWKSHRSYSCQV |
| | | THEGSTVEKTVAPTECS |
| 124 | EBV gp350 GenBank: | MEAALLVCQYTIQSLIHLTGEDPGFFNVEIPEFPFYPTC |
| | AIM62208.1 | NVCTADVNVTINFDVGGKKHQLDLDFGQLTPHTKAVYQP |
| | | RGAFGGSENATNLFLLELLGAGELALTMRSKKLPINVTT |
| | | GEEQQVSLESVDVYFQDVFGTMWCHHAEMQNPVYLIPET |
| | | VPYIKWDNCNSTNITAVVRAQGLDVTLPLSLPTSAQDSN |
| | | FSVKTEMLGNEIDIECIMEDGEISQVLPGDNKFNITCSG |
| | | YESHVPSGGILTSTSPVATPIPGTGYAYSLRLTPRPVSR |
| | | FLGNNSILYVFYSGNGPKASGGDYCIQSNIVFSDEIPAS |
| | | QDMPTNTTDITYVGDNATYSVPMVTSEDANSPNVTVTAF |
| | | WAWPNNTETDFKCKWTLTSGTPSGCENISGAFASNRTFD |
| | | ITVSGLGTAPKTLIITRTATNATTTTHKVIFSKAPESTT |
| | | TSPTSNTTGFAAPNTTTGLPSSTHVPTNLTAPASTGPTV |
| | | STADVTSPTPAGTTSGASPVTPSPSPRDNGTESKAPDMT |
| | | SPTSAVTTPTPNATSPTSAVTTPTPNATSPTPAVTTPTP |
| | | NATSPTLGKTSPTSAVTTPTPNATSPTLGKTSPTSAVTT |
| | | PTPNATSPTLGKTSPTSAVTTPTPNATSPTVGETSPQAN |
| | | TTNHTLGGTSSTPVVTSPPKNATSAVTTGQHNITSSSTS |
| | | SMSLRPSSISETLSPSTSDNSTSHMPLLTSAHPTGGENI |
| | | TQVTPASTSTHHVSTSSPAPRPGTTSQASGPGNSSTSTK |
| | | PGEVNVTKGTPPKNATSPQAPSGQKTAVPTVTSTGGKAN |
| | | STTGGKHTTGHGARTSTEPTTDYGGDSTTPRTRYNATTY |
| | | LPPSTSSELRPRWTFTSPPVTTAQATVPVPPTSQPRFSN |
| | | LSMLVLQWASLAVLTLLLLLVMADCAFRRNLSTSHTYTT |
| | | PPYDDAETYV |
| 125 | gp350 B95-8 amino acids | EAALLVCQYTIQSLIHLTGEDPGFFNVEIPEFPFYPTCN |
| | 2-425 Avi | VCTADVNVTINFDVGGKKHQLDLDFGQLTPHTKAVYQPR |
| | | GAFGGSENATNLFLLELLGAGELALTMRSKKLPINVTTG |
| | | EEQQVSLESVDVYFQDVFGTMWCHHAEMQNPVYLIPETV |
| | | PYIKWDNCNSTNITAVVRAQGLDVTLPLSLPTSAQDSNF |
| | | SVKTEMLGNEIDIECIMEDGEISQVLPGDNKFNITCSGY |
| | | ESHVPSGGILTSTSPVATPIPGTGYAYSLRLTPRPVSRF |
| | | LGNNSILYVFYSGNGPKASGGDYCIQSNIVFSDEIPASQ |
| | | DMPTNTTDITYVGDNATYSVPMVTSEDANSPNVTVTAFW |
| | | AWPNNTETDFKCKWTLTSGTPSGCENISGAFASNRTFDI |
| | | TVSGLGTAPKTLIITRTATNATTTTHKVIFSKAPGS |
| 126 | NAM encoding SEQ ID | gaagctgccctgctcgtgtgccagtacaccatccagagcctgatccacctgaccg |
| | NO: 125 | gcgaggaccccggcttcttcaacgtggaaatccccgagttccccttctaccctacct |
| | | gcaacgtgtgcaccgccgacgtgaacgtgaccatcaacttcgacgtgggcggca |
| | | agaagcaccagctggacctggatttcggccagctgacccctcacaccaaggccgt |
| | | gtatcagcccagaggcgcctttggcggcagcgagaacgccaccaatctgtttctgc |
| | | tggaactcctaggcgccggcgagctggccctgaccatgagaagcaagaaactgc |
| | | ccatcaatgtgaccacaggcgaggaacagcaggtgtccctggaaagcgtgg acg |
| | | tgtactttcaagacgtgttcggcaccatgtggtgccaccacgccgagatgcagaac |
| | | cccgtgtacctgatccccgagacagtgccctacatcaagtgggacaactgcaaca |
| | | gcaccaacatcaccgccgtcgtgcgggcccagggactggatgtgacactgcctct |
| | | gagcctgcctaccagcgcccaggacagcaacttcagcgtgaaaaccgagatgct |
| | | gggcaacgagatcgacatcgagtgcatcatggaagatggcgagatcagccaggt |
| | | gctgcccggcgacaacaagttcaacatcacatgcagcggctacgagagccacgt |
| | | gccatctggcggcatcctgaccagcacaagcccagtggccacacccatccctgg |
| | | cacaggctacgcctacagcctgagactgacccccagacccgtgtccagattcctg |
| | | ggcaacaacagcatcctgtacgtgttctacagcggcaacggccccaaggcctctg |
| | | gcggcgattactgtatccagagcaacatcgtgttcagcgacgagatccccgccag |
| | | ccaggacatgcccaccaataccaccgacatcacgtacgtgggcgacaatgccac |
| | | ctacagcgtgccaatggtcacctccgaggacgccaacagccccaacgtgaccgt |
| | | gacagccttttgggcctggcctaacaacaccgagacagacttcaagtgcaagtgg |
| | | accctgacctccggcacccctagcggctgcgagaatatcagcggagccttcgcca |
| | | gcaaccggaccttcgatatcaccgtgtctggcctgggcaccgcccccaagaccct |
| | | gatcatcaccagaaccgccacaaatgccaccaccacaacccacaaagtgatcttc |
| | | agcaaggcccccggctctggc |
| 127 | gp350 B95-8 (2-425) | EAALLVCQYTIQSLIHLTGEDPGFFNVEIPEFPFYPTCN |
| | Q122N*Avi | VCTADVNVTINFDVGGKKHQLDLDFGQLTPHTKAVYQPR |
| | | GAFGGSENATNLFLLELLGAGELALTMRSKKLPINVTTG |
| | | EEQNVSLESVDVYFQDVFGTMWCHHAEMQNPVYLIPETV |
| | | PYIKWDNCNSTNITAVVRAQGLDVTLPLSLPTSAQDSNF |
| | | SVKTEMLGNEIDIECIMEDGEISQVLPGDNKFNITCSGY |
| | | ESHVPSGGILTSTSPVATPIPGTGYAYSLRLTPRPVSRF |
| | | LGNNSILYVFYSGNGPKASGGDYCIQSNIVFSDEIPASQ |
| | | DMPTNTTDITYVGDNATYSVPMVTSEDANSPNVTVTAFW |
| | | AWPNNTETDFKCKWTLTSGTPSGCENISGAFASNRTFDI |
| | | TVSGLGTAPKTLIITRTATNATTTTHKVIFSKAPGS |
| 128 | NAM encoding SEQ ID | gaagctgccctgctcgtgtgccagtacaccatccagagcctgatccacctgaccg |
| | NO: 127 | gcgaggaccccggcttcttcaacgtggaaatccccgagttccccttctaccctacct |
| | | gcaacgtgtgcaccgccgacgtgaacgtgaccatcaacttcgacgtgggcggca |
| | | agaagcaccagctggacctggatttcggccagctgacccctcacaccaaggccgt |
| | | gtatcagcccagaggcgcctttggcggcagcgagaacgccaccaatctgtttctgc |
| | | tggaactcctaggcgccggcgagctggccctgaccatgagaagcaagaaactgc |
| | | ccatcaatgtgaccacaggcgaggaacagaacgtgtccctggaaagcgtggacg |
| | | tgtactttcaagacgtgttcggcaccatgtggtgccaccacgccgagatgcagaac |
| | | cccgtgtacctgatccccgagacagtgccctacatcaagtgggacaactgcaaca |
| | | gcaccaacatcaccgccgtcgtgcgggcccagggactggatgtgacactgcctct |
| | | gagcctgcctaccagcgcccaggacagcaacttcagcgtgaaaaccgagatgct |
| | | gggcaacgagatcgacatcgagtgcatcatggaagatggcgagatcagccaggt |
| | | gctgcccggcgacaacaagttcaacatcacatgcagcggctacgagagccacgt |
| | | gccatctggcggcatcctgaccagcacaagcccagtggccacacccatccctgg |
| | | cacaggctacgcctacagcctgagactgacccccagacccgtgtccagattcctg |
| | | ggcaacaacagcatcctgtacgtgttctacagcggcaacggccccaaggcctctg |
| | | gcggcgattactgtatccagagcaacatcgtgttcagcgacgagatccccgccag |
| | | ccaggacatgcccaccaataccaccgacatcacgtacgtgggcgacaatgccac |
| | | ctacagcgtgccaatggtcacctccgaggacgccaacagccccaacgtgaccgt |
| | | gacagccttttgggcctggcctaacaacaccgagacagacttcaagtgcaagtgg |
| | | accctgacctccggcacccctagcggctgcgagaatatcagcggagccttcgcca |
| | | gcaaccggaccttcgatatcaccgtgtctggcctgggcaccgcccccaagaccct |
| | | gatcatcaccagaaccgccacaaatgccaccaccacaacccacaaagtgatcttc |
| | | agcaaggcccccggctct |
| 129 | gp350 B95-8 (2-425) | EAALLVCQYTIQSLIHLTGEDPGFFNVEIPEFPFYPTCN |
| | D163N*/164bSAvi | VCTADVNVTINFDVGGKKHQLDLDFGQLTPHTKAVYQPR |
| | | GAFGGSENATNLFLLELLGAGELALTMRSKKLPINVTTG |
| | | EEQQVSLESVDVYFQDVFGTMWCHHAEMQNPVYLIPETV |
| | | PYIKWNNSCNSTNITAVVRAQGLDVTLPLSLPTSAQDSN |
| | | FSVKTEMLGNEIDIECIMEDGEISQVLPGDNKFNITCSG |
| | | YESHVPSGGILTSTSPVATPIPGTGYAYSLRLTPRPVSR |
| | | FLGNNSILYVFYSGNGPKASGGDYCIQSNIVFSDEIPAS |
| | | QDMPTNTTDITYVGDNATYSVPMVTSEDANSPNVTVTAF |
| | | WAWPNNTETDFKCKWTLTSGTPSGCENISGAFASNRTFD |
| | | ITVSGLGTAPKTLIITRTATNATTTTHKVIFSKAPGS |
| 130 | NAM encoding SEQ ID | gaagctgccctgctcgtgtgccagtacaccatccagagcctgatccacctgaccg |
| | NO: 129 | gcgaggaccccggcttcttcaacgtggaaatccccgagttccccttctaccctacct |
| | | gcaacgtgtgcaccgccgacgtgaacgtgaccatcaacttcgacgtgggcggca |
| | | agaagcaccagctggacctggatttcggccagctgacccctcacaccaaggccgt |
| | | gtatcagcccagaggcgcctttggcggcagcgagaacgccaccaatctgtttctgc |
| | | tggaactcctaggcgccggcgagctggccctgaccatgagaagcaagaaactgc |
| | | ccatcaatgtgaccacaggcgaggaacagcaggtgtccctggaaagcgtgg acg |
| | | tgtactttcaagacgtgttcggcaccatgtggtgccaccacgccgagatgcagaac |
| | | cccgtgtacctgatccccgagacagtgccctacatcaagtggaacaacagctgca |
| | | acagcaccaacatcaccgccgtcgtgcgggcccagggactggatgtgacactgc |
| | | ctctgagcctgcctaccagcgcccaggacagcaacttcagcgtgaaaaccgagat |
| | | gctgggcaacgagatcgacatcgagtgcatcatggaagatggcgagatcagcca |
| | | ggtgctgcccggcgacaacaagttcaacatcacatgcagcggctacgagagcca |
| | | cgtgccatctggcggcatcctgaccagcacaagcccagtggccacacccatccct |
| | | ggcacaggctacgcctacagcctgagactgacccccagacccgtgtccagattcc |
| | | tgggcaacaacagcatcctgtacgtgttctacagcggcaacggccccaaggcctc |
| | | tggcggcgattactgtatccagagcaacatcgtgttcagcgacgagatccccgcca |
| | | gccaggacatgcccaccaataccaccgacatcacgtacgtgggcgacaatgcca |
| | | cctacagcgtgccaatggtcacctccgaggacgccaacagccccaacgtgaccg |
| | | tgacagccttttgggcctggcctaacaacaccgagacagacttcaagtgcaagtgg |
| | | accctgacctccggcacccctagcggctgcgagaatatcagcggagccttcgcca |
| | | gcaaccggaccttcgatatcaccgtgtctggcctgggcaccgcccccaagaccct |
| | | gatcatcaccagaaccgccacaaatgccaccaccacaacccacaaagtgatcttc |
| | | agcaaggcccccggctct |
| 131 | gp350 B95-8 (2-425) | EAALLVCQYTIQSLIHLTGEDPGFFNVEIPEFPFYPTCN |
| | D296R Avi | VCTADVNVTINFDVGGKKHQLDLDFGQLTPHTKAVYQPR |
| | | GAFGGSENATNLFLLELLGAGELALTMRSKKLPINVTTG |
| | | EEQQVSLESVDVYFQDVFGTMWCHHAEMQNPVYLIPETV |
| | | PYIKWDNCNSTNITAVVRAQGLDVTLPLSLPTSAQDSNF |
| | | SVKTEMLGNEIDIECIMEDGEISQVLPGDNKFNITCSGY |
| | | ESHVPSGGILTSTSPVATPIPGTGYAYSLRLTPRPVSRF |
| | | LGNNSILYVFYSGNGPKASGGRYCIQSNIVFSDEIPASQ |
| | | DMPTNTTDITYVGDNATYSVPMVTSEDANSPNVTVTAFW |
| | | AWPNNTETDFKCKWTLTSGTPSGCENISGAFASNRTFDI |
| | | TVSGLGTAPKTLIITRTATNATTTTHKVIFSKAPGS |
| 132 | NAM encoding SEQ ID | gaagctgccctgctcgtgtgccagtacaccatccagagcctgatccacctgaccg |
| | NO: 131 | gcgaggaccccggcttcttcaacgtggaaatccccgagttccccttctaccctacct |
| | | gcaacgtgtgcaccgccgacgtgaacgtgaccatcaacttcgacgtgggcggca |
| | | agaagcaccagctggacctggatttcggccagctgacccctcacaccaaggccgt |
| | | gtatcagcccagaggcgcctttggcggcagcgagaacgccaccaatctgtttctgc |
| | | tggaactcctaggcgccggcgagctggccctgaccatgagaagcaagaaactgc |
| | | ccatcaatgtgaccacaggcgaggaacagcaggtgtccctggaaagcgtggacg |
| | | tgtactttcaagacgtgttcggcaccatgtggtgccaccacgccgagatgcagaac |
| | | cccgtgtacctgatccccgagacagtgccctacatcaagtgggacaactgcaaca |
| | | gcaccaacatcaccgccgtcgtgcgggcccagggactggatgtgacactgcctct |
| | | gagcctgcctaccagcgcccaggacagcaacttcagcgtgaaaaccgagatgct |
| | | gggcaacgagatcgacatcgagtgcatcatggaagatggcgagatcagccaggt |
| | | gctgcccggcgacaacaagttcaacatcacatgcagcggctacgagagccacgt |
| | | gccatctggcggcatcctgaccagcacaagcccagtggccacacccatccctgg |
| | | cacaggctacgcctacagcctgagactgacccccagacccgtgtccagattcctg |
| | | ggcaacaacagcatcctgtacgtgttctacagcggcaacggccccaaggcctctg |
| | | gcggccggtactgtatccagagcaacatcgtgttcagcgacgagatccccgccag |
| | | ccaggacatgcccaccaataccaccgacatcacgtacgtgggcgacaatgccac |
| | | ctacagcgtgccaatggtcacctccgaggacgccaacagccccaacgtgaccgt |
| | | gacagccttttgggcctggcctaacaacaccgagacagacttcaagtgcaagtgg |
| | | accctgacctccggcacccctagcggctgcgagaatatcagcggagccttcgcca |
| | | gcaaccggaccttcgatatcaccgtgtctggcctgggcaccgcccccaagaccct |
| | | gatcatcaccagaaccgccacaaatgccaccaccacaacccacaaagtgatcttc |
| | | agcaaggcccccggctct |
| 133 | gp350 B95-8 (2-425) | EAALLVCQYTIQSLIHLTGEDPGFFNVEIPEFPFYPTCN |
| | WDN162-4AAA Avi | VCTADVNVTINFDVGGKKHQLDLDFGQLTPHTKAVYQPR |
| | | GAFGGSENATNLFLLELLGAGELALTMRSKKLPINVTTG |
| | | EEQQVSLESVDVYFQDVFGTMWCHHAEMQNPVYLIPETV |
| | | PYIKAAACNSTNITAVVRAQGLDVTLPLSLPTSAQDSNF |
| | | SVKTEMLGNEIDIECIMEDGEISQVLPGDNKFNITCSGY |
| | | ESHVPSGGILTSTSPVATPIPGTGYAYSLRLTPRPVSRF |
| | | LGNNSILYVFYSGNGPKASGGDYCIQSNIVFSDEIPASQ |
| | | DMPTNTTDITYVGDNATYSVPMVTSEDANSPNVTVTAFW |
| | | AWPNNTETDFKCKWTLTSGTPSGCENISGAFASNRTFDI |
| | | TVSGLGTAPKTLIITRTATNATTTTHKVIFSKAPGS |
| 134 | NAM encoding SEQ ID | gaagctgccctgctcgtgtgccagtacaccatccagagcctgatccacctgaccg |
| | NO: 133 | gcgaggaccccggcttcttcaacgtggaaatccccgagttccccttctaccctacct |
| | | gcaacgtgtgcaccgccgacgtgaacgtgaccatcaacttcgacgtgggcggca |
| | | agaagcaccagctggacctggatttcggccagctgacccctcacaccaaggccgt |
| | | gtatcagcccagaggcgcctttggcggcagcgagaacgccaccaatctgtttctgc |
| | | tggaactcctaggcgccggcgagctggccctgaccatgagaagcaagaaactgc |
| | | ccatcaatgtgaccacaggcgaggaacagcaggtgtccctggaaagcgtgg acg |
| | | tgtactttcaagacgtgttcggcaccatgtggtgccaccacgccgagatgcagaac |
| | | cccgtgtacctgatccccgagacagtgccctacatcaaggccgccgcctgcaaca |
| | | gcaccaacatcaccgccgtcgtgcgggcccagggactggatgtgacactgcctct |
| | | gagcctgcctaccagcgcccaggacagcaacttcagcgtgaaaaccgagatgct |
| | | gggcaacgagatcgacatcgagtgcatcatggaagatggcgagatcagccaggt |
| | | gctgcccggcgacaacaagttcaacatcacatgcagcggctacgagagccacgt |
| | | gccatctggcggcatcctgaccagcacaagcccagtggccacacccatccctgg |
| | | cacaggctacgcctacagcctgagactgacccccagacccgtgtccagattcctg |
| | | ggcaacaacagcatcctgtacgtgttctacagcggcaacggccccaaggcctctg |
| | | gcggcgattactgtatccagagcaacatcgtgttcagcgacgagatccccgccag |
| | | ccaggacatgcccaccaataccaccgacatcacgtacgtgggcgacaatgccac |
| | | ctacagcgtgccaatggtcacctccgaggacgccaacagccccaacgtgaccgt |
| | | gacagccttttgggcctggcctaacaacaccgagacagacttcaagtgcaagtgg |
| | | accctgacctccggcacccctagcggctgcgagaatatcagcggagccttcgcca |
| | | gcaaccggaccttcgatatcaccgtgtctggcctgggcaccgcccccaagaccct |
| | | gatcatcaccagaaccgccacaaatgccaccaccacaacccacaaagtgatcttc |
| | | agcaaggcccccggctct |
| 135 | gp350 B95-8 (2-425) | EAALLVCQYTIQSLIHLTGEDPGFFNVEIPEFPFYPTCN |
| | D296R/1160R Avi | VCTADVNVTINFDVGGKKHQLDLDFGQLTPHTKAVYQPR |
| | | GAFGGSENATNLFLLELLGAGELALTMRSKKLPINVTTG |
| | | EEQQVSLESVDVYFQDVFGTMWCHHAEMQNPVYLIPETV |
| | | PYRKWDNCNSTNITAVVRAQGLDVTLPLSLPTSAQDSNF |
| | | SVKTEMLGNEIDIECIMEDGEISQVLPGDNKFNITCSGY |
| | | ESHVPSGGILTSTSPVATPIPGTGYAYSLRLTPRPVSRF |
| | | LGNNSILYVFYSGNGPKASGGRYCIQSNIVFSDEIPASQ |
| | | DMPTNTTDITYVGDNATYSVPMVTSEDANSPNVTVTAFW |
| | | AWPNNTETDFKCKWTLTSGTPSGCENISGAFASNRTFDI |
| | | TVSGLGTAPKTLIITRTATNATTTTHKVIFSKAPGS |
| 136 | NAM encoding SEQ ID | gaagctgccctgctcgtgtgccagtacaccatccagagcctgatccacctgaccg |
| | NO: 135 | gcgaggaccccggcttcttcaacgtggaaatccccgagttccccttctaccctacct |
| | | gcaacgtgtgcaccgccgacgtgaacgtgaccatcaacttcgacgtgggcggca |
| | | agaagcaccagctggacctggatttcggccagctgacccctcacaccaaggccgt |
| | | gtatcagcccagaggcgcctttggcggcagcgagaacgccaccaatctgtttctgc |
| | | tggaactcctaggcgccggcgagctggccctgaccatgagaagcaagaaactgc |
| | | ccatcaatgtgaccacaggcgaggaacagcaggtgtccctggaaagcgtggacg |
| | | tgtactttcaagacgtgttcggcaccatgtggtgccaccacgccgagatgcagaac |
| | | cccgtgtacctgatccccgagacagtgccctaccggaagtgggacaactgcaaca |
| | | gcaccaacatcaccgccgtcgtgcgggcccagggactggatgtgacactgcctct |
| | | gagcctgcctaccagcgcccaggacagcaacttcagcgtgaaaaccgagatgct |
| | | gggcaacgagatcgacatcgagtgcatcatggaagatggcgagatcagccaggt |
| | | gctgcccggcgacaacaagttcaacatcacatgcagcggctacgagagccacgt |
| | | gccatctggcggcatcctgaccagcacaagcccagtggccacacccatccctgg |
| | | cacaggctacgcctacagcctgagactgacccccagacccgtgtccagattcctg |
| | | ggcaacaacagcatcctgtacgtgttctacagcggcaacggccccaaggcctctg |
| | | gcggccggtactgtatccagagcaacatcgtgttcagcgacgagatccccgccag |
| | | ccaggacatgcccaccaataccaccgacatcacgtacgtgggcgacaatgccac |
| | | ctacagcgtgccaatggtcacctccgaggacgccaacagccccaacgtgaccgt |
| | | gacagccttttgggcctggcctaacaacaccgagacagacttcaagtgcaagtgg |
| | | accctgacctccggcacccctagcggctgcgagaatatcagcggagccttcgcca |
| | | gcaaccggaccttcgatatcaccgtgtctggcctgggcaccgcccccaagaccct |
| | | gatcatcaccagaaccgccacaaatgccaccaccacaacccacaaagtgatcttc |
| | | agcaaggcccccggctct |
| 137 | gp350 B95-8 (2-425) | EAALLVCQYTIQSLIHLTGEDPGFFNVEIPEFPFYPTCN |
| | D296R/P158R Avi | VCTADVNVTINFDVGGKKHQLDLDFGQLTPHTKAVYQPR |
| | | GAFGGSENATNLFLLELLGAGELALTMRSKKLPINVTTG |
| | | EEQQVSLESVDVYFQDVFGTMWCHHAEMQNPVYLIPETV |
| | | RYIKWDNCNSTNITAVVRAQGLDVTLPLSLPTSAQDSNF |
| | | SVKTEMLGNEIDIECIMEDGEISQVLPGDNKFNITCSGY |
| | | ESHVPSGGILTSTSPVATPIPGTGYAYSLRLTPRPVSRF |
| | | LGNNSILYVFYSGNGPKASGGRYCIQSNIVFSDEIPASQ |
| | | DMPTNTTDITYVGDNATYSVPMVTSEDANSPNVTVTAFW |
| | | AWPNNTETDFKCKWTLTSGTPSGCENISGAFASNRTFDI |
| | | TVSGLGTAPKTLIITRTATNATTTTHKVIFSKAPGS |
| 138 | NAM encoding SEQ ID | gaagctgccctgctcgtgtgccagtacaccatccagagcctgatccacctgaccg |
| | NO: 137 | gcgaggaccccggcttcttcaacgtggaaatccccgagttccccttctaccctacct |
| | | gcaacgtgtgcaccgccgacgtgaacgtgaccatcaacttcgacgtgggcggca |
| | | agaagcaccagctggacctggatttcggccagctgacccctcacaccaaggccgt |
| | | gtatcagcccagaggcgcctttggcggcagcgagaacgccaccaatctgtttctgc |
| | | tggaactcctaggcgccggcgagctggccctgaccatgagaagcaagaaactgc |
| | | ccatcaatgtgaccacaggcgaggaacagcaggtgtccctggaaagcgtgg acg |
| | | tgtactttcaagacgtgttcggcaccatgtggtgccaccacgccgagatgcagaac |
| | | cccgtgtacctgatccccgagacagtgcggtacatcaagtgggacaactgcaaca |
| | | gcaccaacatcaccgccgtcgtgcgggcccagggactggatgtgacactgcctct |
| | | gagcctgcctaccagcgcccaggacagcaacttcagcgtgaaaaccgagatgct |
| | | gggcaacgagatcgacatcgagtgcatcatggaagatggcgagatcagccaggt |
| | | gctgcccggcgacaacaagttcaacatcacatgcagcggctacgagagccacgt |
| | | gccatctggcggcatcctgaccagcacaagcccagtggccacacccatccctgg |
| | | cacaggctacgcctacagcctgagactgacccccagacccgtgtccagattcctg |
| | | ggcaacaacagcatcctgtacgtgttctacagcggcaacggccccaaggcctctg |
| | | gcggccggtactgtatccagagcaacatcgtgttcagcgacgagatccccgccag |
| | | ccaggacatgcccaccaataccaccgacatcacgtacgtgggcgacaatgccac |
| | | ctacagcgtgccaatggtcacctccgaggacgccaacagccccaacgtgaccgt |
| | | gacagccttttgggcctggcctaacaacaccgagacagacttcaagtgcaagtgg |
| | | accctgacctccggcacccctagcggctgcgagaatatcagcggagccttcgcca |
| | | gcaaccggaccttcgatatcaccgtgtctggcctgggcaccgcccccaagaccct |
| | | gatcatcaccagaaccgccacaaatgccaccaccacaacccacaaagtgatcttc |
| | | agcaaggcccccggctct |
| 139 | gp350 B95-8 (2-425) | EAALLVCQYTIQSLIHLTGEDPGFFNVEIPEFPFYPTCN |
| | D296R/P158N*/I160T Avi | VCTADVNVTINFDVGGKKHQLDLDFGQLTPHTKAVYQPR |
| | | GAFGGSENATNLFLLELLGAGELALTMRSKKLPINVTTG |
| | | EEQQVSLESVDVYFQDVFGTMWCHHAEMQNPVYLIPETV |
| | | NYTKWDNCNSTNITAVVRAQGLDVTLPLSLPTSAQDSNF |
| | | SVKTEMLGNEIDIECIMEDGEISQVLPGDNKFNITCSGY |
| | | ESHVPSGGILTSTSPVATPIPGTGYAYSLRLTPRPVSRF |
| | | LGNNSILYVFYSGNGPKASGGRYCIQSNIVFSDEIPASQ |
| | | DMPTNTTDITYVGDNATYSVPMVTSEDANSPNVTVTAFW |
| | | AWPNNTETDFKCKWTLTSGTPSGCENISGAFASNRTFDI |
| | | TVSGLGTAPKTLIITRTATNATTTTHKVIFSKAPGS |
| 140 | NAM encoding SEQ ID | gaagctgccctgctcgtgtgccagtacaccatccagagcctgatccacctgaccg |
| | NO: 139 | gcgaggaccccggcttcttcaacgtggaaatccccgagttccccttctaccctacct |
| | | gcaacgtgtgcaccgccgacgtgaacgtgaccatcaacttcgacgtgggcggca |
| | | agaagcaccagctggacctggatttcggccagctgacccctcacaccaaggccgt |
| | | gtatcagcccagaggcgcctttggcggcagcgagaacgccaccaatctgtttctgc |
| | | tggaactcctaggcgccggcgagctggccctgaccatgagaagcaagaaactgc |
| | | ccatcaatgtgaccacaggcgaggaacagcaggtgtccctggaaagcgtggacg |
| | | tgtactttcaagacgtgttcggcaccatgtggtgccaccacgccgagatgcagaac |
| | | cccgtgtacctgatccccgagacagtgaactacaccaagtgggacaactgcaaca |
| | | gcaccaacatcaccgccgtcgtgcgggcccagggactggatgtgacactgcctct |
| | | gagcctgcctaccagcgcccaggacagcaacttcagcgtgaaaaccgagatgct |
| | | gggcaacgagatcgacatcgagtgcatcatggaagatggcgagatcagccaggt |
| | | gctgcccggcgacaacaagttcaacatcacatgcagcggctacgagagccacgt |
| | | gccatctggcggcatcctgaccagcacaagcccagtggccacacccatccctgg |
| | | cacaggctacgcctacagcctgagactgacccccagacccgtgtccagattcctg |
| | | ggcaacaacagcatcctgtacgtgttctacagcggcaacggccccaaggcctctg |
| | | gcggccggtactgtatccagagcaacatcgtgttcagcgacgagatccccgccag |
| | | ccaggacatgcccaccaataccaccgacatcacgtacgtgggcgacaatgccac |
| | | ctacagcgtgccaatggtcacctccgaggacgccaacagccccaacgtgaccgt |
| | | gacagccttttgggcctggcctaacaacaccgagacagacttcaagtgcaagtgg |
| | | accctgacctccggcacccctagcggctgcgagaatatcagcggagccttcgcca |
| | | gcaaccggaccttcgatatcaccgtgtctggcctgggcaccgcccccaagaccct |
| | | gatcatcaccagaaccgccacaaatgccaccaccacaacccacaaagtgatcttc |
| | | agcaaggcccccggctct |
| 141 | gp350 B95-8 (2-425) | EAALLVCQYTIQSLIHLTGEDPGFFNVEIPEFPFYPTCN |
| | D296R/P158R/I160R Avi | VCTADVNVTINFDVGGKKHQLDLDFGQLTPHTKAVYQPR |
| | | GAFGGSENATNLFLLELLGAGELALTMRSKKLPINVTTG |
| | | EEQQVSLESVDVYFQDVFGTMWCHHAEMQNPVYLIPETV |
| | | RYRKWDNCNSTNITAVVRAQGLDVTLPLSLPTSAQDSNF |
| | | SVKTEMLGNEIDIECIMEDGEISQVLPGDNKFNITCSGY |
| | | ESHVPSGGILTSTSPVATPIPGTGYAYSLRLTPRPVSRF |
| | | LGNNSILYVFYSGNGPKASGGRYCIQSNIVFSDEIPASQ |
| | | DMPTNTTDITYVGDNATYSVPMVTSEDANSPNVTVTAFW |
| | | AWPNNTETDFKCKWTLTSGTPSGCENISGAFASNRTFDI |
| | | TVSGLGTAPKTLIITRTATNATTTTHKVIFSKAPGS |
| 142 | NAM encoding SEQ ID | gaagctgccctgctcgtgtgccagtacaccatccagagcctgatccacctgaccg |
| | NO: 141 | gcgaggaccccggcttcttcaacgtggaaatccccgagttccccttctaccctacct |
| | | gcaacgtgtgcaccgccgacgtgaacgtgaccatcaacttcgacgtgggcggca |
| | | agaagcaccagctggacctggatttcggccagctgacccctcacaccaaggccgt |
| | | gtatcagcccagaggcgcctttggcggcagcgagaacgccaccaatctgtttctgc |
| | | tggaactcctaggcgccggcgagctggccctgaccatgagaagcaagaaactgc |
| | | ccatcaatgtgaccacaggcgaggaacagcaggtgtccctggaaagcgtgg acg |
| | | tgtactttcaagacgtgttcggcaccatgtggtgccaccacgccgagatgcagaac |
| | | cccgtgtacctgatccccgagacagtgcggtaccggaagtgggacaactgcaac |
| | | agcaccaacatcaccgccgtcgtgcgggcccagggactggatgtgacactgcct |
| | | ctgagcctgcctaccagcgcccaggacagcaacttcagcgtgaaaaccgagatg |
| | | ctgggcaacgagatcgacatcgagtgcatcatggaagatggcgagatcagccag |
| | | gtgctgcccggcgacaacaagttcaacatcacatgcagcggctacgagagccac |
| | | gtgccatctggcggcatcctgaccagcacaagcccagtggccacacccatccctg |
| | | gcacaggctacgcctacagcctgagactgacccccagacccgtgtccagattcct |
| | | gggcaacaacagcatcctgtacgtgttctacagcggcaacggccccaaggcctct |
| | | ggcggccggtactgtatccagagcaacatcgtgttcagcgacgagatccccgcca |
| | | gccaggacatgcccaccaataccaccgacatcacgtacgtgggcgacaatgcca |
| | | cctacagcgtgccaatggtcacctccgaggacgccaacagccccaacgtgaccg |
| | | tgacagccttttgggcctggcctaacaacaccgagacagacttcaagtgcaagtgg |
| | | accctgacctccggcacccctagcggctgcgagaatatcagcggagccttcgcca |
| | | gcaaccggaccttcgatatcaccgtgtctggcctgggcaccgcccccaagaccct |
| | | gatcatcaccagaaccgccacaaatgccaccaccacaacccacaaagtgatcttc |
| | | agcaaggcccccggctct |
| 143 | gp350 B95-8 (2-425) | EAALLVCQYTIQSLIHLTGEDPGFFNVEIPEFPFYPTCN |
| | Avi-His | VCTADVNVTINFDVGGKKHQLDLDFGQLTPHTKAVYQPR |
| | | GAFGGSENATNLFLLELLGAGELALTMRSKKLPINVTTG |
| | | EEQQVSLESVDVYFQDVFGTMWCHHAEMQNPVYLIPETV |
| | | PYIKWDNCNSTNITAVVRAQGLDVTLPLSLPTSAQDSNF |
| | | SVKTEMLGNEIDIECIMEDGEISQVLPGDNKFNITCSGY |
| | | ESHVPSGGILTSTSPVATPIPGTGYAYSLRLTPRPVSRF |
| | | LGNNSILYVFYSGNGPKASGGDYCIQSNIVFSDEIPASQ |
| | | DMPTNTTDITYVGDNATYSVPMVTSEDANSPNVTVTAFW |
| | | AWPNNTETDFKCKWTLTSGTPSGCENISGAFASNRTFDI |
| | | TVSGLGTAPKTLIITRTATNATTTTHKVIFSKAPGSGLN |
| | | DIFEAQKIEWHEHHHHHH |
| 144 | NAM encoding SEQ ID | gaagctgccctgctcgtgtgccagtacaccatccagagc |
| | NO: 143 | ctgatccacctgaccggcgaggaccccggcttcttcaac |
| | | gtggaaatccccgagttccccttctaccctacctgcaac |
| | | gtgtgcaccgccgacgtgaacgtgaccatcaacttcgac |
| | | gtgggcggcaagaagcaccagctggacctggatttcggc |
| | | cagctgacccctcacaccaaggccgtgtatcagcccaga |
| | | ggcgcctttggcggcagcgagaacgccaccaatctgttt |
| | | ctgctggaactcctaggcgccggcgagctggccctgacc |
| | | atgagaagcaagaaactgcccatcaatgtgaccacaggc |
| | | gaggaacagcaggtgtccctggaaagcgtggacgtgtac |
| | | tttcaagacgtgttcggcaccatgtggtgccaccacgcc |
| | | gagatgcagaaccccgtgtacctgatccccgagacagtg |
| | | ccctacatcaagtgggacaactgcaacagcaccaacatc |
| | | accgccgtcgtgcgggcccagggactggatgtgacactg |
| | | cctctgagcctgcctaccagcgcccaggacagcaacttc |
| | | agcgtgaaaaccgagatgctgggcaacgagatcgacatc |
| | | gagtgcatcatggaagatggcgagatcagccaggtgctg |
| | | cccggcgacaacaagttcaacatcacatgcagcggctac |
| | | gagagccacgtgccatctggcggcatcctgaccagcaca |
| | | agcccagtggccacacccatccctggcacaggctacgcc |
| | | tacagcctgagactgacccccagacccgtgtccagattc |
| | | ctgggcaacaacagcatcctgtacgtgttctacagcggc |
| | | aacggccccaaggcctctggcggcgattactgtatccag |
| | | agcaacatcgtgttcagcgacgagatccccgccagccag |
| | | gacatgcccaccaataccaccgacatcacgtacgtgggc |
| | | gacaatgccacctacagcgtgccaatggtcacctccgag |
| | | gacgccaacagccccaacgtgaccgtgacagccttttgg |
| | | gcctggcctaacaacaccgagacagacttcaagtgcaag |
| | | tggaccctgacctccggcacccctagcggctgcgagaat |
| | | atcagcggagccttcgccagcaaccggaccttcgatatc |
| | | accgtgtctggcctgggcaccgcccccaagaccctgatc |
| | | atcaccagaaccgccacaaatgccaccaccacaacccac |
| | | aaagtgatcttcagcaaggcccccggctctggcctgaac |
| | | gacatttttgaggcccagaagattgagtggcatgaacat |
| | | caccaccaccaccat |
| 145 | gp350 B95-8 (2-425) Q122N* | EAALLVCQYTIQSLIHLTGEDPGFFNVEIPEFPFYPTCN |
| | Avi-His | VCTADVNVTINFDVGGKKHQLDLDFGQLTPHTKAVYQPR |
| | | GAFGGSENATNLFLLELLGAGELALTMRSKKLPINVTTG |
| | | EEQNVSLESVDVYFQDVFGTMWCHHAEMQNPVYLIPETV |
| | | PYIKWDNCNSTNITAVVRAQGLDVTLPLSLPTSAQDSNF |
| | | SVKTEMLGNEIDIECIMEDGEISQVLPGDNKFNITCSGY |
| | | ESHVPSGGILTSTSPVATPIPGTGYAYSLRLTPRPVSRF |
| | | LGNNSILYVFYSGNGPKASGGDYCIQSNIVFSDEIPASQ |
| | | DMPTNTTDITYVGDNATYSVPMVTSEDANSPNVTVTAFW |
| | | AWPNNTETDFKCKWTLTSGTPSGCENISGAFASNRTFDI |
| | | TVSGLGTAPKTLIITRTATNATTTTHKVIFSKAPGSGLN |
| | | DIFEAQKIEWHEHHHHHH |
| 146 | NAM encoding SEQ ID | gaagctgccctgctcgtgtgccagtacaccatccagagc |
| | NO: 145 | ctgatccacctgaccggcgaggaccccggcttcttcaac |
| | | gtggaaatccccgagttccccttctaccctacctgcaac |
| | | gtgtgcaccgccgacgtgaacgtgaccatcaacttcgac |
| | | gtgggcggcaagaagcaccagctggacctggatttcggc |
| | | cagctgacccctcacaccaaggccgtgtatcagcccaga |
| | | ggcgcctttggcggcagcgagaacgccaccaatctgttt |
| | | ctgctggaactcctaggcgccggcgagctggccctgacc |
| | | atgagaagcaagaaactgcccatcaatgtgaccacaggc |
| | | gaggaacagaacgtgtccctggaaagcgtggacgtgtac |
| | | tttcaagacgtgttcggcaccatgtggtgccaccacgcc |
| | | gagatgcagaaccccgtgtacctgatccccgagacagtg |
| | | ccctacatcaagtgggacaactgcaacagcaccaacatc |
| | | accgccgtcgtgcgggcccagggactggatgtgacactg |
| | | cctctgagcctgcctaccagcgcccaggacagcaacttc |
| | | agcgtgaaaaccgagatgctgggcaacgagatcgacatc |
| | | gagtgcatcatggaagatggcgagatcagccaggtgctg |
| | | cccggcgacaacaagttcaacatcacatgcagcggctac |
| | | gagagccacgtgccatctggcggcatcctgaccagcaca |
| | | agcccagtggccacacccatccctggcacaggctacgcc |
| | | tacagcctgagactgacccccagacccgtgtccagattc |
| | | ctgggcaacaacagcatcctgtacgtgttctacagcggc |
| | | aacggccccaaggcctctggcggcgattactgtatccag |
| | | agcaacatcgtgttcagcgacgagatccccgccagccag |
| | | gacatgcccaccaataccaccgacatcacgtacgtgggc |
| | | gacaatgccacctacagcgtgccaatggtcacctccgag |
| | | gacgccaacagccccaacgtgaccgtgacagccttttgg |
| | | gcctggcctaacaacaccgagacagacttcaagtgcaag |
| | | tggaccctgacctccggcacccctagcggctgcgagaat |
| | | atcagcggagccttcgccagcaaccggaccttcgatatc |
| | | accgtgtctggcctgggcaccgcccccaagaccctgatc |
| | | atcaccagaaccgccacaaatgccaccaccacaacccac |
| | | aaagtgatcttcagcaaggcccccggctctggcctgaac |
| | | gacatttttgaggcccagaagattgagtggcatgaacat |
| | | caccaccaccaccat |
| 147 | gp350 B95-8 (2-425) | EAALLVCQYTIQSLIHLTGEDPGFFNVEIPEFPFYPTCN |
| | D163N*/164bS Avi-His | VCTADVNVTINFDVGGKKHQLDLDFGQLTPHTKAVYQPR |
| | | GAFGGSENATNLFLLELLGAGELALTMRSKKLPINVTTG |
| | | EEQQVSLESVDVYFQDVFGTMWCHHAEMQNPVYLIPETV |
| | | PYIKWNNSCNSTNITAVVRAQGLDVTLPLSLPTSAQDSN |
| | | FSVKTEMLGNEIDIECIMEDGEISQVLPGDNKFNITCSG |
| | | YESHVPSGGILTSTSPVATPIPGTGYAYSLRLTPRPVSR |
| | | FLGNNSILYVFYSGNGPKASGGDYCIQSNIVFSDEIPAS |
| | | QDMPTNTTDITYVGDNATYSVPMVTSEDANSPNVTVTAF |
| | | WAWPNNTETDFKCKWTLTSGTPSGCENISGAFASNRTFD |
| | | ITVSGLGTAPKTLIITRTATNATTTTHKVIFSKAPGSGL |
| | | NDIFEAQKIEWHEHHHHHH |
| 148 | NAM encoding SEQ ID | gaagctgccctgctcgtgtgccagtacaccatccagagc |
| | NO: 147 | ctgatccacctgaccggcgaggaccccggcttcttcaac |
| | | gtggaaatccccgagttccccttctaccctacctgcaac |
| | | gtgtgcaccgccgacgtgaacgtgaccatcaacttcgac |
| | | gtgggcggcaagaagcaccagctggacctggatttcggc |
| | | cagctgacccctcacaccaaggccgtgtatcagcccaga |
| | | ggcgcctttggcggcagcgagaacgccaccaatctgttt |
| | | ctgctggaactcctaggcgccggcgagctggccctgacc |
| | | atgagaagcaagaaactgcccatcaatgtgaccacaggc |
| | | gaggaacagcaggtgtccctggaaagcgtggacgtgtac |
| | | tttcaagacgtgttcggcaccatgtggtgccaccacgcc |
| | | gagatgcagaaccccgtgtacctgatccccgagacagtg |
| | | ccctacatcaagtggaacaacagctgcaacagcaccaac |
| | | atcaccgccgtcgtgcgggcccagggactggatgtgaca |
| | | ctgcctctgagcctgcctaccagcgcccaggacagcaac |
| | | ttcagcgtgaaaaccgagatgctgggcaacgagatcgac |
| | | atcgagtgcatcatggaagatggcgagatcagccaggtg |
| | | ctgcccggcgacaacaagttcaacatcacatgcagcggc |
| | | tacgagagccacgtgccatctggcggcatcctgaccagc |
| | | acaagcccagtggccacacccatccctggcacaggctac |
| | | gcctacagcctgagactgacccccagacccgtgtccaga |
| | | ttcctgggcaacaacagcatcctgtacgtgttctacagc |
| | | ggcaacggccccaaggcctctggcggcgattactgtatc |
| | | cagagcaacatcgtgttcagcgacgagatccccgccagc |
| | | caggacatgcccaccaataccaccgacatcacgtacgtg |
| | | ggcgacaatgccacctacagcgtgccaatggtcacctcc |
| | | gaggacgccaacagccccaacgtgaccgtgacagccttt |
| | | tgggcctggcctaacaacaccgagacagacttcaagtgc |
| | | aagtggaccctgacctccggcacccctagcggctgcgag |
| | | aatatcagcggagccttcgccagcaaccggaccttcgat |
| | | atcaccgtgtctggcctgggcaccgcccccaagaccctg |
| | | atcatcaccagaaccgccacaaatgccaccaccacaacc |
| | | cacaaagtgatcttcagcaaggcccccggctctggcctg |
| | | aacgacatttttgaggcccagaagattgagtggcatgaa |
| | | catcaccaccaccaccat |
| 149 | gp350 B95-8 (2-425) D296R | EAALLVCQYTIQSLIHLTGEDPGFFNVEIPEFPFYPTCN |
| | Avi-His | VCTADVNVTINFDVGGKKHQLDLDFGQLTPHTKAVYQPR |
| | | GAFGGSENATNLFLLELLGAGELALTMRSKKLPINVTTG |
| | | EEQQVSLESVDVYFQDVFGTMWCHHAEMQNPVYLIPETV |
| | | PYIKWDNCNSTNITAVVRAQGLDVTLPLSLPTSAQDSNF |
| | | SVKTEMLGNEIDIECIMEDGEISQVLPGDNKFNITCSGY |
| | | ESHVPSGGILTSTSPVATPIPGTGYAYSLRLTPRPVSRF |
| | | LGNNSILYVFYSGNGPKASGGRYCIQSNIVFSDEIPASQ |
| | | DMPTNTTDITYVGDNATYSVPMVTSEDANSPNVTVTAFW |
| | | AWPNNTETDFKCKWTLTSGTPSGCENISGAFASNRTFDI |
| | | TVSGLGTAPKTLIITRTATNATTTTHKVIFSKAPGSGLN |
| | | DIFEAQKIEWHEHHHHHH |
| 150 | NAM encoding SEQ ID | gaagctgccctgctcgtgtgccagtacaccatccagagc |
| | NO: 149 | ctgatccacctgaccggcgaggaccccggcttcttcaac |
| | | gtggaaatccccgagttccccttctaccctacctgcaac |
| | | gtgtgcaccgccgacgtgaacgtgaccatcaacttcgac |
| | | gtgggcggcaagaagcaccagctggacctggatttcggc |
| | | cagctgacccctcacaccaaggccgtgtatcagcccaga |
| | | ggcgcctttggcggcagcgagaacgccaccaatctgttt |
| | | ctgctggaactcctaggcgccggcgagctggccctgacc |
| | | atgagaagcaagaaactgcccatcaatgtgaccacaggc |
| | | gaggaacagcaggtgtccctggaaagcgtggacgtgtac |
| | | tttcaagacgtgttcggcaccatgtggtgccaccacgcc |
| | | gagatgcagaaccccgtgtacctgatccccgagacagtg |
| | | ccctacatcaagtgggacaactgcaacagcaccaacatc |
| | | accgccgtcgtgcgggcccagggactggatgtgacactg |
| | | cctctgagcctgcctaccagcgcccaggacagcaacttc |
| | | agcgtgaaaaccgagatgctgggcaacgagatcgacatc |
| | | gagtgcatcatggaagatggcgagatcagccaggtgctg |
| | | cccggcgacaacaagttcaacatcacatgcagcggctac |
| | | gagagccacgtgccatctggcggcatcctgaccagcaca |
| | | agcccagtggccacacccatccctggcacaggctacgcc |
| | | tacagcctgagactgacccccagacccgtgtccagattc |
| | | ctgggcaacaacagcatcctgtacgtgttctacagcggc |
| | | aacggccccaaggcctctggcggccggtactgtatccag |
| | | agcaacatcgtgttcagcgacgagatccccgccagccag |
| | | gacatgcccaccaataccaccgacatcacgtacgtgggc |
| | | gacaatgccacctacagcgtgccaatggtcacctccgag |
| | | gacgccaacagccccaacgtgaccgtgacagccttttgg |
| | | gcctggcctaacaacaccgagacagacttcaagtgcaag |
| | | tggaccctgacctccggcacccctagcggctgcgagaat |
| | | atcagcggagccttcgccagcaaccggaccttcgatatc |
| | | accgtgtctggcctgggcaccgcccccaagaccctgatc |
| | | atcaccagaaccgccacaaatgccaccaccacaacccac |
| | | aaagtgatcttcagcaaggcccccggctctggcctgaac |
| | | gacatttttgaggcccagaagattgagtggcatgaacat |
| | | caccaccaccaccat |
| 151 | gp350 B95-8 (2-425) | EAALLVCQYTIQSLIHLTGEDPGFFNVEIPEFPFYPTCN |
| | WDN162-4AAA Avi-His | VCTADVNVTINFDVGGKKHQLDLDFGQLTPHTKAVYQPR |
| | | GAFGGSENATNLFLLELLGAGELALTMRSKKLPINVTTG |
| | | EEQQVSLESVDVYFQDVFGTMWCHHAEMQNPVYLIPETV |
| | | PYIKAAACNSTNITAVVRAQGLDVTLPLSLPTSAQDSNF |
| | | SVKTEMLGNEIDIECIMEDGEISQVLPGDNKFNITCSGY |
| | | ESHVPSGGILTSTSPVATPIPGTGYAYSLRLTPRPVSRF |
| | | LGNNSILYVFYSGNGPKASGGDYCIQSNIVFSDEIPASQ |
| | | DMPTNTTDITYVGDNATYSVPMVTSEDANSPNVTVTAFW |
| | | AWPNNTETDFKCKWTLTSGTPSGCENISGAFASNRTFDI |
| | | TVSGLGTAPKTLIITRTATNATTTTHKVIFSKAPGSGLN |
| | | DIFEAQKIEWHEHHHHHH |
| 152 | NAM encoding SEQ ID | gaagctgccctgctcgtgtgccagtacaccatccagagc |
| | NO: 151 | ctgatccacctgaccggcgaggaccccggcttcttcaac |
| | | gtggaaatccccgagttccccttctaccctacctgcaac |
| | | gtgtgcaccgccgacgtgaacgtgaccatcaacttcgac |
| | | gtgggcggcaagaagcaccagctggacctggatttcggc |
| | | cagctgacccctcacaccaaggccgtgtatcagcccaga |
| | | ggcgcctttggcggcagcgagaacgccaccaatctgttt |
| | | ctgctggaactcctaggcgccggcgagctggccctgacc |
| | | atgagaagcaagaaactgcccatcaatgtgaccacaggc |
| | | gaggaacagcaggtgtccctggaaagcgtggacgtgtac |
| | | tttcaagacgtgttcggcaccatgtggtgccaccacgcc |
| | | gagatgcagaaccccgtgtacctgatccccgagacagtg |
| | | ccctacatcaaggccgccgcctgcaacagcaccaacatc |
| | | accgccgtcgtgcgggcccagggactggatgtgacactg |
| | | cctctgagcctgcctaccagcgcccaggacagcaacttc |
| | | agcgtgaaaaccgagatgctgggcaacgagatcgacatc |
| | | gagtgcatcatggaagatggcgagatcagccaggtgctg |
| | | cccggcgacaacaagttcaacatcacatgcagcggctac |
| | | gagagccacgtgccatctggcggcatcctgaccagcaca |
| | | agcccagtggccacacccatccctggcacaggctacgcc |
| | | tacagcctgagactgacccccagacccgtgtccagattc |
| | | ctgggcaacaacagcatcctgtacgtgttctacagcggc |
| | | aacggccccaaggcctctggcggcgattactgtatccag |
| | | agcaacatcgtgttcagcgacgagatccccgccagccag |
| | | gacatgcccaccaataccaccgacatcacgtacgtgggc |
| | | gacaatgccacctacagcgtgccaatggtcacctccgag |
| | | gacgccaacagccccaacgtgaccgtgacagccttttgg |
| | | gcctggcctaacaacaccgagacagacttcaagtgcaag |
| | | tggaccctgacctccggcacccctagcggctgcgagaat |
| | | atcagcggagccttcgccagcaaccggaccttcgatatc |
| | | accgtgtctggcctgggcaccgcccccaagaccctgatc |
| | | atcaccagaaccgccacaaatgccaccaccacaacccac |
| | | aaagtgatcttcagcaaggcccccggctctggcctgaac |
| | | gacatttttgaggcccagaagattgagtggcatgaacat |
| | | caccaccaccaccat |
| 153 | gp350 B95-8 (2-425) | EAALLVCQYTIQSLIHLTGEDPGFFNVEIPEFPFYPTCN |
| | D296R/I160R Avi-His | VCTADVNVTINFDVGGKKHQLDLDFGQLTPHTKAVYQPR |
| | | GAFGGSENATNLFLLELLGAGELALTMRSKKLPINVTTG |
| | | EEQQVSLESVDVYFQDVFGTMWCHHAEMQNPVYLIPETV |
| | | PYRKWDNCNSTNITAVVRAQGLDVTLPLSLPTSAQDSNF |
| | | SVKTEMLGNEIDIECIMEDGEISQVLPGDNKFNITCSGY |
| | | ESHVPSGGILTSTSPVATPIPGTGYAYSLRLTPRPVSRF |
| | | LGNNSILYVFYSGNGPKASGGRYCIQSNIVFSDEIPASQ |
| | | DMPTNTTDITYVGDNATYSVPMVTSEDANSPNVTVTAFW |
| | | AWPNNTETDFKCKWTLTSGTPSGCENISGAFASNRTFDI |
| | | TVSGLGTAPKTLIITRTATNATTTTHKVIFSKAPGSGLN |
| | | DIFEAQKIEWHEHHHHHH |
| 154 | NAM encoding SEQ ID | gaagctgccctgctcgtgtgccagtacaccatccagagc |
| | NO: 153 | ctgatccacctgaccggcgaggaccccggcttcttcaac |
| | | gtggaaatccccgagttccccttctaccctacctgcaac |
| | | gtgtgcaccgccgacgtgaacgtgaccatcaacttcgac |
| | | gtgggcggcaagaagcaccagctggacctggatttcggc |
| | | cagctgacccctcacaccaaggccgtgtatcagcccaga |
| | | ggcgcctttggcggcagcgagaacgccaccaatctgttt |
| | | ctgctggaactcctaggcgccggcgagctggccctgacc |
| | | atgagaagcaagaaactgcccatcaatgtgaccacaggc |
| | | gaggaacagcaggtgtccctggaaagcgtggacgtgtac |
| | | tttcaagacgtgttcggcaccatgtggtgccaccacgcc |
| | | gagatgcagaaccccgtgtacctgatccccgagacagtg |
| | | ccctaccggaagtgggacaactgcaacagcaccaacatc |
| | | accgccgtcgtgcgggcccagggactggatgtgacactg |
| | | cctctgagcctgcctaccagcgcccaggacagcaacttc |
| | | agcgtgaaaaccgagatgctgggcaacgagatcgacatc |
| | | gagtgcatcatggaagatggcgagatcagccaggtgctg |
| | | cccggcgacaacaagttcaacatcacatgcagcggctac |
| | | gagagccacgtgccatctggcggcatcctgaccagcaca |
| | | agcccagtggccacacccatccctggcacaggctacgcc |
| | | tacagcctgagactgacccccagacccgtgtccagattc |
| | | ctgggcaacaacagcatcctgtacgtgttctacagcggc |
| | | aacggccccaaggcctctggcggccggtactgtatccag |
| | | agcaacatcgtgttcagcgacgagatccccgccagccag |
| | | gacatgcccaccaataccaccgacatcacgtacgtgggc |
| | | gacaatgccacctacagcgtgccaatggtcacctccgag |
| | | gacgccaacagccccaacgtgaccgtgacagccttttgg |
| | | gcctggcctaacaacaccgagacagacttcaagtgcaag |
| | | tggaccctgacctccggcacccctagcggctgcgagaat |
| | | atcagcggagccttcgccagcaaccggaccttcgatatc |
| | | accgtgtctggcctgggcaccgcccccaagaccctgatc |
| | | atcaccagaaccgccacaaatgccaccaccacaacccac |
| | | aaagtgatcttcagcaaggcccccggctctggcctgaac |
| | | gacatttttgaggcccagaagattgagtggcatgaacat |
| | | caccaccaccaccat |
| 155 | gp350 B95-8 (2-425) | EAALLVCQYTIQSLIHLTGEDPGFFNVEIPEFPFYPTCN |
| | D296R/P158R Avi-His | VCTADVNVTINFDVGGKKHQLDLDFGQLTPHTKAVYQPR |
| | | GAFGGSENATNLFLLELLGAGELALTMRSKKLPINVTTG |
| | | EEQQVSLESVDVYFQDVFGTMWCHHAEMQNPVYLIPETV |
| | | RYIKWDNCNSTNITAVVRAQGLDVTLPLSLPTSAQDSNF |
| | | SVKTEMLGNEIDIECIMEDGEISQVLPGDNKFNITCSGY |
| | | ESHVPSGGILTSTSPVATPIPGTGYAYSLRLTPRPVSRF |
| | | LGNNSILYVFYSGNGPKASGGRYCIQSNIVFSDEIPASQ |
| | | DMPTNTTDITYVGDNATYSVPMVTSEDANSPNVTVTAFW |
| | | AWPNNTETDFKCKWTLTSGTPSGCENISGAFASNRTFDI |
| | | TVSGLGTAPKTLIITRTATNATTTTHKVIFSKAPGSGLN |
| | | DIFEAQKIEWHEHHHHHH |
| 156 | NAM encoding SEQ ID | gaagctgccctgctcgtgtgccagtacaccatccagagc |
| | NO: 155 | ctgatccacctgaccggcgaggaccccggcttcttcaac |
| | | gtggaaatccccgagttccccttctaccctacctgcaac |
| | | gtgtgcaccgccgacgtgaacgtgaccatcaacttcgac |
| | | gtgggcggcaagaagcaccagctggacctggatttcggc |
| | | cagctgacccctcacaccaaggccgtgtatcagcccaga |
| | | ggcgcctttggcggcagcgagaacgccaccaatctgttt |
| | | ctgctggaactcctaggcgccggcgagctggccctgacc |
| | | atgagaagcaagaaactgcccatcaatgtgaccacaggc |
| | | gaggaacagcaggtgtccctggaaagcgtggacgtgtac |
| | | tttcaagacgtgttcggcaccatgtggtgccaccacgcc |
| | | gagatgcagaaccccgtgtacctgatccccgagacagtg |
| | | cggtacatcaagtgggacaactgcaacagcaccaacatc |
| | | accgccgtcgtgcgggcccagggactggatgtgacactg |
| | | cctctgagcctgcctaccagcgcccaggacagcaacttc |
| | | agcgtgaaaaccgagatgctgggcaacgagatcgacatc |
| | | gagtgcatcatggaagatggcgagatcagccaggtgctg |
| | | cccggcgacaacaagttcaacatcacatgcagcggctac |
| | | gagagccacgtgccatctggcggcatcctgaccagcaca |
| | | agcccagtggccacacccatccctggcacaggctacgcc |
| | | tacagcctgagactgacccccagacccgtgtccagattc |
| | | ctgggcaacaacagcatcctgtacgtgttctacagcggc |
| | | aacggccccaaggcctctggcggccggtactgtatccag |
| | | agcaacatcgtgttcagcgacgagatccccgccagccag |
| | | gacatgcccaccaataccaccgacatcacgtacgtgggc |
| | | gacaatgccacctacagcgtgccaatggtcacctccgag |
| | | gacgccaacagccccaacgtgaccgtgacagccttttgg |
| | | gcctggcctaacaacaccgagacagacttcaagtgcaag |
| | | tggaccctgacctccggcacccctagcggctgcgagaat |
| | | atcagcggagccttcgccagcaaccggaccttcgatatc |
| | | accgtgtctggcctgggcaccgcccccaagaccctgatc |
| | | atcaccagaaccgccacaaatgccaccaccacaacccac |
| | | aaagtgatcttcagcaaggcccccggctctggcctgaac |
| | | gacatttttgaggcccagaagattgagtggcatgaacat |
| | | caccaccaccaccat |
| 157 | gp350 B95-8 (2-425) | EAALLVCQYTIQSLIHLTGEDPGFFNVEIPEFPFYPTCN |
| | D296R/P158N*/I160T | VCTADVNVTINFDVGGKKHQLDLDFGQLTPHTKAVYQPR |
| | Avi-His | GAFGGSENATNLFLLELLGAGELALTMRSKKLPINVTTG |
| | | EEQQVSLESVDVYFQDVFGTMWCHHAEMQNPVYLIPETV |
| | | NYTKWDNCNSTNITAVVRAQGLDVTLPLSLPTSAQDSNF |
| | | SVKTEMLGNEIDIECIMEDGEISQVLPGDNKFNITCSGY |
| | | ESHVPSGGILTSTSPVATPIPGTGYAYSLRLTPRPVSRF |
| | | LGNNSILYVFYSGNGPKASGGRYCIQSNIVFSDEIPASQ |
| | | DMPTNTTDITYVGDNATYSVPMVTSEDANSPNVTVTAFW |
| | | AWPNNTETDFKCKWTLTSGTPSGCENISGAFASNRTFDI |
| | | TVSGLGTAPKTLIITRTATNATTTTHKVIFSKAPGSGLN |
| | | DIFEAQKIEWHEHHHHHH |
| 158 | NAM encoding SEQ ID | gaagctgccctgctcgtgtgccagtacaccatccagagc |
| | NO: 157 | ctgatccacctgaccggcgaggaccccggcttcttcaac |
| | | gtggaaatccccgagttccccttctaccctacctgcaac |
| | | gtgtgcaccgccgacgtgaacgtgaccatcaacttcgac |
| | | gtgggcggcaagaagcaccagctggacctggatttcggc |
| | | cagctgacccctcacaccaaggccgtgtatcagcccaga |
| | | ggcgcctttggcggcagcgagaacgccaccaatctgttt |
| | | ctgctggaactcctaggcgccggcgagctggccctgacc |
| | | atgagaagcaagaaactgcccatcaatgtgaccacaggc |
| | | gaggaacagcaggtgtccctggaaagcgtggacgtgtac |
| | | tttcaagacgtgttcggcaccatgtggtgccaccacgcc |
| | | gagatgcagaaccccgtgtacctgatccccgagacagtg |
| | | aactacaccaagtgggacaactgcaacagcaccaacatc |
| | | accgccgtcgtgcgggcccagggactggatgtgacactg |
| | | cctctgagcctgcctaccagcgcccaggacagcaacttc |
| | | agcgtgaaaaccgagatgctgggcaacgagatcgacatc |
| | | gagtgcatcatggaagatggcgagatcagccaggtgctg |
| | | cccggcgacaacaagttcaacatcacatgcagcggctac |
| | | gagagccacgtgccatctggcggcatcctgaccagcaca |
| | | agcccagtggccacacccatccctggcacaggctacgcc |
| | | tacagcctgagactgacccccagacccgtgtccagattc |
| | | ctgggcaacaacagcatcctgtacgtgttctacagcggc |
| | | aacggccccaaggcctctggcggccggtactgtatccag |
| | | agcaacatcgtgttcagcgacgagatccccgccagccag |
| | | gacatgcccaccaataccaccgacatcacgtacgtgggc |
| | | gacaatgccacctacagcgtgccaatggtcacctccgag |
| | | gacgccaacagccccaacgtgaccgtgacagccttttgg |
| | | gcctggcctaacaacaccgagacagacttcaagtgcaag |
| | | tggaccctgacctccggcacccctagcggctgcgagaat |
| | | atcagcggagccttcgccagcaaccggaccttcgatatc |
| | | accgtgtctggcctgggcaccgcccccaagaccctgatc |
| | | atcaccagaaccgccacaaatgccaccaccacaacccac |
| | | aaagtgatcttcagcaaggcccccggctctggcctgaac |
| | | gacatttttgaggcccagaagattgagtggcatgaacat |
| | | caccaccaccaccat |
| 159 | gp350 B95-8 (2-425) | EAALLVCQYTIQSLIHLTGEDPGFFNVEIPEFPFYPTCN |
| | D296R/P158R/I160R | VCTADVNVTINFDVGGKKHQLDLDFGQLTPHTKAVYQPR |
| | Avi-His | GAFGGSENATNLFLLELLGAGELALTMRSKKLPINVTTG |
| | | EEQQVSLESVDVYFQDVFGTMWCHHAEMQNPVYLIPETV |
| | | RYRKWDNCNSTNITAVVRAQGLDVTLPLSLPTSAQDSNF |
| | | SVKTEMLGNEIDIECIMEDGEISQVLPGDNKFNITCSGY |
| | | ESHVPSGGILTSTSPVATPIPGTGYAYSLRLTPRPVSRF |
| | | LGNNSILYVFYSGNGPKASGGRYCIQSNIVFSDEIPASQ |
| | | DMPTNTTDITYVGDNATYSVPMVTSEDANSPNVTVTAFW |
| | | AWPNNTETDFKCKWTLTSGTPSGCENISGAFASNRTFDI |
| | | TVSGLGTAPKTLIITRTATNATTTTHKVIFSKAPGSGLN |
| | | DIFEAQKIEWHEHHHHHH |
| 160 | NAM encoding SEQ ID | gaagctgccctgctcgtgtgccagtacaccatccagagc |
| | NO: 159 | ctgatccacctgaccggcgaggaccccggcttcttcaac |
| | | gtggaaatccccgagttccccttctaccctacctgcaac |
| | | gtgtgcaccgccgacgtgaacgtgaccatcaacttcgac |
| | | gtgggcggcaagaagcaccagctggacctggatttcggc |
| | | cagctgacccctcacaccaaggccgtgtatcagcccaga |
| | | ggcgcctttggcggcagcgagaacgccaccaatctgttt |
| | | ctgctggaactcctaggcgccggcgagctggccctgacc |
| | | atgagaagcaagaaactgcccatcaatgtgaccacaggc |
| | | gaggaacagcaggtgtccctggaaagcgtggacgtgtac |
| | | tttcaagacgtgttcggcaccatgtggtgccaccacgcc |
| | | gagatgcagaaccccgtgtacctgatccccgagacagtg |
| | | cggtaccggaagtgggacaactgcaacagcaccaacatc |
| | | accgccgtcgtgcgggcccagggactggatgtgacactg |
| | | cctctgagcctgcctaccagcgcccaggacagcaacttc |
| | | agcgtgaaaaccgagatgctgggcaacgagatcgacatc |
| | | gagtgcatcatggaagatggcgagatcagccaggtgctg |
| | | cccggcgacaacaagttcaacatcacatgcagcggctac |
| | | gagagccacgtgccatctggcggcatcctgaccagcaca |
| | | agcccagtggccacacccatccctggcacaggctacgcc |
| | | tacagcctgagactgacccccagacccgtgtccagattc |
| | | ctgggcaacaacagcatcctgtacgtgttctacagcggc |
| | | aacggccccaaggcctctggcggccggtactgtatccag |
| | | agcaacatcgtgttcagcgacgagatccccgccagccag |
| | | gacatgcccaccaataccaccgacatcacgtacgtgggc |
| | | gacaatgccacctacagcgtgccaatggtcacctccgag |
| | | gacgccaacagccccaacgtgaccgtgacagccttttgg |
| | | gcctggcctaacaacaccgagacagacttcaagtgcaag |
| | | tggaccctgacctccggcacccctagcggctgcgagaat |
| | | atcagcggagccttcgccagcaaccggaccttcgatatc |
| | | accgtgtctggcctgggcaccgcccccaagaccctgatc |
| | | atcaccagaaccgccacaaatgccaccaccacaacccac |
| | | aaagtgatcttcagcaaggcccccggctctggcctgaac |
| | | gacatttttgaggcccagaagattgagtggcatgaacat |
| | | caccaccaccaccat |
| 161 | E7 clone Heavy Chain aa | QVQLVQSGADVRKPGASVKVSCKASTYIFTGYYIH |
| | sequence | WVRQAPGRGLEWLGWIHPNSGGTTYSQMFQGRVT |
| | | MTRDRSITTSYMELSRLQSDDTAIYYCATLRFVEYSF |
| | | DSWGQGTLVTVSS |
| 162 | E7 clone Heavy Chain | CAGGTGCAGCTGGTGCAGTCTGGCGCAGACGTGA |
| | nucleotide sequence | GGAAGCCAGGAGCCTCCGTGAAGGTGTCTTGTAA |
| | | GGCCAGCACCTACATCTTCACAGGCTACTATATCC |
| | | ACTGGGTGAGGCAGGCACCAGGAAGGGGCCTGGA |
| | | GTGGCTGGGCTGGATTCACCCTAACTCTGGCGGCA |
| | | CCACATACAGCCAGATGTTTCAGGGCAGAGTGAC |
| | | CATGACACGGGACAGATCCATCACCACATCTTATA |
| | | TGGAGCTGAGCCGGCTGCAGTCCGACGATACCGC |
| | | CATCTACTATTGCGCCACACTGAGATTCGTGGAGT |
| | | ATTCTTTTGATAGCTGGGGCCAGGGCACCCTGGTG |
| | | ACAGTGAGCTCC |
| 163 | E7 clone Heavy Chain CDR1 | TYIFTGY |
| 164 | E7 clone Heavy Chain CDR2 | HPNSGG |
| 165 | E7 clone Heavy Chain CDR3 | LRFVEYSFDS |
| 166 | E7 clone Kappa Chain aa | EIVLTQSPGTLSLSPGERATLSCRASQSISSTYLAWYQ |
| | sequence | QIPGQAPRLLIYGASSRAAGIPDRFSGGGSGTDFTLTI |
| | | SRLEPEDFAVYYCQQYGSSPRSFGQGTKLEIK |
| 167 | E7 clone Kappa Chain | GAGATCGTGCTGACCCAGAGCCCAGGCACACTGA |
| | nucleotide sequence | GCCTGTCCCCAGGAGAGAGGGCCACCCTGTCCTGT |
| | | AGAGCCTCTCAGAGCATCAGCTCCACATACCTGGC |
| | | CTGGTATCAGCAGATCCCAGGACAGGCACCTAGG |
| | | CTGCTGATCTACGGAGCCTCTAGCAGGGCAGCAG |
| | | GCATCCCCGACCGCTTCTCCGGCGGAGGCTCTGGC |
| | | ACCGACTTCACCCTGACAATCTCTCGGCTGGAGCC |
| | | TGAGGACTTCGCCGTGTACTATTGCCAGCAGTATG |
| | | GCTCCTCTCCAAGGTCCTTTGGCCAGGGCACAAAG |
| | | CTGGAGATCAAG |
| 168 | E7 clone Kappa Chain CDR1 | RASQSISSTYLA |
| 169 | E7 clone Kappa Chain CDR2 | GASSRAA |
| 170 | E7 clone Kappa Chain CDR3 | QQYGSSPRS |
| 171 | B8 clone Heavy Chain aa | EVQLLESGGALVQPGGSLRLSCAASGFTFKTYAMSW |
| | sequence | VRQVPGKGLEWVSAISGSGTASYYADSVKGRFTLSR |
| | | DNSKNTLYLQLSSLRDEDTGVYYCARRFLDWFGMD |
| | | VWGLGTTVTVSS |
| 172 | B8 clone Heavy Chain | GAGGTGCAGCTGCTGGAGAGCGGCGGCGCCCTGG |
| | nucleotide sequence | TGCAGCCAGGAGGCAGCCTGCGGCTGTCCTGTGCC |
| | | GCCTCTGGCTTCACCTTTAAGACATACGCCATGTC |
| | | CTGGGTGAGGCAGGTGCCTGGCAAGGGCCTGGAG |
| | | TGGGTGTCTGCCATCTCCGGCTCTGGCACCGCCTC |
| | | TTACTATGCCGACAGCGTGAAGGGCAGGTTCACCC |
| | | TGAGCCGCGATAACTCCAAGAATACACTGTATCTG |
| | | CAGCTGAGCTCCCTGCGGGACGAGGATACCGGCG |
| | | TGTACTATTGCGCCCGGAGATTCCTGGACTGGTTT |
| | | GGCATGGACGTGTGGGGCCTGGGCACCACAGTGA |
| | | CAGTGTCTAGC |
| 173 | B8 clone Heavy Chain CDR1 | GFTFKTY |
| 174 | B8 clone Heavy Chain CDR2 | SGSGTA |
| 175 | B8 clone Heavy Chain CDR3 | RFLDWFGMDV |
| 176 | B8 clone Kappa Chain aa | DIVMTQSPLSLPVTPGEPASISCLSSQSLLQSNGYNYV |
| | sequence | DWYLQKPGQSPQLLIYLGSNRASGVPDRFSGSGSGT |
| | | DFTLKISRVEAEDVGVYYCMVTLHPPTFGQGAKVEI |
| | | K |
| 177 | B8 clone Kappa Chain | GACATCGTGATGACCCAGTCCCCTCTGTCTCTGCC |
| | nucleotide sequence | AGTGACACCCGGCGAGCCTGCCTCTATCAGCTGTC |
| | | TGAGCTCCCAGAGCCTGCTGCAGTCCAACGGCTAC |
| | | AATTATGTGGATTGGTACCTGCAGAAGCCAGGCC |
| | | AGTCCCCCCAGCTGCTGATCTATCTGGGCTCTAAC |
| | | AGGGCCAGCGGCGTGCCCGACAGATTCTCCGGCT |
| | | CTGGCAGCGGCACCGACTTCACCCTGAAGATCTCT |
| | | CGGGTGGAGGCAGAGGACGTGGGCGTGTACTATT |
| | | GCATGGTGACCCTGCACCCACCTACATTCGGCCAG |
| | | GGAGCCAAGGTGGAGATCAAG |
| 178 | B8 clone Kappa Chain CDR1 | LSSQSLLQSNGYNYVD |
| 179 | B8 clone Kappa Chain CDR2 | LGSNRAS |
| 180 | B8 clone Kappa Chain CDR3 | MVTLHPPT |
| 181 | B3 clone Heavy Chain aa | EGQLVQSGGGLVQPGGSLTLSCEVSGFTFKNYEMN |
| | sequence | WVRQAPGKGLEWVSYISSGGIAIFHADSVKGRFTVS |
| | | RDNAKNLLYLQMNSLRVEDTAVYYCARDENNVRR |
| | | PFDHWGQGTLVTVSS |
| 182 | B3 clone Heavy Chain | GAGGGACAGCTGGTGCAGTCCGGCGGAGGCCTGG |
| | nucleotide sequence | TGCAGCCAGGAGGCTCCCTGACCCTGTCTTGTGAG |
| | | GTGAGCGGCTTCACCTTCAAGAACTACGAGATGA |
| | | ATTGGGTGCGGCAGGCACCTGGCAAGGGCCTGGA |
| | | GTGGGTGTCTTATATCAGCTCCGGCGGAATCGCAA |
| | | TCTTCCACGCAGATTCCGTGAAGGGCAGGTTTACC |
| | | GTGTCTCGCGACAACGCCAAGAATCTGCTGTACCT |
| | | GCAGATGAACAGCCTGCGGGTGGAGGACACAGCC |
| | | GTGTACTATTGCGCCAGGGATGAGAACAACGTGC |
| | | GGCGGCCCTTCGACCACTGGGGACAGGGCACCCT |
| | | GGTGACAGTGTCTAGC |
| 183 | B3 clone Heavy Chain CDR1 | GFTFKNY |
| 184 | B3 clone Heavy Chain CDR2 | SSGGIA |
| 185 | B3 clone Heavy Chain CDR3 | DENNVRRPFDH |
| 186 | B3 clone Lambda Chain aa | QSVLTQPPSASGSPGQSVTISCTGSSSDVGAYDFVSW |
| | sequence | FQQYPGQAPKLIIYEVNKRPSGVPARFSGSKSGNTAS |
| | | LTVSGLQAEDEADYFCFSYGGTTNLRVFGGGTKLT |
| 187 | B3 clone Lambda Chain | CAGTCTGTGCTGACCCAGCCACCTAGCGCCTCCGG |
| | nucleotide sequence | CTCTCCCGGCCAGAGCGTGACCATCTCCTGTACAG |
| | | GCAGCTCCTCTGACGTGGGCGCCTACGATTTCGTG |
| | | AGCTGGTTTCAGCAGTATCCAGGCCAGGCCCCCAA |
| | | GCTGATCATCTACGAGGTGAACAAGCGGCCTTCCG |
| | | GCGTGCCAGCCAGATTCAGCGGCTCCAAGTCTGGC |
| | | AATACCGCCTCTCTGACAGTGAGCGGCCTGCAGGC |
| | | AGAGGACGAGGCAGATTACTTCTGCTTTTCTTATG |
| | | GCGGCACCACAAACCTGCGGGTGTTTGGCGGCGG |
| | | CACCAAGCTGACA |
| 188 | B3 clone Kappa Chain CDR1 | TGSSSDVGAYDFVS |
| 189 | B3 clone Kappa Chain CDR2 | EVNKRPS |
| 190 | B3 clone Kappa Chain CDR3 | FSYGGTTNLRV |
| 191 | C7 clone Heavy Chain aa | EVQLVESGGNLVQPGASLRLSCTASRFNFNKYAMH |
| | sequence | WVRQTPGKGLEWVSAISWDSTYIDYGNSVKGRFTIS |
| | | RDNTRNSLYLQMNSLTAEDTALYYCAKCEDYLRLC |
| | | SAYDIWGHGTMVTVSS |
| 192 | C7 clone Heavy Chain | GAGGTGCAGCTGGTGGAGAGCGGCGGAAACCTGG |
| | nucleotide sequence | TGCAGCCAGGAGCCTCTCTGAGGCTGAGCTGTACC |
| | | GCCTCCCGCTTCAACTTTAATAAGTACGCAATGCA |
| | | CTGGGTGCGGCAGACCCCTGGCAAGGGCCTGGAG |
| | | TGGGTGTCTGCCATCAGCTGGGACTCCACATACAT |
| | | CGATTATGGCAACTCCGTGAAGGGCAGGTTCACC |
| | | ATCTCTCGGGACAACACAAGAAATAGCCTGTATCT |
| | | GCAGATGAATTCCCTGACCGCCGAGGATACAGCC |
| | | CTGTACTATTGCGCCAAGTGTGAGGACTACCTGCG |
| | | GCTGTGCTCTGCCTATGATATCTGGGGCCACGGCA |
| | | CCATGGTGACAGTGAGCTCC |
| 193 | C7 clone Heavy Chain CDR1 | RFNFNKY |
| 194 | C7 clone Heavy Chain CDR2 | SWDSTY |
| 195 | C7 clone Heavy Chain CDR3 | CEDYLRLCSAYDI |
| 196 | C7 clone Kappa Chain aa | DIVMTQSPLSLPVTPGESASISCRSSQSLLHSNGKNYL |
| | sequence | SWYLQKPGQSPQLLIDLGSNRASGVSDRFSGSGSGT |
| | | DFTLKISRVEADDVGVYYCMQAVQTPITFGQGTRLA |
| | | IK |
| 197 | C7 clone Kappa Chain | GACATCGTGATGACCCAGTCTCCTCTGAGCCTGCC |
| | nucleotide sequence | CGTGACACCTGGCGAGTCTGCCAGCATCTCCTGTC |
| | | GGAGCTCCCAGAGCCTGCTGCACTCCAACGGCAA |
| | | GAATTACCTGTCTTGGTATCTGCAGAAGCCAGGCC |
| | | AGAGCCCCCAGCTGCTGATCGATCTGGGCTCCAAC |
| | | AGGGCCTCCGGCGTGTCTGACAGATTCTCTGGCAG |
| | | CGGCTCCGGCACCGACTTCACCCTGAAGATCAGCA |
| | | GGGTGGAGGCCGACGATGTGGGCGTGTACTATTG |
| | | CATGCAGGCCGTGCAGACCCCAATCACATTCGGCC |
| | | AGGGAACCCGCCTGGCCATCAAG |
| 198 | C7 clone Kappa Chain CDR1 | RSSQSLLHSNGKNYLS |
| 199 | C7 clone Kappa Chain CDR2 | LGSNRAS |
| 200 | C7 clone Kappa Chain CDR3 | MQAVQTPIT |
| 201 | A9 clone Heavy Chain aa | QVQLVQSGAELKTPGASVKVSCKASGYTFTGYYIH |
| | sequence | WVRQAPGEGLEWTGWINPNSGATRYGQKFQGRVTL |
| | | TSDTSSSTVYMEVSNLTSDDSAVYYCARELSYSIRGT |
| | | GPLGYWGLGTLVTVSS |
| 202 | A9 clone Heavy Chain | CAGGTGCAGCTGGTGCAGTCCGGCGCAGAGCTGA |
| | nucleotide sequence | AGACCCCAGGAGCCAGCGTGAAGGTGTCCTGTAA |
| | | GGCCTCTGGCTACACCTTCACAGGCTACTATATCC |
| | | ACTGGGTGCGGCAGGCACCAGGAGAGGGCCTGGA |
| | | GTGGACCGGCTGGATCAACCCTAATAGCGGCGCC |
| | | ACAAGATACGGCCAGAAGTTTCAGGGCCGCGTGA |
| | | CCCTGACAAGCGACACCAGCTCCTCTACAGTGTAT |
| | | ATGGAGGTGTCCAACCTGACCTCCGACGATTCTGC |
| | | CGTGTACTATTGCGCCCGGGAGCTGTCTTACAGCA |
| | | TCAGAGGAACAGGACCACTGGGATATTGGGGCCT |
| | | GGGCACCCTGGTGACAGTGAGCTCC |
| 203 | A9 clone Heavy Chain CDR1 | GYTFTGY |
| 204 | A9 clone Heavy Chain CDR2 | NPNSGA |
| 205 | A9 clone Heavy Chain CDR3 | ELSYSIRGTGPLGY |
| 206 | A9 clone Kappa Chain aa | EIVLTQSPGTLSLSPGERATLSCRASQSVASKYLAWY |
| | sequence | QQKPGQAPRLLIYGASSRATGIPDRFSGSGSGTDFTL |
| | | TISRLEPEDFAVYYCQYYGSSPLTFGQGTKVEIK |
| 207 | A9 clone Kappa Chain | GAGATCGTGCTGACCCAGTCTCCAGGCACACTGTC |
| | nucleotide sequence | CCTGTCTCCAGGAGAGAGGGCCACCCTGTCTTGTA |
| | | GAGCCAGCCAGTCCGTGGCCAGCAAGTACCTGGC |
| | | CTGGTATCAGCAGAAGCCAGGACAGGCACCTAGG |
| | | CTGCTGATCTACGGAGCCAGCTCCAGGGCAACCG |
| | | GCATCCCCGACCGCTTCTCTGGCAGCGGCTCCGGC |
| | | ACAGACTTCACCCTGACAATCTCCAGGCTGGAGCC |
| | | TGAGGACTTCGCCGTGTACTATTGCCAGTACTATG |
| | | GCTCTAGCCCACTGACCTTTGGCCAGGGCACAAAG |
| | | GTGGAGATCAAG |
| 208 | A9 clone Kappa Chain CDR1 | RASQSVASKYLA |
| 209 | A9 clone Kappa Chain CDR2 | GASSRAT |
| 210 | A9 clone Kappa Chain CDR3 | QYYGSSPLT |
| 211 | A2 clone Heavy Chain aa | EVQLAESGGGVVHPGGSLRLSCTASGFTFSRHSMHW |
| | sequence | VRQAPGKGLEWVAVISHDGSHKFYVDSVKGRFSISR |
| | | DNAKNTLYLQMSSLSGADTAVYYCVKDISSRSYGY |
| | | LAGDSWGQGSLVTVSS |
| 212 | A2 clone Heavy Chain | GAGGTGCAGCTGGCCGAGTCTGGCGGAGGAGTGG |
| | nucleotide sequence | TGCACCCAGGAGGCTCCCTGAGGCTGTCTTGTACC |
| | | GCCAGCGGCTTCACATTTTCTAGGCACAGCATGCA |
| | | CTGGGTGCGCCAGGCACCTGGCAAGGGCCTGGAG |
| | | TGGGTGGCCGTGATCTCCCACGACGGCTCTCACAA |
| | | GTTCTACGTGGATTCCGTGAAGGGCCGGTTTAGCA |
| | | TCTCCAGAGACAACGCCAAGAATACCCTGTATCTG |
| | | CAGATGAGCTCCCTGTCTGGCGCCGACACAGCCGT |
| | | GTACTATTGCGTGAAGGATATCTCTAGCAGGAGCT |
| | | ACGGCTATCTGGCAGGCGATAGCTGGGGACAGGG |
| | | CTCCCTGGTGACCGTGTCCTCT |
| 213 | A2 clone Heavy Chain CDR1 | GFTFSRH |
| 214 | A2 clone Heavy Chain CDR2 | SHDGSH |
| 215 | A2 clone Heavy Chain CDR3 | DISSRSYGYLAGDS |
| 216 | A2 clone Kappa Chain aa | DIQMTQSPSSLSASVGDIITITCRASQSVVTYLNWYQ |
| | sequence | QKPGGAPRLLIYTTSKLQSGVPSRFSGSGSGTLFTLTI |
| | | NGLRPEDFATYYCQQSYGTPPFTFGPGTRVEIN |
| 217 | A2 clone Kappa Chain | GATATTCAGATGACTCAGTCCCCAAGCAGCCTGAG |
| | nucleotide sequence | CGCCTCCGTGGGCGACATCATCACCATCACATGCA |
| | | GGGCCTCTCAGAGCGTGGTGACCTACCTGAACTGG |
| | | TATCAGCAGAAGCCAGGAGGAGCACCTAGGCTGC |
| | | TGATCTACACCACATCCAAGCTGCAGTCTGGCGTG |
| | | CCATCCAGATTCTCCGGCTCTGGCAGCGGCACCCT |
| | | GTTTACCCTGACAATCAATGGCCTGCGGCCCGAGG |
| | | ATTTCGCCACATACTATTGTCAGCAGAGCTATGGA |
| | | ACCCCCCCCTTTACTTTTGGACCAGGCACAAGAGT |
| | | GGAGATTAAC |
| 218 | A2 clone Kappa Chain CDR1 | RASQSVVTYLN |
| 219 | A2 clone Kappa Chain CDR2 | TTSKLQS |
| 220 | A2 clone Kappa Chain CDR3 | QQSYGTPPFT |
| 221 | E1 clone Heavy Chain aa | QVHLQQWGAGLVKPSETLSLTCAVQGGPFSGYYWS |
| | sequence | WIRQPPGKGLEWIGEINHSGNTHYNPSLKSRVTISVD |
| | | TSGNYFSLKLTSVTAADAAVYFCARGQQLLRNYYY |
| | | YSGMDVWGQGTTVTVSS |
| 222 | E1 clone Heavy Chain | CAGGTGCACCTGCAGCAGTGGGGAGCAGGCCTGG |
| | nucleotide sequence | TGAAGCCATCCGAGACACTGTCTCTGACATGTGCA |
| | | GTGCAGGGAGGACCCTTCTCTGGCTACTATTGGAG |
| | | CTGGATCAGGCAGCCACCTGGCAAGGGCCTGGAG |
| | | TGGATCGGCGAGATCAACCACAGCGGCAATACCC |
| | | ACTACAACCCCTCTCTGAAGAGCCGGGTGACCATC |
| | | AGCGTGGACACATCCGGCAATTACTTCTCCCTGAA |
| | | GCTGACCTCTGTGACAGCCGCCGATGCCGCCGTGT |
| | | ATTTTTGCGCCCGGGGCCAGCAGCTGCTGAGAAAC |
| | | TACTATTACTATTCCGGCATGGACGTGTGGGGACA |
| | | GGGAACCACAGTGACAGTGAGCTCC |
| 223 | E1 clone Heavy Chain CDR1 | GGPFSGY |
| 224 | E1 clone Heavy Chain CDR2 | NHSGN |
| 225 | E1 clone Heavy Chain CDR3 | GQQLLRNYYYYSGMDV |
| 226 | E1 clone Kappa Chain aa | EIVLTQSPGTLSLSPGERATLSCRASQSVTSTYLAWY |
| | sequence | QQKLGQPPRLLIFGASNRATGIPDRFSGSGSGTDFTLT |
| | | ITRLEPEDFAVYYCQRYGGSITFGQGTRLEIK |
| 227 | E1 clone Kappa Chain | GAGATCGTGCTGACACAGTCCCCAGGCACCCTGA |
| | nucleotide sequence | GCCTGTCCCCAGGAGAGCGGGCCACACTGTCCTGT |
| | | AGAGCCTCTCAGAGCGTGACCTCTACATACCTGGC |
| | | CTGGTATCAGCAGAAGCTGGGCCAGCCCCCTAGG |
| | | CTGCTGATCTTCGGCGCCTCTAACAGGGCCACAGG |
| | | CATCCCTGACCGCTTCTCCGGCTCTGGCAGCGGCA |
| | | CCGACTTCACCCTGACAATCACCAGACTGGAGCCC |
| | | GAGGACTTCGCCGTGTACTATTGCCAGCGGTACGG |
| | | CGGCAGCATCACATTTGGCCAGGGCACCAGACTG |
| | | GAGATCAAG |
| 228 | E1 clone Kappa Chain CDR1 | RASQSVTSTYLA |
| 229 | E1 clone Kappa Chain CDR2 | GASNRAT |
| 230 | E1 clone Kappa Chain CDR3 | QRYGGSIT |
| 231 | C10 clone Heavy Chain aa | EVQLVQSGGGVVQPRRSLRLSCAASGFTFSNYGMH |
| | sequence | WVRQVPGKGLQWVAIIWYDGSNKHYAASVQGRFRI |
| | | SRDNSKNTVYLQMDGLRAEDTGMYYCVRDATTAT |
| | | TEGTSQYYFDLWGQGALVTVSS |
| 232 | C10 clone Heavy Chain | GAGGTGCAGCTGGTGCAGTCCGGCGGAGGAGTGG |
| | nucleotide sequence | TGCAGCCACGGAGATCTCTGAGGCTGAGCTGTGCC |
| | | GCCTCCGGCTTCACCTTTTCTAACTACGGAATGCA |
| | | CTGGGTGCGCCAGGTGCCTGGCAAGGGCCTGCAG |
| | | TGGGTGGCCATCATCTGGTACGACGGCTCCAATAA |
| | | GCACTATGCCGCCTCTGTGCAGGGCAGGTTCCGCA |
| | | TCTCTCGGGATAACAGCAAGAATACCGTGTATCTG |
| | | CAGATGGACGGCCTGCGGGCCGAGGATACAGGCA |
| | | TGTACTATTGCGTGAGAGACGCCACCACAGCCACC |
| | | ACAGAGGGCACCAGCCAGTACTATTTTGATCTGTG |
| | | GGGACAGGGCGCCCTGGTGACAGTGAGCTCC |
| 233 | C10 clone Heavy Chain CDR1 | GFTFSNY |
| 234 | C10 clone Heavy Chain CDR2 | WYDGSN |
| 235 | C10 clone Heavy Chain CDR3 | DATTATTEGTSQYYFDL |
| 236 | C10 clone Kappa Chain aa | DIVMTQSPDSLAVSLGERATINCKSSQTLLYTSNSKN |
| | sequence | YLAWYQQKVGQPPRLLIYWASTRESGVPDRFSGSGS |
| | | GTDFTLTISSLLAEDVAVYYCQQYYTTPLTFGGGTK |
| | | VEVK |
| 237 | C10 clone Kappa Chain | GACATCGTGATGACCCAGAGCCCCGATTCCCTGGC |
| | nucleotide sequence | CGTGTCTCTGGGAGAGAGGGCAACAATCAACTGT |
| | | AAGAGCTCCCAGACCCTGCTGTACACATCCAACTC |
| | | TAAGAATTACCTGGCCTGGTATCAGCAGAAAGTG |
| | | GGACAGCCACCTAGGCTGCTGATCTATTGGGCCTC |
| | | TACCAGGGAGAGCGGCGTGCCAGACAGATTCAGC |
| | | GGCTCCGGCTCTGGCACAGACTTCACCCTGACAAT |
| | | CTCTAGCCTGCTGGCCGAGGACGTGGCCGTGTACT |
| | | ATTGCCAGCAGTACTATACCACACCCCTGACCTTC |
| | | GGCGGCGGCACAAAGGTGGAGGTGAAG |
| 238 | C10 clone Kappa Chain CDR1 | KSSQTLLYTSNSKNYLA |
| 239 | C10 clone Kappa Chain CDR2 | WASTRES |
| 240 | C10 clone Kappa Chain CDR3 | QQYYTTPLT |
| 241 | B12 clone Heavy Chain aa | EVQLVESGGGVVHPGKSLTLSCEASGFTFNDHGIHW |
| | sequence | VRRAPGKGLEWLALISKDGSKEYSTDSVKGRFTVSR |
| | | DNSRNTVFLQMKSLTTEDTAIYYCAKDMGQCSSPSC |
| | | STMDSYFAMDVWGQGTTVIVSS |
| 242 | B12 clone Heavy Chain | GAGGTGCAGCTGGTGGAGTCCGGCGGAGGAGTGG |
| | nucleotide sequence | TGCACCCTGGCAAGTCTCTGACCCTGAGCTGTGAG |
| | | GCCAGCGGCTTCACCTTCAACGACCACGGCATCCA |
| | | CTGGGTGCGGAGAGCACCTGGCAAGGGCCTGGAG |
| | | TGGCTGGCCCTGATCTCTAAGGACGGCAGCAAGG |
| | | AGTACAGCACCGATTCCGTGAAGGGCCGGTTCAC |
| | | AGTGTCCAGGGATAACTCTCGCAATACCGTGTTTC |
| | | TGCAGATGAAGTCTCTGACCACAGAGGACACAGC |
| | | CATCTACTATTGCGCCAAGGATATGGGCCAGTGCA |
| | | GCTCCCCCTCCTGTTCTACCATGGACAGCTATTTC |
| | | GCAATGGACGTGTGGGGACAGGGAACCACAGTGA |
| | | TCGTGTCTAGC |
| 243 | B12 clone Heavy Chain CDR1 | GFTFNDH |
| 244 | B12 clone Heavy Chain CDR2 | SKDGSK |
| 245 | B12 clone Heavy Chain CDR3 | DMGQCSSPSCSTMDSYFAMDV |
| 246 | B12 clone Kappa Chain aa | DIVMTQSPLSLPVTPGEPASISCRSSQNLRHNNGYNY |
| | sequence | LNWYLQKPGQSPQLLIYLGSIRASGVPDRFSGSGSGT |
| | | DFTLKISRVEAEDVGVYYCMQALQTPPWTFGQGTK |
| | | VDFK |
| 247 | B12 clone Kappa Chain | GACATCGTGATGACCCAGTCCCCTCTGTCTCTGCC |
| | nucleotide sequence | AGTGACACCCGGCGAGCCTGCCTCTATCAGCTGTC |
| | | GGAGCTCCCAGAACCTGAGACACAACAATGGCTA |
| | | CAACTATCTGAATTGGTACCTGCAGAAGCCAGGCC |
| | | AGTCTCCCCAGCTGCTGATCTATCTGGGCAGCATC |
| | | AGGGCCTCCGGCGTGCCCGACCGCTTCTCCGGCTC |
| | | TGGCAGCGGCACCGACTTCACCCTGAAGATCAGC |
| | | CGGGTGGAGGCAGAGGACGTGGGCGTGTACTATT |
| | | GCATGCAGGCCCTGCAGACCCCCCCTTGGACATTC |
| | | GGCCAGGGCACCAAGGTGGACTTCAAG |
| 248 | B12 clone Kappa Chain CDR1 | RSSQNLRHNNGYNYLN |
| 249 | B12 clone Kappa Chain CDR2 | LGSIRAS |
| 250 | B12 clone Kappa Chain CDR3 | MQALQTPPWT |
| 251 | G7 clone Heavy Chain aa | QVQLVQSGAEVKKPGASVKVSCKASGYTFTSHYMH |
| | sequence | WVRQAPGQGLEWMGIISPTGDFTNYAQKFQGRVTL |
| | | TRDTSTSTDYMEVTSLRSEDTAVYYCARDCSAWAP |
| | | DYWGQGTLVTVSS |
| 252 | G7 clone Heavy Chain | CAGGTGCAGCTGGTGCAGTCCGGCGCAGAGGTGA |
| | nucleotide sequence | AGAAGCCAGGAGCCAGCGTGAAGGTGTCCTGTAA |
| | | GGCCTCTGGCTACACCTTCACATCTCACTATATGC |
| | | ACTGGGTGCGGCAGGCACCAGGACAGGGCCTGGA |
| | | GTGGATGGGCATCATCAGCCCTACAGGCGACTTCA |
| | | CCAACTACGCCCAGAAGTTTCAGGGCCGGGTGAC |
| | | CCTGACAAGAGACACCTCTACAAGCACCGATTAT |
| | | ATGGAGGTGACATCCCTGAGGTCTGAGGATACCG |
| | | CCGTGTACTATTGCGCAAGGGACTGTTCCGCCTGG |
| | | GCCCCCGATTACTGGGGACAGGGCACACTGGTGA |
| | | CCGTGAGCTCC |
| 253 | G7 clone Heavy Chain CDR1 | GYTFTSH |
| 254 | G7 clone Heavy Chain CDR2 | SPTGDF |
| 255 | G7 clone Heavy Chain CDR3 | DCSAWAPDY |
| 256 | G7 clone Kappa Chain aa | QSALTRPPSVSRCPGQSITISCSGTSSDVGHDNHVSW |
| | sequence | YQQHPGRAPKLMVYEVRNRPSGVSDRFSGSKSGNT |
| | | ASLTISGLQAEDEATYYCCSYTTTHRYIFGGGTKLT |
| 257 | G7 clone Kappa Chain | CAGTCTGCCCTGACAAGGCCCCCTTCTGTGAGCCG |
| | nucleotide sequence | CTGCCCTGGACAGAGCATCACAATCTCCTGTTCTG |
| | | GCACCAGCTCCGACGTGGGCCACGATAACCACGT |
| | | GTCCTGGTACCAGCAGCACCCAGGAAGGGCACCC |
| | | AAGCTGATGGTGTATGAGGTGCGGAACAGACCAA |
| | | GCGGCGTGTCCGACAGGTTCAGCGGCTCCAAGTCT |
| | | GGCAATACAGCCTCTCTGACCATCAGCGGCCTGCA |
| | | GGCAGAGGATGAGGCAACCTACTATTGCTGTTCTT |
| | | ACACCACAACCCACCGGTATATCTTTGGCGGCGGC |
| | | ACAAAGCTGACC |
| 258 | G7 clone Kappa Chain CDR1 | SGTSSDVGHDNHVS |
| 259 | G7 clone Kappa Chain CDR2 | EVRNRPS |
| 260 | G7 clone Kappa Chain CDR3 | CSYTTTHRYI |
| 261 | E11 clone Heavy Chain aa | QVQLLGSGPGLVKPSETLSLTCTVSGASISSPGYYWG |
| | sequence | FIRQSPGKGLEWIGSMVSGGTTYYNPSLKSRVTISMD |
| | | MSNNQFSLRLNSVTAADTALYYCARGSRQLVRRATI |
| | | DYWGQGALFTVSP |
| 262 | E11 clone Heavy Chain | CAGGTGCAGCTGCTGGGCAGCGGCCCAGGCCTGG |
| | nucleotide sequence | TGAAGCCTTCTGAGACACTGAGCCTGACCTGTACA |
| | | GTGTCTGGCGCCAGCATCAGCTCCCCAGGCTACTA |
| | | TTGGGGCTTCATCAGGCAGAGCCCAGGCAAGGGC |
| | | CTGGAGTGGATCGGCTCCATGGTGTCTGGCGGCAC |
| | | CACATACTATAACCCTAGCCTGAAGTCCCGGGTGA |
| | | CAATCTCCATGGACATGTCTAACAATCAGTTCAGC |
| | | CTGAGGCTGAATTCCGTGACCGCCGCCGATACAGC |
| | | CCTGTACTATTGCGCAAGGGGCTCCCGCCAGCTGG |
| | | TGCGGAGAGCAACCATCGACTACTGGGGACAGGG |
| | | CGCCCTGTTTACAGTGTCTCCC |
| 263 | E11 clone Heavy Chain CDR1 | GASISSPGY |
| 264 | E11 clone Heavy Chain CDR2 | VSGGT |
| 265 | E11 clone Heavy Chain CDR3 | GSRQLVRRATIDY |
| 266 | E11 clone Lambda Chain aa | QSVLTGPPSVSAGPGQQVFISCSGNSSNIGNNYVSWY |
| | sequence | QQLPGTAPKLLIYDSNKRPSGIPDRFSGSKSGTSATLG |
| | | ITGLQTGDEADYYCGTWDSSLSAGVFGGGTKLT |
| 267 | E11 clone Lambda Chain | CAGTCTGTGCTGACCGGACCACCTTCCGTGTCTGC |
| | nucleotide sequence | CGGACCAGGACAGCAGGTGTTCATCAGCTGTTCCG |
| | | GCAACAGCTCCAATATCGGCAACAATTACGTGTCT |
| | | TGGTATCAGCAGCTGCCAGGCACAGCCCCCAAGC |
| | | TGCTGATCTACGACTCTAACAAGCGGCCTAGCGGC |
| | | ATCCCAGATAGATTCTCTGGCAGCAAGTCCGGCAC |
| | | CAGCGCCACACTGGGCATCACCGGCCTGCAGACA |
| | | GGCGACGAGGCAGATTACTATTGCGGAACCTGGG |
| | | ACTCTAGCCTGTCCGCCGGCGTGTTTGGAGGAGGA |
| | | ACCAAGCTGACA |
| 268 | E11 clone Lambda Chain | SGNSSNIGNNYVS |
| | CDR1 | |
| 269 | E11 clone Lambda Chain | DSNKRPS |
| | CDR2 | |
| 270 | E11 clone Lambda Chain | GTWDSSLSAGV |
| | CDR3 | |
| 271 | G5 clone Heavy Chain aa | EVQLVESGGGLVKPGESLRLSCAASGFTFSSYSMSW |
| | sequence | VRQAPGKGLEWVSCITSSGHTYYADSVKGRFAISRD |
| | | NGKNSLYLQMNNLRAEDTAVYFCAKELGAHSGLFY |
| | | NGVFDYWGQGNPVTVSS |
| 272 | G5 clone Heavy Chain | GAGGTGCAGCTGGTGGAGTCCGGCGGAGGCCTGG |
| | nucleotide sequence | TGAAGCCAGGCGAGTCTCTGAGGCTGAGCTGTGC |
| | | CGCCTCCGGCTTCACCTTTAGCTCCTACAGCATGT |
| | | CCTGGGTGCGCCAGGCACCTGGCAAGGGCCTGGA |
| | | GTGGGTGTCCTGCATCACCTCTAGCGGCCACACAT |
| | | ACTATGCCGACTCTGTGAAGGGCCGGTTCGCCATC |
| | | AGCCGGGATAACGGCAAGAATAGCCTGTACCTGC |
| | | AGATGAACAATCTGCGGGCCGAGGACACCGCCGT |
| | | GTATTTTTGTGCAAAGGAGCTGGGAGCACACTCTG |
| | | GCCTGTTCTACAACGGCGTGTTTGATTATTGGGGC |
| | | CAGGGCAATCCCGTGACAGTGTCCTCT |
| 273 | G5 clone Heavy Chain CDR1 | GFTFSSY |
| 274 | G5 clone Heavy Chain CDR2 | TSSGH |
| 275 | G5 clone Heavy Chain CDR3 | ELGAHSGLFYNGVFDY |
| 276 | G5 clone Lambda Chain aa | QSALTQPASVSGSPGQSITISCTGTSSDVGGYNYVSW |
| | sequence | YQQHPGKAPKLMIYEVSNRPSGVSNRFSGSKGNTAS |
| | | LTISGLRGEDEADYYCSSYTSSSTLVVFGGGTKLT |
| 277 | G5 clone Lambda Chain | CAGTCCGCCCTGACCCAGCCAGCCTCCGTGTCTGG |
| | nucleotide sequence | CAGCCCCGGCCAGTCTATCACAATCAGCTGTACCG |
| | | GCACAAGCTCCGACGTGGGCGGCTACAACTACGT |
| | | GAGCTGGTACCAGCAGCACCCAGGCAAGGCACCT |
| | | AAGCTGATGATCTATGAGGTGTCCAACAGGCCAA |
| | | GCGGCGTGTCCAATAGATTCTCCGGCTCTAAGGGC |
| | | AATACCGCCTCCCTGACAATCTCTGGCCTGAGGGG |
| | | AGAGGACGAGGCAGATTACTATTGCTCTAGCTAC |
| | | ACCTCCTCTAGCACACTGGTGGTGTTTGGCGGCGG |
| | | CACCAAGCTGACA |
| 278 | G5 clone Lambda Chain | TGTSSDVGGYNYVS |
| | CDR1 | |
| 279 | G5 clone Lambda Chain | EVSNRPS |
| | CDR2 | |
| 280 | G5 clone Lambda Chain | SSYTSSSTLVV |
| | CDR3 | |
| 281 | H8 clone Heavy Chain aa | QVHLQQWGAGLVKPSETLSLTCAVQGGPFSGYYWS |
| | sequence | WIRQPPGKGLEWIGEINHSGNTHYNPSLKSRVTISVD |
| | | TSGNYFSLKLTSVTAADAAVYFCARGQQLLRNYYY |
| | | YSGMDVWGQGTTVTVSS |
| 282 | H8 clone Heavy Chain | CAGGTGCACCTGCAGCAGTGGGGAGCAGGCCTGG |
| | nucleotide sequence | TGAAGCCATCCGAGACACTGTCTCTGACATGTGCA |
| | | GTGCAGGGAGGACCCTTCTCTGGCTACTATTGGAG |
| | | CTGGATCAGGCAGCCACCTGGCAAGGGCCTGGAG |
| | | TGGATCGGCGAGATCAACCACAGCGGCAATACCC |
| | | ACTACAACCCCTCTCTGAAGAGCCGGGTGACCATC |
| | | AGCGTGGACACATCCGGCAATTACTTCTCCCTGAA |
| | | GCTGACCTCTGTGACAGCCGCCGATGCCGCCGTGT |
| | | ATTTTTGCGCCCGGGGCCAGCAGCTGCTGAGAAAC |
| | | TACTATTACTATTCCGGCATGGACGTGTGGGGACA |
| | | GGGAACCACAGTGACAGTGAGCTCC |
| 283 | H8 clone Heavy Chain CDR1 | GGPFSGY |
| 284 | H8 clone Heavy Chain CDR2 | NHSGN |
| 285 | H8 clone Heavy Chain CDR3 | GQQLLRNYYYYSGMDV |
| 286 | H8 clone Lambda Chain aa | QSALTQPASVSGSPGQSITISCTETSRDVGDYNYVSW |
| | sequence | YQQHPGPAPKLIMYEVHKRPSGISNRFSGSKSGTTAS |
| | | LTISGLQADDEGDYYCSSYTDKNTYVFGSGTQVT |
| 287 | H8 clone Lambda Chain | CAGTCTGCCCTGACCCAGCCAGCCTCTGTGAGCGG |
| | nucleotide sequence | CTCCCCTGGCCAGTCCATCACAATCTCTTGTACCG |
| | | AGACATCTCGGGACGTGGGCGATTACAACTATGT |
| | | GAGCTGGTACCAGCAGCACCCAGGACCTGCACCA |
| | | AAGCTGATCATGTATGAGGTGCACAAGCGGCCCT |
| | | CTGGCATCAGCAATAGATTCTCTGGCAGCAAGTCC |
| | | GGCACCACAGCCAGCCTGACCATCTCCGGCCTGCA |
| | | GGCAGACGATGAGGGCGACTACTATTGCAGCTCC |
| | | TACACCGATAAGAACACATACGTGTTCGGCAGCG |
| | | GCACCCAGGTGACA |
| 288 | H8 clone Lambda Chain | TETSRDVGDYNYVS |
| | CDR1 | |
| 289 | H8 clone Lambda Chain | EVHKRPS |
| | CDR2 | |
| 290 | H8 clone Lambda Chain | SSYTDKNTYV |
| | CDR3 | |
| 291 | gp350 B95-8 (2-425) | EAALLVCQYTIQSLIHLTGEDPGFFNVEIPEFPFYPTC |
| | D296R/I160R Avi-His | NVCTADVNVTINFDVGGKKHQLDLDFGQLTPHTKA |
| | | VYQPRGAFGGSENATNLFLLELLGAGELALTMRSKK |
| | | LPINVTTGEEQQVSLESVDVYFQDVFGTMWCHHAE |
| | | MQNPVYLIPETVPYIDWDNCNSTNITAVVRAQGLDV |
| | | TLPLSLPTSAQDSNFSVKTEMLGNEIDIECIMEDGEIS |
| | | QVLPGDNKFNITCSGYESHVPSGGILTSTSPVATPIPG |
| | | TGYAYSLRLTPRPVSRFLGNNSILYVFYSGNGPKASG |
| | | GRYCIQSNIVFSDEIPASQDMPTNTTDITYVGDNATY |
| | | SVPMVTSEDANSPNVTVTAFWAWPNNTETDFKCKW |
| | | TLTSGTPSGCENISGAFASNRTFDITVSGLGTAPKTLII |
| | | TRTATNATTTTHKVIFSKAPGSGLNDIFEAQKIEWHE |
| | | HHHHHH |
| 292 | NAM encoding SEQ ID | GAAGCTGCCCTGCTCGTGTGCCAGTACACCATCCA |
| | NO: 281 | GAGCCTGATCCACCTGACCGGCGAGGACCCCGGC |
| | | TTCTTCAACGTGGAAATCCCCGAGTTCCCCTTCTA |
| | | CCCTACCTGCAACGTGTGCACCGCCGACGTGAACG |
| | | TGACCATCAACTTCGACGTGGGCGGCAAGAAGCA |
| | | CCAGCTGGACCTGGATTTCGGCCAGCTGACCCCTC |
| | | ACACCAAGGCCGTGTATCAGCCCAGAGGCGCCTTT |
| | | GGCGGCAGCGAGAACGCCACCAATCTGTTTCTGCT |
| | | GGAACTCCTAGGCGCCGGCGAGCTGGCCCTGACC |
| | | ATGAGAAGCAAGAAACTGCCCATCAATGTGACCA |
| | | CAGGCGAGGAACAGCAGGTGTCCCTGGAAAGCGT |
| | | GGACGTGTACTTTCAAGACGTGTTCGGCACCATGT |
| | | GGTGCCACCACGCCGAGATGCAGAACCCCGTGTA |
| | | CCTGATCCCCGAGACAGTGCCCTACATCGATTGGG |
| | | ACAACTGCAACAGCACCAACATCACCGCCGTCGT |
| | | GCGGGCCCAGGGACTGGATGTGACACTGCCTCTG |
| | | AGCCTGCCTACCAGCGCCCAGGACAGCAACTTCA |
| | | GCGTGAAAACCGAGATGCTGGGCAACGAGATCGA |
| | | CATCGAGTGCATCATGGAAGATGGCGAGATCAGC |
| | | CAGGTGCTGCCCGGCGACAACAAGTTCAACATCA |
| | | CATGCAGCGGCTACGAGAGCCACGTGCCATCTGG |
| | | CGGCATCCTGACCAGCACAAGCCCAGTGGCCACA |
| | | CCCATCCCTGGCACAGGCTACGCCTACAGCCTGAG |
| | | ACTGACCCCCAGACCCGTGTCCAGATTCCTGGGCA |
| | | ACAACAGCATCCTGTACGTGTTCTACAGCGGCAAC |
| | | GGCCCCAAGGCCTCTGGCGGCCGGTACTGTATCCA |
| | | GAGCAACATCGTGTTCAGCGACGAGATCCCCGCC |
| | | AGCCAGGACATGCCCACCAATACCACCGACATCA |
| | | CGTACGTGGGCGACAATGCCACCTACAGCGTGCC |
| | | AATGGTCACCTCCGAGGACGCCAACAGCCCCAAC |
| | | GTGACCGTGACAGCCTTTTGGGCCTGGCCTAACAA |
| | | CACCGAGACAGACTTCAAGTGCAAGTGGACCCTG |
| | | ACCTCCGGCACCCCTAGCGGCTGCGAGAATATCA |
| | | GCGGAGCCTTCGCCAGCAACCGGACCTTCGATATC |
| | | ACCGTGTCTGGCCTGGGCACCGCCCCCAAGACCCT |
| | | GATCATCACCAGAACCGCCACAAATGCCACCACC |
| | | ACAACCCACAAAGTGATCTTCAGCAAGGCCCCCG |
| | | GCTCTGGCCTGAACGACATTTTTGAGGCCCAGAAG |
| | | ATTGAGTGGCATGAACATCACCACCACCACCAT |
| 293 | Macaque Light chain | tcgcgcgttt cggtgatgac ggtgaaaacc tctgacacat gcagctcccg |
| | backbone nucleotide | gagacggtca cagcttgtct gtaagcggat gccgggagca gacaagcccg |
| | sequence | tcagggcgcg tcagcgggtg ttggcgggtg tcggggctgg cttaactatg |
| | | cggcatcaga gcagattgta ctgagagtgc accatatgcg gtgtgaaata |
| | | ccgcacagat gcgtaaggag aaaataccgc atcagattgg |
| | | ctattggcca ttgcatacgt tgtatccata tcataatatg tacatttata |
| | | ttggctcatg tccaacatta ccgccatgtt gacattgatt attgactagt |
| | | tattaatagt aatcaattac ggggtcatta gttcatagcc catatatgga |
| | | gttccgcgtt acataactta cggtaaatgg cccgcctggc tgaccgccca |
| | | acgacccccg cccattgacg tcaataatga cgtatgttcc |
| | | catagtaacg ccaataggga ctttccattg acgtcaatgg gtggagtatt |
| | | tacggtaaac tgcccacttg gcagtacatc aagtgtatca tatgccaagt |
| | | acgcccccta ttgacgtcaa tgacggtaaa tggcccgcct ggcattatgc |
| | | ccagtacatg accttatggg actttcctac ttggcagtac atctacgtat |
| | | tagtcatcgc tattaccatg gtgatgcggt tttggcagta |
| | | catcaatggg cgtggatagc ggtttgactc acggggattt ccaagtctcc |
| | | accccattga cgtcaatggg agtttgtttt ggcaccaaaa tcaacgggac |
| | | tttccaaaat gtcgtaacaa ctccgcccca ttgacgcaaa tgggcggtag |
| | | gcgtgtacgg tgggaggtct atataagcag agctcgttta gtgaaccgtc |
| | | agatcgcctg gagacgccat ccacgctgtt ttgacctcca |
| | | tagaagacac cgggaccgat ccagcctcca tcggctcgca tctctccttc |
| | | acgcgcccgc cgccctacct gaggccgcca tccacgccgg ttgagtcgcg |
| | | ttctgccgcc tcccgcctgt ggtgcctcct gaactgcgtc cgccgtctag |
| | | gtaagtttaa agctcaggtc gagaccgggc ctttgtccgg cgctcccttg |
| | | gagcctacct agactcagcc ggctctccac gctttgcctg |
| | | accctgcttg ctcaactcta gttaacggtg gagggcagtg tagtctgagc |
| | | agtacacgtt gctgccgcgc gcgccaccag acataatagc tgacagacta |
| | | acagactgtt cctttccatg ggtcttttct gcagtcaccg tcgtcgacac |
| | | gtgtgatcag atatcgcggc cgctctagac caccatggga tggtcatgta |
| | | tcatcctttt tctagtagca actgcaaccg gtgtacattc ccagtctgcc |
| | | ctgactcagc ctccctctgt gtctgggtct cctggacagt cggtcaccat |
| | | ctcctgcact ggaaccagca gtgacgttga tggttataac tatgtctcct |
| | | ggtaccaaca acatccaggc aaagccccca aactcatgat ttatggtgtc |
| | | agcaatcggc cctcaggggt ctctgatcgc ttctctggct ccaagtctgg |
| | | caacacggcc tccctgacca tctctgggct ccaggctgag gacgaggctg |
| | | attattactg ttgttcatct acaaccagtt acacttacat cttcggaact |
| | | gggaccaagg tcacagtact aggtcagccc aaggctgccc cctcggtcac |
| | | tctcttcccg ccctcctctg aggagcttca agccaacaag gccacactag |
| | | tgtgtctgat cagtgacttc tacccgggag ccgtggaagt ggcctggaag |
| | | gcagatggca gcgctgtcaa cgcgggagtg gagaccacca aaccctccaa |
| | | acagagcaac aacaagtacg cggccagcag ctacctgagc ctgacgtccg |
| | | accagtggaa gtcccacaag agctacagct gccaggtcac |
| | | gcacgaaggg agcaccgtgg agaagacagt ggcccctgca gaatgttcat |
| | | agggatccag atctgctgtg ccttctagtt gccagccatc tgttgtttgc |
| | | ccctcccccg tgccttcctt gaccctggaa ggtgccactc ccactgtcct |
| | | ttcctaataa aatgaggaaa ttgcatcgca ttgtctgagt aggtgtcatt |
| | | ctattctggg gggtggggtg gggcaggaca gcaaggggga |
| | | ggattgggaa gacaatagca ggcatgctgg ggatgcggtg ggctctatgg |
| | | gtacccaggt gctgaagaat tgacccggtt cctcctgggc cagaaagaag |
| | | caggcacatc cccttctctg tgacacaccc tgtccacgcc cctggttctt |
| | | agttccagcc ccactcatag gacactcata gctcaggagg gctccgcctt |
| | | caatcccacc cgctaaagta catggagcgg tctctccctc |
| | | cctcatcagc ccaccaaacc aaacctagcc tccaagagtg ggaagaaatt |
| | | aaagcaagat aggctattaa gtgcagaggg agagaaaatg cctccaacat |
| | | gtgaggaagt aatgagagaa atcatagaat tttaaggcca tgatttaagg |
| | | ccatcatggc cttaatcttc cgcttcctcg ctcactgact cgctgcgctc |
| | | ggtcgttcgg ctgcggcgag cggtatcagc tcactcaaag |
| | | gcggtaatac ggttatccac agaatcaggg gataacgcag gaaagaacat |
| | | gtgagcaaaa ggccagcaaa aggccaggaa ccgtaaaaag gccgcgttgc |
| | | tggcgttttt ccataggctc cgcccccctg acgagcatca caaaaatcga |
| | | cgctcaagtc agaggtggcg aaacccgaca ggactataaa gataccaggc |
| | | gtttccccct ggaagctccc tcgtgcgctc tcctgttccg |
| | | accctgccgc ttaccggata cctgtccgcc tttctccctt cgggaagcgt |
| | | ggcgctttct catagctcac gctgtaggta tctcagttcg gtgtaggtcg |
| | | ttcgctccaa gctgggctgt gtgcacgaac cccccgttca gcccgaccgc |
| | | tgcgccttat ccggtaacta tcgtcttgag tccaacccgg taagacacga |
| | | cttatcgcca ctggcagcag ccactggtaa caggattagc |
| | | agagcgaggt atgtaggcgg tgctacagag ttcttgaagt ggtggcctaa |
| | | ctacggctac actagaagaa cagtatttgg tatctgcgct ctgctgaagc |
| | | cagttacctt cggaaaaaga gttggtagct cttgatccgg caaacaaacc |
| | | accgctggta gcggtggttt ttttgtttgc aagcagcaga ttacgcgcag |
| | | aaaaaaagga tctcaagaag atcctttgat cttttctacg gggtctgacg |
| | | ctcagtggaa cgaaaactca cgttaaggga ttttggtcat gagattatca |
| | | aaaaggatct tcacctagat ccttttaaat taaaaatgaa gttttaaatc |
| | | aatctaaagt atatatgagt aaacttggtc tgacagttac caatgcttaa |
| | | tcagtgaggc acctatctca gcgatctgtc tatttcgttc atccatagtt |
| | | gcctgactcg gggggggggg gcgctgaggt ctgcctcgtg aagaaggtgt |
| | | tgctgactca taccaggcct gaatcgcccc atcatccagc cagaaagtga |
| | | gggagccacg gttgatgaga gctttgttgt aggtggacca gttggtgatt |
| | | ttgaactttt gctttgccac ggaacggtct gcgttgtcgg gaagatgcgt |
| | | gatctgatcc ttcaactcag caaaagttcg atttattcaa caaagccgcc |
| | | gtcccgtcaa gtcagcgtaa tgctctgcca gtgttacaac caattaacca |
| | | attctgatta gaaaaactca tcgagcatca aatgaaactg caatttattc |
| | | atatcaggat tatcaatacc atatttttga aaaagccgtt tctgtaatga |
| | | aggagaaaac tcaccgaggc agttccatag gatggcaaga tcctggtatc |
| | | ggtctgcgat tccgactcgt ccaacatcaa tacaacctat taatttcccc |
| | | tcgtcaaaaa taaggttatc aagtgagaaa tcaccatgag tgacgactga |
| | | atccggtgag aatggcaaaa gcttatgcat ttctttccag acttgttcaa |
| | | caggccagcc attacgctcg tcatcaaaat cactcgcatc aaccaaaccg |
| | | ttattcattc gtgattgcgc ctgagcgaga cgaaatacgc gatcgctgtt |
| | | aaaaggacaa ttacaaacag gaatcgaatg caaccggcgc aggaacactg |
| | | ccagcgcatc aacaatattt tcacctgaat caggatattc ttctaatacc |
| | | tggaatgctg ttttcccggg gatcgcagtg gtgagtaacc atgcatcatc |
| | | aggagtacgg ataaaatgct tgatggtcgg aagaggcata aattccgtca |
| | | gccagtttag tctgaccatc tcatctgtaa catcattggc aacgctacct |
| | | ttgccatgtt tcagaaacaa ctctggcgca tcgggcttcc catacaatcg |
| | | atagattgtc gcacctgatt gcccgacatt atcgcgagcc catttatacc |
| | | catataaatc agcatccatg ttggaattta atcgcggcct cgagcaagac |
| | | gtttcccgtt gaatatggct cataacaccc cttgtattac tgtttatgta |
| | | agcagacagt tttattgttc atgatgatat atttttatct tgtgcaatgt |
| | | aacatcagag attttgagac acaacgtggc tttccccccc cccccattat |
| | | tgaagcattt atcagggtta ttgtctcatg agcggataca tatttgaatg |
| | | tatttagaaa aataaacaaa taggggttcc gcgcacattt ccccgaaaag |
| | | tgccacctga cgtctaagaa accattatta tcatgacatt aacctataaa |
| | | aataggcgta tcacgaggcc ctttcgtc |

bolded/underlined letters indicate amino acids that differ from wild-type.

### BRIEF DESCRIPTION OF THE FIGURES

FIG. 1: Design of gp350 probes that knock out CR2-binding property. Selected residues substantially involved in CR2-binding, but less in antibody (H02) recognition, are defined by structures of gp350-CR2 and gp350-H02 Fab complexes. CR2-knock out (KO) mutations are designed by substitution of one or more residues among the selected residues. Experimentally tested mutations include Q122N (glycan addition at 122); D163N/164bS (glycan addition at 163 and one residue insertion between 164 and 165); D296R; W162A/D163A/N164A; D296R/I160R; D296R/P158R; D296R/P158N/I160T (glycan addition at 158); D296R/P158R/1160R.

FIG. 2: CR2- and antibody-binding profile of gp350 mutants. Binding of CR2-Ig (chimeric molecule of CR2 SCR1-2 domain and IgG Fc region) and a set of gp350-specific monoclonal antibodies were measured by biolayer interferometry using Octet instrument. CR2-Ig or antibodies were immobilized on biosensors through protein A or anti-mouse Fc antibody (for 2L10) and assessed binding properties of various recombinant gp350 mutants. Graph shows percent binding of each mutant relative to its parental (wild-type) gp350. D296R mutation greatly (but not completely) eliminated CR2-binding while retaining binding of all the monoclonal antibodies. An additional mutation on D296R mutant completely abrogated the CR2-binding property of gp350. Among these variants, D296R/1160R retained binding of most monoclonal antibodies and was therefore chosen for further characterization. 2L10 is a murine non-CR2-binding site targeting antibody, used as a control.

FIGS. 3A-3F: Gating strategy of IgG+ B cell subset in human PBMCs. FIG. 3A: Lymphocytes were gated by SSC and FSC. FIG. 3: B cells were gated by CD19 and excluding non-B cell surface markers. FIG. 3C: Surface IgG+ B cells were further gated by IgG and excluding IgM. SSC and FSC denote side- and forward-scatter, respectively. FIGS. 3D-3F: staining profile of IgG+ B cells with various EBV gp350 probes. There were three gp350 variants used to co-stain with anti-CD21 (complement receptor 2, CR2); WT, wild-type gp350 (FIG. 3D); D296R (FIG. 3E); and D296R/1160R (FIG. 3F). All the mutant probes were designed to reduce the CR2-binding property of gp350. WT and D296R gp350 probes bind to virtually all the IgG+ B cells while the D296R/1160R gp350 probe completely knocked down non-specific gp350 staining of B cells.

FIGS. 4A and 4B: Identification of EBV gp350-specific IgG+ B cells in individuals who have high serum neutralization titers. Cryopreserved PMBCs were stained with cell lineage differentiation markers as well as gp350 D296R/1160R probe. The gp350 probe were conjugated with two different fluorochromes (PE and BV785) to eliminate false positive events and increase confidence for specificity of stained B cells. Plots shown were IgG+ B cells gated as in previous figures. Rectangles indicate IgG+B cells stained with gp350 D296R/1160R in two different colors.

FIGS. 5A-5F: Isolation of gp350-specific B cells from Immunized Macaques. FIG. 5A shows rhesus macaque immunization schedule with EBV gp350 nanoparticles followed by B cell sorting. FIG. 5B shows animals Cy137 and Cy651 displayed the highest neutralization titers and were selected for B cell sorting. FIGS. 5C-5F show the results of B cell sorting strategy for non-human primate B cells.

FIGS. 6A and 6B: Identification and analysis of Vaccine-elicited anti-gp350 mAbs. FIG. 6A shows gp350-specific B cell sorting. FIG. 6B shows the neutralization analysis of select antibodies (72A1 antibody from ATCC is shown for reference).

FIG. 7: Clonotype analysis of immunoglobulin heavy chain sequences. Approximately 200 B cells were isolated from each of the two rhesus macaques analyzed. The B cell receptor sequences indicated low clonal expansion in the B cells isolated as judged by the sequence similarity of each B cell receptor.

FIGS. 8A and 8B: Neutralization potency of humanized mAb H02. FIG. 8A shows that humanized H02, as well as murinized H02, neutralize EBV equivalent to the parental macaque H02. FIG. 8B shows that the neutralizing potency of H02 (human, mouse and macaque) is about 50-fold higher than 72A1 antibody (shown for reference).

FIGS. 9A-9C: Targeting the receptor (CR2)-binding site of gp350: Flow cytometry based CR2 epitope mapping. FIG. 9A is a schematic of the competition-based FACS assay. FIG. 9B shows flow cytometry plots of gp350-binding to B cells in the presence and absence of control antibodies. FIG. 9C shows flow cytometry plots of rhesus macaque antibodies described in this disclosure (two non-neutralizing antibodies F11 and G12 are shown for reference).

FIGS. 10A-10C: Analysis of Vaccine-elicited Anti-gp350 mAbs: characterization of antibodies.

FIG. 10A shows the neutralization potency of select antibodies. FIG. 10B shows the affinity of antibodies to gp350. FIG. 10C shows the CDR H3 isoelectric point of select antibodies, indicating that gp350-specific neutralizing antibodies tend to have a higher isoelectric point than typical antibodies (Ebola GP-elected antibodies are shown for reference).

FIG. 11 shows the location of mutations entered into the heavy chain residues of clain A9; and

FIG. 12 shows the location of mutations entered into the light chain residues of clain A9.

FIG. 13 shows the design of the CR2-binding knockout gp350 probe, including specific residues that were substituted to knock down CR2-binding.

FIG. 14 shows the binding properties of the gp350 mutants. In this figure, # indicates no binding.

FIG. 15 shows the B cell staining profile of the new CR2-binding knock-out gp350 probe (D296R/K161D).

FIG. 16 shows the EBV neutralization potency of the two best human monoclonal antibodies (from clones A9 and E11). In these two clones, mutations were introduced in a structure-guided potency improving mutation analysis. For the mAb of clone A9, the introduced mutations were (referring to Kabat numbering) in the heavy chain were: Q1R, Y32R+Y98I, N53F, T70I, T70F, L96R, Y98R, Y98W, I100F, I100W, and Y102E; in the light chain were Y32E, G57D, T56Q, and T56E. For the mAb of clone E11, the introduced mutations were (referring to Kabat numbering) in the heavy chain were: V100F, V100I, V100W, V100R, V100Y, Q98R, Q98Y, and Y58R (residues 30-33 and 53-56 were also targeted for mutagenesis); in the light chain, residues 1, 27, 27a, 30, 32, 49, 50, 52, 53, 56, and 93-95 were targeted for mutagenesis.

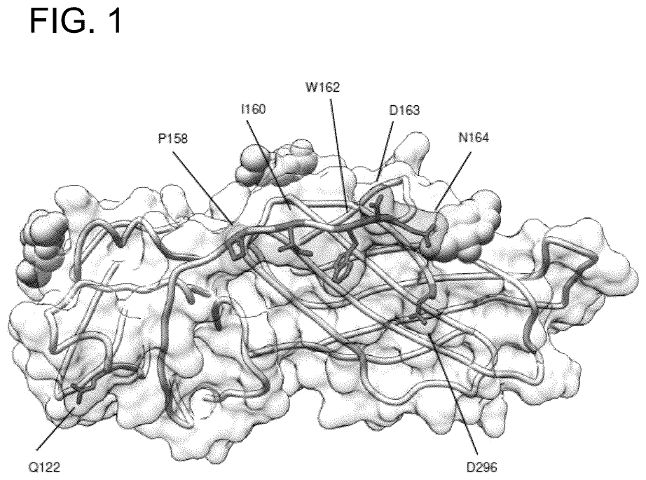

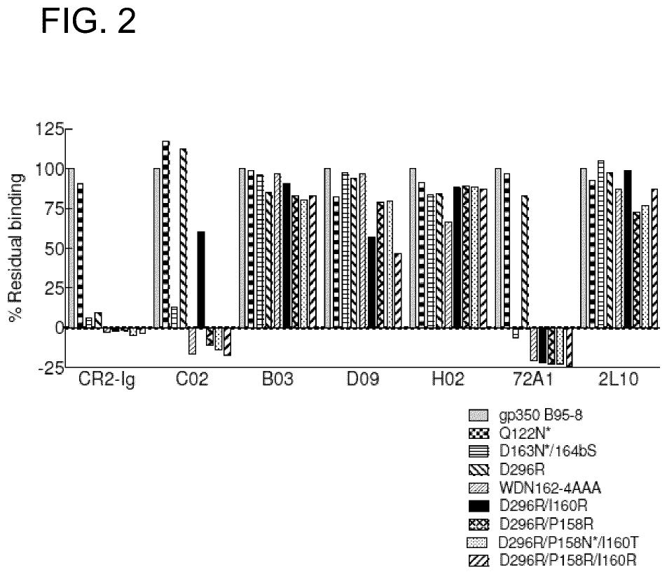

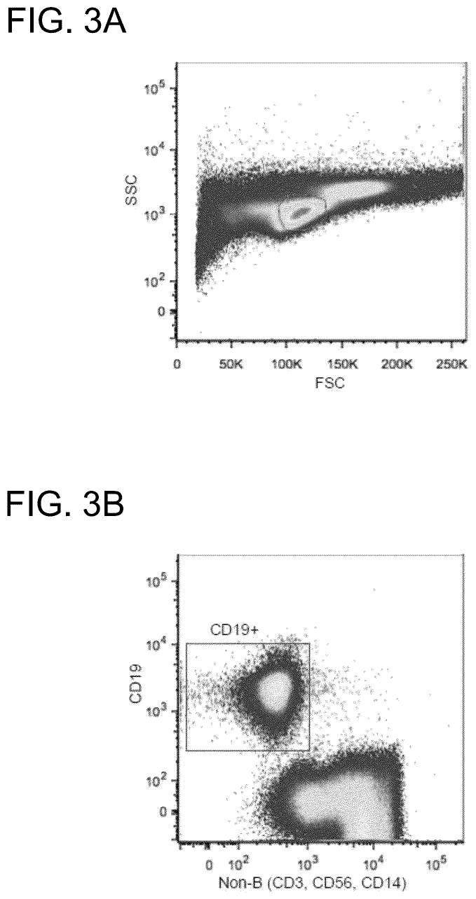

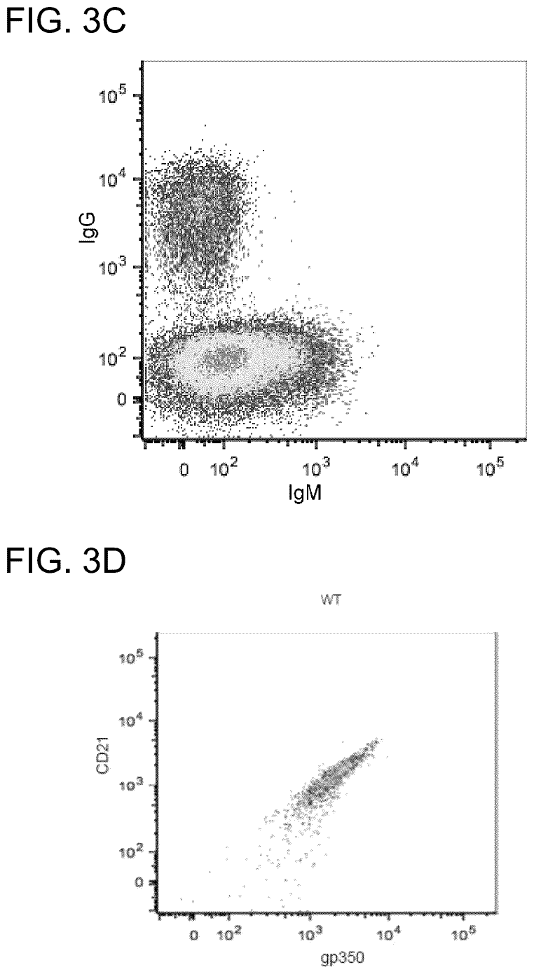

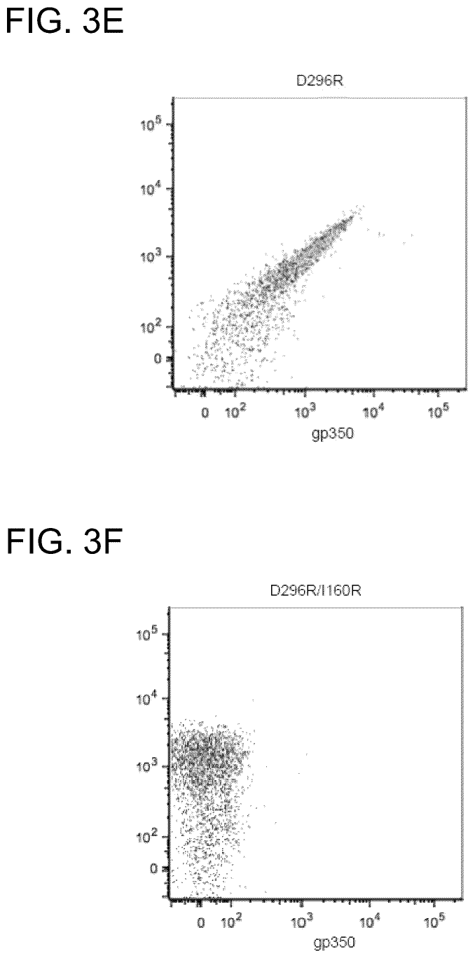

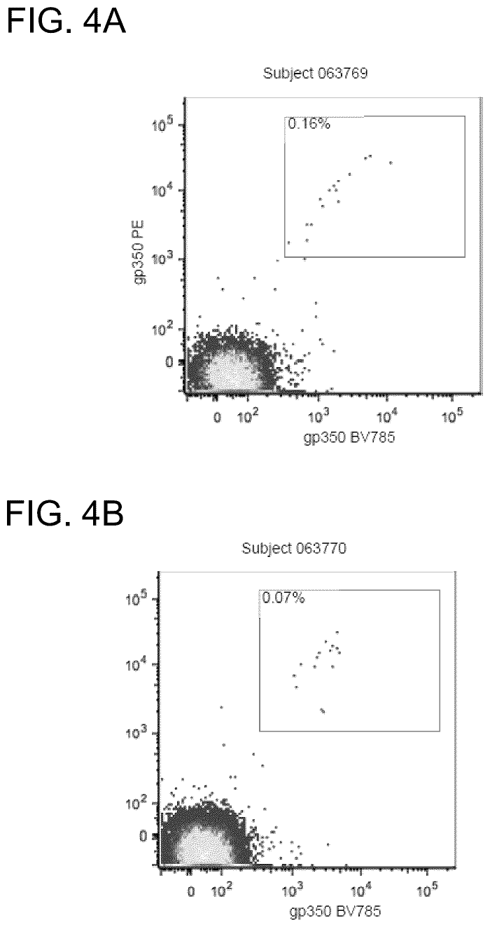

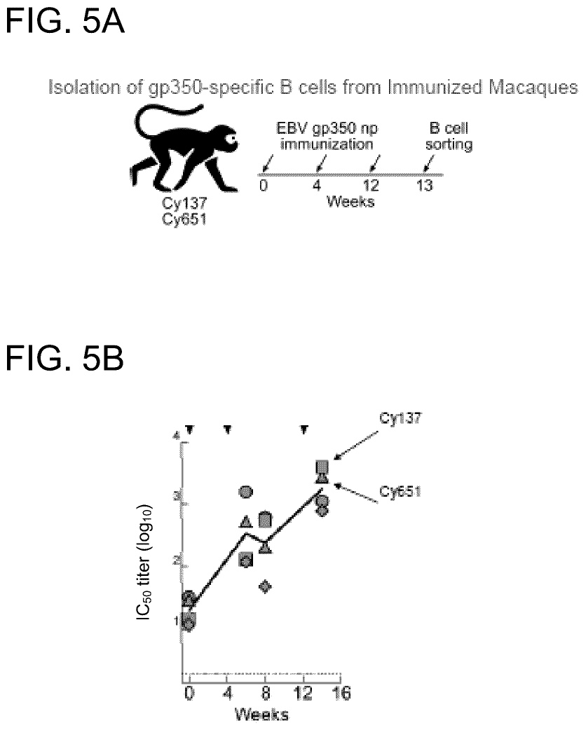

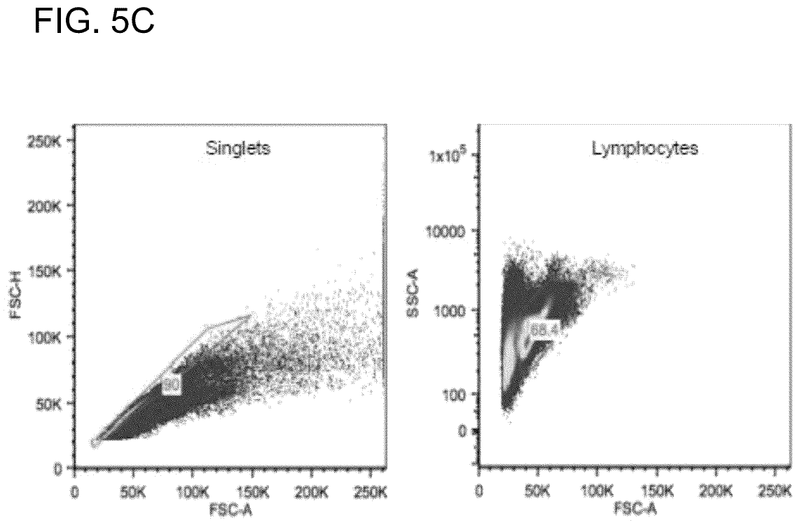

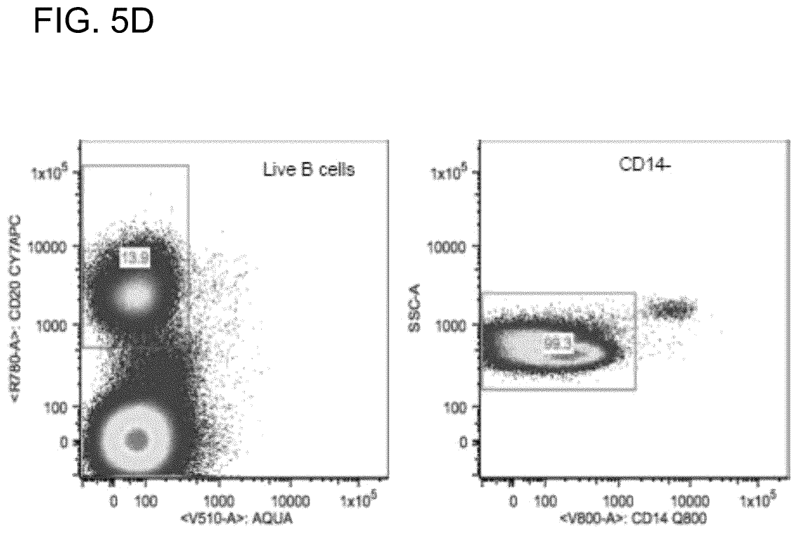

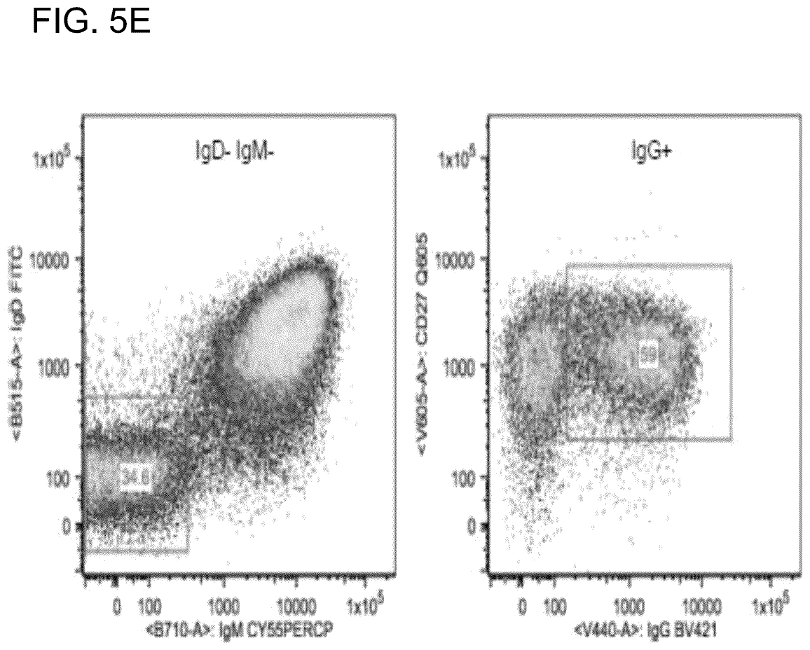

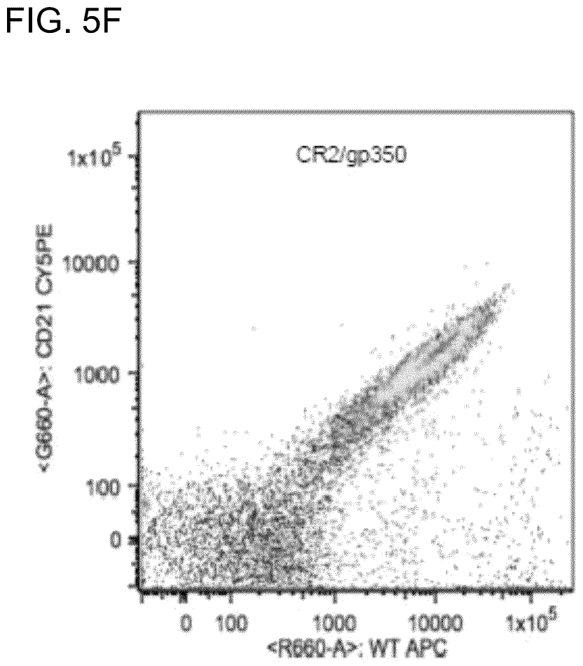

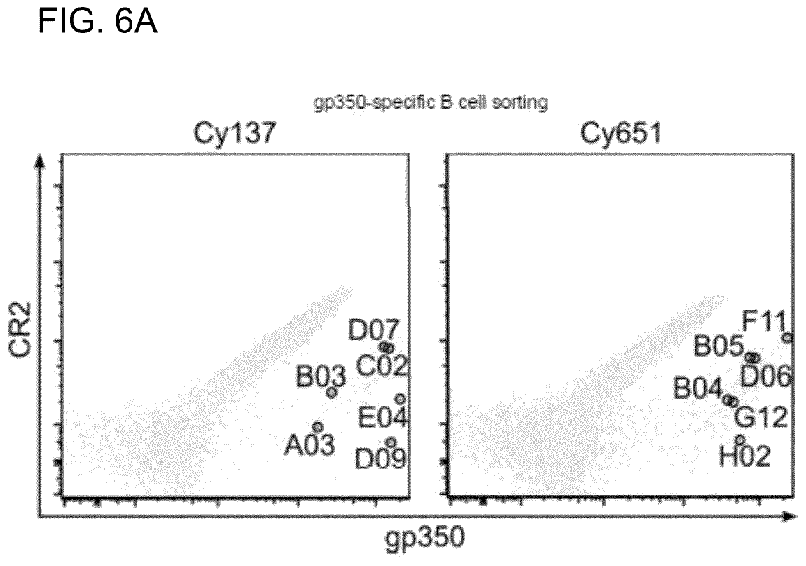

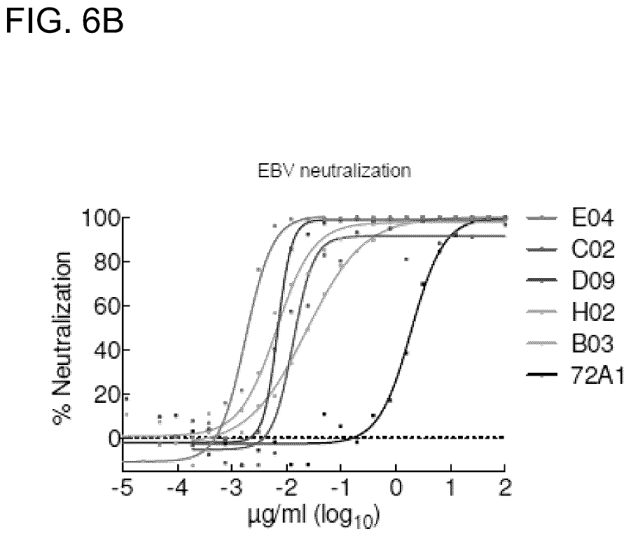

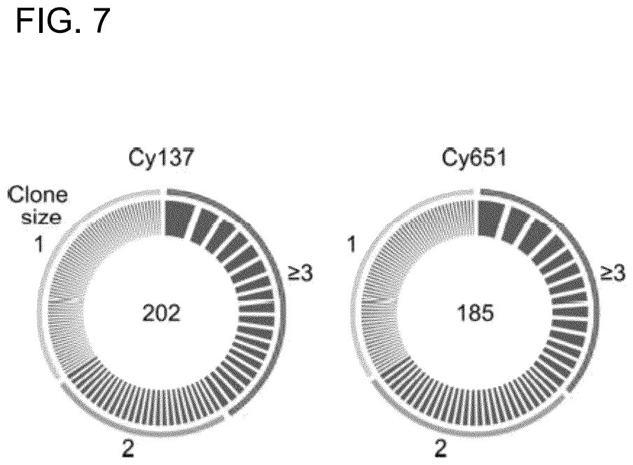

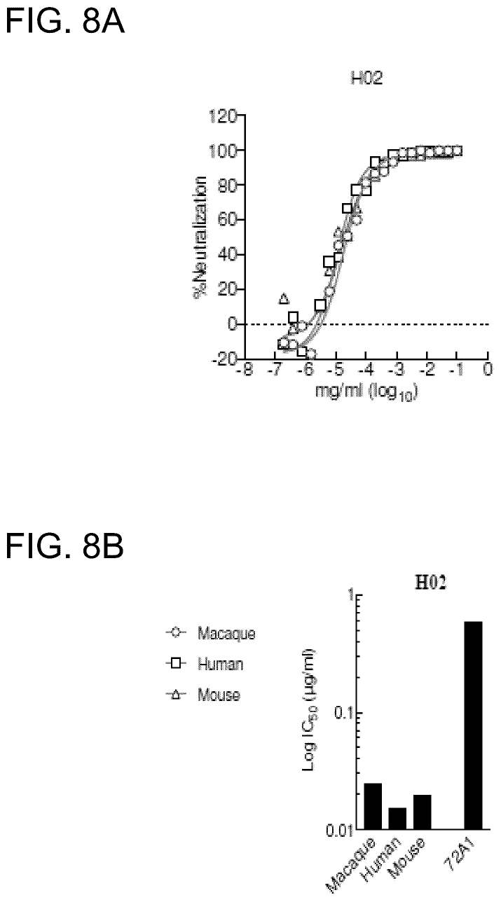

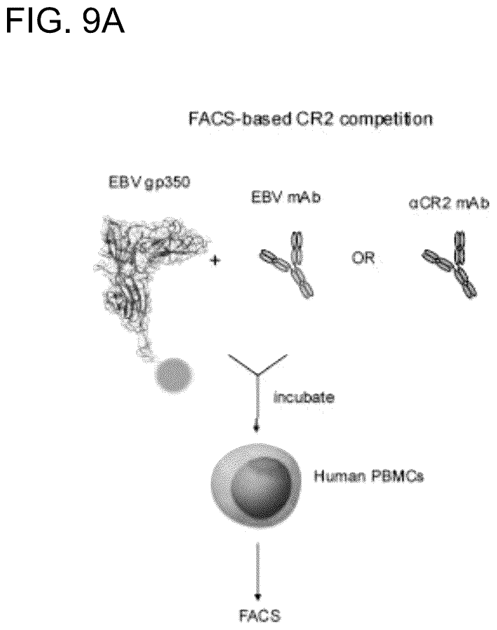

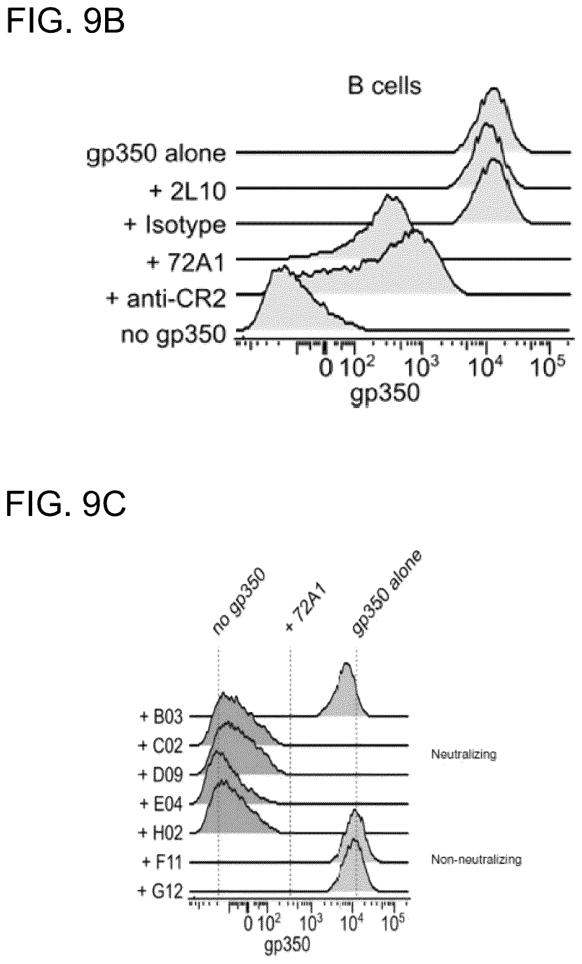

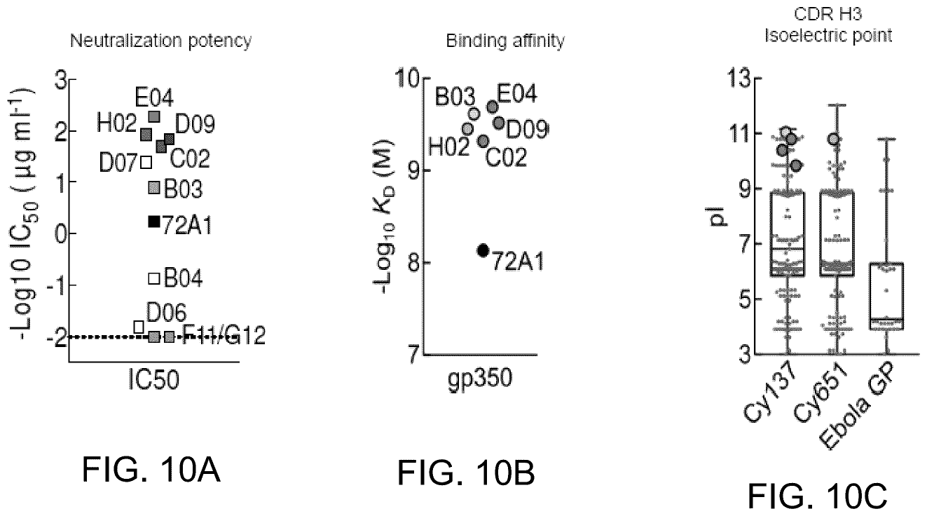

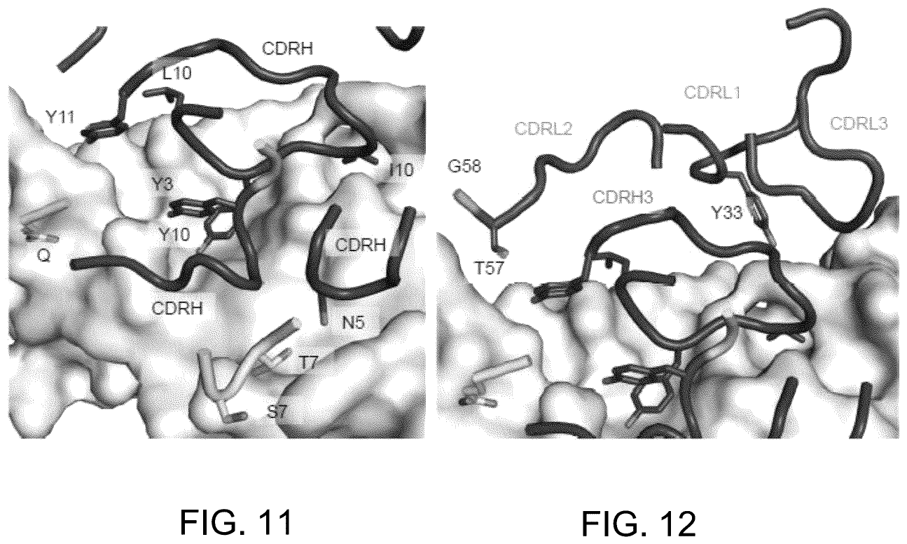

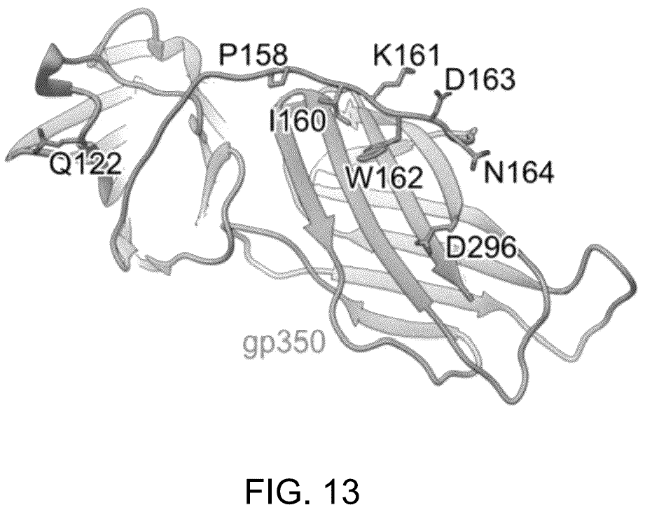

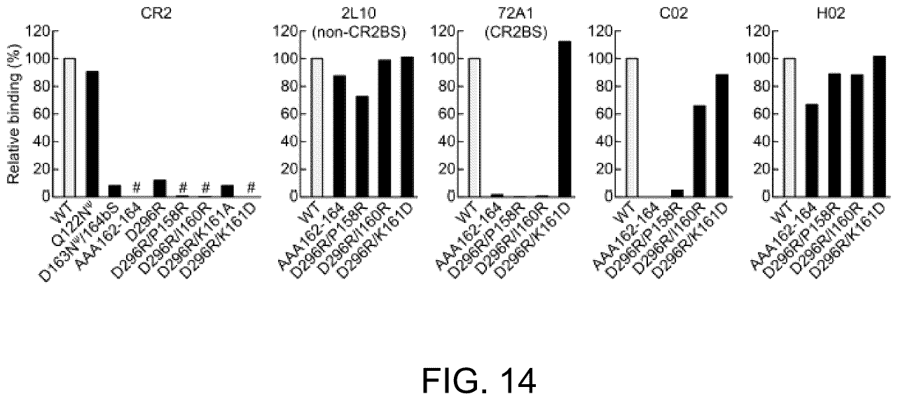

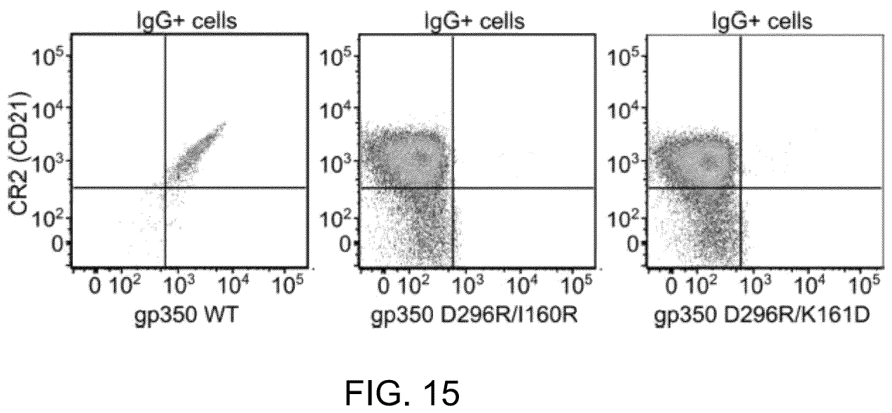

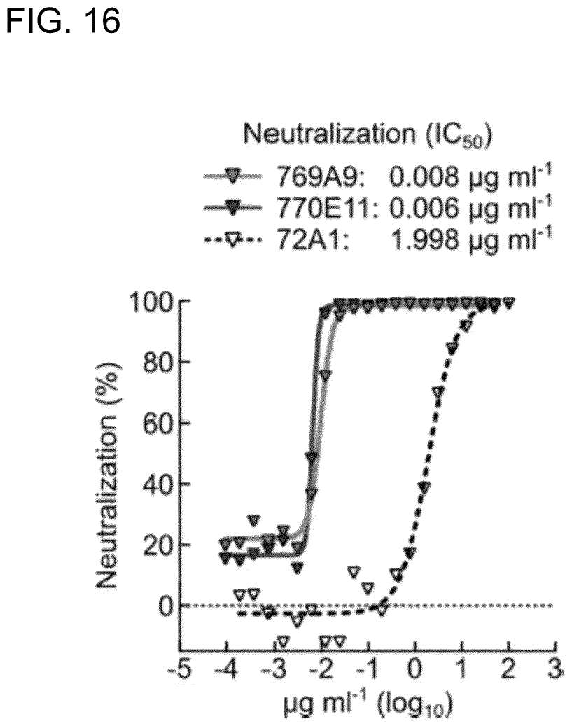

### DETAILED DESCRIPTION

### I. Definitions

The terms “EBV gp350 protein” and “gp350” as used herein, refer to various Epstein Barr Virus polypeptides. The EBV gp350 proteins described herein may be isolated from a variety of sources, such as from human tissue types or from another source, or prepared by recombinant or synthetic methods. The term “EBV gp350 protein” refers to each individual gp350 polypeptide disclosed herein. All disclosures in this specification which refer to the “EBV gp350 protein” refer to each of the polypeptides individually as well as jointly. For example, descriptions of the preparation of, purification of, derivation of, formation of antibodies to or against, formation of gp350 binding oligopeptides to or against, administration of, compositions containing, treatment of a disease with, etc., pertain to each polypeptide of the disclosure individually. The term “EBV gp350 protein” also includes variants of the gp350 polypeptides disclosed herein.

A “native sequence EBV gp350 protein” comprises a polypeptide having the same amino acid sequence as the corresponding EBV gp350 protein derived from nature. Such native sequence EBV gp350 proteins can be isolated from nature or can be produced by recombinant or synthetic means. The term “native sequence EBV gp350 protein” specifically encompasses naturally-occurring truncated or secreted forms of the specific EBV gp350 protein (e.g., an extracellular domain sequence), naturally-occurring variant forms (e.g., alternatively spliced forms) and naturally-occurring allelic variants of the polypeptide. The native sequence EBV gp350 proteins disclosed herein may be mature or full-length native sequence polypeptides comprising the full-length amino acids sequences.

“EBV gp350 protein variant” means an EBV gp350 protein, preferably an active EBV gp350 protein, as defined herein having at least about 80% amino acid sequence identity with a full-length native sequence EBV gp350 protein sequence as disclosed herein, an extracellular domain of an EBV gp350 protein, as disclosed herein or any other fragment of a full-length EBV gp350 protein sequence as disclosed herein (such as those encoded by a nucleic acid that represents only a portion of the complete coding sequence for a full-length EBV gp350 protein). Such EBV gp350 protein variants include, for instance, EBV gp350 proteins wherein one or more amino acid residues are added or deleted, at the N- or C-terminus of the full-length native amino acid sequence. Ordinarily, an EBV gp350 protein variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a full-length native sequence EBV gp350 protein sequence as disclosed herein, or any other specifically defined fragment of a full-length EBV gp350 protein sequence as disclosed herein. Optionally, gp350 variant polypeptides will have no more than one conservative amino acid substitution as compared to the native EBV gp350 protein sequence, alternatively no more than 2, 3, 4, 5, 6, 7, 8, 9, or 10 conservative amino acid substitution as compared to the native EBV gp350 protein sequence.

“Percent (%) amino acid sequence identity” with respect to the EBV gp350 protein sequences identified herein is defined as the percentage of amino acid residues in a candidate sequence that are identical to the amino acid residues in the specific EBV gp350 protein sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, % amino acid sequence identity values may be generated using the sequence comparison computer program ALIGN-2, wherein the complete source code for the ALIGN-2 program is provided in U.S. Pat. No. 7,160,985, which is incorporated herein by reference. The ALIGN-2 sequence comparison computer program was authored by Genentech, Inc. and the source code thereof has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, California or may be compiled from the source code. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as 100 times the fraction X/Y, where X is the number of amino acid residues scored as identical matches by the sequence alignment program ALIGN-2 in that program's alignment of A and B, and where Y is the total number of amino acid residues in B. Where the length of amino acid sequence A is not equal to the length of amino acid sequence B, the % amino acid sequence identity of A to B will not equal the % amino acid sequence identity of B to A.

“gp350 variant polynucleotide” or “gp350 variant nucleic acid sequence” means a nucleic acid molecule which encodes an EBV gp350 protein, preferably an active EBV gp350 protein, as defined herein and which has at least about 80% nucleic acid sequence identity with a nucleic acid sequence encoding a full-length native sequence EBV gp350 protein sequence as disclosed herein, an extracellular domain of an EBV gp350 protein, as disclosed herein or any other fragment of a full-length EBV gp350 protein sequence as disclosed herein (such as those encoded by a nucleic acid that represents only a portion of the complete coding sequence for a full-length EBV gp350 protein). Ordinarily, a gp350 variant polynucleotide will have at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% nucleic acid sequence identity with a nucleic acid sequence encoding a full-length native sequence EBV gp350 protein sequence, an extracellular domain of an EBV gp350 protein or any other fragment of a full-length EBV gp350 protein sequence. Variants do not encompass the native nucleotide sequence.

Ordinarily, gp350 variant polynucleotides are at least about 5 nucleotides in length, alternatively at least about 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length.

“Isolated,” when used to describe the various EBV gp350 proteins disclosed herein, means a polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. The polypeptide may be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or silver stain. Isolated polypeptide includes polypeptide in situ within recombinant cells, since at least one component of the EBV gp350 protein natural environment will not be present. Ordinarily, however, isolated polypeptide will be prepared by at least one purification step.

An “isolated” EBV gp350 protein-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

The term “control sequences” refers to DNA sequences necessary for the expression of an operably linked coding sequence in a host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers (1995).

“Stringent conditions” or “high stringency conditions” as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “epitope tagged” used herein refers to a chimeric polypeptide comprising an EBV gp350 protein or anti-gp350 antibody fused to a “tag polypeptide.” The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

“Active” or “activity” for the purposes herein refers to form(s) of an EBV gp350 protein which retain a biological and/or an immunological activity of native or naturally-occurring gp350, wherein “biological” activity refers to a biological function (either inhibitory or stimulatory) caused by a native or naturally-occurring gp350 other than the ability to induce the production of an antibody against an antigenic epitope possessed by a native or naturally-occurring gp350 and an “immunological” activity refers to the ability to induce the production of an antibody against an antigenic epitope possessed by a native or naturally-occurring gp350.

The term “antagonist” is used in the broadest sense, and includes any molecule that partially or fully blocks, inhibits, or neutralizes a biological activity of a native EBV gp350 protein disclosed herein. In a similar manner, the term “agonist” is used in the broadest sense and includes any molecule that mimics a biological activity of a native EBV gp350 protein disclosed herein. Suitable agonist or antagonist molecules specifically include agonist or antagonist antibodies or antibody fragments, fragments or amino acid sequence variants of native EBV gp350 proteins, peptides, antisense oligonucleotides, small organic molecules, etc. Methods for identifying agonists or antagonists of an EBV gp350 protein may comprise contacting an EBV gp350 protein with a candidate agonist or antagonist molecule and measuring a detectable change in one or more biological activities normally associated with the EBV gp350 protein.

“Treating” or “treatment” or “alleviation” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. A subject or mammal is successfully “treated” for an EBV gp350 protein-expressing viral infection if, after receiving a therapeutic amount of an anti-gp350 antibody or gp350 binding oligopeptide according to the methods of this disclosure, the patient shows observable and/or measurable reduction in or absence of one or more of the following: reduction in the number of infected cells or absence of the infected cells; reduction in the number of infected cells; inhibition (i.e., slow to some extent and preferably stop) of EBV infection including the spread of infection into neurological tissues; inhibition (i.e., slow to some extent and preferably stop) of infection spread; inhibition, to some extent, and/or relief to some extent, of one or more of the symptoms associated with the viral infection; reduced morbidity and mortality, and improvement in quality of life issues. To the extent the anti-gp350 antibody or gp350 binding oligopeptide may prevent growth or infection and/or kill existing infected cells, it may be cytostatic and/or cytotoxic. Reduction of these signs or symptoms may also be felt by the patient. The above parameters for assessing successful treatment and improvement in the EBV-associated diseases and disorders are readily measurable by routine procedures familiar to a medical provider.

“Chronic” administration refers to administration of the agent(s) in a continuous mode as opposed to an acute mode, to maintain the initial therapeutic effect (activity) for an extended period of time. “Intermittent” administration is treatment that is not consecutively done without interruption, but rather is cyclic in nature.

“Mammal” for purposes of the treatment of, alleviating the symptoms of or diagnosis of a viral infection refers to any animal classified as a mammal, including humans, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

By “solid phase” or “solid support” is meant a non-aqueous matrix to which an antibody or gp350 binding oligopeptide of this disclosure can adhere or attach. Examples of solid phases encompassed herein include those formed partially or entirely of glass (e.g., controlled pore glass), polysaccharides (e.g., agarose), polyacrylamides, polystyrene, polyvinyl alcohol and silicones. Depending on the context, the solid phase can comprise the well of an assay plate or a lateral flow assay device; in others, it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as an EBV gp350 protein, an antibody thereto or a gp350 binding oligopeptide) to a mammal. The components of the liposome are commonly arranged in a bilayer formation, similar to the lipid arrangement of biological membranes.

A “small” molecule or “small” organic molecule is defined herein to have a molecular weight below about 500 Daltons.

An “effective amount” of a polypeptide, antibody or gp350 binding oligopeptide, or an agonist or antagonist thereof as disclosed herein is an amount sufficient to carry out a specifically stated purpose. An “effective amount” may be determined empirically and in a routine manner, in relation to the stated purpose.

The term “therapeutically effective amount” refers to an amount of an antibody, polypeptide, or gp350 binding oligopeptide, or other drug effective to “treat” a disease or disorder in a subject or mammal. In the case of an EBV infection, the therapeutically effective amount of the drug may reduce the number of infected cells; inhibit (i.e., slow to some extent and preferably stop) spread of the infection into other cells, such as lymphatic or neurological cells organs; and/or relieve to some extent one or more of the symptoms associated with the infection. See the definition herein of “treating.” To the extent the drug may prevent growth and/or kill existing infected cells, it may be cytostatic, cytotoxic, anti-inflammatory, immunomodulatory, and/or immunosuppressing.

A “growth inhibitory amount” of an anti-gp350 antibody or gp350 binding oligopeptide is an amount capable of inhibiting the growth of a cell, especially virus infected cell, either in vitro or in vivo. A “growth inhibitory amount” of an anti-gp350 antibody or gp350 binding oligopeptide for purposes of inhibiting infected cell growth may be determined empirically and in a routine manner.

A “cytotoxic amount” of an anti-gp350 antibody or gp350 binding is an amount capable of causing the destruction of a cell, especially virus infected cell, either in vitro or in vivo. A “cytotoxic amount” of an anti-gp350 antibody or gp350 binding oligopeptide for purposes of inhibiting cell growth may be determined empirically and in a routine manner.

The term “antibody” is used in the broadest sense and specifically covers, for example, single anti-gp350 monoclonal antibodies (including agonist, antagonist, and neutralizing antibodies), anti-gp350 antibody compositions with polyepitopic specificity, polyclonal antibodies, single chain anti-gp350 antibodies, and fragments of anti-gp350 antibodies (see below) as long as they exhibit the desired biological or immunological activity or specificity. The term “immunoglobulin” (Ig) is used interchangeable with antibody herein.

An “isolated antibody” is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. The antibody may be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain. Isolated antibody includes the antibody in situ within recombinant cells because at least one component of the antibody's natural environment will not be present. Ordinarily, however, isolated antibody will be prepared by at least one purification step.

The basic 4-chain antibody unit is a heterotetrameric glycoprotein composed of two identical light (L) chains and two identical heavy (H) chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, C T, 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses based on relatively minor differences in CH sequence and function, e.g., humans express the following subclasses: IgG1, IgG2, IgG3, IgG4, IgA1, and IgA2.

The term “variable” refers to the fact that certain segments of the variable domains differ extensively in sequence among antibodies. The V domain mediates antigen binding and defines specificity of an antibody for its antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 3-30, or more typically, 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, MD. (1991)). The constant domains are not involved directly in binding an antibody to an antigen, but exhibit various effector functions, such as participation of the antibody in antibody dependent cellular cytotoxicity (ADCC).

The term “monoclonal antibody” as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, useful monoclonal antibodies of this disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein include “chimeric” antibodies in which a portion of the heavy and/or light chain is identical with, or homologous to, corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape, etc.), and human constant region sequences.

An “intact” antibody is one which comprises an antigen-binding site as well as a CL and at least heavy chain constant domains, CH1, CH2 and CH3. The constant domains may be native sequence constant domains (e.g. human native sequence constant domains) or amino acid sequence variant thereof. Preferably, the intact antibody has one or more effector functions.

“Antibody fragments” comprise a portion of an intact antibody, preferably the antigen binding or variable region of the intact antibody. Examples of antibody fragments include Fab, Fab′, F(ab′)2, and Fv fragments; diabodies; linear antibodies (see U.S. Pat. No. 5,641,870, Example 2; Zapata et al., Protein Eng. 8(10): 1057-1062 [1995]); single-chain antibody molecules; and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)2 fragment which roughly corresponds to two disulfide-linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) can recognize and bind antigen, although at a lower affinity than the entire binding site.

“Single-chain Fv” (also abbreviated as “sFv” or “scFv”) are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all at least one, and typically two, variable domains, in which all or substantially all the hypervariable loops correspond to those of a non-human immunoglobulin and all or substantially all the FRs are those of a human immunoglobulin sequence. The humanized antibody optionally also will comprise at least a portion of an immunoglobulin constant region (Fe), typically that of a human immunoglobulin. For further details, see Jones et al., Nature 321:522-25 (1986); Riechmann et al., Nature 332:323-329 (1988); and Presta, Curr. Op. Struct. Biol. 2:593-96 (1992).

A “species-dependent antibody,” e.g., a mammalian anti-human IgE antibody, is an antibody which has a stronger binding affinity for an antigen from a first mammalian species than it has for a homologue of that antigen from a second mammalian species. Normally, the species-dependent antibody “binds specifically” to a human antigen (i.e., has a binding affinity (Kd) value of no more than about 1×10-7 M, preferably no more than about 1×10-8 and most preferably no more than about 1×10-9 M) but has a binding affinity for a homologue of the antigen from a second non-human mammalian species which is at least about 50-fold, or at least about 500-fold, or at least about 1000-fold, weaker than its binding affinity for the human antigen. The species-dependent antibody can be of any of the various types of antibodies as defined above, but preferably is a humanized or human antibody.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, MD. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The phrase “substantially similar,” or “substantially the same,” as used herein, denotes a sufficiently high degree of similarity between two numeric values (generally one associated with an antibody of the disclosure and the other associated with a reference/comparator antibody) such that one of skill in the art would consider the difference between the two values to be of little or no biological and/or statistical significance within the context of the biological characteristic measured by the values (e.g., Kd values). The difference between the two values is preferably less than about 50%, preferably less than about 40%, preferably less than about 30%, preferably less than about 20%, preferably less than about 10% as a function of the value for the reference/comparator antibody.

“Binding affinity” generally refers to the strength of the sum of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of this disclosure. Illustrative embodiments are described in the following.

The “Kd” or “Kd value” according to this disclosure is measured by a radiolabeled antigen binding assay (RIA) performed with the Fab version of an antibody of interest and its antigen as described by the following assay that measures solution binding affinity of Fabs for antigen by equilibrating Fab with a minimal concentration of (125I)-labeled antigen in the presence of a titration series of unlabeled antigen, then capturing bound antigen with an anti-Fab antibody-coated plate (Chen, et al., (1999) J. Mol Biol 293:865-881). To establish conditions for the assay, microtiter plates (Dynex) are coated overnight with 5 mcg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbant plate, 100 pM or 26 pM [125I]-antigen are mixed with serial dilutions of a Fab of interest (e.g., consistent with assessment of an anti-VEGF antibody, Fab-12, in Presta et al., (1997) Cancer Res. 57:4593-4599). The Fab of interest is then incubated overnight; however, the incubation may continue for a longer period (e.g., 65 hours) to ensure that equilibrium is reached. Thereafter, the mixtures are transferred to the capture plate for incubation at room temperature (e.g., for one hour). The solution is then removed and the plate washed eight times with 0.1% Tween-20 in PBS. When the plates have dried, 150 microliter/well of scintillant (MicroScint-20; Packard) is added, and the plates are counted on a Topcount gamma counter (Packard) for ten minutes. Concentrations of each Fab that give less than or equal to 20% of maximal binding are chosen for use in competitive binding assays. The Kd or Kd value may also be measured by using surface plasmon resonance assays using a BIAcore™-2000 or a BIAcore™-3000 (BIAcore, Inc., Piscataway, NJ) at 25° C. with immobilized antigen CM5 chips at approx. 10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, into 5 mcg/ml (approx. 0.2 uM) before injection at a flow rate of 5 microliter/minute to achieve approximately 10 response units (RU) of coupled protein. Following the injection of antigen, 1M ethanolamine is injected to block unreacted groups. For kinetics measurements, two-fold serial dilutions of Fab (0.78 nM to 500 nM) are injected in PBS with 0.05% Tween 20 (PBST) at 25° C. at a flow rate of approximately 25 microliter/min. Association rates (kon) and dissociation rates (koff) are calculated using a simple one-to-one Langmuir binding model (BIAcore Evaluation Software version 3.2) by simultaneous fitting the association and dissociation sensorgram. The equilibrium dissociation constant (Kd) is calculated as the ratio koff/kon. See, e.g., Chen, Y., et al., (1999) J. Mol Biol 293:865-881. If the on-rate exceeds 10⁶ M-1 S-1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrometer (Aviv Instruments) or a 8000-series SLM-Aminco spectrophotometer (ThermoSpectronic) with a stir red cuvette.

An “on-rate” or “rate of association” or “association rate” or “kon” according to this disclosure can also be determined with the same surface plasmon resonance technique described above using a BIAcore™-2000 or a BIAcore™-3000 (BIAcore, Inc., Piscataway, NJ) at 25° C. with immobilized antigen CM5 chips at approx. 10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, into 5 mcg/ml (approx. 0.2 uM) before injection at a flow rate of 5 microliter/minute to achieve approximately 10 response units (RU) of coupled protein. Following the injection of 1M ethanolamine to block unreacted groups. For kinetics measurements, two-fold serial dilutions of Fab (0.78 nM to 500 nM) are injected in PBS with 0.05% Tween 20 (PBST) at 25° C. at a flow rate of approximately 25 microliter/min. Association rates (kon) and dissociation rates (koff) are calculated using a simple one-to-one Langmuir binding model (BIAcore Evaluation Software version 3.2) by simultaneous fitting the association and dissociation sensorgram. The equilibrium dissociation constant (Kd) was calculated as the ratio koff/kon. See, e.g., Chen, Y., et al., (1999) J. Mol Biol 293:865-81. However, if the on-rate exceeds 10⁶ M-1 S-1 by the surface plasmon resonance assay above, then the on-rate is preferably determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-Aminco spectrophotometer (ThermoSpectronic) with a stirred cuvette.

The phrase “substantially reduced,” or “substantially different”, as used herein, denotes a sufficiently high degree of difference between two numeric values (generally one associated with an antibody of the disclosure and the other associated with a reference/comparator antibody) such that one of skill in the art would consider the difference between the two values to be of statistical significance within the context of the biological characteristic measured by the values (e.g., Kd values, HAMA response). The difference between the two values is preferably greater than about 10%, preferably greater than about 20%, preferably greater than about 30%, preferably greater than about 40%, preferably greater than about 50% as a function of the value for the reference/comparator antibody.

An “antigen” is a predetermined antigen to which an antibody can selectively bind. The target antigen may be polypeptide, carbohydrate, nucleic acid, lipid, hapten or other naturally occurring or synthetic compound. Preferably, the target antigen is an EBV gp350 polypeptide. An “acceptor human framework” for the purposes herein is a framework comprising the amino acid sequence of a VL or VH framework derived from a human immunoglobulin framework, or from a human consensus framework. An acceptor human framework “derived from” a human immunoglobulin framework or human consensus framework may comprise the same amino acid sequence thereof, or may contain pre-existing amino acid sequence changes. Where pre-existing amino acid changes are present, preferably no more than 5 and preferably 4 or less, or 3 or less, pre-existing amino acid changes are present. Where pre-existing amino acid changes are present in a VH, preferably those changes are only at three, two, or one of positions 71H, 73H and 78H; for instance, the amino acid residues at those positions may be 71A, 73T and/or 78A. The VL acceptor human framework may be identical in sequence to the VL human immunoglobulin framework sequence or human consensus framework sequence.

Antibodies of this disclosure may be able to compete for binding to the same epitope as is bound by a second antibody. Monoclonal antibodies are considered to share the “same epitope” if each blocks binding of the other by 40% or greater at the same antibody concentration in a standard in vitro antibody competition binding analysis.

A “human consensus framework” is a framework which represents the most commonly occurring amino acid residue in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., supra. For the VL, the subgroup may be subgroup kappa I as in Kabat et al. For the VH, the subgroup is subgroup III as in Kabat et al.

A “VH subgroup III consensus framework” comprises the consensus sequence obtained from the amino acid sequences in variable heavy subgroup III of Kabat et al.

A “VL subgroup I consensus framework” comprises the consensus sequence obtained from the amino acid sequences in variable light kappa subgroup I of Kabat et al.

An “unmodified human framework” is a human framework which has the same amino acid sequence as the acceptor human framework, e.g. lacking human to non-human amino acid substitution(s) in the acceptor human framework.

An “altered hypervariable region” for the purposes herein is a hypervariable region comprising one or more (e.g. one to about 16) amino acid substitution(s) therein.

An “un-modified hypervariable region” for the purposes herein is a hypervariable region having the same amino acid sequence as a non-human antibody from which it was derived, i.e. one which lacks one or more amino acid substitutions therein.

The term “hypervariable region”, “HVR”, “HV” or “CDR”, when used herein refers to the regions of an antibody variable domain which are hypervariable in sequence and/or form structurally defined loops. Generally, antibodies comprise six hypervariable regions; three in the VH (H1, H2, H3), and three in the VL (L1, L2, L3). Several hypervariable region delineations are in use and are encompassed herein. The Kabat Complementarity Determining Regions (CDRs) are based on sequence variability and are the most commonly used. The “contact” hypervariable regions are based on an analysis of the available complex crystal structures. The residues from each of these hypervariable regions are noted below. Unless otherwise denoted, Kabat numbering is employed. Hypervariable region locations are generally: amino acids 24-34 (HVR-L1), amino acids 49-56 (HVR-L2), amino acids 89-97 (HVR-L3), amino acids 26-35A (HVR-H1), amino acids 49-65 (HVR-H2), and amino acids 93-102 (HVR-H3). Hypervariable regions may also comprise “extended hypervariable regions” as follows: amino acids 24-36 (L1), and amino acids 46-56 (L2) in the VL, numbered according to Kabat et al., supra for each of these definitions.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues.

An “affinity matured” antibody is one with one or more alterations in one or more CDRs thereof which result in an improvement in the affinity or binding specificity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-55 (1995); Yelton et al. J. Immunol. 155:1994-2004 (1995); Jackson et al., J. Immunol. 154(7):3310-19 (1995); and Hawkins et al, J. Mol. Biol. 226:889-96 (1992).

A “blocking” antibody or an “antagonist” antibody is one which inhibits or reduces biological activity of the antigen it binds. Preferred blocking antibodies or antagonist antibodies substantially or completely inhibit the biological activity of the antigen.

A “gp350 binding oligopeptide” is an oligopeptide that binds, preferably specifically, to an EBV gp350 protein. gp350 binding oligopeptides may be chemically synthesized using known oligopeptide synthesis methodology or may be prepared and purified using recombinant technology. gp350 binding oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such oligopeptides are capable of binding, preferably specifically, to an EBV gp350 protein. gp350 binding oligopeptides of this disclosure preferably comprise or consist of at least one complementarity determining region (CDR) of the antibodies of this disclosure. gp350 binding oligopeptides may be identified without undue experimentation using well known techniques. In this regard, techniques for screening oligopeptide libraries for oligopeptides that are capable of specifically binding to a polypeptide target are known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

An antibody, oligopeptide or other organic molecule “which binds” an antigen of interest, e.g. an EBV polypeptide antigen target, is one that binds the antigen with sufficient affinity such that the antibody or oligopeptide is useful as a diagnostic and/or therapeutic agent in targeting a viral particle, or a cell or a tissue expressing the antigen, and does not significantly cross-react with other proteins, such as other herpes virus proteins. The extent of binding of the antibody or oligopeptide to a “non-target” protein will often be less than about 10% of the binding of the antibody or oligopeptide to its target protein as determined by fluorescence activated cell sorting (FACS) analysis or radioimmunoprecipitation (RIA). Regarding the binding of an antibody or oligopeptide to a target molecule, the terms “specific binding” or “specifically binds to” or “is specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or “is specific for” a particular polypeptide or an epitope on a particular polypeptide target as used herein can be exhibited, for example, by a molecule having a Kd for the target of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. The term “specific binding” may refer to binding where a molecule binds to a particular polypeptide or epitope on a particular polypeptide without substantially binding to any other polypeptide or polypeptide epitope.

An antibody or oligopeptide that “inhibits the growth of infected cells expressing an EBV gp350 protein” or a “growth inhibitory” antibody or oligopeptide is one which results in measurable growth inhibition of infected cells expressing or overexpressing the appropriate EBV gp350 protein. The EBV gp350 protein may be a transmembrane polypeptide expressed on the surface of an infected cell or may be a polypeptide that is produced and secreted by an infected cell. Preferred growth inhibitory anti-gp350 antibodies or oligopeptides inhibit growth of gp350-expressing cells by greater than 20%, preferably from about 20% to about 50%, and even more preferably, by greater than 50% (e.g., from about 50% to about 100%) as compared to the appropriate control, the control typically being cells not treated with the antibody or oligopeptide being tested. Growth inhibition can be measured at an antibody concentration of about 0.1 to 30 mcg/ml or about 0.5 nM to 200 nM in cell culture, where the growth inhibition is determined 1-10 days after exposure of the cells to the antibody. Growth inhibition of cells in vivo can be determined in various ways such as is described in the Examples section below. The antibody is growth inhibitory in vivo if administration of the anti-gp350 antibody at about 1 μg/kg to about 100 mg/kg body weight results in reduction in infected cells or inhibited EBV proliferation within about 1 day to 3 months from the first administration of the antibody, preferably within about 1 to 5 days.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. (USA) 95:652-56 (1998).

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daeron, Annu. Rev. Immunol. 15:203-234 (1997)). FcRs are reviewed in Ravetch and Kinet, Annu. Rev. Immunol. 9:457-492 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source, e.g., from blood.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202:163 (1996), may be performed.

An antibody or oligopeptide which “induces cell death” is one which causes a viable cell to become nonviable. The cell is one which expresses an EBV gp350 protein or is infected with EBV. Cell death in vitro may be determined in the absence of complement and immune effector cells to distinguish cell death induced by antibody-dependent cell-mediated cytotoxicity (ADCC) or complement dependent cytotoxicity (CDC). Thus, the assay for cell death may be performed using heat inactivated serum (i.e., in the absence of complement) and in the absence of immune effector cells. To determine whether the antibody or oligopeptide can induce cell death, loss of membrane integrity as evaluated by uptake of propidium iodide (PI), trypan blue (see Moore et al. Cytotechnology 17:1-11 (1995)) or 7AAD can be assessed relative to untreated cells.

A “gp350-expressing cell” is a cell which expresses an endogenous or transfected EBV gp350 protein which may include expression either on the cell surface or in a secreted form.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD, or IgM.

The word “label” when used herein refers to a detectable compound or composition which is conjugated directly or indirectly to an antibody or oligopeptide to generate a “labeled” antibody or oligopeptide. The label may be detectable by itself (e.g. radioisotope labels or fluorescent labels) or, in the case of an enzymatic label, may catalyze chemical alteration of a substrate compound or composition which is detectable.

The term “cytotoxic agent” as used herein refers to a substance that inhibits or prevents the function of cells and/or causes destruction of cells. The term is intended to include radioactive isotopes (e.g., At211, I131, I125, Y90, Re186, Re188, Sm153, Bi212, P32 and radioactive isotopes of Lu), chemotherapeutic agents, enzymes and fragments thereof such as nucleolytic enzymes, antibiotics, immune suppressants, and toxins such as small molecule toxins or enzymatically active toxins of bacterial, fungal, plant or animal origin, including fragments and/or variants thereof. An antiviral agent causes destruction of virus-infected cells.

A “growth inhibitory agent” when used herein refers to a compound or composition which inhibits growth of a cell, especially an EBV-infected cell, either in vitro or in vivo. Thus, the growth inhibitory agent may be one which significantly reduces the percentage of EBV-infected cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-2, IL-3, IL-4, IL-5, IL-6, IL-7, IL-8, IL-9, IL-11, IL-12; a tumor necrosis factor such as TNF-α or TNF-ß; and other polypeptide factors including LIF and kit ligand (KL). As used herein, the term cytokine includes proteins from natural sources or from recombinant cell culture and biologically active equivalents of the native sequence cytokines.

The term “package insert” is used to refer to instructions customarily included in commercial packages of therapeutic products, that contain information about the indications, usage, dosage, administration, contraindications and/or warnings concerning the use of such therapeutic products.

### II. Compositions and Methods

### A. Anti-gp350 Antibodies

This disclosure provides anti-gp350 antibodies which may find use herein as therapeutic and/or diagnostic agents. Exemplary antibodies include polyclonal, monoclonal, humanized, bispecific, and heteroconjugate antibodies.

### 1. Polyclonal Antibodies

Polyclonal antibodies are preferably raised in animals by multiple subcutaneous or intraperitoneal injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl2, or R1N═C═NR, where R and R1 are different alkyl groups.

Animals are immunized against the antigen, immunogenic conjugates, or derivatives by combining, e.g., 100 mcg or 5 mcg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to fourteen days later, the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

### 2. Monoclonal Antibodies

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, California USA, and SP-2 and derivatives e.g., X63-Ag8-653 cells available from the American Type Culture Collection, Manassas, Virginia, USA. Human myeloma and mouse-human heteromyeloma cell lines also have been described for producing human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA). The binding affinity of the monoclonal antibody can, for example, be determined by the Scatchard analysis described in Munson et al., Anal. Biochem., 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal e.g., by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Pluckthun, Immunol. Revs. 130:151-188 (1992).

Monoclonal antibodies or antibody fragments may be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res. 21:2265-66 (1993)). Thus, these techniques are viable alternatives to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (CH and CL) sequences for the homologous murine sequences (U.S. Pat. No. 4,816,567; and Morrison, et al., Proc. Natl Acad. Sci. USA, 81:6851 (1984)), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

### 3. Human and Humanized Antibodies

The anti-gp350 antibodies of this disclosure may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fe), typically that of a human immunoglobulin (Jones et al., Nature, 321:522-25 (1986); Riechmann et al., Nature, 332:323-29 (1988); and Presta, Curr. Op. Struct. Biol., 2:593-96 (1992)).

Methods for humanizing non-human antibodies are known in the art. Generally, a humanized antibody has one or more amino acid residues introduced into it from a source which is non-human. These non-human amino acid residues are often referred to as “import” residues, which are typically taken from an “import” variable domain. Humanization can be essentially performed by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is important to reduce antigenicity and HAMA response (human anti-mouse antibody) when the antibody is intended for human therapeutic use. According to the “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol. 151:2296 (1993); Chothia et al., J. Mol. Biol., 196:901 (1987)). Another method uses a framework region derived from the consensus sequence of all human antibodies of a subgroup of light or heavy chains. The same framework may be used for several different humanized antibodies (Carter et al., Proc. Natl. Acad. Sci. USA, 89:4285 (1992); Presta et al., J. Immunol. 151:2623 (1993)).

It is further important that antibodies be humanized with retention of high binding affinity for the antigen and other favorable biological properties. To achieve this goal, humanized antibodies may be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity or specificity for the target antigen(s), is achieved. In general, the hypervariable region residues are directly and most substantially involved in influencing antigen binding.

Various forms of humanized anti-gp350 antibodies are contemplated. For example, the humanized antibody may be an antibody fragment, such as a Fab, which is optionally conjugated with one or more cytotoxic agent(s) to generate an immunoconjugate. Alternatively, the humanized antibody may be an intact antibody, such as an intact IgG1 antibody.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-58 (1993); Bruggemann et al., Year in Immuno. 7:33 (1993); U.S. Pat. Nos. 5,545,806, 5,569,825, 5,591,669 (all of GenPharm); 5,545,807; and WO 97/17852.

Alternatively, phage display technology (McCafferty et al., Nature 348:552-53 (1990)) can be used to produce human antibodies and antibody fragments in vitro, from immunoglobulin variable (V) domain gene repertoires from unimmunized donors. Using this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Current Opinion in Structural Biology 3:564-71 (1993). Several sources of V-gene segments can be used for phage display. Clackson et al., Nature, 352:624-28 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-97 (1991), or Griffith et al., EMBO J. 12:725-34 (1993). See, also, U.S. Pat. Nos. 5,565,332 and 5,573,905.

As discussed above, human antibodies may also be generated in vitro in activated B cells (see, for example, U.S. Pat. Nos. 5,567,610 and 5,229,275).

### 4. Antibody Fragments

In certain circumstances, there are advantages to using antibody fragments, rather than whole antibodies. The smaller size of the fragments allows for rapid clearance, and may lead to improved access to EBV-infected cells or organs in a mammal.

Various techniques have been developed to produce antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., Bio/Technology 10:163-67 (1992)). Using another approach, F(ab′)2 fragments can be isolated directly from recombinant host cell culture. Fab and F(ab′)2 fragment with increased in vivo half-life comprising a salvage receptor binding epitope residues are described in U.S. Pat. No. 5,869,046. Other techniques to produce antibody fragments will be apparent to the skilled practitioner. The antibody of choice may also be a single chain Fv fragment (scFv). See WO 93/16185; U.S. Pat. Nos. 5,571,894; and 5,587,458. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870. Such linear antibody fragments may be monospecific or bispecific.

### 5. Bispecific Antibodies

Bispecific antibodies have binding specificities for at least two different epitopes. Exemplary bispecific antibodies may bind to two different epitopes of a gp350 protein. Other such antibodies may combine a gp350 binding site with a binding site for another protein. Alternatively, an anti-gp350 arm may be combined with an arm which binds to a triggering molecule on a leukocyte such as a T-cell receptor molecule (e.g. CD3) (see, e.g., Baeuerle, et al., Curr. Opin. Mol. Ther. 11(1):22-30 (2009)), or Fc receptors for IgG (FcγR), such as FcγRI (CD64), FcγRII (CD32) and FcγRIII (CD16), to focus and localize cellular defense mechanisms to the gp350-expressing cell. Bispecific antibodies may also be used to localize cytotoxic agents to EBV-infected cells which express gp350. These antibodies possess a gp350-binding arm and an arm which binds the cytotoxic agent (e.g., saporin, anti-interferon-α, vinca alkaloid, ricin A chain, methotrexate or radioactive isotope hapten). Bispecific antibodies can be prepared as full length antibodies or antibody fragments (e.g., F(ab′)2 bispecific antibodies).

WO 96/16673 describes a bispecific anti-ErbB2/anti-FcγRIII antibody and U.S. Pat. No. 5,837,234 discloses a bispecific anti-ErbB2/anti-FcγRI antibody. A bispecific anti-ErbB2/Fcα antibody is shown in WO98/02463. U.S. Pat. Nos. 5,821,337 and 6,407,213 teach bispecific anti-ErbB2/anti-CD3 antibodies. Additional bispecific antibodies that bind an epitope on the CD3 antigen and a second epitope have been described in U.S. Pat. No. 5,078,998 (anti-CD3/tumor cell antigen); U.S. Pat. No. 5,601,819 (anti-CD3/IL-2R; anti-CD3/CD28; anti-CD3/CD45); U.S. Pat. No. 6,129,914 (anti-CD3/malignant B cell antigen); 7,112,324 (anti-CD3/CD19); U.S. Pat. No. 6,723,538 (anti-CD3/CCR5); U.S. Pat. No. 7,235,641 (anti-CD3/EpCAM); U.S. Pat. No. 7,262,276 (anti-CD3/ovarian tumor antigen); and U.S. Pat. No. 5,731,168 (anti-CD3/CD4IgG).

Methods for making bispecific antibodies are known in the art. Traditional production of full length bispecific antibodies is based on the co-expression of two immunoglobulin heavy chain-light chain pairs, where the two chains have different specificities (Millstein et al., Nature 305:537-39 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J. 10:3655-59 (1991).

Using a different approach, antibody variable domains with the desired binding specificities (antibody-antigen combining sites) are fused to immunoglobulin constant domain sequences. Preferably, the fusion is with an Ig heavy chain constant domain, comprising at least part of the hinge, CH2, and CH3 regions. It is preferred to have the first heavy-chain constant region (CH1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three-polypeptide fragment when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

Preferably, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology 121:210 (1986).

Using another approach described in U.S. Pat. No. 5,731,168, the interface between a pair of antibody molecules can be engineered to maximize the percentage of heterodimers which are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with several cross-linking techniques.

Techniques for generating bispecific antibodies from antibody fragments have also been described in the literature. For example, bispecific antibodies can be prepared using chemical linkage. Brennan et al., Science 229:81 (1985) describe a procedure wherein intact antibodies are proteolytically cleaved to generate F(ab′)2 fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med. 175: 217-25 (1992) describe the production of a fully humanized bispecific antibody F(ab′)2 molecule. Each Fab′ fragment was separately secreted from E. coli and subjected to directed chemical coupling in vitro to form the bispecific antibody. The bispecific antibody thus formed could bind to cells overexpressing the ErbB2 receptor and normal human T cells, as well as trigger the lytic activity of human cytotoxic lymphocytes against human breast tumor targets. Various techniques for making and isolating bispecific antibody fragments directly from recombinant cell culture have also been described. For example, bispecific antibodies have been produced using leucine zippers. Kostelny et al., J. Immunol. 148(5):1547-53 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized to produce antibody homodimers. The “diabody” technology described by Hollinger et al., Proc. Natl. Acad. Sci. USA 90:6444-48 (1993) has provided an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments using single-chain Fv (sFv) dimers has also been reported. See Gruber et al., J. Immunol., 152:5368 (1994).

Antibodies with more than two valencies are contemplated. For example, trispecific antibodies can be prepared. Tutt et al., J. Immunol. 147:60 (1991).

### 6. Heteroconjugate Antibodies

Heteroconjugate antibodies are also within the scope of this disclosure. Heteroconjugate antibodies are composed of two covalently joined antibodies. Such antibodies have been proposed to target immune system cells to unwanted cells (see U.S. Pat. No. 4,676,980), and for treatment of HIV infection (see WO 91/00360; WO 92/200373; EP 03089). It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed in U.S. Pat. No. 4,676,980.

### 7. Multivalent Antibodies

A multivalent antibody may be internalized (and/or catabolized) faster than a bivalent antibody by a cell expressing an antigen to which the antibodies bind. The antibodies of this disclosure may be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody preferably further comprises at least two (and preferably four) light chain variable domain polypeptides. The multivalent antibody may comprise from about two to about eight light chain variable domain polypeptides. The light chain variable domain polypeptides contemplated here comprise a light chain variable domain and, optionally, further comprise a CL domain.

### 8. Effector Function Engineering

It may be desirable to modify the antibody of the disclosure with respect to effector function, e.g., to enhance antigen-dependent cell-mediated cytotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-95 (1992) and Shopes, B. J. Immunol. 148:2918-22 (1992). Homodimeric antibodies with enhanced anti-viral activity may also be prepared using heterobifunctional cross-linkers as described in Wolff et al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-30 (1989).

To increase the serum half-life of the antibody, a salvage receptor binding epitope may be incorporated into the antibody (especially an antibody fragment) as described in U.S. Pat. No. 5,739,277. The term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

### 9. Immunoconjugates

The disclosure also pertains to immunoconjugates comprising an antibody conjugated to a cytotoxic agent such as a chemotherapeutic agent, a growth inhibitory agent, a toxin (e.g., an enzymatically active toxin of bacterial, fungal, plant, or animal origin, or fragments thereof), or a radioactive isotope (i.e., a radioconjugate). Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

This disclosure further contemplates an immunoconjugate formed between an antibody and a compound with nucleolytic activity (e.g., a ribonuclease or a DNA endonuclease such as a deoxyribonuclease; DNase).

For selective destruction of an EBV-infected cell, the antibody may comprise a radioactive atom. A variety of radioactive isotopes are available to produce radioconjugated anti-gp350 antibodies. Examples include At211, 1131,1125, Y90, Re186, Re188, Sm153, Bi212, P32, Pb212 and radioactive isotopes of Lu. When the conjugate is used for diagnosis, it may comprise a radioactive atom for scintigraphic studies, for example tc99m or 1123, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc99m or 1123, Re186, Re188 and In111 can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes other methods in detail.

Conjugates of the antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

The compounds of the disclosure expressly contemplate, but are not limited to, an ADC prepared with cross-linker reagents: BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone) benzoate) which are commercially available from Pierce Biotechnology, Inc., Rockford, IL).

Alternatively, a fusion protein comprising the anti-EBV gp350 antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

The antibody may also be conjugated to a “receptor” (such streptavidin) for utilization in pre-targeting of viral infected cells, wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

### 10. Immunoliposomes

The anti-gp350 antibodies disclosed herein may also be formulated as immunoliposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antibody are prepared by methods known in the art, such as described in Epstein et al., Proc. Natl. Acad. Sci. USA 82:3688 (1985); Hwang et al., Proc. Natl Acad. Sci. USA 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of this disclosure can be conjugated to the liposomes as described in Martin et al., J. Biol. Chem. 257:286-288 (1982) via a disulfide interchange reaction. A chemotherapeutic agent is optionally contained within the liposome. See Gabizon et al., J. National Cancer Inst. 81(19):1484 (1989).

B. gp350 Binding Oligopeptides

gp350 binding oligopeptides of this disclosure are oligopeptides that bind, preferably specifically, to an EBV gp350 protein. gp350 binding oligopeptides may be chemically synthesized using known oligopeptide synthesis methodology or may be prepared and purified using recombinant technology. gp350 binding oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such oligopeptides that are capable of binding, preferably specifically, to an EBV gp350 protein. gp350 binding oligopeptides may be identified without undue experimentation using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

Bacteriophage (phage) display is one known technique used to screen large oligopeptide libraries to identify member(s) of those libraries which are capable of specifically binding to a polypeptide target. Phage display is a technique by which variant polypeptides are displayed as fusion proteins to the coat protein on the surface of bacteriophage particles (Scott, J. K. and Smith, G. P. (1990) Science 249:386). The utility of phage display lies in the fact that large libraries of selectively randomized protein variants (or randomly cloned cDNAs) can be rapidly and efficiently sorted for those sequences that bind to a target molecule with high affinity. Display of peptide (Cwirla, S. E. et al. (1990) Proc. Natl. Acad. Sci. USA, 87:6378) or protein (Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363) libraries on phage have been used for screening millions of polypeptides or oligopeptides for ones with specific binding properties (Smith, G. P. (1991) Current Opin. Biotechnol., 2:668). Sorting phage libraries of random mutants requires a strategy for constructing and propagating a large number of variants, a procedure for affinity purification using the target receptor, and a means of evaluating the results of binding enrichments. U.S. Pat. Nos. 5,223,409, 5,403,484, 5,571,689, and 5,663,143.

Although most phage display methods have used filamentous phage, lambdoid phage display systems (WO 95/34683; U.S. Pat. No. 5,627,024), T4 phage display systems (Ren et al., Gene, 215:439 (1998); Zhu et al., Cancer Research, 58(15): 3209-14 (1998); Jiang et al., Infection & Immunity, 65(11): 4770-77 (1997); Ren et al., Gene, 195(2):303-11 (1997); Ren, Protein Sci., 5: 1833 (1996); Efimov et al., Virus Genes, 10:173 (1995)) and T7 phage display systems (Smith and Scott, Methods in Enzymology, 217: 228-57 (1993); U.S. Pat. No. 5,766,905) are also known.

Many other improvements and variations of the basic phage display concept have now been developed. These improvements enhance the ability of display systems to screen peptide libraries for binding to selected target molecules and to display functional proteins with the potential of screening these proteins for desired properties. Combinatorial reaction devices for phage display reactions have been developed (WO 98/14277) and phage display libraries have been used to analyze and control bimolecular interactions (WO 98/20169; WO 98/20159) and properties of constrained helical peptides (WO 98/20036). WO 97/35196 describes a method of isolating an affinity ligand in which a phage display library is contacted with one solution in which the ligand will bind to a target molecule and a second solution in which the affinity ligand will not bind to the target molecule, to selectively isolate binding ligands. WO 97/46251 describes a method of biopanning a random phage display library with an affinity purified antibody and then isolating binding phage, followed by a micropanning process using microplate wells to isolate high affinity binding phage. The use of Staphlylococcus aureus protein A as an affinity tag has also been reported (Li et al. (1998) Mol Biotech., 9:187). WO 97/47314 describes the use of substrate subtraction libraries to distinguish enzyme specificities using a combinatorial library which may be a phage display library. A method for selecting enzymes suitable for use in detergents using phage display is described in WO 97/09446. Additional methods of selecting specific binding proteins are described in U.S. Pat. Nos. 5,498,538, 5,432,018, and WO 98/15833.

Methods of generating peptide libraries and screening these libraries are also disclosed in U.S. Pat. Nos. 5,723,286, 5,432,018, 5,580,717, 5,427,908, 5,498,530, 5,770,434, 5,734,018, 5,698,426, 5,763,192, and 5,723,323.

C. Screening for Anti-Gp350 Antibodies and Gp350 Binding Oligopeptides with the Desired Properties

Techniques for generating antibodies or oligopeptides that bind to EBV gp350 proteins have been described above. One may further select antibodies or oligopeptides with certain biological characteristics, as desired.

The growth inhibitory effects of an anti-gp350 antibody of this disclosure may be assessed by methods known in the art, e.g., using cells which express an EBV gp350 protein either endogenously or following transfection with the gp350 gene. For example, appropriate EBV infected cells may be treated with an anti-gp350 monoclonal antibody or oligopeptide of this disclosure at various concentrations for a few days (e.g., 2-7) and stained with crystal violet or MTT or analyzed by some other colorimetric assay. Another method of measuring proliferation would be by comparing 3H-thymidine uptake by the cells treated in the presence or absence an anti-gp350 antibody, or gp350 binding oligopeptide of the disclosure. After treatment, the cells are harvested and the amount of radioactivity incorporated into the DNA quantitated in a scintillation counter. Appropriate positive controls include treatment of a selected cell line with a growth inhibitory antibody known to inhibit growth of that cell line. Growth inhibition of infected cells in vivo can be determined in various ways known in the art. Preferably, the anti-gp350 antibody, or gp350 binding oligopeptide will inhibit cell proliferation of an EBV infected cell in vitro or in vivo by about 25-100% compared to the untreated infected cell, more preferably, by about 30-100%, and even more preferably by about 50-100% or 70-100%. Growth inhibition can be measured at an antibody concentration of about 0.5 to 30 μg/ml or about 0.5 nM to 200 nM in cell culture, where the growth inhibition is determined 1-10 days after exposure of the cells to the antibody. The antibody is growth inhibitory in vivo if administration of the anti-gp350 antibody at about 1 μg/kg to about 100 mg/kg body weight results in reduction in cell growth or proliferation within about 5 days to 3 months from the first administration of the antibody, preferably within about 5 to 30 days.

To select for an anti-gp350 antibody, gp350 binding oligopeptide which induces cell death, loss of membrane integrity as indicated by, e.g., propidium iodide (PI), trypan blue or 7AAD uptake may be assessed relative to control. A PI uptake assay can be performed in the absence of complement and immune effector cells. EBV gp350 protein-expressing cells are incubated with medium alone or medium containing the appropriate anti-gp350 antibody (e.g., at about 10 g/ml), gp350 binding oligopeptide. The cells are incubated for a 3-day time period. Following each treatment, cells are washed and aliquoted into 35 mm strainer-capped 12×75 tubes (1 ml per tube, 3 tubes per treatment group) for removal of cell clumps. Tubes then receive PI (10 g/ml). Samples may be analyzed using a FACSCAN® flow cytometer and FACSCONVERT® CellQuest software (Becton Dickinson). Those anti-gp350 antibodies, or gp350 binding oligopeptides that induce statistically significant levels of cell death as determined by PI uptake may be selected as cell death-inducing anti-gp350 antibodies or gp350 binding oligopeptides.

To screen for antibodies or oligopeptides which bind to an epitope on an EBV gp350 protein bound by an antibody of interest, a routine cross-blocking assay such as that described in Antibodies, A Laboratory Manual, Cold Spring Harbor Laboratory, Ed Harlow and David Lane (1988), can be performed. This assay can be used to determine if a test antibody or oligopeptide binds the same site or epitope as a known anti-gp350 antibody. Alternatively or additionally, epitope mapping can be performed by methods known in the art. For example, the antibody sequence can be mutagenized such as by alanine scanning, to identify contact residues. The mutant antibody is initially tested for binding with polyclonal antibody to ensure proper folding. In a different method, peptides corresponding to different regions of an EBV gp350 protein can be used in competition assays with the test antibodies or with a test antibody and an antibody with a characterized or known epitope.

### D. Anti-Gp350 Antibody and EBV Gp350 Binding Oligopeptide Variants

In addition to the anti-gp350 antibodies described herein, it is contemplated that anti-gp350 antibody variants can be prepared. Anti-gp350 antibody variants can be prepared by introducing appropriate nucleotide changes into the encoding DNA, and/or by synthesis of the desired antibody or polypeptide. Those skilled in the art will appreciate that amino acid changes may alter post-translational processes of the anti-gp350 antibody, such as changing the number or position of glycosylation sites or altering the membrane anchoring characteristics.

Variations in the anti-gp350 antibodies described herein can be made, for example, using any of the techniques and guidelines for conservative and non-conservative mutations set forth, for instance, in U.S. Pat. No. 5,364,934. Variations may be a substitution, deletion, or insertion of one or more codons encoding the antibody or polypeptide that results in a change in the amino acid sequence as compared with the native sequence antibody or polypeptide. Optionally, the variation is by substitution of at least one amino acid with any other amino acid in one or more of the domains of the anti-gp350 antibody. Guidance in determining which amino acid residue may be inserted, substituted or deleted without adversely affecting the desired activity may be found by comparing the sequence of the anti-gp350 antibody with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions, or substitutions of amino acids in the sequence and testing the resulting variants for activity exhibited by the full-length or mature native sequence.

Anti-gp350 antibody fragments are provided herein. Such fragments may be truncated at the N-terminus or C-terminus, or may lack internal residues, for example, when compared with a full length native antibody. Certain fragments lack amino acid residues that are not essential for a desired biological activity of the anti-gp350 antibody.

Anti-gp350 antibody fragments may be prepared by any of a number of conventional techniques. Desired peptide fragments may be chemically synthesized. An alternative approach involves generating antibody or polypeptide fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired antibody or polypeptide fragment, by polymerase chain reaction (PCR). Oligonucleotides that define the desired termini of the DNA fragment are employed at the 5′ and 3′ primers in the PCR. Preferably, anti-gp350 antibody fragments share at least one biological and/or immunological activity with the native anti-gp350 antibodies disclosed herein.

Conservative substitutions of interest are shown in Table 1 under the heading of preferred substitutions. If such substitutions result in a change in biological activity, then more substantial changes, denominated exemplary substitutions in Table 1, or as further described below in reference to amino acid classes, are introduced and the products screened.

TABLE 1

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in function or immunological identity of the anti-gp350 antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: Norleucine, Met, Ala, Val, Leu, Ile;
(2) neutral hydrophilic: Cys, Ser, Thr; Asn; Gln
(3) acidic: Asp, Glu;
(4) basic: His, Lys, Arg;
(5) residues that influence chain orientation: Gly, Pro; and
(6) aromatic: Trp, Tyr, Phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis (Carter et al., Nucl. Acids Res., 13:4331 (1986); Zoller et al., Nucl. Acids Res., 10:6487 (1987)), cassette mutagenesis (Wells et al., Gene, 34:315 (1985)), restriction selection mutagenesis (Wells et al., Philos. Trans. R. Soc. London SerA, 317:415 (1986)) or other known techniques can be performed on the cloned DNA to produce the anti-gp350 antibody variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant (Cunningham and Wells, Science, 244:1081-85 (1989)). Alanine is also typically preferred because it is the most common amino acid. Further, it is frequently found in both buried and exposed positions (Creighton, The Proteins, (W.H. Freeman & Co., N.Y.); Chothia, J. Mol. Biol., 150:1 (1976)). If alanine substitution does not yield adequate amounts of variant, an isoteric amino acid can be used.

Any cysteine residue not involved in maintaining the proper conformation of the anti-gp350 antibody also may be substituted, generally with serine, to improve the oxidative stability of the molecule and prevent aberrant crosslinking. Conversely, cysteine bond(s) may be added to the anti-gp350 antibody to improve its stability (particularly where the antibody is an antibody fragment such as an Fv fragment).

A particularly preferred type of substitutional variant involves substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. To identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and EBV gp350 protein. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Nucleic acid molecules encoding amino acid sequence variants of the anti-gp350 antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-gp350 antibody.

### E. Modifications of Anti-gp350 Antibodies

Covalent modifications of anti-gp350 antibodies and EBV gp350 proteins are included within the scope of this disclosure. One type of covalent modification includes reacting targeted amino acid residues of an anti-gp350 antibody with an organic derivatizing agent that can react with selected side chains or the N- or C-terminal residues of the anti-gp350 antibody. Derivatization with bifunctional agents is useful, for instance, for crosslinking anti-gp350 antibody to a water-insoluble support matrix or surface for use in purifying anti-gp350 antibodies, or detection of gp350 protein in biological samples, or EBV diagnostic assays. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis (succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the a-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Another type of covalent modification of the anti-gp350 antibody included within the scope of this disclosure comprises altering the native glycosylation pattern of the antibody or polypeptide. “Altering the native glycosylation pattern” is intended for purposes herein to mean deleting one or more carbohydrate moieties found in native sequence anti-gp350 antibody (either by removing the underlying glycosylation site or by deleting the glycosylation by chemical and/or enzymatic means), and/or adding one or more glycosylation sites that are not present in the native sequence anti-gp350 antibody. In addition, the phrase includes qualitative changes in the glycosylation of the native proteins, involving a change in the nature and proportions of the various carbohydrate moieties present.

Glycosylation of antibodies and other polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetyl galactosamine, galactose, or xylose to a hydroxyamino acid, most commonly serine or threonine, although 5-hydroxyproline or 5-hydroxylysine may also be used.

Addition of glycosylation sites to the anti-gp350 antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original anti-gp350 antibody (for O-linked glycosylation sites). The anti-gp350 antibody amino acid sequence may optionally be altered through changes at the DNA level, particularly by mutating the DNA encoding the anti-gp350 antibody at preselected bases such that codons are generated that will translate into the desired amino acids.

Another means of increasing the number of carbohydrate moieties on the anti-gp350 antibody is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the anti-gp350 antibody may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved using a variety of endo- and exo-glycosidases as described by Thotakura et al., Meth. Enzymol., 138:350 (1987).

Another type of covalent modification of anti-gp350 antibody comprises linking the antibody or polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337. The antibody or polypeptide also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

The anti-gp350 antibody of this disclosure may also be modified in a way to form chimeric molecules comprising an anti-gp350 antibody fused to another, heterologous polypeptide or amino acid sequence.

Such a chimeric molecule may comprise a fusion of the anti-gp350 antibody with a tag polypeptide which provides an epitope to which an anti-tag antibody can selectively bind. The epitope tag is generally placed at the amino- or carboxyl-terminus of the anti-gp350 antibody. The presence of such epitope-tagged forms of the anti-gp350 antibody can be detected using an antibody against the tag polypeptide. Also, provision of the epitope tag enables the anti-gp350 antibody to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto (Evan et al., Molecular and Cellular Biology, 5:3610-16 (1985)); and the Herpes Simplex virus glycoprotein D (gD) tag and its antibody (Paborsky et al., Protein Engineering, 3(6):547-553 (1990)). Other tag polypeptides include the Flag-peptide (Hopp et al., BioTechnology, 6:1204-10 (1988)); the KT3 epitope peptide (Martin et al., Science, 255:192-194 (1992)); an α-tubulin epitope peptide (Skinner et al., J. Biol. Chem., 266:15163-15166 (1991)); and the T7 gene 10 protein peptide tag (Lutz-Freyermuth et al., Proc. Natl. Acad. Sci. USA, 87:6393-6397 (1990)).

Alternatively, the chimeric molecule may comprise a fusion of the anti-gp350 antibody with an immunoglobulin or a region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of an anti-gp350 antibody in place of at least one variable region within an Ig molecule. Preferably, the immunoglobulin fusion includes the hinge, CH2 and CH3, or the hinge, CH1, CH2 and CH3 regions of an IgG1 molecule. For the production of immunoglobulin fusions, see also U.S. Pat. No. 5,428,130.

### F. Preparation of Anti-Gp350 Antibodies and EBV Gp350 Binding Oligopeptides

The description below relates primarily to production of anti-gp350 antibodies and EBV gp350 binding oligopeptides by culturing cells transformed or transfected with a vector containing anti-gp350 antibody-, or EBV gp350 binding oligopeptide-encoding nucleic acid. It is, of course, contemplated that alternative methods, which are well known in the art, may be employed to prepare anti-gp350 antibodies and EBV gp350 binding oligopeptides. For instance, the appropriate amino acid sequence, or portions thereof, may be produced by direct peptide synthesis using solid-phase techniques (e.g., Stewart et al., Solid-Phase Peptide Synthesis, W.H. Freeman Co., San Francisco, C A (1969); Merrifield, J. Am. Chem. Soc., 85:2149-54 (1963)). In vitro protein synthesis may be performed using manual techniques or by automation. Automated synthesis may be accomplished, for instance, using an Applied Biosystems Peptide Synthesizer (Foster City, CA) using manufacturer's instructions. Various portions of the anti-gp350 antibody may be chemically synthesized separately and combined using chemical or enzymatic methods to produce the desired anti-gp350 antibody.

### 1. Isolation of DNA Encoding Anti-Gp350 Antibody

DNA encoding anti-gp350 antibody may be obtained from a cDNA library prepared from tissue believed to possess the anti-gp350 antibody mRNA and to express it at a detectable level. Accordingly, human anti-gp350 antibody DNA can be conveniently obtained from a cDNA library prepared from human tissue. The anti-gp350 antibody-encoding gene may also be obtained from a genomic library or by known synthetic procedures (e.g., automated nucleic acid synthesis).

Libraries can be screened with probes (such as oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding anti-gp350 antibody is to use PCR methodology [Sambrook et al., supra; Dieffenbach et al., PCR Primer: A Laboratory Manual (Cold Spring Harbor Laboratory Press, 1995)].

Techniques for screening a cDNA library are well known in the art. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like 32P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acids having protein coding sequences may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

### 2. Selection and Transformation of Host Cells

Host cells are transfected or transformed with expression or cloning vectors described herein for anti-gp350 antibody production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂), CaPO4, liposome-mediated, and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kanr; E. coli W3110 strain 37D6, which has the complete genotype tonA ptr3 phoA E15 (argF-lac)169 degP ompT rbs7 ilvG kanr; E. coli W3110 strain 40B4, which is strain 37D6 with a non-kanamycin resistant degP deletion mutation; and an E. coli strain having mutant periplasmic protease disclosed in U.S. Pat. No. 4,946,783. Alternatively, in vitro methods of cloning, e.g., PCR or other nucleic acid polymerase reactions, are suitable.

Full length antibody, antibody fragments, and antibody fusion proteins can be produced in bacteria, in particular when glycosylation and Fc effector function are not needed, such as when the therapeutic antibody is conjugated to a cytotoxic agent (e.g., a toxin) and the immunoconjugate by itself shows effectiveness in EBV or EBV-infected cell destruction. Full length antibodies have greater half-life in circulation. Production in E. coli is faster and more cost efficient. For expression of antibody fragments and polypeptides in bacteria, see, e.g., U.S. Pat. No. 5,648,237 (Carter et. al.), U.S. Pat. No. 5,789,199 (Joly et al.), and U.S. Pat. No. 5,840,523 (Simmons et al.) which describes translation initiation region (TIR) and signal sequences for optimizing expression and secretion, these patents incorporated herein by reference. After expression, the antibody is isolated from the E. coli cell paste in a soluble fraction and can be purified through, e.g., a protein A or G column depending on the isotype. Final purification can be carried out similar to the process for purifying antibody expressed, e.g., in CHO cells.

In addition to prokaryotes, eukaryotic microbes such as filamentous fungi or yeast are suitable cloning or expression hosts for anti-gp350 antibody-encoding vectors. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated anti-gp350 antibody are derived from multicellular organisms. Examples of invertebrate cells include insect cells such as Drosophila S2 and Spodoptera Sf9, as well as plant cells, such as cell cultures of cotton, corn, potato, soybean, petunia, tomato, and tobacco. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to this disclosure, particularly for transfection of Spodoptera frugiperda cells.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for anti-gp350 antibody production and cultured in conventional nutrient media modified as appropriate for inducing promoters, selecting transformants, or amplifying the genes encoding the desired sequences.

### 3. Selection and Use of a Replicable Vector

The nucleic acid (e.g., cDNA or genomic DNA) encoding anti-gp350 antibody may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The anti-gp350 monoclonal antibodies may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the anti-gp350 antibody-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion, the signal sequence may be, e.g., the yeast invertase leader, alpha factor leader (including Saccharomyces and Kluyveromyces a-factor leaders, the latter described in U.S. Pat. No. 5,010,182), or acid phosphatase leader, the C. albicans glucoamylase leader (EP 362,179), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the anti-gp350 antibody-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene, 7:141 (1979); Tschemper et al., Gene, 10:157 (1980)]. The trp1 gene provides a selection marker for a mutant strain of yeast lacking the ability to grow in tryptophan, for example, ATCC No. 44076 or PEP4-1 (Jones, Genetics, 85:12 (1977)).

Expression and cloning vectors usually contain a promoter operably linked to the anti-gp350 antibody-encoding nucleic acid sequence to direct mRNA synthesis. Promoters recognized by a variety of potential host cells are well known. Promoters suitable for use with prokaryotic hosts include the β-lactamase and lactose promoter systems (Chang et al., Nature, 275:615 (1978); Goeddel et al., Nature, 281:544 (1979)), alkaline phosphatase, a tryptophan (trp) promoter system (Goeddel, Nucleic Acids Res., 8:4057 (1980)), and hybrid promoters such as the tac promoter (deBoer et al., Proc. Natl. Acad. Sci. USA, 80:21-25 (1983)). Promoters for use in bacterial systems also will contain a Shine-Dalgarno sequence operably linked to the DNA encoding anti-gp350 antibody.

Examples of suitable promoting sequences for use with yeast hosts include the promoters for 3-phosphoglycerate kinase (Hitzeman et al., J. Biol. Chem., 255:2073 (1980)) or other glycolytic enzymes (Hess et al., J. Adv. Enzyme Reg., 7:149 (1968); Holland, Biochemistry, 17:4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

Anti-gp350 antibody transcription from vectors in mammalian host cells is controlled, for example, by promoters obtained from the genomes of viruses such as polyoma virus, fowlpox virus (UK 2,211,504), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the anti-gp350 antibody by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, a-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the anti-gp350 antibody coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding anti-gp350 antibody.

Still other methods, vectors, and host cells suitable for adaptation to the synthesis of anti-gp350 antibody in recombinant vertebrate cell culture are described in Gething et al., Nature, 293:620-25 (1981); Mantei et al., Nature, 281:40-46 (1979); EP 117,060; and EP 117,058.

### 4. Culturing the Host Cells

The host cells used to produce the anti-gp350 antibody of this disclosure may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamycin), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### 5. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence EBV gp350 protein or against a synthetic peptide based on the DNA sequences provided herein or against exogenous sequence fused to gp350 DNA and encoding a specific antibody epitope.

### 6. Purification of Anti-Gp350 Antibodies and Gp350 Binding Oligopeptides

Forms of anti-gp350 antibody may be recovered from culture medium or from host cell lysates. If membrane-bound, it can be released from the membrane using a suitable detergent solution (e.g. Triton-X 100) or by enzymatic cleavage. Cells employed in expression of anti-gp350 antibody can be disrupted by various physical or chemical means, such as freeze-thaw cycling, sonication, mechanical disruption, or cell lysing agents.

It may be desired to purify anti-gp350 antibody from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the anti-gp350 antibody and EBV gp350 protein. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the anti-gp350 antibody produced.

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2 or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™resin (J. T. Baker, Phillipsburg, NJ) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### G. Pharmaceutical Formulations

Therapeutic formulations of the anti-gp350 antibodies or gp350 binding oligopeptides of this disclosure are prepared for storage by mixing the antibody, polypeptide, or oligopeptide having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as acetate, Tris, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g., Zn-protein complexes); and/or non-ionic surfactants such as TWEEN®, PLURONICS® or polyethylene glycol (PEG). The antibody preferably comprises the antibody at a concentration of between 5-200 mg/ml, preferably between 10-100 mg/ml.

The formulations herein may also contain more than one active compound as necessary for the indication being treated, preferably those with complementary activities that do not adversely affect each other. For example, in addition to an anti-gp350 antibody or gp350 binding oligopeptide, it may be desirable to include in the one formulation, an additional antibody, e.g., a second anti-gp350 antibody which binds a different epitope on the EBV gp350 protein. Alternatively, or additionally, the composition may further comprise a cytokine, an anti-inflammatory agent, or an interferon. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. Ed. (1980).

Sustained-release preparations may be prepared. Suitable examples of sustained-release preparations include semi-permeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

H. Diagnosis and Treatment with Anti-Gp350 Antibodies or Gp350 Binding Olilopeptides

gp350 expression may be evaluated using an in vivo diagnostic assay, e.g., by administering a molecule (such as an anti-gp350 antibody or gp350 binding oligopeptide) which binds the molecule to be detected and is tagged with a detectable label (e.g., a radioactive isotope or a fluorescent label) and externally scanning the patient for localization of the label.

As described above, the anti-gp350 antibodies or oligopeptides of this disclosure have various non-therapeutic applications. The anti-gp350 antibodies or oligopeptides of this disclosure are useful for diagnosis and staging of EBV infections. The antibodies or oligopeptides are also useful for purification or immunoprecipitation of EBV gp350 protein from cells, for detection and quantitation of EBV gp350 protein in vitro, e.g., in an ELISA or a Western blot, to kill and eliminate gp350-expressing cells from a population of mixed cells as a step in the purification of other cells.

Currently, EBV infection prevention and treatment involves preventing transmission of the virus, vaccination, or administration of interferons. Anti-gp350 antibody or oligopeptide therapy (such as by passive immunotherapy) may be especially desirable in elderly patients or immunocompromised patients or pregnant patients who may not tolerate the side effects of vaccination or vaccine components or interferons, or who cannot mount an immunological response.

A conjugate comprising an anti-gp350 antibody or oligopeptide conjugated with a cytotoxic agent may be administered to the patient. Preferably, the immunoconjugate bound to the anti-gp350 antibody is internalized by the cell, resulting in increased therapeutic efficacy of the immunoconjugate in killing the infected cell to which it binds. Preferably, the cytotoxic agent targets or interferes with the nucleic acid in the infected cell. The anti-gp350 antibodies or oligopeptides or conjugates thereof are administered to a human patient, in accord with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. Intravenous or subcutaneous administration of the antibody or oligopeptide is preferred.

Other therapeutic regimens may be combined with the administration of the anti-gp350 antibody or oligopeptide. The combined administration includes co-administration, using separate formulations or a single pharmaceutical formulation, and consecutive administration in either order, wherein preferably there is a time period while both (or all) active agents simultaneously exert their biological activities. Preferably such combined therapy results in a synergistic therapeutic effect.

It may also be desirable to combine administration of the anti-gp350 antibody or antibodies or oligopeptides with administration of an antibody directed against another EBV antigen.

The therapeutic treatment methods of this disclosure may include the combined administration of an anti-gp350 antibody (or antibodies) or oligopeptides and an interferon.

For the prevention or treatment of EBV infection or EBV-associated disease, the dosage and mode of administration of these antibodies and therapeutic proteins will be chosen by the medical provider according to known criteria. The appropriate dosage of antibody or oligopeptide will depend on the type of disease to be treated, as defined above, the severity and course of the disease, whether the antibody or oligopeptide is administered for preventive or therapeutic purposes, previous therapy, the patient's clinical history and response to the antibody or oligopeptide and the discretion of the medical provider. The antibody or oligopeptide is suitably administered to the patient at one time or over a series of treatments. Preferably, the antibody or oligopeptide is administered by intravenous infusion or by subcutaneous injections. Depending on the type and severity of the disease, about 1 mcg/kg to about 50 mg/kg body weight (e.g., about 0.1-15 mg/kg/dose) of antibody can be an initial candidate dosage for administration to the patient, whether, for example, by one or more separate administrations, or by continuous infusion. A dosing regimen can comprise administering an initial loading dose of about 4 mg/kg, followed by a weekly maintenance dose of about 2 mg/kg of the anti-gp350 antibody. However, other dosage regimens may be useful. A typical daily dosage might range from about 1 mcg/kg to 100 mg/kg or more, depending on the factors mentioned above. For repeated administrations over several days or longer, depending on the condition, the treatment is sustained until a desired suppression of disease symptoms occurs. The progress of this therapy can be readily monitored by conventional methods and assays and based on criteria known to medical providers of skill in the art.

Aside from administration of the anti-gp350 antibody to a patient, this disclosure contemplates administration of the antibody by gene therapy. Such administration of nucleic acid encoding the antibody is encompassed by the expression “administering a therapeutically effective amount of an antibody.” See, for example, WO96/07321 concerning the use of gene therapy to generate intracellular antibodies.

There are two major approaches to getting the nucleic acid (optionally contained in a vector) into the patient's cells; in vivo and ex vivo. For in vivo delivery, the nucleic acid is injected directly into the patient, usually at the site where the antibody is required. For ex vivo treatment, the patient's cells are removed, the nucleic acid is introduced into these isolated cells and the modified cells are administered to the patient either directly or, for example, encapsulated within porous membranes which are implanted into the patient (see, e.g., U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retroviral vector.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). For review of the currently known gene marking and gene therapy protocols see Anderson et al., Science 256:808-813 (1992). See also WO 93/25673 and the references cited therein.

The anti-gp350 antibodies of the disclosure can be in the different forms encompassed by the definition of “antibody” herein. Thus, the antibodies include full length or intact antibody, antibody fragments, native sequence antibody or amino acid variants, humanized, chimeric or fusion antibodies, immunoconjugates, and functional fragments thereof. In fusion antibodies, an antibody sequence is fused to a heterologous polypeptide sequence. The antibodies can be modified in the Fc region to provide desired effector functions. As discussed in more detail above, with the appropriate Fc regions, the naked antibody bound on the cell surface can induce cytotoxicity, e.g., via antibody-dependent cellular cytotoxicity (ADCC) or by recruiting complement in complement dependent cytotoxicity, or some other mechanism. Alternatively, where it is desirable to eliminate or reduce effector function, to minimize side effects or therapeutic complications, certain other Fc regions may be used.

These antibodies may include an antibody that competes for binding or binds substantially to, the same epitope as the antibodies of the disclosure. Antibodies having the biological characteristics of the present anti-gp350 antibodies of this disclosure are also contemplated, specifically including the in vivo targeting, and infection inhibiting or preventing, or cytotoxic characteristics.

The present anti-gp350 antibodies or oligopeptides are useful for treating an EBV infection or alleviating one or more symptoms of the infection in a mammal. The antibody or oligopeptide can bind to at least a portion of an infected cell that express EBV gp350 protein in the mammal. Preferably, the antibody or oligopeptide is effective to destroy or kill gp350-expressing cells or inhibit the growth of such cells, in vitro or in vivo, upon binding to EBV gp350 protein on the cell. Such an antibody includes a naked anti-gp350 antibody (not conjugated to any agent). Naked antibodies that have cytotoxic or cell growth inhibition properties can be further harnessed with a cytotoxic agent to render them even more potent in EBV or EBV-infected cell destruction. Cytotoxic properties can be conferred to an anti-gp350 antibody by, e.g., conjugating the antibody with a cytotoxic agent, to form an immunoconjugate as described herein. The cytotoxic agent or a growth inhibitory agent is preferably a small molecule.

This disclosure also provides a composition comprising an anti-gp350 antibody or oligopeptide of the disclosure, and a carrier. For the purposes of treating EBV infection, compositions can be administered to the patient in need of such treatment, wherein the composition can comprise one or more anti-gp350 antibodies present as an immunoconjugate or as the naked antibody. The compositions may comprise these antibodies or oligopeptides in combination with other therapeutic agents. The formulation may be a therapeutic formulation comprising a pharmaceutically acceptable carrier.

This disclosure also provides isolated nucleic acids encoding the anti-gp350 antibodies. Nucleic acids encoding both the H and L chains and especially the hypervariable region residues, chains which encode the native sequence antibody as well as variants, modifications and humanized versions of the antibody, are encompassed.

The disclosure also provides methods useful for treating an EBV infection or alleviating one or more symptoms of the infection in a mammal, comprising administering a therapeutically effective amount of an anti-gp350 antibody or oligopeptide of this disclosure to the mammal. The antibody or oligopeptide therapeutic compositions can be administered short term (acutely) or chronically, or intermittently as directed by a medical professional. Also provided are methods of inhibiting the growth of, and killing an EBV gp350 protein-expressing cell.

This disclosure also provides methods useful for treating or preventing post-transplant lymphoproliferative disorder (PTLD) in a mammal. In these methods, the antibodies or fragments thereof, are particularly effective for PTLD in EBV-seronegative persons (i.e., not previously infected with EBV) and given within a few days of solid organ or bone marrow transplant. In these methods, multiple doses of the antibodies, or functional fragments thereof, may be given over time (for example, in persons undergoing solid organ transplants, these proteins may be given within 72 hours of transplant and additional doses may be given at 1, 4, 6, 8, 12, and 16 weeks after transplant). Virtually all transplant recipients are screened for EBV and CMV serology prior to transplant. The antibodies, or functional fragments thereof, may also be administered to EBV-seropositive persons, but the rate of PTLD is lower is EBV seropositive persons than seronegative persons. In these methods, the antibodies, or functional fragments thereof, and an EBV vaccine may be co-administered. In these co-administration methods, the antibodies, or functional fragments thereof, and the vaccine(s) may be administered on the same day at different sites, as is done for combined treatment with vaccine and immunoglobulin for rabies, hepatitis B, or tetanus exposure. In these methods, a single dose of the antibodies, or functional fragments thereof, would likely be given because the recipient would be expected to produce antibody in response to the vaccine.

### J. Articles of Manufacture and Kits

This disclosure also provides assay devices, kits, and articles of manufacture comprising at least one anti-gp350 antibody or oligopeptide of this disclosure, optionally linked to a label, such as a fluorescent or radiolabel. The articles of manufacture may contain materials useful for the detection, diagnosis, or treatment of EBV infection. A preferred device is a lateral flow assay device which provides for point-of-care detection and/or diagnosis of an EBV infection. The article of manufacture comprises a container and a label or package insert on or associated with the container. Suitable containers include, for example, bottles, vials, syringes, etc. The containers may be formed from a variety of materials such as glass or plastic. The container holds a composition which is effective for detecting or treating the EBV infection and may have a sterile access port (for example the container may be an intravenous solution bag or a vial having a stopper pierceable by a hypodermic injection needle). At least one active agent in the composition is an anti-gp350 antibody or oligopeptide of this disclosure. The label or package insert indicates that the composition is used for detecting or treating EBV infection. The label or package insert may further comprise instructions for using the antibody or oligopeptide composition, e.g., in the testing or treating of the infected patient. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for EBV-infected cell killing assays, for purification, or immunoprecipitation of EBV gp350 protein from cells. For isolation and purification of EBV gp350 protein, the kit can contain an anti-gp350 antibodies or oligopeptides coupled to beads (e.g., sepharose beads). Kits can be provided which contain the antibodies or oligopeptides for detection and quantitation of EBV gp350 protein in vitro, e.g., in an ELISA or a Western blot. As with the article of manufacture, the kit comprises a container and a label or package insert on or associated with the container. The container holds a composition comprising at least one anti-gp350 antibody or oligopeptide of the disclosure. Additional containers may be included that contain, e.g., diluents and buffers, control antibodies. The label or package insert may provide a description of the composition as well as instructions for the intended in vitro or diagnostic use.

The anti-gp350 antibody or oligopeptide of this disclosure may also be provided as part of an assay device. Such assay devices include lateral flow assay devices. A common type of disposable lateral flow assay device includes a zone or area for receiving the liquid sample, a conjugate zone, and a reaction zone. These assay devices are commonly known as lateral flow test strips. They employ a porous material, e.g., nitrocellulose, defining a path for fluid flow capable of supporting capillary flow. Examples include those described in U.S. Pat. Nos. 5,559,041, 5,714,389, 5,120,643, and 6,228,660 all of which are incorporated herein by reference in their entireties. The anti-gp350 antibody or oligopeptide of this disclosure may also be used in a lateral flow assay device in conjunction with other antibodies to detect multiple EBV proteins or other herpesvirus proteins using a single biological sample from a subject or patient being tested on one portable, point-of-care device.

Another type of assay device is a non-porous assay device having projections to induce capillary flow. Examples of such assay devices include the open lateral flow device as disclosed in PCT International Publication Nos. WO 2003/103835, WO 2005/089082, WO 2005/118139, and WO 2006/137785, all of which are incorporated herein by reference in their entireties.

### K. Anti-Gp350 Antibody Probes and B-Cell Probes

Anti-gp350 antibody and anti-gp350 B-cell probes of this disclosure are polypeptides that bind, preferably specifically, to an anti-gp350 antibody, or a B-cell expressing an anti-gp350 B cell receptor (BCR) (anti-gp350 B cell). Accordingly, the three-dimensional structure of such a probe resembles the three-dimensional structure of at least a portion of the EBV gp350 protein. The three-dimensional structure of an anti-gp350 antibody probe, or an anti-gp350 B-cell probe, of this disclosure may resemble the three-dimensional structure of the CR2-binding region of an EBV gp350 protein. An anti-gp350 antibody probe, or an anti-gp350 B-cell probe, of this disclosure may comprise an amino acid sequence from the CR2-binding region of an EBV gp350 protein. An anti-gp350 antibody probe, or an anti-gp350 B-cell probe, of this disclosure may comprise at least 10, at least 20, at least 30, at least 40, at east 50, at least 75, at least 100, at least 150, at least 200, at least 250, or at least 350 contiguous amino acids from the amino acid sequence forming the CR2-binding region of an EBV gp350 protein, wherein the probe has the ability to bind an anti-gp350 antibody, or a B-cell expressing an anti-gp350 BCR, and wherein the binding of the B-cell is through the BCR. An anti-gp350 antibody probe, or anti-gp350 B-cell probe, of this disclosure may comprise an amino acid sequence at least 90%, at least 95%, at least 97% or at least 99% identical to the amino acid sequence of the CR2-binding region of EBV gp350, wherein the probe can bind an anti-gp350 antibody, or a B-cell expressing an anti-gp350 BCR, and wherein the binding of the B-cell is through the BCR.

Anti-gp350 antibody probes, and anti-gp350 B-cell probes, of this disclosure can be any size sufficient to bind to an anti-gp350 antibody or an anti-gp350 B-cell. An anti-gp350 antibody probe, or an anti-gp350 B-cell probe, of this disclosure may comprise at least 10, at least 20, at least 30, at least 40, at east 50, at least 75, at least 100, at least 150, at least 200, at least 250, at last 300, at least 350, at least 400, or at least 450 amino acids in length. An anti-gp350 antibody probe, or an anti-gp350 B-cell probe, of this disclosure may comprise at least 10, at least 20, at least 30, at least 40, at east 50, at least 75, at least 100, at least 150, at least 200, at least 250, at last 300, at east 350, at least 400, or at least 450 contiguous amino acids from the amino acid sequence forming the CR2-binding region of and EBV gp350 protein, wherein the probe has the ability to bind an anti-gp350 antibody, or a B-cell expressing an anti-gp350 BCR, and wherein the binding of the B-cell is through the BCR.

Using an anti-gp350 antibody probe, or anti-gp350 B-cell probe, of this disclosure having a sequence identical to that of EBV gp35, to isolate gp350-specific B-cells might be problematic, because most B-cells, including memory B-cells, constitutively express CR2 on their surface. Consequently, probes having a three-dimensional structure, and/or a sequence, identical to the three-dimensional structure, and/or sequence, of the CR2-binding region of EBV gp350 will bind to almost all B-cells. Thus, anti-gp350 antibody probes, and anti-gp350 B-cell probes, of this disclosure may lack the ability to bind CR2. Such anti-gp350 antibody probes, and anti-gp350 B-cell probes, can be constructed, for example, by using the sequence of the CR2-binding region of an EBV gp350 protein to construct the probe, and then altering the sequence so that it no longer binds CR2. Thus, anti-gp350 antibody probes, or an anti-gp350 B-cell probes, of this disclosure may comprise a variant sequence of an EBV gp350 CR2 binding region. An anti-gp350 antibody probe, or anti-gp350 B-cell probe, of this disclosure comprises the sequence of an EBV gp350 CR2 binding region, wherein the sequence has been altered to reduce, or eliminate, binding of the probe to CR2. Such alterations include, but are not limited to, substitutions, insertions, and deletions of amino acid residues. Such probes preferentially bind to an anti-gp350 antibody, or an anti-gp350 BCR, but do not bind CR2. An anti-gp350 antibody probe, or anti-gp350 B-cell probe, of this disclosure may comprise an amino acid sequence at least 80% identical, at last 85% identical, at least 90% identical, at least 95% identical, at least 97% identical, or at least 99% identical to an EBV gp350 CR2 binding region, wherein the probe binds to an anti-gp350 antibody, or an anti-gp350 BCR, and wherein the probe does not bind CR2. An anti-gp350 antibody probe, or anti-gp350 B-cell probe, of this disclosure may comprise an amino acid sequence at least 80% identical, at last 85% identical, at least 90% identical, at least 95% identical, at least 97% identical, or at least 99% identical to SEQ ID NO:124, wherein the probe binds to an anti-gp350 antibody, or an anti-gp350 BCR, and wherein the probe, does not bind CR2.

An anti-gp350 antibody probe, or an anti-gp350 B-cell probe, of the disclosure, that binds to an anti-gp350 antibody or an anti-gp350 B cell expressing an anti-gp350 BCR, but that does not bind CR2, can be designed using methods known to those skilled in the art. For example, nucleic acid molecules encoding the CR2-binding region of an EBV gp350 protein can be randomly mutated, the mutated molecules inserted into expression vectors, the vectors introduced into cells capable of expressing them, and the cells cultured such that the encoded, mutated probes are expressed on the cells surface. The cells could then be screened to identify antibodies, B-cells, that bind to the expressed probes. Following identification of such antibodies, and/or B-cells, the identified antibodies, and/or B-cells, can then be tested for their ability to bind CR2. In an alternative method, the three-dimensional model of a complex between EBV gp350 and an anti-gp350 antibody is determined, and the amino acid residues involved in binding of the two molecules determined. A second molecule of a complex between gp350 and CR2 could also be produced, and the amino acid residues involved in formation of this complex determined. Using these models, a person skilled in the art could then chose specific locations within the gp350 protein that could be altered. For example, amino acid residues involved in pairing of gp350 and CR2, but which are not involved in pairing of gp350 and an anti-gp350 antibody, could be chosen for alteration. Once a variant sequence has been designed, nucleic acid molecules encoding the variant proteins can be synthesized, or produced from wild-type sequences using mutagenesis techniques E.g., PCR), and the encoded proteins expressed and tested for activity.

Using methodology such as that described above, the present inventors have identified amino acid residues involved in binding of EBV gp350 to CR2 and to an anti-gp350 antibody. In particular, the inventors have determined that at least residues Q122, P158, 1160, K161, W162, D163, N164, and D296 of the EBV gp350 protein (amino acid number 1 is the first residue of the protein represented by SEQ ID NO:124) are involved in binding of the protein with CR2. Thus, an anti-gp350 antibody probe, or an anti-gp350 B-cell probe, of this disclosure may comprise an amino acid sequence from the CR2-binding region an EBV gp350 protein, wherein at least one amino acid corresponding to amino acid Q122, P158, 1160, K161, W162, D163, N164, or D296, of SEQ ID NO:124 has been deleted, or has been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein at least one amino acid corresponding to amino acid Q122, P158, 1160, K161, W162, D163, N164, or D296, of SEQ ID NO:124 has been deleted, or has been substituted with one or more amino acid residues, wherein the probe lacks the ability to bind CR2, and wherein the probe specifically binds to an anti-gp350 antibody or an anti-gp350 B-cell. When an amino acid residue is substituted, it is preferred that that the substituting amino acid residue have binding properties (e.g., ionic charge, hydrophobicity, etc.) different from the amino acid residue being replaced.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to the amino acid sequence of an EBV gp350 Cr2-binding region, wherein the amino acid corresponding to amino acid Q122 of SEQ ID NO:124 has been deleted, or has been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid Q122 of SEQ ID NO:124 has been deleted, or has been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid Q122 of SEQ ID NO:124 has been substituted with an amino acid residue selected from the group consisting of methionine, leucine, isoleucine, glycine, alanine, valine, phenylalanine, tyrosine, tryptophan, lysine, arginine, histidine, glutamate, and aspartate, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid Q122 of SEQ ID NO:124 has been substituted with asparagine, wherein the probe lacks the ability to bind CR2, and wherein the probe specifically binds to an anti-gp350 antibody or an anti-gp350 B-cell.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to the amino acid sequence of an EBV gp350 CR2-binding region, wherein the amino acid corresponding to amino acid P158 of SEQ ID NO:124 has been deleted, or has been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid P158 of SEQ ID NO:124 has been deleted, or has been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid P158 of SEQ ID NO:124 has been substituted with an amino acid residue selected from the group consisting of methionine, leucine, isoleucine, glycine, alanine, valine, phenylalanine, tyrosine, tryptophan, lysine, arginine, histidine, glutamate, and aspartate, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid Q122 of SEQ ID NO:124 has been substituted with arginine, wherein the probe lacks the ability to bind CR2, and wherein the probe specifically binds to an anti-gp350 antibody or an anti-gp350 B-cell.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to the amino acid sequence of an EBV gp350 CR2-binding region, wherein the amino acid corresponding to amino acid I160 of SEQ ID NO:124 has been deleted, or has been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid I160 of SEQ ID NO:124 has been deleted, or has been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid I160 of SEQ ID NO:124 has been substituted with an amino acid residue selected from the group consisting of serine, threonine, cysteine, proline, asparagine, glutamine, tyrosine, phenylalanine, tryptophan, glutamate, aspartate, arginine, glutamine, histidine and lysine, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid I160 of SEQ ID NO:124 has been substituted with arginine, wherein the probe lacks the ability to bind CR2, and wherein the probe specifically binds to an anti-gp350 antibody or an anti-gp350 B-cell.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to the amino acid sequence of an EBV gp350 CR2-binding region, wherein the amino acid corresponding to amino acid W162 of SEQ ID NO:124 has been deleted, or has been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid W162 of SEQ ID NO:124 has been deleted, or has been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid W162 of SEQ ID NO:124 has been substituted with an amino acid residue selected from the group consisting of serine, threonine, cysteine, proline, asparagine, glutamine, aspartate, glutamate, asparagine, arginine, glutamate, histidine, glycine, alanine, valine, leucine, methionine, isoleucine, and lysine, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid W162 of SEQ ID NO:124 has been substituted with alanine, wherein the probe lacks the ability to bind CR2, and wherein the probe specifically binds to an anti-gp350 antibody or an anti-gp350 B-cell.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to the amino acid sequence of an EBV gp350 CR2-binding region, wherein the amino acid corresponding to amino acid D163 of SEQ ID NO:124 has been deleted, or has been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid D163 of SEQ ID NO:124 has been deleted, or has been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid D163 of SEQ ID NO:124 has been substituted with an amino acid residue selected from the group consisting of tyrosine, phenylalanine, tryptophan, serine, threonine, cysteine, proline, asparagine, glutamine, lysine, arginine, histidine, glycine, alanine, valine, leucine, methionine, and isoleucine, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid D163 of SEQ ID NO:124 has been substituted with asparagine, wherein the probe lacks the ability to bind CR2, and wherein the probe specifically binds to an anti-gp350 antibody or an anti-gp350 B-cell.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to the amino acid sequence of an EBV gp350 CR2-binding region, wherein the amino acid corresponding to amino acid N164 of SEQ ID NO:124 has been deleted, or has been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid N164 of SEQ ID NO:124 has been deleted, or has been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid N164 of SEQ ID NO:124 has been substituted with an amino acid residue selected from the group consisting of methionine, leucine, isoleucine, glycine, alanine, valine, phenylalanine, tyrosine, tryptophan, lysine, arginine, histidine, glutamate, serine, and aspartate, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid N164 of SEQ ID NO:124 has been substituted with a serine residue, wherein the probe lacks the ability to bind CR2, and wherein the probe specifically binds to an anti-gp350 antibody or an anti-gp350 B-cell.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to the amino acid sequence of an EBV gp350 CR2-binding region, wherein the amino acid corresponding to amino acid D296 of SEQ ID NO:124 has been deleted, or has been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid D296 of SEQ ID NO:124 has been deleted, or has been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid D296 of SEQ ID NO:124 has been substituted with an amino acid residue selected from the group consisting of tyrosine, phenylalanine, tryptophan, serine, threonine, cysteine, proline, asparagine, glutamine, lysine, arginine, histidine, glycine, alanine, valine, leucine, methionine, and isoleucine, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid D296 of SEQ ID NO:124 has been substituted with arginine, wherein the probe lacks the ability to bind CR2, and wherein the probe specifically binds to an anti-gp350 antibody or an anti-gp350 B-cell.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to the amino acid sequence of an EBV gp350 CR2-binding region, wherein one or more amino acids has been inserted between the amino acid corresponding to amino acid N164 of SEQ ID NO:124 and the amino acid corresponding to C165 of SEQ ID NO:124, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein one or more amino acids has been inserted between the amino acid corresponding to amino acid N164 of SEQ ID NO:124 and the amino acid corresponding to C165 of SEQ ID NO:124, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein one or more amino acids selected from the group consisting of serine, threonine, cysteine, proline, asparagine, and glutamine, has been inserted between the amino acid corresponding to amino acid N164 of SEQ ID NO:124 and the amino acid corresponding to C165 of SEQ ID NO:124, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein a serine residue has been inserted between the amino acid corresponding to amino acid N164 of SEQ ID NO:124 and the amino acid corresponding to C165 of SEQ ID NO:124, wherein the probe lacks the ability to bind CR2, and wherein the probe specifically binds to an anti-gp350 antibody or an anti-gp350 B-cell.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to the amino acid sequence of an EBV gp350 CR2-binding region, wherein the amino acid corresponding to amino acid D163 of SEQ ID NO:124 has been deleted, or has been substituted with one or more amino acid residues, wherein one or more amino acids has been inserted between the amino acid corresponding to amino acid N164 of SEQ ID NO:124 and the amino acid corresponding to C165 of SEQ ID NO:124, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid D163 of SEQ ID NO:124 has been deleted, or has been substituted with one or more amino acid residues, wherein one or more amino acids has been inserted between the amino acid corresponding to amino acid N164 of SEQ ID NO:124 and the amino acid corresponding to C165 of SEQ ID NO:124, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid D163 of SEQ ID NO:124 has been substituted with an amino acid residue selected from the group consisting of tyrosine, phenylalanine, tryptophan, serine, threonine, cysteine, proline, asparagine, glutamine, lysine, arginine, histidine, glycine, alanine, valine, leucine, methionine, and isoleucine, wherein one or more amino acids selected from the group consisting of serine, threonine, cysteine, proline, asparagine, and glutamine, has been inserted between the amino acid corresponding to amino acid N164 of SEQ ID NO:124 and the amino acid corresponding to C165 of SEQ ID NO:124, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid W162 of SEQ ID NO:124 has been substituted with asparagine, wherein a serine residue has been inserted between the amino acid corresponding to amino acid N164 of SEQ ID NO:124 and the amino acid corresponding to C165 of SEQ ID NO:124, wherein the probe lacks the ability to bind CR2, and wherein the probe specifically binds to an anti-gp350 antibody or an anti-gp350 B-cell.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to the amino acid sequence of an EBV gp350 Cr2-binding region, wherein the amino acids corresponding to amino acids W162-N164 of SEQ ID NO:124 have been deleted, or have been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acids corresponding to amino acids W162-N164 of SEQ ID NO:124 have been deleted, or have been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acids corresponding to amino acids W162-N164 of SEQ ID NO:124 have been deleted, or have been substituted with an amino acid residue selected from the group consisting of serine, threonine, cysteine, proline, asparagine, glutamine, aspartate, glutamate, asparagine, arginine, glutamate, histidine, glycine, alanine, valine, leucine, methionine, isoleucine, and lysine, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acids corresponding to amino acids W162-N164 of SEQ ID NO:124 have each been substituted with an alanine residue, wherein the probe lacks the ability to bind CR2, and wherein the probe specifically binds to an anti-gp350 antibody or an anti-gp350 B-cell.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to the amino acid sequence of an EBV gp350 CR2-binding region, wherein the amino acid corresponding to amino acid D296 of SEQ ID NO:124 has substituted with one or more amino acid residues, wherein the amino acid corresponding to amino acid 1160 of SEQ ID NO:124 has been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid D296 of SEQ ID NO:124 has been substituted with one or more amino acid residues, wherein the amino acid corresponding to amino acid I160 of SEQ ID NO:124 has substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid D296 of SEQ ID NO:124 has been substituted with an amino acid residue selected from the group consisting of tyrosine, phenylalanine, tryptophan, serine, threonine, cysteine, proline, asparagine, glutamine, lysine, arginine, histidine, glycine, alanine, valine, leucine, methionine, and isoleucine, wherein the amino acid corresponding to amino acid I160 of SEQ ID NO:124 has been substituted with an amino acid residue selected from the group consisting of serine, threonine, cysteine, proline, asparagine, glutamine, tyrosine, phenylalanine, tryptophan, glutamate, aspartate, arginine, glutamine, histidine and lysine, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid D296 of SEQ ID NO:124 has been substituted with arginine, wherein the amino acid corresponding to amino acid I160 of SEQ ID NO:124 has been substituted with arginine, wherein the probe lacks the ability to bind CR2, and wherein the probe specifically binds to an anti-gp350 antibody or an anti-gp350 B-cell.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to the amino acid sequence of an EBV gp350 CR2-binding region, wherein the amino acid corresponding to amino acid D296 of SEQ ID NO:124 has substituted with one or more amino acid residues, wherein the amino acid corresponding to amino acid P158 of SEQ ID NO:124 has been substituted with one or more amino acid residues, wherein the amino acid corresponding to amino acid I160 of SEQ ID NO:124 has substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid D296 of SEQ ID NO:124 has substituted with one or more amino acid residues, wherein the amino acid corresponding to amino acid P158 of SEQ ID NO:124 has been substituted with one or more amino acid residues, wherein the amino acid corresponding to amino acid I160 of SEQ ID NO:124 has substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid D296 of SEQ ID NO:124 has been substituted with an amino acid residue selected from the group consisting of tyrosine, phenylalanine, tryptophan, serine, threonine, cysteine, proline, asparagine, glutamine, lysine, arginine, histidine, glycine, alanine, valine, leucine, methionine, and isoleucine, wherein the amino acid corresponding to amino acid P158 of SEQ ID NO:124 has been substituted with an amino acid residue selected from the group consisting of methionine, leucine, isoleucine, glycine, alanine, valine, phenylalanine, tyrosine, tryptophan, lysine, arginine, histidine, glutamate, and aspartate, wherein the amino acid corresponding to amino acid 1160 of SEQ ID NO:124 has been substituted with an amino acid residue selected from the group consisting of serine, threonine, cysteine, proline, asparagine, glutamine, tyrosine, phenylalanine, tryptophan, glutamate, aspartate, arginine, glutamine, histidine and lysine, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid D296 of SEQ ID NO:124 has been substituted with arginine, wherein the amino acid corresponding to amino acid P158 of SEQ ID NO:124 has been substituted with asparagine, wherein the amino acid corresponding to amino acid I160 of SEQ ID NO:124 has been substituted with threonine, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid D296 of SEQ ID NO:124 has been substituted with arginine, wherein the amino acid corresponding to amino acid P158 of SEQ ID NO:124 has been substituted with arginine, wherein the amino acid corresponding to amino acid I160 of SEQ ID NO:124 has been substituted with arginine, wherein the probe lacks the ability to bind CR2, and wherein the probe specifically binds to an anti-gp350 antibody or an anti-gp350 B-cell.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to the amino acid sequence of an EBV gp350 CR2-binding region, wherein the amino acid corresponding to amino acid K161 of SEQ ID NO:124 has been deleted, or has been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid K161 of SEQ ID NO:124 has been deleted, or has been substituted with one or more amino acid residues, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:125, wherein the amino acid corresponding to amino acid K161 of SEQ ID NO:124 has been substituted with an amino acid residue selected from the group consisting of methionine, leucine, isoleucine, glycine, alanine, valine, phenylalanine, tyrosine, tryptophan, lysine, arginine, histidine, glutamate, serine, and aspartate, wherein the probe lacks the ability to bind CR2, and wherein the probe specifically binds to an anti-gp350 antibody or an anti-gp350 B-cell.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:127 or SEQ ID NO:145, wherein the amino acid corresponding to amino acid Q122 of SEQ ID NO:124 is asparagine, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise SEQ ID NO:127 or SEQ ID NO:145.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:129 or SEQ ID NO:147, wherein the amino acid corresponding to amino acid D163 of SEQ ID NO:124 is asparagine, and wherein a serine residue has been inserted between the amino acid corresponding to amino acid N164 of SEQ ID NO:124 and the amino acid corresponding to C165 of SEQ ID NO:124, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise SEQ ID NO:129 or SEQ ID NO:147.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:131 or SEQ ID NO:149, wherein the amino acid corresponding to amino acid D296 of SEQ ID NO:124 is arginine, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise SEQ ID NO:131 or SEQ ID NO:149.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:133 or SEQ ID NO:151, wherein the amino acids corresponding to amino acids W162-N164 of SEQ ID NO:124 have each been substituted with an alanine residue, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise SEQ ID NO:133 or SEQ ID NO:151.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:135 or SEQ ID NO:153, wherein the amino acid corresponding to amino acid D296 of SEQ ID NO:124 is arginine, wherein the amino acid corresponding to amino acid I160 of SEQ ID NO:124 is arginine, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise SEQ ID NO:135 or SEQ ID NO:153.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:137 or SEQ ID NO:155, wherein the amino acid corresponding to amino acid D296 of SEQ ID NO:124 is arginine, wherein the amino acid corresponding to amino acid P158 of SEQ ID NO:124 is arginine, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise SEQ ID NO:137 or SEQ ID NO:155.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:139 or SEQ ID NO:157, wherein the amino acid corresponding to amino acid D296 of SEQ ID NO:124 is arginine, wherein the amino acid corresponding to amino acid P158 of SEQ ID NO:124 is asparagine, wherein the amino acid corresponding to amino acid I160 of SEQ ID NO:124 is threonine, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise SEQ ID NO:139 or SEQ ID NO:157.

An anti-gp350 antibody probe, or an anti-gp350 B-cell probe, of this disclosure may comprise an amino acid sequence at least 80% identical, at least 85% identical, at least 90% identical, at least 95% identical, at least 97% identical, or at least 99% identical to SEQ ID NO:141 or SEQ ID NO:159, wherein the amino acid corresponding to amino acid D296 of SEQ ID NO:124 is arginine, wherein the amino acid corresponding to amino acid P158 of SEQ ID NO:124 is arginine, wherein the amino acid corresponding to amino acid I160 of SEQ ID NO:124 is arginine, wherein the probe lacks the ability to bind CR2, and wherein the probe specifically binds to an anti-gp350 antibody or an anti-gp350 B-cell. An anti-gp350 antibody probe, or an anti-gp350 B-cell probe, of this disclosure may comprise SEQ ID NO:141 or SEQ ID NO:159.

Thus, this disclosure includes a nucleic acid molecule encoding an anti-gp350 antibody probe, or an anti-gp350 B-cell probe, disclosed herein. Such nucleic acid molecule includes DNA, RNA, mixtures thereof, and modified forms thereof. This disclosure also includes nucleic acid vectors, such as, for example, plasmids, expression vectors, viral vectors, etc., as well as cells comprising such nucleic acid vectors. Methods of producing nucleic acid molecules and nucleic acid vectors of the invention have been described herein.

Anti-gp350 antibody and anti-gp350 B-cell probes of this disclosure can be produced chemically (e.g., oligopeptide synthesis methodology), or they can be produced using recombinant technology. For example, an anti-gp350 antibody probe, or anti-gp350 B-cell probe can be deigned, by producing a nucleic acid molecule encoding the probe, and the nucleic acid molecule introduced into a cell where it can be expressed.

This disclosure includes methods of identifying anti-gp350 antibodies, comprising contacting a test solution containing antibodies with an anti-gp350 antibody probe, or an anti-gp350 B-cell probe, of the disclosure, and isolating an antibody that specifically binds to the anti-gp350 antibody probe, or the anti-gp350 B-cell probe, thereby identifying an anti-gp350 antibody. The anti-gp350 antibody probe may be joined to an affinity tag such as, for example, a His tag, an epitope tag or a labeled tag (e.g., fluorescent label). The anti-gp350 antibody probe, or the anti-gp350 B-cell, may be immobilized on a surface.

This disclosure also includes methods of identifying an anti-gp350 B-cell (i.e., a B-cell expressing an anti-gp350 B-cell receptor), comprising contacting a test solution containing B-cells with an anti-gp350 antibody probe, or an anti-gp350 B-cell probe, of the disclosure, and isolating a B-cell that specifically binds to the anti-gp350 antibody probe, or the anti-gp350 B-cell probe, thereby identifying a B-cell expressing an anti-gp350 B-cell receptor. The anti-gp350 antibody probe, or the anti-gp350 B-cell probe, may be joined to an affinity tag such as, for example, a His tag, an epitope tag or a labeled tag (e.g., fluorescent label). The anti-gp350 antibody probe, or the anti-gp350 B-cell probe, may be immobilized on a surface.

All patent and literature references cited in the present specification are hereby incorporated by reference in their entirety.

The foregoing disclosure is sufficient to enable one skilled in the art to practice the invention. The present invention is not to be limited in scope by the constructs described, because the described embodiments are intended as illustrations of certain aspects of the invention and any constructs that are functionally equivalent are within the scope of this invention.

### Example

The inventors isolated and characterized a set of human monoclonal antibodies from two blood bank donors who have extraordinally high serum neutralizing antibody titers to EBV. Using the receptor-binding null gp350 probes described in this invention they successfully identified gp350-specific B cells in peripheral blood mononuclear cells without having issues with non-targeting memory B cells that ubiquitously express CR2 (i.e. gp350 receptor) on their surface. Of those monoclonal antibodies, antibodies named A9 and E11 derived from 2 independent donors possessed the highest neutralization activity against EBV, and hence, they were structurally characterized. Structures of antigen-binding fragment (Fab) of A9 and E11 were solved to resolution of 2.4 and 1.8 Å, respectively. Additionally, the structure of Fab A9 in complex with gp350 receptor-binding domain was solved to 3.5 Å. Based on the structural information they designed a set of mutations on both antibodies A9 and E11 that potentially improve affinity and therefore, enhance neutralization potency.

These highly potent neutralizing monoclonal antibodies are useful in prophylaxis to prevent EBV infection in EBV-naïve transplantation recipients who revive organs/bone marrow from EBV-positive donors. These EBV-naïve transplantation recipients are at high risk for developing lymphoproliferative disease caused by EBV. Also, patients with certain genetic disorders (e.g. XLP1) are at high risk of developing fatal mononucleosis after primary infection with the virus. Currently, there are no such countermeasures that specifically target EBV.

These antibodies are also useful as therapeutics to treat infectious mononucleosis or to prevent reactivation of virus in persistently EBV infected individuals.

1. An isolated antibody that binds Epstein Barr Virus (EBV) gp350 protein, comprising a variable heavy (VH) domain and a variable light (VL) domain, wherein:
(a) the VH domain comprises the complementarity determining region 1 (CDR1), CDR2 and CDR3 sequences of SEQ ID NO: 1, and the VL domain comprises the CDR1, CDR2 and CDR3 sequences of SEQ ID NO: 6;
(b) the VH domain comprises the CDR1, CDR2 and CDR3 sequences of SEQ ID NO: 11, and the VL domain comprises the CDR1, CDR2 and CDR3 sequences of SEQ ID NO: 16;
(c) the VH domain comprises the CDR1, CDR2 and CDR3 sequences of SEQ ID NO: 21, and the VL domain comprises the CDR1, CDR2 and CDR3 sequences of SEQ ID NO: 26;
(d) the VH domain comprises the CDR1, CDR2 and CDR3 sequences of SEQ ID NO: 31, and the VL domain comprises the CDR1, CDR2 and CDR3 sequences of SEQ ID NO: 36;
(e) the VH domain comprises the CDR1, CDR2 and CDR3 sequences of SEQ ID NO: 41, and the VL domain comprises the CDR1, CDR2 and CDR3 sequences of SEQ ID NO: 46;
(f) the VH domain comprises the CDR1, CDR2 and CDR3 sequences of SEQ ID NO: 161, and the VL domain comprises the CDR1, CDR2 and CDR3 sequences of SEQ ID NO: 166, wherein the antibody is an engineered antibody;
(g) the VH domain comprises the CDR1, CDR2 and CDR3 sequences of SEQ ID NO: 171, and the VL domain comprises the CDR1, CDR2 and CDR3 sequences of SEQ ID NO: 176, wherein the antibody is an engineered antibody;
(h) the VH domain comprises the CDR1, CDR2 and CDR3 sequences of SEQ ID NO: 181, the VL domain comprises the CDR1, CDR2 and CDR3 sequences of SEQ ID NO: 186, wherein the antibody is an engineered antibody;
(i) the VH domain comprises the CDR1, CDR2 and CDR3 sequences of SEQ ID NO: 191, and the VL domain comprises the CDR1, CDR2 and CDR3 sequences of SEQ ID NO: 196, wherein the antibody is an engineered antibody;
(j) the VH domain comprises the CDR1, CDR2 and CDR3 sequences of SEQ ID NO: 201, and the VL domain comprises the CDR1, CDR2 and CDR3 sequences of SEQ ID NO: 206, wherein the antibody is an engineered antibody;
(k) the VH domain comprises the CDR1, CDR2 and CDR3 sequences of SEQ ID NO: 211, and the VL domain comprises the CDR1, CDR2 and CDR3 sequences of SEQ ID NO: 216, wherein the antibody is an engineered antibody;
(l) the VH domain comprises the CDR1, CDR2 and CDR3 sequences of SEQ ID NO: 221, and the VL domain comprises the CDR1, CDR2 and CDR3 sequences of SEQ ID NO: 226, wherein the antibody is an engineered antibody;
(m) the VH domain comprises the CDR1, CDR2 and CDR3 sequences of SEQ ID NO: 231, and the VL domain comprises the CDR1, CDR2 and CDR3 sequences of SEQ ID NO: 236, wherein the antibody is an engineered antibody;
(n) the VH domain comprises the CDR1, CDR2 and CDR3 sequences of SEQ ID NO: 241, and the VL domain comprises the CDR1, CDR2 and CDR3 sequences of SEQ ID NO: 246, wherein the antibody is an engineered antibody;
(o) the VH domain comprises the CDR1, CDR2 and CDR3 sequences of SEQ ID NO: 251, and the VH domain comprises the CDR1, CDR2 and CDR3 sequences of SEQ ID NO: 256, wherein the antibody is an engineered antibody;
(p) the VH domain comprises the CDR1, CDR2 and CDR3 sequences of SEQ ID NO: 261, and the VL domain comprises the CDR1, CDR2 and CDR3 sequences of SEQ ID NO: 266, wherein the antibody is an engineered antibody;
(q) the VH domain comprises the CDR1, CDR2 and CDR3 sequences of SEQ ID NO: 271, and the VL domain comprises the CDR1, CDR2 and CDR3 sequences of SEQ ID NO: 276, wherein the antibody is an engineered antibody; or
(r) the VH domain comprises the CDR1, CDR2 and CDR3 sequences of SEQ ID NO: 281, and the VL domain comprises the CDR1, CDR2 and CDR3 sequences of SEQ ID NO: 286, wherein the antibody is an engineered antibody.

2. The antibody of claim 1, which is conjugated to a solid support selected from a support formed partially or entirely of glass, a polysaccharide, a polyacrylamide, a polystyrene, a polyvinyl alcohol, a silicone, an assay plate, and a purification column.

3. A composition comprising the antibody of claim 1 and a carrier.

4. The composition of claim 3, wherein the carrier comprises a pharmaceutically acceptable carrier.

5. An article of manufacture comprising a container and the composition of claim 4 contained within the container.

6. The article of manufacture of claim 5, further comprising a label affixed to the container, or a package insert included with the container, referring to the use of the composition of matter for the therapeutic treatment of or the diagnostic detection of an EBV infection.

7. An expression vector comprising a nucleic acid molecule encoding the antibody of claim 1, operably linked to a promoter.

8. An isolated host cell comprising the expression vector of claim 7.

9. The isolated host cell of claim 8, which is a mammalian cell, a bacterial cell, or a yeast cell.

10. A process for producing an antibody, comprising culturing the host cell of claim 8 under conditions suitable for expression of the antibody and recovering the antibody from the cell culture.

11. A method of determining the presence of an EBV gp350 protein in a sample suspected of containing the protein, comprising exposing the sample to the antibody of claim 1 and detecting binding of the antibody to a protein in the sample, wherein binding of the antibody to a protein is indicative of the presence of EBV gp350 protein in the sample.

12. The method of claim 11, wherein the antibody is detectably labeled.

13. The method of claim 12, wherein the label is selected from a radioisotope and a fluorescent label.

14. The method of claim 11, wherein the antibody is conjugated to a solid support selected from a support formed partially or entirely of glass, a polysaccharide, a polyacrylamide, a polystyrene, a polyvinyl alcohol, a silicone, an assay plate, and a purification column.

15. The method of claim 11, wherein the sample comprises a cell suspected of containing an EBV gp350 protein, and the cell is an EBV-infected cell, an epithelial cell, a B lymphocyte, an oropharyngeal cell, a nasopharyngeal cell, or a cancer cell.

16. A method of diagnosing and treating the presence of an EBV infection in a mammal, comprising determining the level of expression of a gene encoding an EBV gp350 protein in a test sample of tissue cells obtained from the mammal and in a control sample of known normal cells of the same tissue origin, wherein a higher level of expression of the EBV gp350 protein in the test sample, as compared to the control sample, is indicative of the presence of an EBV infection in the mammal from which the test sample was obtained, and wherein the mammal determined to have the presence of the EBV infection is administered a therapeutically effective amount of an antibody of claim 1.

17. A protein comprising a polypeptide at least 85% identical to the amino acid sequence of the complement receptor 2 (CR2)-binding region of Epstein-Barr virus (EBV) gp350 set forth as amino acid residues 2-425 of SEQ ID NO: 124, wherein:
the amino acid residue corresponding to amino acid I160 of SEQ ID NO: 124 is substituted with arginine, serine, threonine, cysteine, proline, asparagine, glutamine, tyrosine, phenylalanine, tryptophan, glutamate, aspartate, glutamine, histidine, or lysine; and/or
the amino acid corresponding to amino acid K161 of SEQ ID NO: 124 is substituted with methionine, leucine, isoleucine, glycine, alanine, valine, phenylalanine, tyrosine, tryptophan, lysine, arginine, histidine, glutamate, serine, or aspartate,
wherein the protein can bind an anti-gp350 antibody, or a B-cell expressing an anti-gp350 B-cell receptor (BCR), wherein binding of the B-cell is through the BCR, and wherein binding of the protein to CR2 is substantially reduced compared to binding of wild-type gp350 to CR2.

18. The protein of claim 17, wherein:
(a) the amino acid corresponding to amino acid Q122 of SEQ ID NO: 124 is substituted with asparagine, methionine, leucine, isoleucine, glycine, alanine, valine, phenylalanine, tyrosine, tryptophan, lysine, arginine, histidine, glutamate, or aspartate;
(b) the amino acid corresponding to amino acid P158 of SEQ ID NO: 124 is substituted with arginine, methionine, leucine, isoleucine, glycine, alanine, valine, phenylalanine, tyrosine, tryptophan, lysine, histidine, glutamate, or aspartate;
(c) the amino acid corresponding to amino acid W162 of SEQ ID NO: 124 is substituted with alanine, serine, threonine, cysteine, proline, asparagine, glutamine, aspartate, glutamate, asparagine, arginine, glutamate, histidine, glycine, valine, leucine, methionine, isoleucine, or lysine;
(d) the amino acid corresponding to amino acid D163 of SEQ ID NO: 124 is substituted with asparagine, tyrosine, phenylalanine, tryptophan, serine, threonine, cysteine, proline, glutamine, lysine, arginine, histidine, glycine, alanine, valine, leucine, methionine, or isoleucine;
(e) the amino acid corresponding to amino acid N164 of SEQ ID NO: 124 is substituted with serine, methionine, leucine, isoleucine, glycine, alanine, valine, phenylalanine, tyrosine, tryptophan, lysine, arginine, histidine, glutamate, or aspartate;
(f) a serine, threonine, cysteine, proline, asparagine, or glutamine residue is inserted between the amino acid corresponding to amino acid N164 of SEQ ID NO: 124 and the amino acid corresponding to C165 of SEQ ID NO: 124;
(g) the amino acids corresponding to amino acids W162-N164 of SEQ ID NO: 124 are each substituted with an alanine residue; and/or
(h) the amino acid corresponding to amino acid D296 of SEQ ID NO: 124 is deleted, or is substituted with one or more amino acid residues selected from the group consisting of tyrosine, phenylalanine, tryptophan, serine, threonine, cysteine, proline, asparagine, glutamine, lysine, arginine, histidine, glycine, alanine, valine, leucine, methionine, and isoleucine.

19. A nucleic acid molecule encoding the protein of claim 17.

20. A method for identifying an anti-gp350 antibody, or an anti-gp350 B cell, comprising:
contacting the protein of claim 17 with a solution comprising antibodies or B cells; and
isolating antibodies or B cells that specifically bind to the protein.


SEQUENCE LISTING (293 sequences)

SEQ ID NO: 1 - protein, 121 aa, Homo sapiens
QVQLQESGPGLVKPAETLSLTCTVSGGSFSSYWWGWIRQSPGKGLEWIGHISSGGNNYLNPSLKSRVTLSLDTSKNQFSLKLNSVTAADSAVYYCARAPRIVVRGRYFDQWGQGVLVTVSS

SEQ ID NO: 2 - DNA, 363 bp, Homo sapiens
  source 1..363: mol_type = genomic DNA
caggtgcagctgcaggagtcgggcccaggactggtgaagcctgcggagaccctgtccctcacctgcactgtctctggtggctctttcagcagttactggtggggctggatccgtcagtccccagggaagggactggagtggattgggcatatcagtagtggtggaaacaactaccttaatccgtccctcaagagtcgagtcaccctgtcactagacacgtccaagaaccagttctccctgaagctgaactctgtgaccgccgcggactcggccgtgtattactgtgccagagccccccgtattgttgttagaggccgatactttgaccaatggggccagggagtcctggtcaccgtctcctca

SEQ ID NO: 3 - protein, 8 aa, Homo sapiens
GGSFSSYW

SEQ ID NO: 4 - protein, 7 aa, Homo sapiens
ISSGGNN

SEQ ID NO: 5 - protein, 16 aa, Homo sapiens
APAPRIVVRGRYFDQW

SEQ ID NO: 6 - protein, 107 aa, Homo sapiens
DIQMTQSPSSLSASVGDTVTITCRASQGINIYLNWFQQRPGKAPKLLIYAATTLQSGVPSRFSGSGSGTDFTLTISSLQPEDFATYYCLQCESYPLTFGGGTKVEIK

SEQ ID NO: 7 - DNA, 321 bp, Homo sapiens
  source 1..321: mol_type = genomic DNA
gacatccagatgacccagtctccatcctccctgtctgcatctgtaggagacactgtcaccatcacttgccgggcaagtcagggtatcaatatctacctgaattggtttcagcagagaccagggaaagcccctaaactcctgatctatgctgcgaccactttacaaagtggggtcccatcaagattcagcggcagtggatctgggacagatttcactctcaccatcagcagcctgcagcctgaagatttcgcaacttattactgtctacagtgtgaaagttatccgctcactttcggcggagggaccaaggtggagatcaaa

SEQ ID NO: 8 - protein, 6 aa, Homo sapiens
QGINIY

SEQ ID NO: 9 - skipped

SEQ ID NO: 10 - protein, 9 aa, Homo sapiens
LQCESYPLT

SEQ ID NO: 11 - protein, 123 aa, Homo sapiens
QVQLQESGPGLVKPSETLSLTCTVSGGSISGAYYYWSWIRQPPGKGLDWIGYIYGSFGSAYYNPSLKSRATISKDTPKNQFSLKLSSVTAADTAVYYCARGRRLGYSNWFDVWGPGVLVTVSS

SEQ ID NO: 12 - DNA, 369 bp, Homo sapiens
  source 1..369: mol_type = genomic DNA
caggtgcaactgcaggagtcgggcccaggactggtgaagccttcggagaccctgtccctcacctgcactgtctctggtggctccatcagcggtgcttactactactggagctggattcgacagcccccggggaagggactggactggattggatatatctatggaagttttgggagtgcctactacaacccctccctcaagagtcgagccaccatttcaaaagacacgcccaagaaccagttctccctgaaactgagctctgtgaccgccgcggacacggccgtgtattactgtgcgagaggaaggcgactaggctattcgaactggttcgatgtctggggcccgggagtcctggtcaccgtctcctca

SEQ ID NO: 13 - protein, 10 aa, Homo sapiens
GGSISGAYYY

SEQ ID NO: 14 - protein, 8 aa, Homo sapiens
IYGSFGSA

SEQ ID NO: 15 - protein, 15 aa, Homo sapiens
ARGRRLGYSNWFDVW

SEQ ID NO: 16 - protein, 107 aa, Homo sapiens
DIQMTQSPSSLSASVGDKVTITCRTSQDVSSYLAWYQQKPGKAPQLLIYAASSLQSGVPSRFTGSGSGAEFTLTISSLQPEDFASYYCQQYKNLPLTFGGGTKVEIK

SEQ ID NO: 17 - DNA, 321 bp, Homo sapiens
  source 1..321: mol_type = genomic DNA
gacatccagatgacccagtctccatcttccctgtctgcatctgtaggagacaaagtcaccatcacttgtcggacaagtcaggacgttagcagttatttagcctggtatcagcagaaaccagggaaagcccctcagctcctgatctatgctgcatccagtttgcaaagtggggtcccatcaaggttcaccggcagtggatctggggcagaattcactctcaccatcagcagccttcagcctgaagattttgcatcatattactgtcaacagtataaaaatctcccgctcactttcggcggagggaccaaagtggagatcaaa

SEQ ID NO: 18 - protein, 6 aa, Homo sapiens
QDVSSY

SEQ ID NO: 19 - skipped

SEQ ID NO: 20 - protein, 9 aa, Homo sapiens
QQYKNLPLT

SEQ ID NO: 21 - protein, 129 aa, Homo sapiens
QVQLQESGPGLVKPSETLSLTCDVSGGSFSGDFYWSWIRQPPGKGLDWIGNIHGSSAGTKYKPSLKSRVTISKDTSKNQFSLKLSSVTAADTAVYYCTRGPLSRIVAGFGRGINWFDVWGPGVLVTVSS

SEQ ID NO: 22 - DNA, 387 bp, Homo sapiens
  source 1..387: mol_type = genomic DNA
caggtgcagctgcaggagtcgggcccaggactggtgaagccttcggagaccctgtccctcacctgcgatgtctctggtggctccttcagcggtgatttctactggagctggatccgccagcccccagggaagggactggactggattgggaatatccatggcagcagtgcgggcaccaaatacaagccctccctcaagagtcgagtcaccatttcaaaagacacgtccaagaaccagttctccctgaaactgagctctgtgaccgccgcggacacggccgtctattactgtacgagaggcccccttagtaggatagtagctggttttgggagggggattaactggttcgatgtctggggcccgggagtcctggtcaccgtctcctca

SEQ ID NO: 23 - protein, 9 aa, Homo sapiens
GGSFSGDFY

SEQ ID NO: 24 - protein, 8 aa, Homo sapiens
IHGSSAGT

SEQ ID NO: 25 - protein, 22 aa, Homo sapiens
TRGPLSRIVAGFGRGINWFDVW

SEQ ID NO: 26 - protein, 116 aa, Homo sapiens
QPVLTQPTSLSASPGASVRLSCTLSSGINVGSYSIFWYQQKPGSPPRYLLFYFSDSSKHQGSGVPSRFSGSKDTSANAGLLLISGLQSEDEADYYCAIWHSSASVLFGGGTRLTVL

SEQ ID NO: 27 - DNA, 348 bp, Homo sapiens
  source 1..348: mol_type = genomic DNA
cagcctgtgctgacccagccaacctccctctcagcatctccgggagcatcagtcagactcagctgcaccttgagcagtggcatcaatgttggtagttacagcatattctggtaccagcagaagccagggagtcctccccggtaccttctgttctatttctcagactcaagtaagcaccagggctctggagtccccagccgtttctctggatccaaggatacttcagccaatgcagggcttttactgatctctgggctccagtctgaagatgaggctgactattactgtgccatatggcacagcagcgcttctgtgttattcggaggagggacccggctgacagtacta

SEQ ID NO: 28 - protein, 14 aa, Homo sapiens
TLSSGINVGSYSIF

SEQ ID NO: 29 - protein, 7 aa, Homo sapiens
YFSDSSK

SEQ ID NO: 30 - protein, 10 aa, Homo sapiens
AIWHSSASVL

SEQ ID NO: 31 - protein, 120 aa, Homo sapiens
QLQLQESGPGLVKPSETLSLTCAVSGGSISGYYWSWIRQPPGKGPEWIGFIDGNTVGTNYNPSLKSRVTLSKDTSKNQFSLKVSSVTAADTAVYYCARKPLRRYFWFDVWGPGVLVTVSS

SEQ ID NO: 32 - DNA, 360 bp, Homo sapiens
  source 1..360: mol_type = genomic DNA
cagctgcagctgcaggagtcgggcccaggactggtgaagccttcggagaccctgtccctcacctgcgctgtctctggtggctccatcagcggttactactggagttggattcgccagcccccagggaagggaccggagtggattgggtttattgatggtaatactgtgggcaccaactacaacccctccctcaagagtcgagtcaccctttcaaaagacacgtccaagaatcagttctccctgaaggtgagttctgtgaccgccgcggacacggccgtgtattactgtgcgaggaagccgctacgccgttatttctggttcgatgtctggggcccgggagtcctggtcaccgtctcctca

SEQ ID NO: 33 - protein, 8 aa, Homo sapiens
GGSISGYY

SEQ ID NO: 34 - protein, 8 aa, Homo sapiens
IDGNTVGT

SEQ ID NO: 35 - protein, 14 aa, Homo sapiens
ARKPLRRYFWFDVW

SEQ ID NO: 36 - protein, 111 aa, Homo sapiens
QSVLTQPPSVSGDPGQRVTISCTGSSSNIGAGYYVYWYQQFPGTAPKLLIYQDNKRPSGVSDRFSGSKSGTSASLTITGLQPGDEADYYCSAWDSSLSAVMFGRGTRLTVL

SEQ ID NO: 37 - DNA, 333 bp, Homo sapiens
  source 1..333: mol_type = genomic DNA
cagtctgtgctgacgcagccgccctcagtgtctggggaccccgggcagagggtcaccatctcgtgcactgggagcagctccaacatcggggcgggttattatgtatactggtaccagcagttcccaggaacggcccccaaactcctcatctatcaagataataagcgaccctcaggggtttctgaccgattctctggctccaagtctggtacctcagcctccctgaccatcactgggctccagcctggggatgaggctgattattactgctcagcatgggatagcagcctgagtgctgttatgttcggaagaggcaccaggctgacagtacta

SEQ ID NO: 38 - protein, 9 aa, Homo sapiens
SSNIGAGYY

SEQ ID NO: 39 - skipped

SEQ ID NO: 40 - protein, 11 aa, Homo sapiens
SAWDSSLSAVM

SEQ ID NO: 41 - protein, 120 aa, Homo sapiens
QLQLQESGPGVVKPSETLSLTCTISGGSFSTYYWTWIRQPPGKGLEWVGYIGNGGRSLNYNPSLKSRITLSVDASKNQFSLKVTSVTAADTAVYYCGRARGLRGNWFDVWGPGVLVTVSS

SEQ ID NO: 42 - DNA, 360 bp, Homo sapiens
  source 1..360: mol_type = genomic DNA
caactgcagttgcaggagtcgggcccaggagtggtgaagccttcggagaccctgtccctcacctgcactatctctggtggctccttcagtacttactactggacctggattcgccagcccccagggaagggactggagtgggttgggtatatcggtaatggtggtcgtagcctcaactacaacccctccctcaagagtcgcatcaccctgtcagtagacgcgtccaagaaccagttctccctgaaggtgacctctgtgaccgccgcggacacggccgtctattactgtgggagagccaggggactccgcggaaactggttcgatgtctggggcccgggagtcctggtcaccgtctcctca

SEQ ID NO: 43 - protein, 8 aa, Homo sapiens
GGSFSTYY

SEQ ID NO: 44 - protein, 8 aa, Homo sapiens
IGNGGRSL

SEQ ID NO: 45 - protein, 14 aa, Homo sapiens
GRARGLRGNWFDVW

SEQ ID NO: 46 - protein, 110 aa, Homo sapiens
QAALTQPPSVSGSPGQSVTISCTGTSSDIGGYNYVSWYQQHPGKAPKVMIYEVSKRPSGVSDRFSGSKSGNIASLTISGLQAEDEADYYCSSYAGSNTFLFGGGTRLTVL

SEQ ID NO: 47 - DNA, 330 bp, Homo sapiens
  source 1..330: mol_type = genomic DNA
caggctgccctgactcagcctccctctgtgtctgggtctcctggacagtcggtcaccatctcctgcactggaaccagcagtgacatcggtggttataactatgtctcctggtaccaacaacacccaggcaaagcccccaaagtcatgatttatgaggtcagtaagcggccctcaggggtctctgatcgcttctctggttccaaatctggcaacatagcctccctgaccatctctgggctccaggctgaggacgaggctgattattactgcagctcatatgcaggcagcaacactttcttattcggaggagggacccggctgacagtacta

SEQ ID NO: 48 - protein, 9 aa, Homo sapiens
SSDIGGYNY

SEQ ID NO: 49 - skipped

SEQ ID NO: 50 - protein, 10 aa, Homo sapiens
SSYAGSNTFL

SEQ ID NO: 51 - protein, 123 aa, Homo sapiens
QVQLQESGPGLVKPSETLSLTCAVSGGSISSNYWSWIRQPPGKGLEWIGRIYGSGGSTDYNPSLKSRVTISTDTSKNQFSLKVSSVTAADTAVYYCARVRIQWVQLRGWFDVWGPGVLVTVSS

SEQ ID NO: 52 - DNA, 369 bp, Homo sapiens
  source 1..369: mol_type = genomic DNA
caggtgcagctgcaggagtcgggcccaggactggtgaagccttcggagaccctgtccctcacctgcgctgtctctggtggctccatcagcagtaactactggagctggatccgccagcccccagggaagggactggagtggattggacgtatctatggtagtggtgggagcaccgactacaacccctccctcaagagtcgagtcaccatttcaacagacacgtccaagaaccagttctccctgaaggtgagctctgtgaccgccgcggacacggccgtgtattactgtgcgagagtgcggatacagtgggtacagttgcgaggctggttcgatgtctggggcccgggagtcctggtcaccgtctcctca

SEQ ID NO: 53 - protein, 10 aa, Homo sapiens
GGSISSNYWS

SEQ ID NO: 54 - protein, 8 aa, Homo sapiens
IYGSGGST

SEQ ID NO: 55 - protein, 17 aa, Homo sapiens
ARVRIQWVQLRGWFDVW

SEQ ID NO: 56 - protein, 107 aa, Homo sapiens
YIQMTQSPSSLSASVGDTVTFTGPASQSFSSSLAWYQQKPGKAPNLLIYSASSLQCGVRSRFSGSKSGTDFTLTISSLQPEDIASYYCQQYYSYPFTFGPGTKLDIK

SEQ ID NO: 57 - DNA, 321 bp, Homo sapiens
  source 1..321: mol_type = genomic DNA
tacatacagatgacgcagtctccatcctccctgtctgcatctgtaggagacacagtcaccttcactggccccgcaagtcagagctttagtagtagtttagcctggtatcagcagaaaccagggaaagcccctaacctcctgatctatagtgcatccagtttgcaatgtggggttcgttcgaggttcagtggcagtaagtctgggacagatttcactctcaccatcagcagcctgcagcctgaagatattgctagttattactgtcaacagtattacagttatccattcactttcggccccgggaccaaactggatatcaaa

SEQ ID NO: 58 - protein, 6 aa, Homo sapiens
QSFSSS

SEQ ID NO: 59 - skipped

SEQ ID NO: 60 - protein, 9 aa, Homo sapiens
QQYYSYPFT

SEQ ID NO: 61 - protein, 119 aa, Homo sapiens
QVQLQESGPGLVRPSETLSLTCAVSGGSISSNYWSWIRQPPGKGLEWIGYISGSTGSTYQNPSLKSRVTVSKDTSKNQFSLKLNSVTAADTAVYYCARSGRRGSSLDLWGRGVLVTVSS

SEQ ID NO: 62 - DNA, 357 bp, Homo sapiens
  source 1..357: mol_type = genomic DNA
caggtgcagctgcaggagtcgggcccaggactggtgaggccttcggagaccctatccctcacctgcgctgtctctggtggctccatcagcagtaactactggagctggattcgccagcccccagggaaggggctggagtggattgggtatatctctggtagtactgggagcacctaccagaacccctccctcaagagtcgagtcaccgtttcaaaagacacgtctaagaaccagttctccctgaagctgaattctgtgaccgccgcggacacggccgtgtattactgtgcgagaagtgggagaagaggcagctcattggatttgtggggccggggagttctggtcaccgtctcctca

SEQ ID NO: 63 - protein, 8 aa, Homo sapiens
GGSISSNY

SEQ ID NO: 64 - protein, 8 aa, Homo sapiens
ISGSTGST

SEQ ID NO: 65 - protein, 13 aa, Homo sapiens
ARSGRRGSSLDLW

SEQ ID NO: 66 - protein, 111 aa, Homo sapiens
QSVLTQPPSVSGDPGQRVTISCTGSSSNIGAGYYVYWYQQFPGTAPKLLIYQDNKRPSGVSDRFSGSKSGTSASLTITGLQPGDEADYYCSAWDSSLSAVFFGGGTRLTVL

SEQ ID NO: 67 - DNA, 333 bp, Homo sapiens
  source 1..333: mol_type = genomic DNA
cagtctgtgctgacgcagccgccctcagtgtctggggaccccgggcagagggtcaccatctcgtgcactgggagcagctccaacatcggggcgggttattatgtatactggtaccagcagttcccaggaacggcccccaaactcctcatctatcaagataataagcgaccctcaggggtttctgaccgattctctggctccaagtctggtacctcagcctccctgaccatcactgggctccagcctggggatgaggctgattattactgctcagcatgggatagcagcctgagtgctgtgttcttcggaggagggacccggctgacagtacta

SEQ ID NO: 68 - protein, 9 aa, Homo sapiens
SSNIGAGYY

SEQ ID NO: 69 - skipped

SEQ ID NO: 70 - protein, 11 aa, Homo sapiens
SAWDSSLSAVF

SEQ ID NO: 71 - protein, 121 aa, Homo sapiens
QLQESGPGLVKPSETLSLTCTVSGGSISDTYRWSWIRQSPGKGLEWIAYIYGTTTSTNYNPSLKSRLTISKDTSKNQFSLNLRSVTAADTAVYYCARGDSGGRSAHVFHFWGQGLRVTVSS

SEQ ID NO: 72 - DNA, 363 bp, Homo sapiens
  source 1..363: mol_type = genomic DNA
cagctgcaggagtcgggcccaggactggtgaagccttcggagaccctgtccctcacctgcactgtctctggtggctccatcagcgatacttaccggtggagctggattcgccagtccccagggaagggactggagtggattgcctacatctatggtactactacgagcaccaactacaacccctccctcaagagtcgactcaccatttcaaaagacacgtccaagaaccagttctccttgaacctgaggtctgtgaccgccgcggacacggccgtgtattactgtgcgagaggggatagcggtggccggtcagcgcatgtttttcatttctggggccaagggctcagggtcaccgtctcttca

SEQ ID NO: 73 - protein, 9 aa, Homo sapiens
GGSISDTYR

SEQ ID NO: 74 - protein, 8 aa, Homo sapiens
IYGTTTST

SEQ ID NO: 75 - protein, 16 aa, Homo sapiens
ARGDSGGRSAHVFHFW

SEQ ID NO: 76 - protein, 111 aa, Homo sapiens
QSVLTQPPSVFGDPGQRITISCTGSSSNIGAGYYVYWYQQFPGTAPKLLIYQDNKRPSGVSDRFSGSKSGSSASLTITGLQPGDEADYYCSAWDSSLSVRVFGGGTRLTVL

SEQ ID NO: 77 - DNA, 333 bp, Homo sapiens
  source 1..333: mol_type = genomic DNA
cagagtgttctgacgcagccgccctcagtgtttggggaccccgggcagaggatcaccatctcgtgcactgggagcagctccaacatcggggcgggttattatgtatactggtaccagcagttcccaggaacggcccccaaactcctcatctatcaagataataagcgaccctcaggggtttctgaccgattttctggctccaagtctggttcctcagcctccctgaccatcactgggctccagcctggggatgaggctgattattactgctcagcatgggatagcagcctgagtgtacgggttttcggaggagggacccggctgacagtacta

SEQ ID NO: 78 - protein, 9 aa, Homo sapiens
SSNIGAGYY

SEQ ID NO: 79 - skipped

SEQ ID NO: 80 - protein, 11 aa, Homo sapiens
SAWDSSLSVRV

SEQ ID NO: 81 - protein, 122 aa, Homo sapiens
EVQLVESGGGLVQPGGSLRLSCAASGFTFSSYGMNWVRQAPGKGLEWVSFITNTGKTTYYADSVRGRFTISRDNAKKSVSLQMSSLRAEDTAVYYCTRGRGRHGWSSGVFDFWGQGLRVTVS

SEQ ID NO: 82 - DNA, 368 bp, Homo sapiens
  source 1..368: mol_type = genomic DNA
gaggtgcaactggtggagtctggaggaggcttggtccagcctggagggtccctgagactctcctgtgcagcctctggattcaccttcagtagttacggcatgaactgggtccgccaggctccgggaaaggggctggagtgggtctcattcattactaacactggtaaaaccacatactacgctgactctgtgaggggccgattcaccatctccagagacaacgccaagaagtcggtgtctctacaaatgagtagcctgagagccgaggacacggccgtctattactgtactaggggaagaggtagacacggctggtccagtggtgtttttgatttctggggccaaggtctcagggtcaccgtctcttc

SEQ ID NO: 83 - protein, 8 aa, Homo sapiens
GFTFSSYG

SEQ ID NO: 84 - protein, 8 aa, Homo sapiens
ITNTGKTT

SEQ ID NO: 85 - protein, 16 aa, Homo sapiens
TGRGRHGWSSGVFDFW

SEQ ID NO: 86 - protein, 111 aa, Homo sapiens
QSVLTQPPSVFGDPGQRITISCTGSSSNIGAGYYVYWYQQFPGTAPKLLIYQDNKRPSGVSDRFSGSKFGSSASLTITGVQRGDEGDYYCSAWDSSLSVRVLGGGTRLTVL

SEQ ID NO: 87 - DNA, 333 bp, Homo sapiens
  source 1..333: mol_type = genomic DNA
cagtctgttctgacgcagccgccctcagtgttcggggaccccgggcagaggatcaccatttcgtgcactgggagcagctccaacatcggggcgggttattatgtatactggtaccagcagttcccaggaacggcccccaaactcctcatctatcaagataataagcgaccctcaggggtttctgaccgattctctggctccaagtttggttcctcagcctccctgaccatcactggggtccagcgtggggatgagggtgattattactgctcagcatgggatagcagcctgagtgtacgggttttgggaggagggacccggctgacagtacta

SEQ ID NO: 88 - protein, 9 aa, Homo sapiens
SSNIGAGYY

SEQ ID NO: 89 - skipped

SEQ ID NO: 90 - protein, 11 aa, Homo sapiens
SAWDSSLSVRV

SEQ ID NO: 91 - protein, 121 aa, Homo sapiens
EVQLVESGGGLVQPGGSLRLSCVASGFTFSDRYIDWVRQAPGKGLEWVSTISTGSGDTALYSDSVKGRFTISRDNAKNTLYLQMNSLRAEDTAVYYCARHSGTFYTHFDYWGQGVLVTVSS

SEQ ID NO: 92 - DNA, 363 bp, Homo sapiens
  source 1..363: mol_type = genomic DNA
gaagtgcagttggtggagtctgggggaggcttggtacagccgggggggtccctgagactctcctgtgtagcctctggattcaccttcagtgaccgctacatagactgggtccgccaggctccagggaagggcctggagtgggtctcaactattagcactggtagtggtgataccgcattgtactcagactctgtcaagggccgattcaccatctccagagacaacgccaagaacacactgtatctgcaaatgaacagcctgagagccgaagacacggctgtctattactgtgcgagacatagtggtactttttacacccactttgactactggggccagggagtcctggtcaccgtctcctca

SEQ ID NO: 93 - protein, 8 aa, Homo sapiens
GFTFSDRY

SEQ ID NO: 94 - protein, 9 aa, Homo sapiens
ISTGSGDTA

SEQ ID NO: 95 - protein, 14 aa, Homo sapiens
ARHSGTFYTHFDYW

SEQ ID NO: 96 - protein, 107 aa, Homo sapiens
DIQMTQSPSSLSASVGDTVTFTCRASRSISSWLAWYQQKPGRAPKVLIYKASSLQSGVPSRFSGSGSGTDFTLTISSLQSEDFATYYCQQYSSRPPTFGQGTKVEIR

SEQ ID NO: 97 - DNA, 321 bp, Homo sapiens
  source 1..321: mol_type = genomic DNA
gacatccagatgacccagtctccatcctccctgtctgcatctgtaggagacacagtcaccttcacctgccgggcgagtcggagtattagcagctggttagcctggtatcagcagaaaccagggagagcccctaaagtcctgatctataaggcgtccagtttgcaaagtggggttccttcaaggttcagcggcagtggatctgggacagacttcactctcaccatcagcagcctacagtctgaagattttgcaacatattattgtcaacagtatagtagtcgccctccgacgttcggccaagggaccaaggtggaaatcaga

SEQ ID NO: 98 - protein, 6 aa, Homo sapiens
RSISSW

SEQ ID NO: 99 - skipped

SEQ ID NO: 100 - protein, 9 aa, Homo sapiens
QQYSSRPPT

SEQ ID NO: 101 - protein, 119 aa, Homo sapiens
QVQLQESGPGLLKPSETLSLTCAVSGGSFSSFWWSWLRQPPEKGLEWIGEINGDSGSTNYNPSLKSRVTISKDASKNQFSLKLTSVTAADTAVFYCARVRRILRSLDVWGRGVLVTVSS

SEQ ID NO: 102 - DNA, 357 bp, Homo sapiens
  source 1..357: mol_type = genomic DNA
caggtgcagctgcaggagtcgggcccaggactgctgaagccttcggagaccctgtccctcacctgcgctgtctctggtggctccttcagtagtttctggtggagctggctccgccagcccccagaaaagggactggagtggattggggagatcaatggtgatagtgggagcaccaactacaacccctccctcaagagtcgagtcaccatttcaaaagacgcgtccaagaaccagttctccctgaaactgacctctgtgaccgccgcggacacggccgttttttactgtgcgagagttcggcgaattctgaggtcattggatgtctggggccggggagttctggtcaccgtctcctca

SEQ ID NO: 103 - protein, 8 aa, Homo sapiens
GGSFSSFW

SEQ ID NO: 104 - protein, 8 aa, Homo sapiens
INGDSGST

SEQ ID NO: 105 - protein, 13 aa, Homo sapiens
ARVRRILRSLDVW

SEQ ID NO: 106 - protein, 107 aa, Homo sapiens
DIQMTQSPFSLFAFVGDRVTITCQASQGISHLLAWYQQKPGKAPKLLIYSASTLQSGVPSRFSGSGFGTEFTLTISSLQPEDFATYYCQQHNSYPRTFGQGTKVEIK

SEQ ID NO: 107 - DNA, 321 bp, Homo sapiens
  source 1..321: mol_type = genomic DNA
gacatccagatgacccagtctcctttttccttgtttgcatttgtaggagacagagtcaccatcacttgccaagccagtcagggtattagccacttgttagcttggtatcagcagaaaccagggaaagcccctaagctccttatttattctgcatccactttgcaaagtggggtcccatcaaggttcagcggcagtggatttgggacggaattcactctcaccatcagcagcctgcagcctgaagattttgcaacttattactgtcaacagcataatagttaccctcggacgttcggccaagggaccaaggtggaaatcaaa

SEQ ID NO: 108 - protein, 6 aa, Homo sapiens
QGISHL

SEQ ID NO: 109 - skipped

SEQ ID NO: 110 - protein, 9 aa, Homo sapiens
QQHNSYPRT

SEQ ID NO: 111 - DNA, 5845 bp, Homo sapiens
  source 1..5845: mol_type = genomic DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagaccaccatgggatggtcatgtatcatcctttttctagtagcaactgcaaccggtgtacattcccaggtgcagctggtgcagctgcacgagtcgggcccaggactggtgaagccttcggagaccctgtccctcacctgcgctgtctctggtggctctatcagcagtagctactggagctggatccgccaggccccagggaagggactggagtggattgggtatgtctatggtagtggtcgtgacaccaacgacaacccctccctcaagagtcgagtcaccctgtcagtagacacgtccaagaaccagctctccctgaagctgagatctgtgaccgccgcggacacggccgtgtattactgtgcgagcagcggctggcctcctgggttggactactggggccagggagtcacggtcaccgtctcctcagctagcaccaagggccctagtgtgtttcctctggcccctagcagcagaagcacatctgaatctacagccgccctgggctgcctggtgaaagattacttccccgagcccgtgaccgtgtcttggaatagcggctctctgaccagcggcgtgcacacatttccagctgtgctgcagagcagcggcctgtattctctgagcagcgtggtgacagtgccaagcagctctctgggcacccagacctacgtgtgcaacgtgaaccacaagcccagcaacaccaaggtggacaagcgggtggaaatcaagacctgtggcggcggaagcaagcctcctacctgtcctccttgtaccagccctgaactgctgggcggacctagcgtgttcctgttccccccaaagcccaaggacaccctgatgatcagcagaacccccgaagtgacctgcgtggtggtggatgtgtcccaggaagatcccgacgtgaagttcaattggtacgtgaacggcgccgaggtgcaccatgcccagacaaagcccagagagacacagtacaacagcacctaccgggtggtgtctgtgctgaccgtgacacaccaggactggctgaacggcaaagagtacacatgcaaggtgtccaacaaggccctgcctgcccccatccagaaaaccatcagcaaggacaagggccagcccagagaacctcaggtgtacacactgccccccagcagagaggaactgaccaagaatcaggtgtccctgacctgtctggtgaaaggcttctaccccagcgacatcgtggtggaatgggagtctagcggacagcccgagaacacctacaagaccacccctccagtgctggatagcgacggcagctacttcctgtacagcaagctgaccgtggacaagagcagatggcagcagggcaacgtgttcagctgctctgtgatgcacgaggccctgcacaaccactacacccagaagtctctgagcctgagccccggaaagtgatgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 112 - protein, 473 aa, Homo sapiens
MGWSCIILFLVATATGVHSQVQLVQLHESGPGLVKPSETLSLTCAVSGGSISSSYWSWIRQAPGKGLEWIGYVYGSGRDTNDNPSLKSRVTLSVDTSKNQLSLKLRSVTAADTAVYYCASSGWPPGLDYWGQGVTVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 113 - DNA, 5100 bp, Homo sapiens
  source 1..5100: mol_type = genomic DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtacacgttgctgccgcgcgcgccaccagacataatagctgacagactaacagactgttcctttccatgggtcttttctgcagtcaccgtcgtcgacacgtgtgatcagatatcgcggccgctctagaccaccatgggatggtcatgtatcatcctttttctagtagcaactgcaaccggtgtacattcagaaattgtgttgacacagtctccaggcaccctgtctttgtctccaggggaaacagccatcatctcttgtcggaccagtcagtatggttccttagcctggtatcaacagaggcccggccaggcccccaggctcgtcatctattcgggctctactcgggccgctggcatcccagacaggttcagcggcagtcggtgggggccagactacaatctcaccatcagcaacctggagtcgggagattttggtgtttattattgccagcagtatgaattttttggccaggggaccaaggtccaggtcgacattaaacgtacggtggctgcaccatctgtcttcatcttcccgccatctgaggatcaggtgaaatctggaactgtctctgttgtgtgcctgctgaataacttctatcccagagaggccagcgtaaagtggaaggtggatggtgccctcaaaacgggtaactcccaggagagtgtcacagagcaggacagcaaggacaacacctacagcctgagcagcaccctgacgctgagcagcacagagtaccagagtcacaaagtctatgcctgcgaagtcacccatcagggcctgagctcgcccgtcaccaagagcttcaacagaggagagtgttagggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacatggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 114 - protein, 229 aa, Homo sapiens
MGWSCIILFLVATATGVHSEIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 115 - protein, 235 aa, Homo sapiens
MGWSCIILFLVATATGVHSQSALTQPPSVSGSPGQSVTISCTGTSSDVDGYNYVSWYQQHPGKAPKLMIYGVSNRPSGVSDRFSGSKSGNTASLTISGLQAEDEADYYCCSSTTSYTYIFGTGTKVTVLGQPKAAPSVTLFPPSSEELQANKATLVCLISDFYPGAVEVAWKADGSAVNAGVETTKPSKQSNNKYAASSYLSLTSDQWKSHKSYSCQVTHEGSTVEKTVAPAECS

SEQ ID NO: 116 - DNA, 5842 bp, Homo sapiens
  source 1..5842: mol_type = genomic DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagaccaccatgggatggtcatgtatcatcctttttctagtagcaactgcaaccggtgtacattcccaactgcagttgcaggagtcgggcccaggagtggtgaagccttcggagaccctgtccctcacctgcactatctctggtggctccttcagtacttactactggacctggattcgccagcccccagggaagggactggagtgggttgggtatatcggtaatggtggtcgtagcctcaactacaacccctccctcaagagtcgcatcaccctgtcagtagacgcgtccaagaaccagttctccctgaaggtgacctctgtgaccgccgcggacacggccgtctattactgtgggagagccaggggactccgcggaaactggttcgatgtctggggcccgggagtcctggtcaccgtctcctcagctagcaccaagggccctagtgtgtttcctctggcccctagcagcagaagcacatctgaatctacagccgccctgggctgcctggtgaaagattacttccccgagcccgtgaccgtgtcttggaatagcggctctctgaccagcggcgtgcacacatttccagctgtgctgcagagcagcggcctgtattctctgagcagcgtggtgacagtgccaagcagctctctgggcacccagacctacgtgtgcaacgtgaaccacaagcccagcaacaccaaggtggacaagcgggtggaaatcaagacctgtggcggcggaagcaagcctcctacctgtcctccttgtaccagccctgaactgctgggcggacctagcgtgttcctgttccccccaaagcccaaggacaccctgatgatcagcagaacccccgaagtgacctgcgtggtggtggatgtgtcccaggaagatcccgacgtgaagttcaattggtacgtgaacggcgccgaggtgcaccatgcccagacaaagcccagagagacacagtacaacagcacctaccgggtggtgtctgtgctgaccgtgacacaccaggactggctgaacggcaaagagtacacatgcaaggtgtccaacaaggccctgcctgcccccatccagaaaaccatcagcaaggacaagggccagcccagagaacctcaggtgtacacactgccccccagcagagaggaactgaccaagaatcaggtgtccctgacctgtctggtgaaaggcttctaccccagcgacatcgtggtggaatgggagtctagcggacagcccgagaacacctacaagaccacccctccagtgctggatagcgacggcagctacttcctgtacagcaagctgaccgtggacaagagcagatggcagcagggcaacgtgttcagctgctctgtgatgcacgaggccctgcacaaccactacacccagaagtctctgagcctgagccccggaaagtgatgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 117 - protein, 472 aa, Homo sapiens
MGWSCIILFLVATATGVHSQLQLQESGPGVVKPSETLSLTCTISGGSFSTYYWTWIRQPPGKGLEWVGYIGNGGRSLNYNPSLKSRITLSVDASKNQFSLKVTSVTAADTAVYYCGRARGLRGNWFDVWGPGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 118 - DNA, 5118 bp, Homo sapiens
  source 1..5118: mol_type = genomic DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtacacgttgctgccgcgcgcgccaccagacataatagctgacagactaacagactgttcctttccatgggtcttttctgcagtcaccgtcgtcgacacgtgtgatcagatatcgcggccgctctagaccaccatgggatggtcatgtatcatcctttttctagtagcaactgcaaccggtgtacattcccaggctgccctgactcagcctccctctgtgtctgggtctcctggacagtcggtcaccatctcctgcactggaaccagcagtgacatcggtggttataactatgtctcctggtaccaacaacacccaggcaaagcccccaaagtcatgatttatgaggtcagtaagcggccctcaggggtctctgatcgcttctctggttccaaatctggcaacatagcctccctgaccatctctgggctccaggctgaggacgaggctgattattactgcagctcatatgcaggcagcaacactttcttattcggaggagggacccggctgacagtactaggtcagcccaaggctgccccctcggtcactctcttcccgccctcctctgaggagcttcaagccaacaaggccacactagtgtgtctgatcagtgacttctacccgggagccgtggaagtggcctggaaggcagatggcagcgctgtcaacgcgggagtggagaccaccaaaccctccaaacagagcaacaacaagtacgcggccagcagctacctgagcctgacgtccgaccagtggaagtcccacaagagctacagctgccaggtcacgcacgaagggagcaccgtggagaagacagtggcccctgcagaatgttcatagggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacatggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 119 - protein, 235 aa, Homo sapiens
MGWSCIILFLVATATGVHSQAALTQPPSVSGSPGQSVTISCTGTSSDIGGYNYVSWYQQHPGKAPKVMIYEVSKRPSGVSDRFSGSKSGNIASLTISGLQAEDEADYYCSSYAGSNTFLFGGGTRLTVLGQPKAAPSVTLFPPSSEELQANKATLVCLISDFYPGAVEVAWKADGSAVNAGVETTKPSKQSNNKYAASSYLSLTSDQWKSHKSYSCQVTHEGSTVEKTVAPAECS

SEQ ID NO: 120 - DNA, 5823 bp, Homo sapiens
  source 1..5823: mol_type = genomic DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagaccaccatgggatggtcatgtatcatcctttttctagtagcaactgcaaccggtgtacattcccaactgcagttgcaggagtcgggcccaggagtggtgaagccttcggagaccctgtccctcacctgcactatctctggtggctccttcagtacttactactggacctggattcgccagcccccagggaagggactggagtgggttgggtatatcggtaatggtggtcgtagcctcaactacaacccctccctcaagagtcgcatcaccctgtcagtagacgcgtccaagaaccagttctccctgaaggtgacctctgtgaccgccgcggacacggccgtctattactgtgggagagccaggggactccgcggaaactggttcgatgtctggggcccgggagtcctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatgaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 121 - protein, 469 aa, Homo sapiens
MGWSCIILFLVATATGVHSQLQLQESGPGVVKPSETLSLTCTISGGSFSTYYWTWIRQPPGKGLEWVGYIGNGGRSLNYNPSLKSRITLSVDASKNQFSLKVTSVTAADTAVYYCGRARGLRGNWFDVWGPGVLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - DNA, 5118 bp, Homo sapiens
  source 1..5118: mol_type = genomic DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagaccaccatgggatggtcatgtatcatcctttttctagtagcaactgcaaccggtgtacattcccaggctgccctgactcagcctccctctgtgtctgggtctcctggacagtcggtcaccatctcctgcactggaaccagcagtgacatcggtggttataactatgtctcctggtaccaacaacacccaggcaaagcccccaaagtcatgatttatgaggtcagtaagcggccctcaggggtctctgatcgcttctctggttccaaatctggcaacatagcctccctgaccatctctgggctccaggctgaggacgaggctgattattactgcagctcatatgcaggcagcaacactttcttattcggaggagggacccggctgacagtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatgaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 123 - protein, 235 aa, Homo sapiens
MGWSCIILFLVATATGVHSQAALTQPPSVSGSPGQSVTISCTGTSSDIGGYNYVSWYQQHPGKAPKVMIYEVSKRPSGVSDRFSGSKSGNIASLTISGLQAEDEADYYCSSYAGSNTFLFGGGTRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 124 - protein, 907 aa, Homo sapiens
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTSNTTGFAAPNTTTGLPSSTHVPTNLTAPASTGPTVSTADVTSPTPAGTTSGASPVTPSPSPRDNGTESKAPDMTSPTSAVTTPTPNATSPTSAVTTPTPNATSPTPAVTTPTPNATSPTLGKTSPTSAVTTPTPNATSPTLGKTSPTSAVTTPTPNATSPTLGKTSPTSAVTTPTPNATSPTVGETSPQANTTNHTLGGTSSTPVVTSPPKNATSAVTTGQHNITSSSTSSMSLRPSSISETLSPSTSDNSTSHMPLLTSAHPTGGENITQVTPASTSTHHVSTSSPAPRPGTTSQASGPGNSSTSTKPGEVNVTKGTPPKNATSPQAPSGQKTAVPTVTSTGGKANSTTGGKHTTGHGARTSTEPTTDYGGDSTTPRTRYNATTYLPPSTSSELRPRWTFTSPPVTTAQATVPVPPTSQPRFSNLSMLVLQWASLAVLTLLLLLVMADCAFRRNLSTSHTYTTPPYDDAETYV

SEQ ID NO: 125 - protein, 426 aa, Homo sapiens
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGS

SEQ ID NO: 126 - DNA, 1281 bp, Homo sapiens
  source 1..1281: mol_type = genomic DNA
gaagctgccctgctcgtgtgccagtacaccatccagagcctgatccacctgaccggcgaggaccccggcttcttcaacgtggaaatccccgagttccccttctaccctacctgcaacgtgtgcaccgccgacgtgaacgtgaccatcaacttcgacgtgggcggcaagaagcaccagctggacctggatttcggccagctgacccctcacaccaaggccgtgtatcagcccagaggcgcctttggcggcagcgagaacgccaccaatctgtttctgctggaactcctaggcgccggcgagctggccctgaccatgagaagcaagaaactgcccatcaatgtgaccacaggcgaggaacagcaggtgtccctggaaagcgtggacgtgtactttcaagacgtgttcggcaccatgtggtgccaccacgccgagatgcagaaccccgtgtacctgatccccgagacagtgccctacatcaagtgggacaactgcaacagcaccaacatcaccgccgtcgtgcgggcccagggactggatgtgacactgcctctgagcctgcctaccagcgcccaggacagcaacttcagcgtgaaaaccgagatgctgggcaacgagatcgacatcgagtgcatcatggaagatggcgagatcagccaggtgctgcccggcgacaacaagttcaacatcacatgcagcggctacgagagccacgtgccatctggcggcatcctgaccagcacaagcccagtggccacacccatccctggcacaggctacgcctacagcctgagactgacccccagacccgtgtccagattcctgggcaacaacagcatcctgtacgtgttctacagcggcaacggccccaaggcctctggcggcgattactgtatccagagcaacatcgtgttcagcgacgagatccccgccagccaggacatgcccaccaataccaccgacatcacgtacgtgggcgacaatgccacctacagcgtgccaatggtcacctccgaggacgccaacagccccaacgtgaccgtgacagccttttgggcctggcctaacaacaccgagacagacttcaagtgcaagtggaccctgacctccggcacccctagcggctgcgagaatatcagcggagccttcgccagcaaccggaccttcgatatcaccgtgtctggcctgggcaccgcccccaagaccctgatcatcaccagaaccgccacaaatgccaccaccacaacccacaaagtgatcttcagcaaggcccccggctctggc

SEQ ID NO: 127 - protein, 426 aa, Homo sapiens
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQNVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGS

SEQ ID NO: 128 - DNA, 1278 bp, Homo sapiens
  source 1..1278: mol_type = genomic DNA
gaagctgccctgctcgtgtgccagtacaccatccagagcctgatccacctgaccggcgaggaccccggcttcttcaacgtggaaatccccgagttccccttctaccctacctgcaacgtgtgcaccgccgacgtgaacgtgaccatcaacttcgacgtgggcggcaagaagcaccagctggacctggatttcggccagctgacccctcacaccaaggccgtgtatcagcccagaggcgcctttggcggcagcgagaacgccaccaatctgtttctgctggaactcctaggcgccggcgagctggccctgaccatgagaagcaagaaactgcccatcaatgtgaccacaggcgaggaacagaacgtgtccctggaaagcgtggacgtgtactttcaagacgtgttcggcaccatgtggtgccaccacgccgagatgcagaaccccgtgtacctgatccccgagacagtgccctacatcaagtgggacaactgcaacagcaccaacatcaccgccgtcgtgcgggcccagggactggatgtgacactgcctctgagcctgcctaccagcgcccaggacagcaacttcagcgtgaaaaccgagatgctgggcaacgagatcgacatcgagtgcatcatggaagatggcgagatcagccaggtgctgcccggcgacaacaagttcaacatcacatgcagcggctacgagagccacgtgccatctggcggcatcctgaccagcacaagcccagtggccacacccatccctggcacaggctacgcctacagcctgagactgacccccagacccgtgtccagattcctgggcaacaacagcatcctgtacgtgttctacagcggcaacggccccaaggcctctggcggcgattactgtatccagagcaacatcgtgttcagcgacgagatccccgccagccaggacatgcccaccaataccaccgacatcacgtacgtgggcgacaatgccacctacagcgtgccaatggtcacctccgaggacgccaacagccccaacgtgaccgtgacagccttttgggcctggcctaacaacaccgagacagacttcaagtgcaagtggaccctgacctccggcacccctagcggctgcgagaatatcagcggagccttcgccagcaaccggaccttcgatatcaccgtgtctggcctgggcaccgcccccaagaccctgatcatcaccagaaccgccacaaatgccaccaccacaacccacaaagtgatcttcagcaaggcccccggctct

SEQ ID NO: 129 - protein, 427 aa, Homo sapiens
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWNNSCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGS

SEQ ID NO: 130 - DNA, 1281 bp, Homo sapiens
  source 1..1281: mol_type = genomic DNA
gaagctgccctgctcgtgtgccagtacaccatccagagcctgatccacctgaccggcgaggaccccggcttcttcaacgtggaaatccccgagttccccttctaccctacctgcaacgtgtgcaccgccgacgtgaacgtgaccatcaacttcgacgtgggcggcaagaagcaccagctggacctggatttcggccagctgacccctcacaccaaggccgtgtatcagcccagaggcgcctttggcggcagcgagaacgccaccaatctgtttctgctggaactcctaggcgccggcgagctggccctgaccatgagaagcaagaaactgcccatcaatgtgaccacaggcgaggaacagcaggtgtccctggaaagcgtggacgtgtactttcaagacgtgttcggcaccatgtggtgccaccacgccgagatgcagaaccccgtgtacctgatccccgagacagtgccctacatcaagtggaacaacagctgcaacagcaccaacatcaccgccgtcgtgcgggcccagggactggatgtgacactgcctctgagcctgcctaccagcgcccaggacagcaacttcagcgtgaaaaccgagatgctgggcaacgagatcgacatcgagtgcatcatggaagatggcgagatcagccaggtgctgcccggcgacaacaagttcaacatcacatgcagcggctacgagagccacgtgccatctggcggcatcctgaccagcacaagcccagtggccacacccatccctggcacaggctacgcctacagcctgagactgacccccagacccgtgtccagattcctgggcaacaacagcatcctgtacgtgttctacagcggcaacggccccaaggcctctggcggcgattactgtatccagagcaacatcgtgttcagcgacgagatccccgccagccaggacatgcccaccaataccaccgacatcacgtacgtgggcgacaatgccacctacagcgtgccaatggtcacctccgaggacgccaacagccccaacgtgaccgtgacagccttttgggcctggcctaacaacaccgagacagacttcaagtgcaagtggaccctgacctccggcacccctagcggctgcgagaatatcagcggagccttcgccagcaaccggaccttcgatatcaccgtgtctggcctgggcaccgcccccaagaccctgatcatcaccagaaccgccacaaatgccaccaccacaacccacaaagtgatcttcagcaaggcccccggctct

SEQ ID NO: 131 - protein, 426 aa, Homo sapiens
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGRYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGS

SEQ ID NO: 132 - DNA, 1278 bp, Homo sapiens
  source 1..1278: mol_type = genomic DNA
gaagctgccctgctcgtgtgccagtacaccatccagagcctgatccacctgaccggcgaggaccccggcttcttcaacgtggaaatccccgagttccccttctaccctacctgcaacgtgtgcaccgccgacgtgaacgtgaccatcaacttcgacgtgggcggcaagaagcaccagctggacctggatttcggccagctgacccctcacaccaaggccgtgtatcagcccagaggcgcctttggcggcagcgagaacgccaccaatctgtttctgctggaactcctaggcgccggcgagctggccctgaccatgagaagcaagaaactgcccatcaatgtgaccacaggcgaggaacagcaggtgtccctggaaagcgtggacgtgtactttcaagacgtgttcggcaccatgtggtgccaccacgccgagatgcagaaccccgtgtacctgatccccgagacagtgccctacatcaagtgggacaactgcaacagcaccaacatcaccgccgtcgtgcgggcccagggactggatgtgacactgcctctgagcctgcctaccagcgcccaggacagcaacttcagcgtgaaaaccgagatgctgggcaacgagatcgacatcgagtgcatcatggaagatggcgagatcagccaggtgctgcccggcgacaacaagttcaacatcacatgcagcggctacgagagccacgtgccatctggcggcatcctgaccagcacaagcccagtggccacacccatccctggcacaggctacgcctacagcctgagactgacccccagacccgtgtccagattcctgggcaacaacagcatcctgtacgtgttctacagcggcaacggccccaaggcctctggcggccggtactgtatccagagcaacatcgtgttcagcgacgagatccccgccagccaggacatgcccaccaataccaccgacatcacgtacgtgggcgacaatgccacctacagcgtgccaatggtcacctccgaggacgccaacagccccaacgtgaccgtgacagccttttgggcctggcctaacaacaccgagacagacttcaagtgcaagtggaccctgacctccggcacccctagcggctgcgagaatatcagcggagccttcgccagcaaccggaccttcgatatcaccgtgtctggcctgggcaccgcccccaagaccctgatcatcaccagaaccgccacaaatgccaccaccacaacccacaaagtgatcttcagcaaggcccccggctct

SEQ ID NO: 133 - protein, 426 aa, Homo sapiens
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKAAACNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGS

SEQ ID NO: 134 - DNA, 1278 bp, Homo sapiens
  source 1..1278: mol_type = genomic DNA
gaagctgccctgctcgtgtgccagtacaccatccagagcctgatccacctgaccggcgaggaccccggcttcttcaacgtggaaatccccgagttccccttctaccctacctgcaacgtgtgcaccgccgacgtgaacgtgaccatcaacttcgacgtgggcggcaagaagcaccagctggacctggatttcggccagctgacccctcacaccaaggccgtgtatcagcccagaggcgcctttggcggcagcgagaacgccaccaatctgtttctgctggaactcctaggcgccggcgagctggccctgaccatgagaagcaagaaactgcccatcaatgtgaccacaggcgaggaacagcaggtgtccctggaaagcgtggacgtgtactttcaagacgtgttcggcaccatgtggtgccaccacgccgagatgcagaaccccgtgtacctgatccccgagacagtgccctacatcaaggccgccgcctgcaacagcaccaacatcaccgccgtcgtgcgggcccagggactggatgtgacactgcctctgagcctgcctaccagcgcccaggacagcaacttcagcgtgaaaaccgagatgctgggcaacgagatcgacatcgagtgcatcatggaagatggcgagatcagccaggtgctgcccggcgacaacaagttcaacatcacatgcagcggctacgagagccacgtgccatctggcggcatcctgaccagcacaagcccagtggccacacccatccctggcacaggctacgcctacagcctgagactgacccccagacccgtgtccagattcctgggcaacaacagcatcctgtacgtgttctacagcggcaacggccccaaggcctctggcggcgattactgtatccagagcaacatcgtgttcagcgacgagatccccgccagccaggacatgcccaccaataccaccgacatcacgtacgtgggcgacaatgccacctacagcgtgccaatggtcacctccgaggacgccaacagccccaacgtgaccgtgacagccttttgggcctggcctaacaacaccgagacagacttcaagtgcaagtggaccctgacctccggcacccctagcggctgcgagaatatcagcggagccttcgccagcaaccggaccttcgatatcaccgtgtctggcctgggcaccgcccccaagaccctgatcatcaccagaaccgccacaaatgccaccaccacaacccacaaagtgatcttcagcaaggcccccggctct

SEQ ID NO: 135 - protein, 426 aa, Homo sapiens
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYRKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGRYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGS

SEQ ID NO: 136 - DNA, 1278 bp, Homo sapiens
  source 1..1278: mol_type = genomic DNA
gaagctgccctgctcgtgtgccagtacaccatccagagcctgatccacctgaccggcgaggaccccggcttcttcaacgtggaaatccccgagttccccttctaccctacctgcaacgtgtgcaccgccgacgtgaacgtgaccatcaacttcgacgtgggcggcaagaagcaccagctggacctggatttcggccagctgacccctcacaccaaggccgtgtatcagcccagaggcgcctttggcggcagcgagaacgccaccaatctgtttctgctggaactcctaggcgccggcgagctggccctgaccatgagaagcaagaaactgcccatcaatgtgaccacaggcgaggaacagcaggtgtccctggaaagcgtggacgtgtactttcaagacgtgttcggcaccatgtggtgccaccacgccgagatgcagaaccccgtgtacctgatccccgagacagtgccctaccggaagtgggacaactgcaacagcaccaacatcaccgccgtcgtgcgggcccagggactggatgtgacactgcctctgagcctgcctaccagcgcccaggacagcaacttcagcgtgaaaaccgagatgctgggcaacgagatcgacatcgagtgcatcatggaagatggcgagatcagccaggtgctgcccggcgacaacaagttcaacatcacatgcagcggctacgagagccacgtgccatctggcggcatcctgaccagcacaagcccagtggccacacccatccctggcacaggctacgcctacagcctgagactgacccccagacccgtgtccagattcctgggcaacaacagcatcctgtacgtgttctacagcggcaacggccccaaggcctctggcggccggtactgtatccagagcaacatcgtgttcagcgacgagatccccgccagccaggacatgcccaccaataccaccgacatcacgtacgtgggcgacaatgccacctacagcgtgccaatggtcacctccgaggacgccaacagccccaacgtgaccgtgacagccttttgggcctggcctaacaacaccgagacagacttcaagtgcaagtggaccctgacctccggcacccctagcggctgcgagaatatcagcggagccttcgccagcaaccggaccttcgatatcaccgtgtctggcctgggcaccgcccccaagaccctgatcatcaccagaaccgccacaaatgccaccaccacaacccacaaagtgatcttcagcaaggcccccggctct

SEQ ID NO: 137 - protein, 426 aa, Homo sapiens
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVRYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGRYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGS

SEQ ID NO: 138 - DNA, 1278 bp, Homo sapiens
  source 1..1278: mol_type = genomic DNA
gaagctgccctgctcgtgtgccagtacaccatccagagcctgatccacctgaccggcgaggaccccggcttcttcaacgtggaaatccccgagttccccttctaccctacctgcaacgtgtgcaccgccgacgtgaacgtgaccatcaacttcgacgtgggcggcaagaagcaccagctggacctggatttcggccagctgacccctcacaccaaggccgtgtatcagcccagaggcgcctttggcggcagcgagaacgccaccaatctgtttctgctggaactcctaggcgccggcgagctggccctgaccatgagaagcaagaaactgcccatcaatgtgaccacaggcgaggaacagcaggtgtccctggaaagcgtggacgtgtactttcaagacgtgttcggcaccatgtggtgccaccacgccgagatgcagaaccccgtgtacctgatccccgagacagtgcggtacatcaagtgggacaactgcaacagcaccaacatcaccgccgtcgtgcgggcccagggactggatgtgacactgcctctgagcctgcctaccagcgcccaggacagcaacttcagcgtgaaaaccgagatgctgggcaacgagatcgacatcgagtgcatcatggaagatggcgagatcagccaggtgctgcccggcgacaacaagttcaacatcacatgcagcggctacgagagccacgtgccatctggcggcatcctgaccagcacaagcccagtggccacacccatccctggcacaggctacgcctacagcctgagactgacccccagacccgtgtccagattcctgggcaacaacagcatcctgtacgtgttctacagcggcaacggccccaaggcctctggcggccggtactgtatccagagcaacatcgtgttcagcgacgagatccccgccagccaggacatgcccaccaataccaccgacatcacgtacgtgggcgacaatgccacctacagcgtgccaatggtcacctccgaggacgccaacagccccaacgtgaccgtgacagccttttgggcctggcctaacaacaccgagacagacttcaagtgcaagtggaccctgacctccggcacccctagcggctgcgagaatatcagcggagccttcgccagcaaccggaccttcgatatcaccgtgtctggcctgggcaccgcccccaagaccctgatcatcaccagaaccgccacaaatgccaccaccacaacccacaaagtgatcttcagcaaggcccccggctct

SEQ ID NO: 139 - protein, 426 aa, Homo sapiens
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVNYTKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGRYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGS

SEQ ID NO: 140 - DNA, 1278 bp, Homo sapiens
  source 1..1278: mol_type = genomic DNA
gaagctgccctgctcgtgtgccagtacaccatccagagcctgatccacctgaccggcgaggaccccggcttcttcaacgtggaaatccccgagttccccttctaccctacctgcaacgtgtgcaccgccgacgtgaacgtgaccatcaacttcgacgtgggcggcaagaagcaccagctggacctggatttcggccagctgacccctcacaccaaggccgtgtatcagcccagaggcgcctttggcggcagcgagaacgccaccaatctgtttctgctggaactcctaggcgccggcgagctggccctgaccatgagaagcaagaaactgcccatcaatgtgaccacaggcgaggaacagcaggtgtccctggaaagcgtggacgtgtactttcaagacgtgttcggcaccatgtggtgccaccacgccgagatgcagaaccccgtgtacctgatccccgagacagtgaactacaccaagtgggacaactgcaacagcaccaacatcaccgccgtcgtgcgggcccagggactggatgtgacactgcctctgagcctgcctaccagcgcccaggacagcaacttcagcgtgaaaaccgagatgctgggcaacgagatcgacatcgagtgcatcatggaagatggcgagatcagccaggtgctgcccggcgacaacaagttcaacatcacatgcagcggctacgagagccacgtgccatctggcggcatcctgaccagcacaagcccagtggccacacccatccctggcacaggctacgcctacagcctgagactgacccccagacccgtgtccagattcctgggcaacaacagcatcctgtacgtgttctacagcggcaacggccccaaggcctctggcggccggtactgtatccagagcaacatcgtgttcagcgacgagatccccgccagccaggacatgcccaccaataccaccgacatcacgtacgtgggcgacaatgccacctacagcgtgccaatggtcacctccgaggacgccaacagccccaacgtgaccgtgacagccttttgggcctggcctaacaacaccgagacagacttcaagtgcaagtggaccctgacctccggcacccctagcggctgcgagaatatcagcggagccttcgccagcaaccggaccttcgatatcaccgtgtctggcctgggcaccgcccccaagaccctgatcatcaccagaaccgccacaaatgccaccaccacaacccacaaagtgatcttcagcaaggcccccggctct

SEQ ID NO: 141 - protein, 426 aa, Homo sapiens
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVRYRKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGRYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGS

SEQ ID NO: 142 - DNA, 1278 bp, Homo sapiens
  source 1..1278: mol_type = genomic DNA
gaagctgccctgctcgtgtgccagtacaccatccagagcctgatccacctgaccggcgaggaccccggcttcttcaacgtggaaatccccgagttccccttctaccctacctgcaacgtgtgcaccgccgacgtgaacgtgaccatcaacttcgacgtgggcggcaagaagcaccagctggacctggatttcggccagctgacccctcacaccaaggccgtgtatcagcccagaggcgcctttggcggcagcgagaacgccaccaatctgtttctgctggaactcctaggcgccggcgagctggccctgaccatgagaagcaagaaactgcccatcaatgtgaccacaggcgaggaacagcaggtgtccctggaaagcgtggacgtgtactttcaagacgtgttcggcaccatgtggtgccaccacgccgagatgcagaaccccgtgtacctgatccccgagacagtgcggtaccggaagtgggacaactgcaacagcaccaacatcaccgccgtcgtgcgggcccagggactggatgtgacactgcctctgagcctgcctaccagcgcccaggacagcaacttcagcgtgaaaaccgagatgctgggcaacgagatcgacatcgagtgcatcatggaagatggcgagatcagccaggtgctgcccggcgacaacaagttcaacatcacatgcagcggctacgagagccacgtgccatctggcggcatcctgaccagcacaagcccagtggccacacccatccctggcacaggctacgcctacagcctgagactgacccccagacccgtgtccagattcctgggcaacaacagcatcctgtacgtgttctacagcggcaacggccccaaggcctctggcggccggtactgtatccagagcaacatcgtgttcagcgacgagatccccgccagccaggacatgcccaccaataccaccgacatcacgtacgtgggcgacaatgccacctacagcgtgccaatggtcacctccgaggacgccaacagccccaacgtgaccgtgacagccttttgggcctggcctaacaacaccgagacagacttcaagtgcaagtggaccctgacctccggcacccctagcggctgcgagaatatcagcggagccttcgccagcaaccggaccttcgatatcaccgtgtctggcctgggcaccgcccccaagaccctgatcatcaccagaaccgccacaaatgccaccaccacaacccacaaagtgatcttcagcaaggcccccggctct

SEQ ID NO: 143 - protein, 447 aa, Homo sapiens
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGSGLNDIFEAQKIEWHEHHHHHH

SEQ ID NO: 144 - DNA, 1341 bp, Homo sapiens
  source 1..1341: mol_type = genomic DNA
gaagctgccctgctcgtgtgccagtacaccatccagagcctgatccacctgaccggcgaggaccccggcttcttcaacgtggaaatccccgagttccccttctaccctacctgcaacgtgtgcaccgccgacgtgaacgtgaccatcaacttcgacgtgggcggcaagaagcaccagctggacctggatttcggccagctgacccctcacaccaaggccgtgtatcagcccagaggcgcctttggcggcagcgagaacgccaccaatctgtttctgctggaactcctaggcgccggcgagctggccctgaccatgagaagcaagaaactgcccatcaatgtgaccacaggcgaggaacagcaggtgtccctggaaagcgtggacgtgtactttcaagacgtgttcggcaccatgtggtgccaccacgccgagatgcagaaccccgtgtacctgatccccgagacagtgccctacatcaagtgggacaactgcaacagcaccaacatcaccgccgtcgtgcgggcccagggactggatgtgacactgcctctgagcctgcctaccagcgcccaggacagcaacttcagcgtgaaaaccgagatgctgggcaacgagatcgacatcgagtgcatcatggaagatggcgagatcagccaggtgctgcccggcgacaacaagttcaacatcacatgcagcggctacgagagccacgtgccatctggcggcatcctgaccagcacaagcccagtggccacacccatccctggcacaggctacgcctacagcctgagactgacccccagacccgtgtccagattcctgggcaacaacagcatcctgtacgtgttctacagcggcaacggccccaaggcctctggcggcgattactgtatccagagcaacatcgtgttcagcgacgagatccccgccagccaggacatgcccaccaataccaccgacatcacgtacgtgggcgacaatgccacctacagcgtgccaatggtcacctccgaggacgccaacagccccaacgtgaccgtgacagccttttgggcctggcctaacaacaccgagacagacttcaagtgcaagtggaccctgacctccggcacccctagcggctgcgagaatatcagcggagccttcgccagcaaccggaccttcgatatcaccgtgtctggcctgggcaccgcccccaagaccctgatcatcaccagaaccgccacaaatgccaccaccacaacccacaaagtgatcttcagcaaggcccccggctctggcctgaacgacatttttgaggcccagaagattgagtggcatgaacatcaccaccaccaccat

SEQ ID NO: 145 - protein, 447 aa, Homo sapiens
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQNVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGSGLNDIFEAQKIEWHEHHHHHH

SEQ ID NO: 146 - DNA, 1341 bp, Homo sapiens
  source 1..1341: mol_type = genomic DNA
gaagctgccctgctcgtgtgccagtacaccatccagagcctgatccacctgaccggcgaggaccccggcttcttcaacgtggaaatccccgagttccccttctaccctacctgcaacgtgtgcaccgccgacgtgaacgtgaccatcaacttcgacgtgggcggcaagaagcaccagctggacctggatttcggccagctgacccctcacaccaaggccgtgtatcagcccagaggcgcctttggcggcagcgagaacgccaccaatctgtttctgctggaactcctaggcgccggcgagctggccctgaccatgagaagcaagaaactgcccatcaatgtgaccacaggcgaggaacagaacgtgtccctggaaagcgtggacgtgtactttcaagacgtgttcggcaccatgtggtgccaccacgccgagatgcagaaccccgtgtacctgatccccgagacagtgccctacatcaagtgggacaactgcaacagcaccaacatcaccgccgtcgtgcgggcccagggactggatgtgacactgcctctgagcctgcctaccagcgcccaggacagcaacttcagcgtgaaaaccgagatgctgggcaacgagatcgacatcgagtgcatcatggaagatggcgagatcagccaggtgctgcccggcgacaacaagttcaacatcacatgcagcggctacgagagccacgtgccatctggcggcatcctgaccagcacaagcccagtggccacacccatccctggcacaggctacgcctacagcctgagactgacccccagacccgtgtccagattcctgggcaacaacagcatcctgtacgtgttctacagcggcaacggccccaaggcctctggcggcgattactgtatccagagcaacatcgtgttcagcgacgagatccccgccagccaggacatgcccaccaataccaccgacatcacgtacgtgggcgacaatgccacctacagcgtgccaatggtcacctccgaggacgccaacagccccaacgtgaccgtgacagccttttgggcctggcctaacaacaccgagacagacttcaagtgcaagtggaccctgacctccggcacccctagcggctgcgagaatatcagcggagccttcgccagcaaccggaccttcgatatcaccgtgtctggcctgggcaccgcccccaagaccctgatcatcaccagaaccgccacaaatgccaccaccacaacccacaaagtgatcttcagcaaggcccccggctctggcctgaacgacatttttgaggcccagaagattgagtggcatgaacatcaccaccaccaccat

SEQ ID NO: 147 - protein, 448 aa, Homo sapiens
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWNNSCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGSGLNDIFEAQKIEWHEHHHHHH

SEQ ID NO: 148 - DNA, 1344 bp, Homo sapiens
  source 1..1344: mol_type = genomic DNA
gaagctgccctgctcgtgtgccagtacaccatccagagcctgatccacctgaccggcgaggaccccggcttcttcaacgtggaaatccccgagttccccttctaccctacctgcaacgtgtgcaccgccgacgtgaacgtgaccatcaacttcgacgtgggcggcaagaagcaccagctggacctggatttcggccagctgacccctcacaccaaggccgtgtatcagcccagaggcgcctttggcggcagcgagaacgccaccaatctgtttctgctggaactcctaggcgccggcgagctggccctgaccatgagaagcaagaaactgcccatcaatgtgaccacaggcgaggaacagcaggtgtccctggaaagcgtggacgtgtactttcaagacgtgttcggcaccatgtggtgccaccacgccgagatgcagaaccccgtgtacctgatccccgagacagtgccctacatcaagtggaacaacagctgcaacagcaccaacatcaccgccgtcgtgcgggcccagggactggatgtgacactgcctctgagcctgcctaccagcgcccaggacagcaacttcagcgtgaaaaccgagatgctgggcaacgagatcgacatcgagtgcatcatggaagatggcgagatcagccaggtgctgcccggcgacaacaagttcaacatcacatgcagcggctacgagagccacgtgccatctggcggcatcctgaccagcacaagcccagtggccacacccatccctggcacaggctacgcctacagcctgagactgacccccagacccgtgtccagattcctgggcaacaacagcatcctgtacgtgttctacagcggcaacggccccaaggcctctggcggcgattactgtatccagagcaacatcgtgttcagcgacgagatccccgccagccaggacatgcccaccaataccaccgacatcacgtacgtgggcgacaatgccacctacagcgtgccaatggtcacctccgaggacgccaacagccccaacgtgaccgtgacagccttttgggcctggcctaacaacaccgagacagacttcaagtgcaagtggaccctgacctccggcacccctagcggctgcgagaatatcagcggagccttcgccagcaaccggaccttcgatatcaccgtgtctggcctgggcaccgcccccaagaccctgatcatcaccagaaccgccacaaatgccaccaccacaacccacaaagtgatcttcagcaaggcccccggctctggcctgaacgacatttttgaggcccagaagattgagtggcatgaacatcaccaccaccaccat

SEQ ID NO: 149 - protein, 447 aa, Homo sapiens
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGRYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGSGLNDIFEAQKIEWHEHHHHHH

SEQ ID NO: 150 - DNA, 1341 bp, Homo sapiens
  source 1..1341: mol_type = genomic DNA
gaagctgccctgctcgtgtgccagtacaccatccagagcctgatccacctgaccggcgaggaccccggcttcttcaacgtggaaatccccgagttccccttctaccctacctgcaacgtgtgcaccgccgacgtgaacgtgaccatcaacttcgacgtgggcggcaagaagcaccagctggacctggatttcggccagctgacccctcacaccaaggccgtgtatcagcccagaggcgcctttggcggcagcgagaacgccaccaatctgtttctgctggaactcctaggcgccggcgagctggccctgaccatgagaagcaagaaactgcccatcaatgtgaccacaggcgaggaacagcaggtgtccctggaaagcgtggacgtgtactttcaagacgtgttcggcaccatgtggtgccaccacgccgagatgcagaaccccgtgtacctgatccccgagacagtgccctacatcaagtgggacaactgcaacagcaccaacatcaccgccgtcgtgcgggcccagggactggatgtgacactgcctctgagcctgcctaccagcgcccaggacagcaacttcagcgtgaaaaccgagatgctgggcaacgagatcgacatcgagtgcatcatggaagatggcgagatcagccaggtgctgcccggcgacaacaagttcaacatcacatgcagcggctacgagagccacgtgccatctggcggcatcctgaccagcacaagcccagtggccacacccatccctggcacaggctacgcctacagcctgagactgacccccagacccgtgtccagattcctgggcaacaacagcatcctgtacgtgttctacagcggcaacggccccaaggcctctggcggccggtactgtatccagagcaacatcgtgttcagcgacgagatccccgccagccaggacatgcccaccaataccaccgacatcacgtacgtgggcgacaatgccacctacagcgtgccaatggtcacctccgaggacgccaacagccccaacgtgaccgtgacagccttttgggcctggcctaacaacaccgagacagacttcaagtgcaagtggaccctgacctccggcacccctagcggctgcgagaatatcagcggagccttcgccagcaaccggaccttcgatatcaccgtgtctggcctgggcaccgcccccaagaccctgatcatcaccagaaccgccacaaatgccaccaccacaacccacaaagtgatcttcagcaaggcccccggctctggcctgaacgacatttttgaggcccagaagattgagtggcatgaacatcaccaccaccaccat

SEQ ID NO: 151 - protein, 447 aa, Homo sapiens
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKAAACNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGSGLNDIFEAQKIEWHEHHHHHH

SEQ ID NO: 152 - DNA, 1341 bp, Homo sapiens
  source 1..1341: mol_type = genomic DNA
gaagctgccctgctcgtgtgccagtacaccatccagagcctgatccacctgaccggcgaggaccccggcttcttcaacgtggaaatccccgagttccccttctaccctacctgcaacgtgtgcaccgccgacgtgaacgtgaccatcaacttcgacgtgggcggcaagaagcaccagctggacctggatttcggccagctgacccctcacaccaaggccgtgtatcagcccagaggcgcctttggcggcagcgagaacgccaccaatctgtttctgctggaactcctaggcgccggcgagctggccctgaccatgagaagcaagaaactgcccatcaatgtgaccacaggcgaggaacagcaggtgtccctggaaagcgtggacgtgtactttcaagacgtgttcggcaccatgtggtgccaccacgccgagatgcagaaccccgtgtacctgatccccgagacagtgccctacatcaaggccgccgcctgcaacagcaccaacatcaccgccgtcgtgcgggcccagggactggatgtgacactgcctctgagcctgcctaccagcgcccaggacagcaacttcagcgtgaaaaccgagatgctgggcaacgagatcgacatcgagtgcatcatggaagatggcgagatcagccaggtgctgcccggcgacaacaagttcaacatcacatgcagcggctacgagagccacgtgccatctggcggcatcctgaccagcacaagcccagtggccacacccatccctggcacaggctacgcctacagcctgagactgacccccagacccgtgtccagattcctgggcaacaacagcatcctgtacgtgttctacagcggcaacggccccaaggcctctggcggcgattactgtatccagagcaacatcgtgttcagcgacgagatccccgccagccaggacatgcccaccaataccaccgacatcacgtacgtgggcgacaatgccacctacagcgtgccaatggtcacctccgaggacgccaacagccccaacgtgaccgtgacagccttttgggcctggcctaacaacaccgagacagacttcaagtgcaagtggaccctgacctccggcacccctagcggctgcgagaatatcagcggagccttcgccagcaaccggaccttcgatatcaccgtgtctggcctgggcaccgcccccaagaccctgatcatcaccagaaccgccacaaatgccaccaccacaacccacaaagtgatcttcagcaaggcccccggctctggcctgaacgacatttttgaggcccagaagattgagtggcatgaacatcaccaccaccaccat

SEQ ID NO: 153 - protein, 447 aa, Homo sapiens
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYRKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGRYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGSGLNDIFEAQKIEWHEHHHHHH

SEQ ID NO: 154 - DNA, 1341 bp, Homo sapiens
  source 1..1341: mol_type = genomic DNA
gaagctgccctgctcgtgtgccagtacaccatccagagcctgatccacctgaccggcgaggaccccggcttcttcaacgtggaaatccccgagttccccttctaccctacctgcaacgtgtgcaccgccgacgtgaacgtgaccatcaacttcgacgtgggcggcaagaagcaccagctggacctggatttcggccagctgacccctcacaccaaggccgtgtatcagcccagaggcgcctttggcggcagcgagaacgccaccaatctgtttctgctggaactcctaggcgccggcgagctggccctgaccatgagaagcaagaaactgcccatcaatgtgaccacaggcgaggaacagcaggtgtccctggaaagcgtggacgtgtactttcaagacgtgttcggcaccatgtggtgccaccacgccgagatgcagaaccccgtgtacctgatccccgagacagtgccctaccggaagtgggacaactgcaacagcaccaacatcaccgccgtcgtgcgggcccagggactggatgtgacactgcctctgagcctgcctaccagcgcccaggacagcaacttcagcgtgaaaaccgagatgctgggcaacgagatcgacatcgagtgcatcatggaagatggcgagatcagccaggtgctgcccggcgacaacaagttcaacatcacatgcagcggctacgagagccacgtgccatctggcggcatcctgaccagcacaagcccagtggccacacccatccctggcacaggctacgcctacagcctgagactgacccccagacccgtgtccagattcctgggcaacaacagcatcctgtacgtgttctacagcggcaacggccccaaggcctctggcggccggtactgtatccagagcaacatcgtgttcagcgacgagatccccgccagccaggacatgcccaccaataccaccgacatcacgtacgtgggcgacaatgccacctacagcgtgccaatggtcacctccgaggacgccaacagccccaacgtgaccgtgacagccttttgggcctggcctaacaacaccgagacagacttcaagtgcaagtggaccctgacctccggcacccctagcggctgcgagaatatcagcggagccttcgccagcaaccggaccttcgatatcaccgtgtctggcctgggcaccgcccccaagaccctgatcatcaccagaaccgccacaaatgccaccaccacaacccacaaagtgatcttcagcaaggcccccggctctggcctgaacgacatttttgaggcccagaagattgagtggcatgaacatcaccaccaccaccat

SEQ ID NO: 155 - protein, 447 aa, Homo sapiens
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVRYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGRYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGSGLNDIFEAQKIEWHEHHHHHH

SEQ ID NO: 156 - DNA, 1341 bp, Homo sapiens
  source 1..1341: mol_type = genomic DNA
gaagctgccctgctcgtgtgccagtacaccatccagagcctgatccacctgaccggcgaggaccccggcttcttcaacgtggaaatccccgagttccccttctaccctacctgcaacgtgtgcaccgccgacgtgaacgtgaccatcaacttcgacgtgggcggcaagaagcaccagctggacctggatttcggccagctgacccctcacaccaaggccgtgtatcagcccagaggcgcctttggcggcagcgagaacgccaccaatctgtttctgctggaactcctaggcgccggcgagctggccctgaccatgagaagcaagaaactgcccatcaatgtgaccacaggcgaggaacagcaggtgtccctggaaagcgtggacgtgtactttcaagacgtgttcggcaccatgtggtgccaccacgccgagatgcagaaccccgtgtacctgatccccgagacagtgcggtacatcaagtgggacaactgcaacagcaccaacatcaccgccgtcgtgcgggcccagggactggatgtgacactgcctctgagcctgcctaccagcgcccaggacagcaacttcagcgtgaaaaccgagatgctgggcaacgagatcgacatcgagtgcatcatggaagatggcgagatcagccaggtgctgcccggcgacaacaagttcaacatcacatgcagcggctacgagagccacgtgccatctggcggcatcctgaccagcacaagcccagtggccacacccatccctggcacaggctacgcctacagcctgagactgacccccagacccgtgtccagattcctgggcaacaacagcatcctgtacgtgttctacagcggcaacggccccaaggcctctggcggccggtactgtatccagagcaacatcgtgttcagcgacgagatccccgccagccaggacatgcccaccaataccaccgacatcacgtacgtgggcgacaatgccacctacagcgtgccaatggtcacctccgaggacgccaacagccccaacgtgaccgtgacagccttttgggcctggcctaacaacaccgagacagacttcaagtgcaagtggaccctgacctccggcacccctagcggctgcgagaatatcagcggagccttcgccagcaaccggaccttcgatatcaccgtgtctggcctgggcaccgcccccaagaccctgatcatcaccagaaccgccacaaatgccaccaccacaacccacaaagtgatcttcagcaaggcccccggctctggcctgaacgacatttttgaggcccagaagattgagtggcatgaacatcaccaccaccaccat

SEQ ID NO: 157 - protein, 447 aa, Homo sapiens
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVNYTKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGRYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGSGLNDIFEAQKIEWHEHHHHHH

SEQ ID NO: 158 - DNA, 1341 bp, Homo sapiens
  source 1..1341: mol_type = genomic DNA
gaagctgccctgctcgtgtgccagtacaccatccagagcctgatccacctgaccggcgaggaccccggcttcttcaacgtggaaatccccgagttccccttctaccctacctgcaacgtgtgcaccgccgacgtgaacgtgaccatcaacttcgacgtgggcggcaagaagcaccagctggacctggatttcggccagctgacccctcacaccaaggccgtgtatcagcccagaggcgcctttggcggcagcgagaacgccaccaatctgtttctgctggaactcctaggcgccggcgagctggccctgaccatgagaagcaagaaactgcccatcaatgtgaccacaggcgaggaacagcaggtgtccctggaaagcgtggacgtgtactttcaagacgtgttcggcaccatgtggtgccaccacgccgagatgcagaaccccgtgtacctgatccccgagacagtgaactacaccaagtgggacaactgcaacagcaccaacatcaccgccgtcgtgcgggcccagggactggatgtgacactgcctctgagcctgcctaccagcgcccaggacagcaacttcagcgtgaaaaccgagatgctgggcaacgagatcgacatcgagtgcatcatggaagatggcgagatcagccaggtgctgcccggcgacaacaagttcaacatcacatgcagcggctacgagagccacgtgccatctggcggcatcctgaccagcacaagcccagtggccacacccatccctggcacaggctacgcctacagcctgagactgacccccagacccgtgtccagattcctgggcaacaacagcatcctgtacgtgttctacagcggcaacggccccaaggcctctggcggccggtactgtatccagagcaacatcgtgttcagcgacgagatccccgccagccaggacatgcccaccaataccaccgacatcacgtacgtgggcgacaatgccacctacagcgtgccaatggtcacctccgaggacgccaacagccccaacgtgaccgtgacagccttttgggcctggcctaacaacaccgagacagacttcaagtgcaagtggaccctgacctccggcacccctagcggctgcgagaatatcagcggagccttcgccagcaaccggaccttcgatatcaccgtgtctggcctgggcaccgcccccaagaccctgatcatcaccagaaccgccacaaatgccaccaccacaacccacaaagtgatcttcagcaaggcccccggctctggcctgaacgacatttttgaggcccagaagattgagtggcatgaacatcaccaccaccaccat

SEQ ID NO: 159 - protein, 447 aa, Homo sapiens
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVRYRKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGRYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGSGLNDIFEAQKIEWHEHHHHHH

SEQ ID NO: 160 - DNA, 1341 bp, Homo sapiens
  source 1..1341: mol_type = genomic DNA
gaagctgccctgctcgtgtgccagtacaccatccagagcctgatccacctgaccggcgaggaccccggcttcttcaacgtggaaatccccgagttccccttctaccctacctgcaacgtgtgcaccgccgacgtgaacgtgaccatcaacttcgacgtgggcggcaagaagcaccagctggacctggatttcggccagctgacccctcacaccaaggccgtgtatcagcccagaggcgcctttggcggcagcgagaacgccaccaatctgtttctgctggaactcctaggcgccggcgagctggccctgaccatgagaagcaagaaactgcccatcaatgtgaccacaggcgaggaacagcaggtgtccctggaaagcgtggacgtgtactttcaagacgtgttcggcaccatgtggtgccaccacgccgagatgcagaaccccgtgtacctgatccccgagacagtgcggtaccggaagtgggacaactgcaacagcaccaacatcaccgccgtcgtgcgggcccagggactggatgtgacactgcctctgagcctgcctaccagcgcccaggacagcaacttcagcgtgaaaaccgagatgctgggcaacgagatcgacatcgagtgcatcatggaagatggcgagatcagccaggtgctgcccggcgacaacaagttcaacatcacatgcagcggctacgagagccacgtgccatctggcggcatcctgaccagcacaagcccagtggccacacccatccctggcacaggctacgcctacagcctgagactgacccccagacccgtgtccagattcctgggcaacaacagcatcctgtacgtgttctacagcggcaacggccccaaggcctctggcggccggtactgtatccagagcaacatcgtgttcagcgacgagatccccgccagccaggacatgcccaccaataccaccgacatcacgtacgtgggcgacaatgccacctacagcgtgccaatggtcacctccgaggacgccaacagccccaacgtgaccgtgacagccttttgggcctggcctaacaacaccgagacagacttcaagtgcaagtggaccctgacctccggcacccctagcggctgcgagaatatcagcggagccttcgccagcaaccggaccttcgatatcaccgtgtctggcctgggcaccgcccccaagaccctgatcatcaccagaaccgccacaaatgccaccaccacaacccacaaagtgatcttcagcaaggcccccggctctggcctgaacgacatttttgaggcccagaagattgagtggcatgaacatcaccaccaccaccat

SEQ ID NO: 161 - protein, 119 aa, Homo sapiens
QVQLVQSGADVRKPGASVKVSCKASTYIFTGYYIHWVRQAPGRGLEWLGWIHPNSGGTTYSQMFQGRVTMTRDRSITTSYMELSRLQSDDTAIYYCATLRFVEYSFDSWGQGTLVTVSS

SEQ ID NO: 162 - DNA, 357 bp, Homo sapiens
  source 1..357: mol_type = genomic DNA
caggtgcagctggtgcagtctggcgcagacgtgaggaagccaggagcctccgtgaaggtgtcttgtaaggccagcacctacatcttcacaggctactatatccactgggtgaggcaggcaccaggaaggggcctggagtggctgggctggattcaccctaactctggcggcaccacatacagccagatgtttcagggcagagtgaccatgacacgggacagatccatcaccacatcttatatggagctgagccggctgcagtccgacgataccgccatctactattgcgccacactgagattcgtggagtattcttttgatagctggggccagggcaccctggtgacagtgagctcc

SEQ ID NO: 163 - protein, 7 aa, Homo sapiens
TYIFTGY

SEQ ID NO: 164 - protein, 6 aa, Homo sapiens
HPNSGG

SEQ ID NO: 165 - protein, 10 aa, Homo sapiens
LRFVEYSFDS

SEQ ID NO: 166 - protein, 108 aa, Homo sapiens
EIVLTQSPGTLSLSPGERATLSCRASQSISSTYLAWYQQIPGQAPRLLIYGASSRAAGIPDRFSGGGSGTDFTLTISRLEPEDFAVYYCQQYGSSPRSFGQGTKLEIK

SEQ ID NO: 167 - DNA, 324 bp, Homo sapiens
  source 1..324: mol_type = genomic DNA
gagatcgtgctgacccagagcccaggcacactgagcctgtccccaggagagagggccaccctgtcctgtagagcctctcagagcatcagctccacatacctggcctggtatcagcagatcccaggacaggcacctaggctgctgatctacggagcctctagcagggcagcaggcatccccgaccgcttctccggcggaggctctggcaccgacttcaccctgacaatctctcggctggagcctgaggacttcgccgtgtactattgccagcagtatggctcctctccaaggtcctttggccagggcacaaagctggagatcaag

SEQ ID NO: 168 - protein, 12 aa, Homo sapiens
RASQSISSTYLA

SEQ ID NO: 169 - protein, 7 aa, Homo sapiens
GASSRAA

SEQ ID NO: 170 - protein, 9 aa, Homo sapiens
QQYGSSPRS

SEQ ID NO: 171 - protein, 119 aa, Homo sapiens
EVQLLESGGALVQPGGSLRLSCAASGFTFKTYAMSWVRQVPGKGLEWVSAISGSGTASYYADSVKGRFTLSRDNSKNTLYLQLSSLRDEDTGVYYCARRFLDWFGMDVWGLGTTVTVSS

SEQ ID NO: 172 - DNA, 357 bp, Homo sapiens
  source 1..357: mol_type = genomic DNA
gaggtgcagctgctggagagcggcggcgccctggtgcagccaggaggcagcctgcggctgtcctgtgccgcctctggcttcacctttaagacatacgccatgtcctgggtgaggcaggtgcctggcaagggcctggagtgggtgtctgccatctccggctctggcaccgcctcttactatgccgacagcgtgaagggcaggttcaccctgagccgcgataactccaagaatacactgtatctgcagctgagctccctgcgggacgaggataccggcgtgtactattgcgcccggagattcctggactggtttggcatggacgtgtggggcctgggcaccacagtgacagtgtctagc

SEQ ID NO: 173 - protein, 7 aa, Homo sapiens
GFTFKTY

SEQ ID NO: 174 - protein, 6 aa, Homo sapiens
SGSGTA

SEQ ID NO: 175 - protein, 10 aa, Homo sapiens
RFLDWFGMDV

SEQ ID NO: 176 - protein, 111 aa, Homo sapiens
DIVMTQSPLSLPVTPGEPASISCLSSQSLLQSNGYNYVDWYLQKPGQSPQLLIYLGSNRASGVPDRFSGSGSGTDFTLKISRVEAEDVGVYYCMVTLHPPTFGQGAKVEIK

SEQ ID NO: 177 - DNA, 333 bp, Homo sapiens
  source 1..333: mol_type = genomic DNA
gacatcgtgatgacccagtcccctctgtctctgccagtgacacccggcgagcctgcctctatcagctgtctgagctcccagagcctgctgcagtccaacggctacaattatgtggattggtacctgcagaagccaggccagtccccccagctgctgatctatctgggctctaacagggccagcggcgtgcccgacagattctccggctctggcagcggcaccgacttcaccctgaagatctctcgggtggaggcagaggacgtgggcgtgtactattgcatggtgaccctgcacccacctacattcggccagggagccaaggtggagatcaag

SEQ ID NO: 178 - protein, 16 aa, Homo sapiens
LSSQSLLQSNGYNYVD

SEQ ID NO: 179 - protein, 7 aa, Homo sapiens
LGSNRAS

SEQ ID NO: 180 - protein, 8 aa, Homo sapiens
MVTLHPPT

SEQ ID NO: 181 - protein, 120 aa, Homo sapiens
EGQLVQSGGGLVQPGGSLTLSCEVSGFTFKNYEMNWVRQAPGKGLEWVSYISSGGIAIFHADSVKGRFTVSRDNAKNLLYLQMNSLRVEDTAVYYCARDENNVRRPFDHWGQGTLVTVSS

SEQ ID NO: 182 - DNA, 360 bp, Homo sapiens
  source 1..360: mol_type = genomic DNA
gagggacagctggtgcagtccggcggaggcctggtgcagccaggaggctccctgaccctgtcttgtgaggtgagcggcttcaccttcaagaactacgagatgaattgggtgcggcaggcacctggcaagggcctggagtgggtgtcttatatcagctccggcggaatcgcaatcttccacgcagattccgtgaagggcaggtttaccgtgtctcgcgacaacgccaagaatctgctgtacctgcagatgaacagcctgcgggtggaggacacagccgtgtactattgcgccagggatgagaacaacgtgcggcggcccttcgaccactggggacagggcaccctggtgacagtgtctagc

SEQ ID NO: 183 - protein, 7 aa, Homo sapiens
GFTFKNY

SEQ ID NO: 184 - protein, 6 aa, Homo sapiens
SSGGIA

SEQ ID NO: 185 - protein, 11 aa, Homo sapiens
DENNVRRPFDH

SEQ ID NO: 186 - protein, 109 aa, Homo sapiens
QSVLTQPPSASGSPGQSVTISCTGSSSDVGAYDFVSWFQQYPGQAPKLIIYEVNKRPSGVPARFSGSKSGNTASLTVSGLQAEDEADYFCFSYGGTTNLRVFGGGTKLT

SEQ ID NO: 187 - DNA, 327 bp, Homo sapiens
  source 1..327: mol_type = genomic DNA
cagtctgtgctgacccagccacctagcgcctccggctctcccggccagagcgtgaccatctcctgtacaggcagctcctctgacgtgggcgcctacgatttcgtgagctggtttcagcagtatccaggccaggcccccaagctgatcatctacgaggtgaacaagcggccttccggcgtgccagccagattcagcggctccaagtctggcaataccgcctctctgacagtgagcggcctgcaggcagaggacgaggcagattacttctgcttttcttatggcggcaccacaaacctgcgggtgtttggcggcggcaccaagctgaca

SEQ ID NO: 188 - protein, 14 aa, Homo sapiens
TGSSSDVGAYDFVS

SEQ ID NO: 189 - protein, 7 aa, Homo sapiens
EVNKRPS

SEQ ID NO: 190 - protein, 11 aa, Homo sapiens
FSYGGTTNLRV

SEQ ID NO: 191 - protein, 122 aa, Homo sapiens
EVQLVESGGNLVQPGASLRLSCTASRFNFNKYAMHWVRQTPGKGLEWVSAISWDSTYIDYGNSVKGRFTISRDNTRNSLYLQMNSLTAEDTALYYCAKCEDYLRLCSAYDIWGHGTMVTVSS

SEQ ID NO: 192 - DNA, 366 bp, Homo sapiens
  source 1..366: mol_type = genomic DNA
gaggtgcagctggtggagagcggcggaaacctggtgcagccaggagcctctctgaggctgagctgtaccgcctcccgcttcaactttaataagtacgcaatgcactgggtgcggcagacccctggcaagggcctggagtgggtgtctgccatcagctgggactccacatacatcgattatggcaactccgtgaagggcaggttcaccatctctcgggacaacacaagaaatagcctgtatctgcagatgaattccctgaccgccgaggatacagccctgtactattgcgccaagtgtgaggactacctgcggctgtgctctgcctatgatatctggggccacggcaccatggtgacagtgagctcc

SEQ ID NO: 193 - protein, 7 aa, Homo sapiens
RFNFNKY

SEQ ID NO: 194 - protein, 6 aa, Homo sapiens
SWDSTY

SEQ ID NO: 195 - protein, 13 aa, Homo sapiens
CEDYLRLCSAYDI

SEQ ID NO: 196 - protein, 112 aa, Homo sapiens
DIVMTQSPLSLPVTPGESASISCRSSQSLLHSNGKNYLSWYLQKPGQSPQLLIDLGSNRASGVSDRFSGSGSGTDFTLKISRVEADDVGVYYCMQAVQTPITFGQGTRLAIK

SEQ ID NO: 197 - DNA, 336 bp, Homo sapiens
  source 1..336: mol_type = genomic DNA
gacatcgtgatgacccagtctcctctgagcctgcccgtgacacctggcgagtctgccagcatctcctgtcggagctcccagagcctgctgcactccaacggcaagaattacctgtcttggtatctgcagaagccaggccagagcccccagctgctgatcgatctgggctccaacagggcctccggcgtgtctgacagattctctggcagcggctccggcaccgacttcaccctgaagatcagcagggtggaggccgacgatgtgggcgtgtactattgcatgcaggccgtgcagaccccaatcacattcggccagggaacccgcctggccatcaag

SEQ ID NO: 198 - protein, 16 aa, Homo sapiens
RSSQSLLHSNGKNYLS

SEQ ID NO: 199 - protein, 7 aa, Homo sapiens
LGSNRAS

SEQ ID NO: 200 - protein, 9 aa, Homo sapiens
MQAVQTPIT

SEQ ID NO: 201 - protein, 123 aa, Homo sapiens
QVQLVQSGAELKTPGASVKVSCKASGYTFTGYYIHWVRQAPGEGLEWTGWINPNSGATRYGQKFQGRVTLTSDTSSSTVYMEVSNLTSDDSAVYYCARELSYSIRGTGPLGYWGLGTLVTVSS

SEQ ID NO: 202 - DNA, 369 bp, Homo sapiens
  source 1..369: mol_type = genomic DNA
caggtgcagctggtgcagtccggcgcagagctgaagaccccaggagccagcgtgaaggtgtcctgtaaggcctctggctacaccttcacaggctactatatccactgggtgcggcaggcaccaggagagggcctggagtggaccggctggatcaaccctaatagcggcgccacaagatacggccagaagtttcagggccgcgtgaccctgacaagcgacaccagctcctctacagtgtatatggaggtgtccaacctgacctccgacgattctgccgtgtactattgcgcccgggagctgtcttacagcatcagaggaacaggaccactgggatattggggcctgggcaccctggtgacagtgagctcc

SEQ ID NO: 203 - protein, 7 aa, Homo sapiens
GYTFTGY

SEQ ID NO: 204 - protein, 6 aa, Homo sapiens
NPNSGA

SEQ ID NO: 205 - protein, 14 aa, Homo sapiens
ELSYSIRGTGPLGY

SEQ ID NO: 206 - protein, 108 aa, Homo sapiens
EIVLTQSPGTLSLSPGERATLSCRASQSVASKYLAWYQQKPGQAPRLLIYGASSRATGIPDRFSGSGSGTDFTLTISRLEPEDFAVYYCQYYGSSPLTFGQGTKVEIK

SEQ ID NO: 207 - DNA, 324 bp, Homo sapiens
  source 1..324: mol_type = genomic DNA
gagatcgtgctgacccagtctccaggcacactgtccctgtctccaggagagagggccaccctgtcttgtagagccagccagtccgtggccagcaagtacctggcctggtatcagcagaagccaggacaggcacctaggctgctgatctacggagccagctccagggcaaccggcatccccgaccgcttctctggcagcggctccggcacagacttcaccctgacaatctccaggctggagcctgaggacttcgccgtgtactattgccagtactatggctctagcccactgacctttggccagggcacaaaggtggagatcaag

SEQ ID NO: 208 - protein, 12 aa, Homo sapiens
RASQSVASKYLA

SEQ ID NO: 209 - protein, 7 aa, Homo sapiens
GASSRAT

SEQ ID NO: 210 - protein, 9 aa, Homo sapiens
QYYGSSPLT

SEQ ID NO: 211 - protein, 123 aa, Homo sapiens
EVQLAESGGGVVHPGGSLRLSCTASGFTFSRHSMHWVRQAPGKGLEWVAVISHDGSHKFYVDSVKGRFSISRDNAKNTLYLQMSSLSGADTAVYYCVKDISSRSYGYLAGDSWGQGSLVTVSS

SEQ ID NO: 212 - DNA, 369 bp, Homo sapiens
  source 1..369: mol_type = genomic DNA
gaggtgcagctggccgagtctggcggaggagtggtgcacccaggaggctccctgaggctgtcttgtaccgccagcggcttcacattttctaggcacagcatgcactgggtgcgccaggcacctggcaagggcctggagtgggtggccgtgatctcccacgacggctctcacaagttctacgtggattccgtgaagggccggtttagcatctccagagacaacgccaagaataccctgtatctgcagatgagctccctgtctggcgccgacacagccgtgtactattgcgtgaaggatatctctagcaggagctacggctatctggcaggcgatagctggggacagggctccctggtgaccgtgtcctct

SEQ ID NO: 213 - protein, 7 aa, Homo sapiens
GFTFSRH

SEQ ID NO: 214 - protein, 6 aa, Homo sapiens
SHDGSH

SEQ ID NO: 215 - protein, 14 aa, Homo sapiens
DISSRSYGYLAGDS

SEQ ID NO: 216 - protein, 108 aa, Homo sapiens
DIQMTQSPSSLSASVGDIITITCRASQSVVTYLNWYQQKPGGAPRLLIYTTSKLQSGVPSRFSGSGSGTLFTLTINGLRPEDFATYYCQQSYGTPPFTFGPGTRVEIN

SEQ ID NO: 217 - DNA, 324 bp, Homo sapiens
  source 1..324: mol_type = genomic DNA
gatattcagatgactcagtccccaagcagcctgagcgcctccgtgggcgacatcatcaccatcacatgcagggcctctcagagcgtggtgacctacctgaactggtatcagcagaagccaggaggagcacctaggctgctgatctacaccacatccaagctgcagtctggcgtgccatccagattctccggctctggcagcggcaccctgtttaccctgacaatcaatggcctgcggcccgaggatttcgccacatactattgtcagcagagctatggaacccccccctttacttttggaccaggcacaagagtggagattaac

SEQ ID NO: 218 - protein, 11 aa, Homo sapiens
RASQSVVTYLN

SEQ ID NO: 219 - protein, 7 aa, Homo sapiens
TTSKLQS

SEQ ID NO: 220 - protein, 10 aa, Homo sapiens
QQSYGTPPFT

SEQ ID NO: 221 - protein, 124 aa, Homo sapiens
QVHLQQWGAGLVKPSETLSLTCAVQGGPFSGYYWSWIRQPPGKGLEWIGEINHSGNTHYNPSLKSRVTISVDTSGNYFSLKLTSVTAADAAVYFCARGQQLLRNYYYYSGMDVWGQGTTVTVSS

SEQ ID NO: 222 - DNA, 372 bp, Homo sapiens
  source 1..372: mol_type = genomic DNA
caggtgcacctgcagcagtggggagcaggcctggtgaagccatccgagacactgtctctgacatgtgcagtgcagggaggacccttctctggctactattggagctggatcaggcagccacctggcaagggcctggagtggatcggcgagatcaaccacagcggcaatacccactacaacccctctctgaagagccgggtgaccatcagcgtggacacatccggcaattacttctccctgaagctgacctctgtgacagccgccgatgccgccgtgtatttttgcgcccggggccagcagctgctgagaaactactattactattccggcatggacgtgtggggacagggaaccacagtgacagtgagctcc

SEQ ID NO: 223 - protein, 7 aa, Homo sapiens
GGPFSGY

SEQ ID NO: 224 - protein, 5 aa, Homo sapiens
NHSGN

SEQ ID NO: 225 - protein, 16 aa, Homo sapiens
GQQLLRNYYYYSGMDV

SEQ ID NO: 226 - protein, 107 aa, Homo sapiens
EIVLTQSPGTLSLSPGERATLSCRASQSVTSTYLAWYQQKLGQPPRLLIFGASNRATGIPDRFSGSGSGTDFTLTITRLEPEDFAVYYCQRYGGSITFGQGTRLEIK

SEQ ID NO: 227 - DNA, 321 bp, Homo sapiens
  source 1..321: mol_type = genomic DNA
gagatcgtgctgacacagtccccaggcaccctgagcctgtccccaggagagcgggccacactgtcctgtagagcctctcagagcgtgacctctacatacctggcctggtatcagcagaagctgggccagccccctaggctgctgatcttcggcgcctctaacagggccacaggcatccctgaccgcttctccggctctggcagcggcaccgacttcaccctgacaatcaccagactggagcccgaggacttcgccgtgtactattgccagcggtacggcggcagcatcacatttggccagggcaccagactggagatcaag

SEQ ID NO: 228 - protein, 12 aa, Homo sapiens
RASQSVTSTYLA

SEQ ID NO: 229 - protein, 7 aa, Homo sapiens
GASNRAT

SEQ ID NO: 230 - protein, 8 aa, Homo sapiens
QRYGGSIT

SEQ ID NO: 231 - protein, 126 aa, Homo sapiens
EVQLVQSGGGVVQPRRSLRLSCAASGFTFSNYGMHWVRQVPGKGLQWVAIIWYDGSNKHYAASVQGRFRISRDNSKNTVYLQMDGLRAEDTGMYYCVRDATTATTEGTSQYYFDLWGQGALVTVSS

SEQ ID NO: 232 - DNA, 378 bp, Homo sapiens
  source 1..378: mol_type = genomic DNA
gaggtgcagctggtgcagtccggcggaggagtggtgcagccacggagatctctgaggctgagctgtgccgcctccggcttcaccttttctaactacggaatgcactgggtgcgccaggtgcctggcaagggcctgcagtgggtggccatcatctggtacgacggctccaataagcactatgccgcctctgtgcagggcaggttccgcatctctcgggataacagcaagaataccgtgtatctgcagatggacggcctgcgggccgaggatacaggcatgtactattgcgtgagagacgccaccacagccaccacagagggcaccagccagtactattttgatctgtggggacagggcgccctggtgacagtgagctcc

SEQ ID NO: 233 - protein, 7 aa, Homo sapiens
GFTFSNY

SEQ ID NO: 234 - protein, 6 aa, Homo sapiens
WYDGSN

SEQ ID NO: 235 - protein, 17 aa, Homo sapiens
DATTATTEGTSQYYFDL

SEQ ID NO: 236 - protein, 113 aa, Homo sapiens
DIVMTQSPDSLAVSLGERATINCKSSQTLLYTSNSKNYLAWYQQKVGQPPRLLIYWASTRESGVPDRFSGSGSGTDFTLTISSLLAEDVAVYYCQQYYTTPLTFGGGTKVEVK

SEQ ID NO: 237 - DNA, 339 bp, Homo sapiens
  source 1..339: mol_type = genomic DNA
gacatcgtgatgacccagagccccgattccctggccgtgtctctgggagagagggcaacaatcaactgtaagagctcccagaccctgctgtacacatccaactctaagaattacctggcctggtatcagcagaaagtgggacagccacctaggctgctgatctattgggcctctaccagggagagcggcgtgccagacagattcagcggctccggctctggcacagacttcaccctgacaatctctagcctgctggccgaggacgtggccgtgtactattgccagcagtactataccacacccctgaccttcggcggcggcacaaaggtggaggtgaag

SEQ ID NO: 238 - protein, 17 aa, Homo sapiens
KSSQTLLYTSNSKNYLA

SEQ ID NO: 239 - protein, 7 aa, Homo sapiens
WASTRES

SEQ ID NO: 240 - protein, 9 aa, Homo sapiens
QQYYTTPLT

SEQ ID NO: 241 - protein, 130 aa, Homo sapiens
EVQLVESGGGVVHPGKSLTLSCEASGFTFNDHGIHWVRRAPGKGLEWLALISKDGSKEYSTDSVKGRFTVSRDNSRNTVFLQMKSLTTEDTAIYYCAKDMGQCSSPSCSTMDSYFAMDVWGQGTTVIVSS

SEQ ID NO: 242 - DNA, 390 bp, Homo sapiens
  source 1..390: mol_type = genomic DNA
gaggtgcagctggtggagtccggcggaggagtggtgcaccctggcaagtctctgaccctgagctgtgaggccagcggcttcaccttcaacgaccacggcatccactgggtgcggagagcacctggcaagggcctggagtggctggccctgatctctaaggacggcagcaaggagtacagcaccgattccgtgaagggccggttcacagtgtccagggataactctcgcaataccgtgtttctgcagatgaagtctctgaccacagaggacacagccatctactattgcgccaaggatatgggccagtgcagctccccctcctgttctaccatggacagctatttcgcaatggacgtgtggggacagggaaccacagtgatcgtgtctagc

SEQ ID NO: 243 - protein, 7 aa, Homo sapiens
GFTFNDH

SEQ ID NO: 244 - protein, 6 aa, Homo sapiens
SKDGSK

SEQ ID NO: 245 - protein, 21 aa, Homo sapiens
DMGQCSSPSCSTMDSYFAMDV

SEQ ID NO: 246 - protein, 113 aa, Homo sapiens
DIVMTQSPLSLPVTPGEPASISCRSSQNLRHNNGYNYLNWYLQKPGQSPQLLIYLGSIRASGVPDRFSGSGSGTDFTLKISRVEAEDVGVYYCMQALQTPPWTFGQGTKVDFK

SEQ ID NO: 247 - DNA, 339 bp, Homo sapiens
  source 1..339: mol_type = genomic DNA
gacatcgtgatgacccagtcccctctgtctctgccagtgacacccggcgagcctgcctctatcagctgtcggagctcccagaacctgagacacaacaatggctacaactatctgaattggtacctgcagaagccaggccagtctccccagctgctgatctatctgggcagcatcagggcctccggcgtgcccgaccgcttctccggctctggcagcggcaccgacttcaccctgaagatcagccgggtggaggcagaggacgtgggcgtgtactattgcatgcaggccctgcagacccccccttggacattcggccagggcaccaaggtggacttcaag

SEQ ID NO: 248 - protein, 16 aa, Homo sapiens
RSSQNLRHNNGYNYLN

SEQ ID NO: 249 - protein, 7 aa, Homo sapiens
LGSIRAS

SEQ ID NO: 250 - protein, 10 aa, Homo sapiens
MQALQTPPWT

SEQ ID NO: 251 - protein, 118 aa, Homo sapiens
QVQLVQSGAEVKKPGASVKVSCKASGYTFTSHYMHWVRQAPGQGLEWMGIISPTGDFTNYAQKFQGRVTLTRDTSTSTDYMEVTSLRSEDTAVYYCARDCSAWAPDYWGQGTLVTVSS

SEQ ID NO: 252 - DNA, 354 bp, Homo sapiens
  source 1..354: mol_type = genomic DNA
caggtgcagctggtgcagtccggcgcagaggtgaagaagccaggagccagcgtgaaggtgtcctgtaaggcctctggctacaccttcacatctcactatatgcactgggtgcggcaggcaccaggacagggcctggagtggatgggcatcatcagccctacaggcgacttcaccaactacgcccagaagtttcagggccgggtgaccctgacaagagacacctctacaagcaccgattatatggaggtgacatccctgaggtctgaggataccgccgtgtactattgcgcaagggactgttccgcctgggcccccgattactggggacagggcacactggtgaccgtgagctcc

SEQ ID NO: 253 - protein, 7 aa, Homo sapiens
GYTFTSH

SEQ ID NO: 254 - protein, 6 aa, Homo sapiens
SPTGDF

SEQ ID NO: 255 - protein, 9 aa, Homo sapiens
DCSAWAPDY

SEQ ID NO: 256 - protein, 108 aa, Homo sapiens
QSALTRPPSVSRCPGQSITISCSGTSSDVGHDNHVSWYQQHPGRAPKLMVYEVRNRPSGVSDRFSGSKSGNTASLTISGLQAEDEATYYCCSYTTTHRYIFGGGTKLT

SEQ ID NO: 257 - DNA, 324 bp, Homo sapiens
  source 1..324: mol_type = genomic DNA
cagtctgccctgacaaggcccccttctgtgagccgctgccctggacagagcatcacaatctcctgttctggcaccagctccgacgtgggccacgataaccacgtgtcctggtaccagcagcacccaggaagggcacccaagctgatggtgtatgaggtgcggaacagaccaagcggcgtgtccgacaggttcagcggctccaagtctggcaatacagcctctctgaccatcagcggcctgcaggcagaggatgaggcaacctactattgctgttcttacaccacaacccaccggtatatctttggcggcggcacaaagctgacc

SEQ ID NO: 258 - protein, 14 aa, Homo sapiens
SGTSSDVGHDNHVS

SEQ ID NO: 259 - protein, 7 aa, Homo sapiens
EVRNRPS

SEQ ID NO: 260 - protein, 10 aa, Homo sapiens
CSYTTTHRYI

SEQ ID NO: 261 - protein, 123 aa, Homo sapiens
QVQLLGSGPGLVKPSETLSLTCTVSGASISSPGYYWGFIRQSPGKGLEWIGSMVSGGTTYYNPSLKSRVTISMDMSNNQFSLRLNSVTAADTALYYCARGSRQLVRRATIDYWGQGALFTVSP

SEQ ID NO: 262 - DNA, 369 bp, Homo sapiens
  source 1..369: mol_type = genomic DNA
caggtgcagctgctgggcagcggcccaggcctggtgaagccttctgagacactgagcctgacctgtacagtgtctggcgccagcatcagctccccaggctactattggggcttcatcaggcagagcccaggcaagggcctggagtggatcggctccatggtgtctggcggcaccacatactataaccctagcctgaagtcccgggtgacaatctccatggacatgtctaacaatcagttcagcctgaggctgaattccgtgaccgccgccgatacagccctgtactattgcgcaaggggctcccgccagctggtgcggagagcaaccatcgactactggggacagggcgccctgtttacagtgtctccc

SEQ ID NO: 263 - protein, 9 aa, Homo sapiens
GASISSPGY

SEQ ID NO: 264 - protein, 5 aa, Homo sapiens
VSGGT

SEQ ID NO: 265 - protein, 13 aa, Homo sapiens
GSRQLVRRATIDY

SEQ ID NO: 266 - protein, 108 aa, Homo sapiens
QSVLTGPPSVSAGPGQQVFISCSGNSSNIGNNYVSWYQQLPGTAPKLLIYDSNKRPSGIPDRFSGSKSGTSATLGITGLQTGDEADYYCGTWDSSLSAGVFGGGTKLT

SEQ ID NO: 267 - DNA, 324 bp, Homo sapiens
  source 1..324: mol_type = genomic DNA
cagtctgtgctgaccggaccaccttccgtgtctgccggaccaggacagcaggtgttcatcagctgttccggcaacagctccaatatcggcaacaattacgtgtcttggtatcagcagctgccaggcacagcccccaagctgctgatctacgactctaacaagcggcctagcggcatcccagatagattctctggcagcaagtccggcaccagcgccacactgggcatcaccggcctgcagacaggcgacgaggcagattactattgcggaacctgggactctagcctgtccgccggcgtgtttggaggaggaaccaagctgaca

SEQ ID NO: 268 - protein, 13 aa, Homo sapiens
SGNSSNIGNNYVS

SEQ ID NO: 269 - protein, 7 aa, Homo sapiens
DSNKRPS

SEQ ID NO: 270 - protein, 11 aa, Homo sapiens
GTWDSSLSAGV

SEQ ID NO: 271 - protein, 124 aa, Homo sapiens
EVQLVESGGGLVKPGESLRLSCAASGFTFSSYSMSWVRQAPGKGLEWVSCITSSGHTYYADSVKGRFAISRDNGKNSLYLQMNNLRAEDTAVYFCAKELGAHSGLFYNGVFDYWGQGNPVTVSS

SEQ ID NO: 272 - DNA, 372 bp, Homo sapiens
  source 1..372: mol_type = genomic DNA
gaggtgcagctggtggagtccggcggaggcctggtgaagccaggcgagtctctgaggctgagctgtgccgcctccggcttcacctttagctcctacagcatgtcctgggtgcgccaggcacctggcaagggcctggagtgggtgtcctgcatcacctctagcggccacacatactatgccgactctgtgaagggccggttcgccatcagccgggataacggcaagaatagcctgtacctgcagatgaacaatctgcgggccgaggacaccgccgtgtatttttgtgcaaaggagctgggagcacactctggcctgttctacaacggcgtgtttgattattggggccagggcaatcccgtgacagtgtcctct

SEQ ID NO: 273 - protein, 7 aa, Homo sapiens
GFTFSSY

SEQ ID NO: 274 - protein, 5 aa, Homo sapiens
TSSGH

SEQ ID NO: 275 - protein, 16 aa, Homo sapiens
ELGAHSGLFYNGVFDY

SEQ ID NO: 276 - protein, 108 aa, Homo sapiens
QSALTQPASVSGSPGQSITISCTGTSSDVGGYNYVSWYQQHPGKAPKLMIYEVSNRPSGVSNRFSGSKGNTASLTISGLRGEDEADYYCSSYTSSSTLVVFGGGTKLT

SEQ ID NO: 277 - DNA, 324 bp, Homo sapiens
  source 1..324: mol_type = genomic DNA
cagtccgccctgacccagccagcctccgtgtctggcagccccggccagtctatcacaatcagctgtaccggcacaagctccgacgtgggcggctacaactacgtgagctggtaccagcagcacccaggcaaggcacctaagctgatgatctatgaggtgtccaacaggccaagcggcgtgtccaatagattctccggctctaagggcaataccgcctccctgacaatctctggcctgaggggagaggacgaggcagattactattgctctagctacacctcctctagcacactggtggtgtttggcggcggcaccaagctgaca

SEQ ID NO: 278 - protein, 14 aa, Homo sapiens
TGTSSDVGGYNYVS

SEQ ID NO: 279 - protein, 7 aa, Homo sapiens
EVSNRPS

SEQ ID NO: 280 - protein, 11 aa, Homo sapiens
SSYTSSSTLVV

SEQ ID NO: 281 - protein, 124 aa, Homo sapiens; identical to SEQ ID NO: 221

SEQ ID NO: 282 - DNA, 372 bp, Homo sapiens; identical to SEQ ID NO: 222
  source 1..372: mol_type = genomic DNA

SEQ ID NO: 283 - protein, 7 aa, Homo sapiens
GGPFSGY

SEQ ID NO: 284 - protein, 5 aa, Homo sapiens
NHSGN

SEQ ID NO: 285 - protein, 16 aa, Homo sapiens
GQQLLRNYYYYSGMDV

SEQ ID NO: 286 - protein, 108 aa, Homo sapiens
QSALTQPASVSGSPGQSITISCTETSRDVGDYNYVSWYQQHPGPAPKLIMYEVHKRPSGISNRFSGSKSGTTASLTISGLQADDEGDYYCSSYTDKNTYVFGSGTQVT

SEQ ID NO: 287 - DNA, 324 bp, Homo sapiens
  source 1..324: mol_type = genomic DNA
cagtctgccctgacccagccagcctctgtgagcggctcccctggccagtccatcacaatctcttgtaccgagacatctcgggacgtgggcgattacaactatgtgagctggtaccagcagcacccaggacctgcaccaaagctgatcatgtatgaggtgcacaagcggccctctggcatcagcaatagattctctggcagcaagtccggcaccacagccagcctgaccatctccggcctgcaggcagacgatgagggcgactactattgcagctcctacaccgataagaacacatacgtgttcggcagcggcacccaggtgaca

SEQ ID NO: 288 - protein, 14 aa, Homo sapiens
TETSRDVGDYNYVS

SEQ ID NO: 289 - protein, 7 aa, Homo sapiens
EVHKRPS

SEQ ID NO: 290 - protein, 10 aa, Homo sapiens
SSYTDKNTYV

SEQ ID NO: 291 - protein, 447 aa, Homo sapiens
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIDWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGRYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGSGLNDIFEAQKIEWHEHHHHHH

SEQ ID NO: 292 - DNA, 1341 bp, Homo sapiens
  source 1..1341: mol_type = genomic DNA
gaagctgccctgctcgtgtgccagtacaccatccagagcctgatccacctgaccggcgaggaccccggcttcttcaacgtggaaatccccgagttccccttctaccctacctgcaacgtgtgcaccgccgacgtgaacgtgaccatcaacttcgacgtgggcggcaagaagcaccagctggacctggatttcggccagctgacccctcacaccaaggccgtgtatcagcccagaggcgcctttggcggcagcgagaacgccaccaatctgtttctgctggaactcctaggcgccggcgagctggccctgaccatgagaagcaagaaactgcccatcaatgtgaccacaggcgaggaacagcaggtgtccctggaaagcgtggacgtgtactttcaagacgtgttcggcaccatgtggtgccaccacgccgagatgcagaaccccgtgtacctgatccccgagacagtgccctacatcgattgggacaactgcaacagcaccaacatcaccgccgtcgtgcgggcccagggactggatgtgacactgcctctgagcctgcctaccagcgcccaggacagcaacttcagcgtgaaaaccgagatgctgggcaacgagatcgacatcgagtgcatcatggaagatggcgagatcagccaggtgctgcccggcgacaacaagttcaacatcacatgcagcggctacgagagccacgtgccatctggcggcatcctgaccagcacaagcccagtggccacacccatccctggcacaggctacgcctacagcctgagactgacccccagacccgtgtccagattcctgggcaacaacagcatcctgtacgtgttctacagcggcaacggccccaaggcctctggcggccggtactgtatccagagcaacatcgtgttcagcgacgagatccccgccagccaggacatgcccaccaataccaccgacatcacgtacgtgggcgacaatgccacctacagcgtgccaatggtcacctccgaggacgccaacagccccaacgtgaccgtgacagccttttgggcctggcctaacaacaccgagacagacttcaagtgcaagtggaccctgacctccggcacccctagcggctgcgagaatatcagcggagccttcgccagcaaccggaccttcgatatcaccgtgtctggcctgggcaccgcccccaagaccctgatcatcaccagaaccgccacaaatgccaccaccacaacccacaaagtgatcttcagcaaggcccccggctctggcctgaacgacatttttgaggcccagaagattgagtggcatgaacatcaccaccaccaccat

SEQ ID NO: 293 - DNA, 5118 bp, Homo sapiens
  source 1..5118: mol_type = genomic DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtacacgttgctgccgcgcgcgccaccagacataatagctgacagactaacagactgttcctttccatgggtcttttctgcagtcaccgtcgtcgacacgtgtgatcagatatcgcggccgctctagaccaccatgggatggtcatgtatcatcctttttctagtagcaactgcaaccggtgtacattcccagtctgccctgactcagcctccctctgtgtctgggtctcctggacagtcggtcaccatctcctgcactggaaccagcagtgacgttgatggttataactatgtctcctggtaccaacaacatccaggcaaagcccccaaactcatgatttatggtgtcagcaatcggccctcaggggtctctgatcgcttctctggctccaagtctggcaacacggcctccctgaccatctctgggctccaggctgaggacgaggctgattattactgttgttcatctacaaccagttacacttacatcttcggaactgggaccaaggtcacagtactaggtcagcccaaggctgccccctcggtcactctcttcccgccctcctctgaggagcttcaagccaacaaggccacactagtgtgtctgatcagtgacttctacccgggagccgtggaagtggcctggaaggcagatggcagcgctgtcaacgcgggagtggagaccaccaaaccctccaaacagagcaacaacaagtacgcggccagcagctacctgagcctgacgtccgaccagtggaagtcccacaagagctacagctgccaggtcacgcacgaagggagcaccgtggagaagacagtggcccctgcagaatgttcatagggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacatggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc